(19) United States
(12) Patent Application Publication
(10) Pub. No.: US 2025/0288693 A1
(43) Pub. Date: Sep. 18, 2025

(54) INSULIN AND GLUCOKINASE GENE THERAPY COMPOSITIONS AND ITS USE FOR TREATING DIABETES

(71) Applicant: Kriya Therapeutics, Inc., Morrisville, NC (US)

(72) Inventors: Michele STONE, Apex, NC (US); Melissa RHODES, Raleigh, NC (US); Chari SMITH, Durham, NC (US); Wallace HARRINGTON, Cary, NC (US)

(21) Appl. No.: 18/861,111

(22) PCT Filed: Apr. 28, 2023
(86) PCT No.: PCT/US2023/066350

§ 371 Date: Oct. 28, 2024

Related U.S. Application Data
(60) Provisional application No. 63/336,635, filed on Apr. 29, 2022.

(51) Int. Cl. A61K 48/00; A61P 3/10; C07K 14/62; C12N 9/12; C12N 15/86

(52) U.S. Cl. CPC A61K 48/0058; A61P 3/10; C07K 14/62; C12N 9/1205; C12N 15/86; C12Y 207/01002; C12N 2750/14143

(57) ABSTRACT

The present disclosure relates to a combination therapy comprising a first AAV vector genome comprising an insulin expression cassette; and a second AAV vector genome comprising a glucokinase expression cassette; wherein the first AAV vector genome and the second AAV vector genome are in a ratio selected from the group consisting of 1:0.25-0.75, 1:1.75-2.25, and 1:3.75-4.25, and methods for using the same for treating diabetes.

### CROSS-REFERENCE TO RELATED APPLICATIONS

This application claims the benefit of U.S. Provisional Application No. 63/336,635, filed Apr. 29, 2022, which is incorporated herein by reference in its entirety.

### REFERENCE TO SEQUENCE LISTING SUBMITTED ELECTRONICALLY

The content of the electronically submitted sequence listing in ASCII text file (4525_085PC01_SequenceListing_ST26; Size: 297,916 bytes; and Date of Creation: Apr. 18, 2023) filed with the application is incorporated herein by reference in its entirety.

### BACKGROUND

The two main forms of diabetes mellitus are type 1 (T1DM) and type 2 (T2DM) (Diabetes care, 1997, 20-1183-1197).

T1DM is characterized by a severe lack of insulin production due to specific destruction of the pancreatic B-cells. B-cell loss in T1DM is the result of an autoimmune mediated process, in which a chronic inflammation called insulitis causes β-cell destruction (Eizirik D. L. et al, 2001, Diabetologia, 44:2115-2133 and Mathis D et al, 2001, Nature, 414:792-798). T1DM is one of the most common endocrine and metabolic conditions of childhood, and incidence is rapidly increasing, especially among young children. T1DM is diagnosed when the autoimmune-mediated β-cell destruction is almost complete causing patients to need insulin-replacement therapy to survive. T1DM in an adult may present itself similarly to T2DM, with a slow deterioration in metabolic control, and subsequent progression to insulin dependency. This form is called latent autoimmune diabetes mellitus in adults (LADA) (Diabetes Atlas 4th edition, 2009, International Diabetes Federation).

T2DM is the most common form of diabetes mellitus and has been attributed to an interaction between genetic, environmental, and behavioral risk factors. T2DM is characterized by insulin insensitivity, declining insulin production, and eventual pancreatic β-cell failure (Olokoba, A. et al, 2012, Oman Med. J. 27(4):269-273).

Lifelong insulin treatment is often the therapy of choice for both T1DM and T2DM. While lifelong treatment with exogenous insulin has been largely successful in managing diabetes, diabetic complications can still occur due to difficulties with maintaining tight glycemic control. States of prolonged hyperglycemia can lead to severe microvascular or macrovascular complications, most commonly presenting as retinopathies, neuropathies, nephropathies, cerebrovascular accidents, or myocardial infarctions. These devastating complications can be prevented with improvements in glycemic control. Of note, brittle diabetes, which is a particularly labile form, can be very difficult to manage even with lifelong exogenous insulin.

Additionally, in many underdeveloped countries, access to self-care tools and to insulin can be limited, which may lead to severe handicap and early death in diabetic children (Diabetes Atlas 4th edition, 2009, International Diabetes Federation, Beran D. et al 2006, Lancet, 368:1689-1695, and Gale E. A., et al, 2006, Lancet, 368:1626-1628). The most common cause of death in a child with diabetes, from a global perspective, is lack of access to insulin. Thus, the availability of a one-time gene therapy approach could have a profound effect in situations where access to insulin is limited (Greenwood H. L. et al, 2006, PLOS Med 3.e381).

The reduction of hyperglycemia and maintenance of normoglycemia is a goal of any therapeutic approach to T1DM and T2DM. The current therapy for all T1DM and a large subset of T2DM tic patients is based on regular subcutaneous injections of both of short-acting and long-acting insulin preparations.

### FIELD OF DISCLOSURE

The present disclosure pertains to the medical field, including gene therapy compositions comprising modified nucleic acids encoding insulin and/or glucokinase for use in treatment of Diabetes.

### BRIEF SUMMARY

Certain aspects Certain aspects of the disclosure are directed to combination therapy comprising: (a) a first AAV vector genome comprising an insulin expression cassette comprising a first promoter operably linked to a polynucleotide encoding a human insulin (hIns) protein, wherein the insulin expression cassette is flanked by inverted terminal repeats (ITRs); and (b) a second AAV vector genome comprising a glucokinase expression cassette comprising a second promoter operably linked to a polynucleotide encoding a human glucokinase (hGck) protein, wherein the glucokinase expression cassette is flanked by inverted terminal repeats (ITRs); wherein the first AAV vector genome and the second AAV vector genome are in a vector ratio selected from the group consisting of 1:0.25-0.75, 1:1.75-2.25, or 1:3.75-4.25. In some aspects, the vector ratio is selected from 1:0.4-0.6, 1:1.9-2.1, or 1:3.9-4.1. In some aspects, the ratio is about 1:0.25-0.75, 1:0.3-0.75, 1:0.35-0.75, 1:0.40-0.75, 1:0.45-0.75, 1:0.5-0.75, 1:0.3-0.7, 1:0.3-0.65, 1:0.3-0.6, 1:0.3-0.55, 1:0.3-0.5, 1:0.35-0.7, 1:0.35-0.65, 1:0.35-0.6, 1:0.35-0.55, 1:0.35-0.5, 1:0.4-0.7, 1:0.4-0.65, 1:0.4-0.6, 1:0.4-0.55, 1:0.4-0.5, 1:0.45-0.7, 1:0.45-0.65, 1:0.45-0.6, 1:0.45-0.55, or 1:0.45-0.5. In some aspects, the vector ratio is about 1:0.5, about 1:2, and about 1:4. In some aspects, the vector ratio is 1:0.4-0.6. In some aspects, the vector ratio is 1:0.5. In some aspects the first and second promoter are the same or substantially the same promoter; or in some aspects the first and second promoters are different promoters that drive substantially equivalent levels of transgene expression.

Certain aspects of the disclosure are directed to method of treating or ameliorating the symptoms associated with diabetes in a subject in need thereof, comprising administering a combination therapy to the subject comprising: (a) a first AAV vector genome comprising an insulin expression cassette comprising a first promoter operably linked to a polynucleotide encoding a human insulin (hIns) protein, wherein the insulin expression cassette is flanked by inverted terminal repeats (ITRs); and (b) a second AAV vector genome comprising a glucokinase expression cassette comprising a second promoter operably linked to a polynucleotide encoding a human glucokinase (hGck) protein, wherein the glucokinase expression cassette is flanked by inverted terminal repeats (ITRs); wherein the first AAV vector genome and the second AAV vector genome are administered at a vector ratio selected from the group consisting of about 1:0.5, about 1:2, or about 1:4. In some aspects, the vector ratio is 1:0.5.

In some aspects, the polynucleotide encoding the hIns protein comprises an open reading frame (ORF) comprising: a nucleotide sequence at least 85%, 90%, 95%, 99%, or 100% identical to any one of: (i) nucleic acids 73-330 of any of SEQ ID NOs: 43-57, 110-116, 150-151, 154-155 or 157-159, nucleic acids 88-345 of any of SEQ ID NOs: 117-122, 152 or 156, or nucleic acids 79-336 of SEQ ID NO: 153; or (ii) SEQ ID NO: 43-57, SEQ ID NO: 110-122, or SEQ ID NO: 150-159; and/or (b) the polynucleotide encoding the human glucokinase hGck protein, comprises an ORF comprising (i) a nucleotide sequence at least 85%, 90%, 95%, 99%, or 100% identical to a sequence selected from any of (a) nucleic acids 1-1398 of any of SEQ ID NO: 61-80 or 162; or (ii) SEQ ID NO: 61-80 and 162.

In some aspects, the hIns protein comprises the amino acid sequence of any of amino acids 25-110 of SEQ ID NO: 41, amino acids 25-110 of SEQ ID NO: 144, amino acids 25-110 of SEQ ID NO: 145, SEQ ID NO: 41, SEQ ID NO: 144, or SEQ ID NO: 145.

In some aspects, the hIns protein comprises a signal peptide. In some aspects, the signal peptide is a wild-type preproinsulin signal sequence, an IL-6 signal sequence, a fibronectin signal sequence, or a non-wild-type preproinsulin signal sequence. In some aspects, the signal peptide comprises amino acids 25-110 of SEQ ID NO: 41, amino acids 25-110 of SEQ ID NO: 144, or amino acids 25-110 of SEQ ID NO: 145.

In some aspects, the signal peptide is a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid position in wild-type proinsulin.

In some aspects, the polynucleotide encoding the hIns protein further comprises a 5′ UTR comprising a nucleotide sequence at least 85%, 90%, 95%, 99%, or 100% identical to nucleic acids 5-329 of SEQ ID NO: 42.

In some aspects, the polynucleotide encoding the hIns protein further comprises a 5′ UTR comprising a nucleotide sequence at least 85%, 90%, 95%, 99%, or 100% identical to SEQ ID NO: 42, SEQ ID NO: 83, SEQ ID NO: 146, or SEQ ID NO: 148.

In some aspects, the polynucleotide encoding the hIns protein further comprises a 3′ UTR comprising a nucleotide sequence at least 85%, 90%, 95%, 99%, or 100% identical to SEQ ID NO: 60, SEQ ID NO: 97, SEQ ID NO: 98, SEQ ID NO: 99, SEQ ID NO: 100, or SEQ ID NO: 101, or SEQ ID NO: 149.

In some aspects, the encoded hGck protein comprises the amino acid sequence of SEQ ID NO: 82.

In some aspects, the polynucleotide encoding the hGck protein further comprises a 5′ UTR comprising a nucleotide sequence at least 85%, 90%, 95%, 99%, or 100% identical to nucleic acids 5-329 of SEQ ID NO: 42.

In some aspects, the polynucleotide encoding the hGck protein further comprises a 5′ UTR comprising a nucleotide sequence at least 85%, 90%, 95%, 99%, or 100% identical to SEQ ID NO: 42, SEQ ID NO: 83, SEQ ID NO: 146, or SEQ ID NO: 148.

In some aspects, the polynucleotide encoding the hGck protein further comprises a 3′ UTR comprising a nucleotide sequence at least 85%, 90%, 95%, 99%, or 100% identical to SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149.

In some aspects, the promoter is a eukaryotic promoter. In some aspects, the promoter is a CMV promoter. In some aspects, the promoter is used together with an intronic sequence. In some aspects, the CMV promoter is a mini CMV promoter.

In some aspects, the insulin expression cassette comprises a polyadenylation (polyA) element. In some aspects, the glucokinase expression cassette comprises a polyadenylation (polyA) element.

In some aspects, the first recombinant AAV (rAAV) particle comprises the first AAV vector genome comprising the insulin expression cassette disclosed herein.

In some aspects, the second recombinant AAV (rAAV) particle comprises the second AAV vector genome comprising the glucokinase expression cassette disclosed herein.

In some aspects, the AAV serotype is selected from the group consisting of AAV1, AAV2, AAV3, AAV4, AAV5, AAV6, AAV7, AAV8, AAVrh8, AAVrh9, AAV9, AAVrh10, AAV10, AAV11, and AAV12.

Certain aspects of the disclosure are directed to a method of treating or ameliorating the symptoms associated with diabetes in a subject in need thereof, comprising delivering to the subject a therapeutically effective amount of the combination therapy disclosed herein thereby treating diabetes in the subject.

Certain aspects of the disclosure are directed to a method of producing human Ins protein and human Gck protein in a subject in need thereof and/or treating or ameliorating the symptoms associate with diabetes in a subject in need thereof comprising administering to the subject the combination disclosed herein, thereby producing human Ins protein and human Gck protein and/or treating diabetes in the subject.

In some aspects, the diabetes is diabetes mellitus type 1 (T1DM) or diabetes mellitus type 2 (T2DM). In some aspects, the diabetes is diabetes mellitus type 1 (T1DM). In some aspects, the diabetes is diabetes mellitus type 2 (T2DM).

In some aspects, the first rAAV particle and the second rAAV particle are administered simultaneously or sequentially.

In some aspects, the delivery and/or administration is intramuscular.

In some aspects, the method results in (i) glycated blood hemoglobin (HbA1c) levels are reduced and/or regulated in the subject; (ii) circulating ketones are reduced in the subject, (iii) triglycerides are reduced in the subject, or (iv) any combination thereof.

Certain aspects of the disclosure are directed to a composition comprising: (a) a first recombinant AAV (rAAV) particle comprising a first AAV vector genome comprising an insulin expression cassette comprising a first promoter operably linked to a polynucleotide encoding a human insulin (hIns) protein, wherein the insulin expression cassette is flanked by inverted terminal repeats (ITRs); and (b) a second recombinant AAV (rAAV) particle comprising a second AAV vector genome comprising a glucokinase expression cassette comprising a second promoter operably linked to a polynucleotide encoding a human glucokinase (hGck) protein, wherein the glucokinase expression cassette is flanked by inverted terminal repeats (ITRs);
wherein the first rAAV particle and the second rAAV particle are in a ratio selected from the group consisting of 1:0.25-0.75, 1:1.75-2.25, and 1:3.75-4.25.

In some aspects, the ratio is selected from the group consisting of 1:0.4-0.6, 1:1.9-2.1, and 1:3.9-4.1. In some aspects, the ratio is about 1:0.25-0.75, 1:0.25-0.7, 1:0.25-0.65, 1:0.25-0.60, 1:0.25-0.55, and 1:0.25-0.5. In some aspects, the ratio is about 1:0.25-0.75, 1:0.3-0.75, 1:0.35-0.75, 1:0.40-0.75, 1:0.45-0.75, 1:0.5-0.75, 1:0.3-0.7, 1:0.3-0.65, 1:0.3-0.6, 1:0.3-0.55, 1:0.3-0.5, 1:0.35-0.7, 1:0.35-0.65, 1:0.35-0.6, 1:0.35-0.55, 1:0.35-0.5, 1:0.4-0.7, 1:0.4-0.65, 1:0.4-0.6, 1:0.4-0.55, 1:0.4-0.5, 1:0.45-0.7, 1:0.45-0.65, 1:0.45-0.6, 1:0.45-0.55, or 1:0.45-0.5. In some aspects, the ratio is selected from the group consisting of about 1:0.5, about 1:2, and about 1:4. In some aspects, the vector ratio is 1:0.4-0.6. In some aspects, the ratio is about 1:0.5.

### BRIEF DESCRIPTION OF FIGURES

FIGS. 1A-1B are graphs showing the levels of blood glucose in non-diabetic control mice, streptozotocin (STZ) treated control mice, or STZ mice administered AAV1-hINS/AAV1-hGCK. FIG. 1A shows the blood glucose levels of non-diabetic control mice, STZ treated control mice, or STZ mice administered low-dose (9.0E10 vg AAV1-hINS and 9.0E10 vg AAV1-hGCK for a total dose of 1.8E11 vg), mid-dose (1.2E11 vg AAV1-hIns and 1.2E11 vg AAV1-hGck for a total dose of 2.4E11 vg), or high-dose (2.4E11 vg AAV1-hIns and 2.4E11 vg AAV1-hGck for total dose of 4.8E11vg) of AAV1-hINS/AAV1-hGCK at a 1:1 ratio. FIG. 1B shows the blood glucose levels of non-diabetic control mice, STZ treated control mice, or STZ mice administered a mid-dose of AAV1-hINS/AAV1-hGCK at a 1:1 ratio or AAV1-hINS/AAV1-hGCK at a 1:0.5 (1.2E11 vg AAV1-hIns and 6.0E10 vg AAV1-hGck for a total dose of 1.8E11 vg), 1:2 (1.2E11 vg AAV1-hIns and 2.4E11 vg AAV1-hGck for a total dose of 3.6E11 vg), or 1:4 (1.2E11 vg AAV1-hIns and 4.8E11 vg AAV1-hGck for a total dose of 6.0E11 vg) ratio based on the mid-dose of hINS.

FIG. 2 is a graph showing fasted blood glucose levels (mg/dL) in non-diabetic control mice; STZ treated control mice; or mice administered a low-dose (9.0E10 vg AAV1-hINS and 9.0E10 vg AAV1-hGCK for a total dose of 1.8E11 vg), mid-dose (1.2E11 vg AAV1-hIns and 1.2E11 vg AAV1-hGck for a total dose of 2.4E11 vg), or high-dose (2.4E11 vg AAV1-hIns and 2.4E11 vg AAV1-hGck for total dose of 4.8E11 vg) of AAV1-hINS/AAV1-hGCK at a 1:1 ratio or AAV1-hINS/AAV1-hGCK at a 1:0.5 (1.2E11 vg AAV1-hIns and 6.0E10 vg AAV1-hGck for a total dose of 1.8E11 vg), 1:2 (1.2E11 vg AAV1-hIns and 2.4E11 vg AAV1-hGck for a total dose of 3.6E11 vg), or 1:4 (1.2E11 vg AAV1-hIns and 4.8E11 vg AAV1-hGck for a total dose of 6.0E11 vg) ratio based on the mid-dose of hINS (6E10 vg). Glucose levels were collected at day 29 and day 56 after AAV injection.

FIGS. 3A-3D are graphs showing the results of an oral glucose tolerance test (OGTT) in non-diabetic control mice, STZ treated control mice, or mice administered AAV1-hINS/AAV1-hGCK. FIG. 3A is a graph showing the blood glucose levels of non-diabetic control mice, STZ treated control mice, or mice administered low-dose (9.0E10 vg AAV1-hINS and 9.0E10 vg AAV1-hGCK for a total dose of 1.8E11 vg), mid-dose (1.2E11 vg AAV1-hIns and 1.2E11 vg AAV1-hGck for a total dose of 2.4E11 vg), or high-dose (2.4E11 vg AAV1-hIns and 2.4E11 vg AAV1-hGck for total dose of 4.8E11vg) of AAV1-hINS/AAV1-hGCK at a 1:1 ratio over 120 minutes. FIG. 3B is a graph showing the blood glucose levels of non-diabetic control mice, STZ treated control mice, or mice administered a mid-dose of AAV1-hINS/AAV1-hGCK at a 1:1 ratio or AAV1-hINS/AAV1-hGCK at a 1:0.5 (1.2E11 vg AAV1-hIns and 6.0E10 vg AAV1-hGck for a total dose of 1.8E11 vg), 1:2 (1.2E11 vg AAV1-hIns and 2.4E11 vg AAV1-hGck for a total dose of 3.6E11 vg), or 1:4 (1.2E11 vg AAV1-hIns and 4.8E11 vg AAV1-hGck for a total dose of 6.0E11 vg) ratio based on the mid-dose of hINS over 120 minutes. FIG. 3C are graphs showing the area under the curve (AUC) of non-diabetic control mice, STZ treated control mice, or mice administered low-dose (9.0E10 vg AAV1-hINS and 9.0E10 vg AAV1-hGCK for a total dose of 1.8E11 vg), mid-dose (1.2E11 vg AAV1-hIns and 1.2E11 vg AAV1-hGck for a total dose of 2.4E11 vg), or high-dose (2.4E11 vg AAV1-hIns and 2.4E11 vg AAV1-hGck for total dose of 4.8E11 vg) of AAV1-hINS/AAV1-hGCK at a 1:1 ratio or AAV1-hINS/AAV1-hGCK at a 1:0.5 (1.2E11 vg AAV1-hIns and 6.0E10 vg AAV1-hGck for a total dose of 1.8E11 vg), 1:2 (1.2E11 vg AAV1-hIns and 2.4E11 vg AAV1-hGck for a total dose of 3.6E11 vg), or 1:4 (1.2E11 vg AAV1-hIns and 4.8E11 vg AAV1-hGck for a total dose of 6.0E11 vg) ratio based on the mid-dose of hINS. FIG. 3D shows the corresponding AUC leverage plot.

FIG. 4 is a graph showing the HbA1c in non-diabetic control mice, STZ treated control mice, or mice administered low-dose (9.0E10 vg AAV1-hINS and 9.0E10 vg AAV1-hGCK for a total dose of 1.8E11 vg), mid-dose (1.2E11 vg AAV1-hIns and 1.2E11 vg AAV1-hGck for a total dose of 2.4E11 vg), or high-dose (2.4E11 vg AAV1-hIns and 2.4E11 vg AAV1-hGck for total dose of 4.8E11 vg) of AAV1-hINS/AAV1-hGCK at a 1:1 ratio or AAV1-hINS/AAV1-hGCK at a 1:0.5 (1.2E11 vg AAV1-hIns and 6.0E10 vg AAV1-hGck for a total dose of 1.8E11 vg), 1:2 (1.2E11 vg AAV1-hIns and 2.4E11 vg AAV1-hGck for a total dose of 3.6E11 vg), or 1:4 (1.2E11 vg AAV1-hIns and 4.8E11 vg AAV1-hGck for a total dose of 6.0E11 vg) ratio based on the mid-dose of hINS

FIG. 5 is a graph showing circulating insulin levels in non-diabetic control mice, STZ treated control mice, or mice administered low-dose (9.0E10 vg AAV1-hINS and 9.0E10 vg AAV1-hGCK for a total dose of 1.8E11 vg), mid-dose (1.2E11 vg AAV1-hIns and 1.2E11 vg AAV1-hGck for a total dose of 2.4E11 vg), or high-dose (2.4E11 vg AAV1-hIns and 2.4E11 vg AAV1-hGck for total dose of 4.8E11 vg) of AAV1-hINS/AAV1-hGCK at a 1:1 ratio or AAV1-hINS/AAV1-hGCK at a 1:0.5 (1.2E11 vg AAV1-hIns and 6.0E10 vg AAV1-hGck for a total dose of 1.8E11 vg), 1:2 (1.2E11 vg AAV1-hIns and 2.4E11 vg AAV1-hGck for a total dose of 3.6E11 vg), or 1:4 (1.2E11 vg AAV1-hIns and 4.8E11 vg AAV1-hGck for a total dose of 6.0E11 vg) ratio based on the mid-dose of hINS at day 29 and day 60 after AAV injection.

FIG. 6 is a graph showing serum electrolyte (Na, K, and Cl) levels in non-diabetic control mice, STZ treated control mice, or mice administered low-dose (9.0E10 vg AAV1-hINS and 9.0E10 vg AAV1-hGCK for a total dose of 1.8E11 vg), mid-dose (1.2E11 vg AAV1-hIns and 1.2E11 vg AAV1-hGck for a total dose of 2.4E11 vg), or high-dose (2.4E11 vg AAV1-hIns and 2.4E11 vg AAV1-hGck for total dose of 4.8E11 vg) of AAV1-hINS/AAV1-hGCK at a 1:1 ratio or AAV1-hINS/AAV1-hGCK at a 1:0.5 (1.2E11 vg AAV1-hIns and 6.0E10 vg AAV1-hGck for a total dose of 1.8E11 vg), 1:2 (1.2E11 vg AAV1-hIns and 2.4E11 vg AAV1-hGck for a total dose of 3.6E11 vg), or 1:4 (1.2E11 vg AAV1-hIns and 4.8E11 vg AAV1-hGck for a total dose of 6.0E11 vg) ratio based on the mid-dose of hINS.

FIG. 7 is a graph showing triglyceride levels in non-diabetic control mice, STZ treated control mice, or mice administered low-dose (9.0E10 vg AAV1-hINS and 9.0E10 vg AAV1-hGCK for a total dose of 1.8E11vg), mid-dose (1.2E11 vg AAV1-hIns and 1.2E11 vg AAV1-hGck for a total dose of 2.4E11 vg), or high-dose (2.4E11 vg AAV1-hIns and 2.4E11 vg AAV1-hGck for total dose of 4.8E11 vg) of AAV1-hINS/AAV1-hGCK at a 1:1 ratio or AAV1-hINS/AAV1-hGCK at a 1:0.5 (1.2E11 vg AAV1-hIns and 6.0E10 vg AAV1-hGck for a total dose of 1.8E11 vg), 1:2 (1.2E11 vg AAV1-hIns and 2.4E11 vg AAV1-hGck for a total dose of 3.6E11 vg), or 1:4 (1.2E11 vg AAV1-hIns and 4.8E11 vg AAV1-hGck for a total dose of 6.0E11 vg) ratio based on the mid-dose of hINS.

FIGS. 8A-8B are graphs showing the pre- and post-prandial blood glucose levels (mg/dl) of two non-human primates, NHP-1 (FIG. 8A) and NHP-2 (FIG. 8B), taken daily from 28 days prior to administration through 60 days after administration of AAV1-hIns+AAV1-hGck at a 1:0.5 ratio (1.9E+13 vg AAV1-hIns and 9.6E+12 vg AAV-hGck). The graphs also shows the amount of exogenous insulin administered (U/kg/day) daily to the two primates from 28 days prior to administration through 60 days after administration of AAV1-hIns+AAV1-hGck at a 1:0.5 ratio (1.9E+13 vg AAV1-hIns and 9.6E+12 vg AAV-hGck).

FIGS. 9A-9B are graphs showing the C-peptide (ng/ml) levels of two non-human primates, NHP-1 (FIG. 9A) and NHP-2 (FIG. 9B), taken weekly after administration of AAV1-hIns/AAV1-hGck at a 1:0.5 ratio (1.9E+13 vg AAV1-hIns and 9.6E+12 vg AAV-hGck).

FIGS. 10A-10B are graphs showing the change in glycated hemoglobin A1C of two non-human primates, NHP-1 (FIG. 10A) and NHP-2 (FIG. 10B), taken every two weeks from 42 days prior to and through 56 days after administration of AAV1-hIns/AAV1-hGck at a 1:0.5 ratio (1.9E+13 vg AAV1-hIns and 9.6E+12 vg AAV-hGck).

FIGS. 11A-11B are graphs showing the results of intravenous glucose tolerance tests (IVGTT) of two non-human primates, NHP-1 (FIG. 11A) and NHP-2 (FIG. 11B), prior to treatment with STZ (pre STZ), after treatment with STZ (post STZ), and 56 days after administration of AAV1-hIns/AAV1-hGck at a 1:0.5 ratio (1.9E+13 vg AAV1-hIns and 9.6E+12 vg AAV-hGck) (D+56).

### DETAILED DESCRIPTION OF THE DISCLOSURE

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless indicated otherwise.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with, 37 CFR § 1.822 and established usage.

“Polynucleotide” or “nucleic acid” as used herein means a sequence of nucleotides connected by phosphodiester linkages. Polynucleotides are presented herein in the direction from the 5′ to the 3′ direction. A polynucleotide of the present disclosure can be a deoxyribonucleic acid (DNA) molecule or ribonucleic acid (RNA) molecule. Nucleotide bases are indicated herein by a single letter code: adenine (A), guanine (G), thymine (T), cytosine (C), inosine (I) and uracil (U).

As used herein, the term “polypeptide” encompasses both peptides and proteins, unless indicated otherwise.

The term “coding sequence” or “sequence encoding” is used herein to mean a DNA or RNA region (the transcribed region) which “encodes” a particular protein, e.g., such as an insulin or a glucokinase. A coding sequence is transcribed (DNA) and translated (RNA) into a polypeptide, in vitro or in vivo, when placed under the control of an appropriate regulatory region, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotes or eukaryotes, genomic DNA from prokaryotes or eukaryotes, and synthetic DNA sequences. A transcription termination sequence can be located 3′ to the coding sequence.

In some aspects, an expression cassette can comprise several operably linked fragments, such as one or more of a promoter, a 5′-untranslated sequence, a leader sequence, an intron, a coding sequence and a 3′-untranslated sequence (e.g., comprising a polyadenylation site or a signal sequence). As used herein, “expression of a gene” refers to the process wherein a gene is transcribed into an RNA and/or translated into an active protein.

An open reading frame (ORF) as used herein is the part of a reading frame that has the ability to be translated. An ORF is a continuous stretch of codons that begins with a start codon and ends at a stop codon. In some aspects, an ORF sequence can be shown or referenced with or without the start codon sequence and/or the stop codon sequence.

A Kozak consensus sequence, Kozak consensus or Kozak sequence, is known as a sequence which occurs on eukaryotic mRNA and has the consensus (gcc) gccRccAUGG, where R is a purine (adenine or guanine) three bases upstream of the start codon (AUG), which is followed by another “G.” In some aspects, the polynucleotide comprises a nucleic acid sequence having at least 95%, at least 99% sequence identity, or more to the Kozak consensus sequence. In some aspects, the polynucleotide comprises a Kozak consensus sequence.

The term “sequence identity” is used herein to mean a relationship between two or more amino acid (polypeptide or protein) sequences or two or more nucleic acid (polynucleotide) sequences, as determined by comparing the sequences. In certain aspects, sequence identity is calculated based on the full length of two given SEQ ID NO or on part thereof. Part thereof can mean at least 10%, 20%, 30%, 40%, 50%, 60%, 70%, 80%, 90%, or 100% of both SEQ ID NO, or any other specified percentage. The term “identity” can also mean the degree of sequence relatedness between amino acid or nucleic acid sequences, as the case may be, as determined by the match between strings of such sequences.

In certain aspects, methods to determine identity are designed to give the largest match between the sequences tested. Methods to determine identity and similarity are codified in publicly available computer programs.

“Substantial homology” or “substantial similarity,” means, when referring to a nucleic acid or fragment thereof, indicates that, when optimally aligned with appropriate nucleotide insertions or deletions with another nucleic acid (or its complementary strand), there is nucleotide sequence identity in at least about 95% to 99% of the sequence.

As used herein, and unless otherwise indicated, the term “complementary,” when used to describe a first nucleic acid sequence in relation to a second nucleic acid sequence, refers to the ability of an oligonucleotide or polynucleotide comprising the first nucleic acid sequence to hybridize and form a duplex structure under certain conditions with an oligonucleotide or polynucleotide comprising the second nucleic acid sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can be used. The skilled person will be able to determine the set of conditions most appropriate for a test of complementarity of two sequences in accordance with the ultimate application of the hybridized nucleotides.

The term “promoter” is used herein to mean a nucleic acid sequence or fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions, or in some aspects an inducible promoter can be induced by an exogenous molecule (e.g., a chemical or drug) or other exogenous stimulus (e.g., light, or radiation). A “tissue specific” promoter is preferentially active in specific types of differentiated cells/tissues.

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene. The term “operably linked” means that a DNA sequence and a regulatory sequence(s) are connected in such a way as to permit gene expression when the appropriate molecules (e.g., transcriptional activator proteins) are bound to the regulatory sequence(s). The term “operably inserted” means that the DNA of interest introduced into the cell is positioned adjacent a DNA sequence which directs transcription and translation of the introduced DNA (i.e., facilitates the production of, e.g., a polypeptide encoded by a DNA of interest).

The term “transgene” is used herein to mean a gene or a nucleic acid molecule that is introduced into a cell. An example of a transgene is a nucleic acid encoding a therapeutic polypeptide (e.g., a gene encoding an insulin and/or a gene encoding a glucokinase). In some embodiments, the gene can be present but in some cases normally not expressed or expressed at an insufficient level in the cell. In this context, “insufficient” means that although said gene, e.g., insulin and/or glucokinase, is normally expressed in a cell, a condition and/or disease as disclosed herein (e.g., diabetes) could still be developed. In certain aspects, the transgene allows for the increased expression or over-expression of the gene, e.g., an insulin and/or a glucokinase. The transgene can comprise sequences that are native to the cell, comprise sequences that do not naturally occur in the cell, or it can comprise combinations of both. In certain aspects, the transgene can comprise modified sequences coding for an insulin, a glucokinase, both an insulin and a glucokinase, and/or additional protein(s) that can be operably linked to appropriate regulatory sequences for expression of the sequences coding for an insulin, a glucokinase, or both an insulin and a glucokinase in the cell. In some aspects, the transgene is not integrated into the host cell's genome.

The terms “modified genes”, “modified nucleic acids”, and the like are used interchangeably herein to mean the introduction of one or more modifications or changes relative to the in the natural sequence of the genes or nucleic acid sequence. Such modifications may or may not result in mutations to the encoded protein sequence. In some embodiments, the modified nucleic acid encodes a wild-type or mutant protein sequence or fragment thereof.

The term “derived from,” as used herein, refers to a component that is isolated from or made using a specified molecule or organism, or information (e.g., amino acid or nucleic acid sequence) from the specified molecule or organism. For example, a nucleic acid sequence (e.g., a modified human insulin gene) that is derived from a second nucleic acid sequence (e.g., a wild-type human insulin gene) can include a nucleotide sequence or portion thereof that is identical or substantially similar to the nucleotide sequence of the second nucleic acid sequence. In some aspects, mutants, analogs or derivatives can be derived from a wild-type sequence.

In the case of a polynucleotide, the derived species can be obtained by, for example, naturally occurring mutagenesis, artificial directed mutagenesis or artificial random mutagenesis. The mutagenesis used to derive polynucleotides can be intentionally directed or intentionally random, or a mixture of each.

As used herein, the term “delivery vector” or “vector” includes any genetic element, such as a plasmid, phage, transposon, cosmid, chromosome, artificial chromosome, virus, virion, etc., which is capable of replication when associated with the proper control elements and which can transfer gene or nucleic acid sequences between cells. Thus, the term includes cloning and expression vehicles, as well as viral vectors. In some aspects, useful vectors are contemplated to be those vectors in which the nucleic acid segment to be transcribed is positioned under the transcriptional control of a promoter. In some aspects, the delivery vector is selected from the group consisting of a viral vector, a plasmid, lipid, and a lysosome.

In some aspects, the biological vectors include viruses, particularly attenuated and/or replication-deficient viruses. In some embodiments, chemical vectors include lipid complexes and naked DNA constructs.

As used herein, the term “naked DNA” or “naked nucleic acid” and the like refers to a nucleic acid molecule that is not contained within a viral particle, bacterial cell, or other encapsulating means that facilitates delivery of nucleic acid into the cytoplasm of the target cell. Naked nucleic acid can be associated with means for facilitating delivery of the nucleic acid to the site of the target cell (e.g., to facilitate travel into the target cell of the nucleic acid through the alimentary canal, protect the nucleic acid from stomach acid, and/or serve to penetrate intestinal mucus) and/or to the surface of the target epithelial cell.

A “viral genome” or “vector genome” or “viral vector” refers to a sequence that comprises one or more polynucleotide regions encoding or comprising a molecule of interest, e.g., a protein, a peptide, and a polynucleotide or a plurality thereof. Viral vectors are used to deliver genetic materials into cells. Viral vectors can be modified for specific applications. In some aspects, the delivery vectors comprises a viral vector selected from the group consisting of an adeno-associated viral (AAV) vector, an adenoviral vector, a lentiviral vector, or a retroviral vector.

The term “adeno-associated virus vector” or “AAV vector” as used herein refers to any vector which comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV genome” and “AAV vector” can be used interchangeably.

As used herein, a “recombinant AAV particle” or “rAAV particle” is an AAV virus that comprises a capsid protein and an AAV vector having at least one payload region (e.g., an expression cassette including a polynucleotide encoding insulin and/or Gck) and at least one inverted terminal repeat (ITR) region. In some aspects, the terms “AAV vectors of the present disclosure” or “AAV vectors” refer to AAV vectors comprising a polynucleotide encoding an insulin, a Gck, or a combination thereof, e.g., encapsulated in an AAV capsid.

“Transduction” of a cell by a virus means that there is transfer of a nucleic acid from the virus particle to the cell. In some aspects, transduction refers to the delivery of a nucleic acid or nucleic acids encoding an insulin and/or a glucokinase into a recipient host cell by a viral vector. For example, transduction of a target cell by a rAAV vector of the disclosure leads to transfer of the rAAV genome (e.g., comprising a polynucleotide of the disclosure) contained in that vector into the transduced cell.

“Transfection” of a cell means that genetic material is introduced into a cell for the purpose of genetically modifying the cell. Transfection can be accomplished by a variety of means known in the art, e.g., transduction or electroporation.

“Vector” as used herein means a recombinant plasmid or virus that comprises a polynucleotide to be delivered into a host cell, either in vitro or in vivo.

The term “host cell” or “target cell” is used herein to mean the cell into which the polynucleotide delivery takes place, either in vitro or in vivo. AAV vectors are able to transduce both dividing and non-dividing cells.

“Recombinant” means distinct from that generally found in nature.

“Serotype” with respect to vector or virus capsid is defined by a distinct immunological profile based on the capsid protein sequences and capsid structure.

“AAV Cap” means AAV Cap proteins, VP1, VP2 and VP3 and analogs thereof.

“AAV Rep” means AAV Rep proteins and analogs thereof.

“Flanked,” with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., ITRs), indicates that one element is located 5′ to the sequence and the other is located 3′ to the sequence; however, there may be intervening sequences between.

As used herein, the terms “effective amount,” “therapeutically effective amount,” and a “sufficient amount” of, e.g., a gene therapy composition comprising a rAAV particle and/or a polynucleotide disclosed herein, refer to a quantity sufficient to, when administered to the subject, including a human, effect beneficial or desired results, including clinical results, and, as such, an “effective amount” or synonym thereto depends on the context in which it is being applied.

The amount of a given therapeutic agent or composition will correspond to such an amount will vary depending upon various factors, such as the given agent, the pharmaceutical formulation, the route of administration, the type of disease or disorder, the identity of the subject (e.g., age, sex, and/or weight) or host being treated, and the like.

The term “ratio” refers to the comparison of two or more numbers that indicates their quantitative relation to each other. In some aspects, a ratios can be used to compare two portions within a whole or total amount.

As used herein, the term “vector ratio” refers to the amount, in vector genomes (vg), of one AAV vector compared to the amount, in vector genomes, of another AAV vector. In some aspects, the vector ratio refers to the amount of AAV1-hINS vg to the amount of AAV1-hGCK vg. For example, equal amounts of AAV1-hINS vg and AAV1-hGCK vg can also be understood as a one to one vector ratio or a 1:1 vector ratio.

As used herein, the term “gene therapy” is the insertion of nucleic acid sequences (e.g., a nucleic acid comprising a promoter operably linked to a polynucleotide encoding a therapeutic molecule as defined herein) into an individual's cells and/or tissues to treat a disease or condition. Gene therapy also includes insertion of a transgene that is inhibitory in nature, i.e., that inhibit, decrease or reduce expression, activity or function of an endogenous gene or protein, such as an undesirable or aberrant (e.g., pathogenic) gene or protein. Such transgenes can be exogenous. An exogenous molecule or sequence is understood to be molecule or sequence not normally occurring in the cell, tissue and/or individual to be treated. Both acquired and congenital diseases can be amenable to gene therapy.

In some aspects, the disclosure provides polynucleotides encoding wild-type or mutant insulin and/or wild-type glucokinase or a functional fragment thereof. The disclosure also provides nucleic acid constructs that include as part of their sequence the polynucleotides encoding wild-type or mutant insulin and/or wild-type glucokinase or a functional fragment thereof. For example, the disclosure includes expression cassettes, plasmids and/or other vectors that include the polynucleotides along with other elements, such as regulatory elements. In some aspects, the disclosure provides a packaged gene delivery vehicle, such as a viral capsid, including the polynucleotides encoding wild-type or mutant insulin and/or wild-type glucokinase or a functional fragment thereof. The disclosure also includes methods of expressing wild-type or mutant insulin and/or wild-type glucokinase or a functional fragment thereof by delivering the polynucleotides into a cell along with elements required to promote expression in the cell. The disclosure also provides gene therapy methods in which the polynucleotides encoding wild-type or mutant insulin and/or wild-type glucokinase or a functional fragment thereof is/are administered to a subject, e.g., as a component of one or more vectors and/or packaged as a component of one or more viral gene delivery vehicles. Treatment can, for example, be effected to treat or reduce the symptoms of diabetes in a subject in need thereof. Each of these aspects of the disclosure is discussed in further detail herein.

### Combination Therapy

Certain aspects of the disclosure are directed to a combination gene therapy, e.g., a combination AAV gene therapy for delivery of a polynucleotide encoding an insulin (Ins) protein and a polynucleotide encoding a glucokinase (Gck) protein. In some aspects, the combination therapy includes separate administration of the Ins and Gck encoding polynucleotides. In some aspects, the combination therapy includes administration of the insulin and glucokinase encoding polynucleotides in a single formulation, e.g., two separate rAAV particles in the same pharmaceutical composition.

In some aspects, the present disclosure is directed to a combination therapy comprising a) a first AAV vector genome comprising an insulin expression cassette comprising a first promoter operably linked to a polynucleotide encoding a human insulin (hIns) protein, wherein the insulin expression cassette is flanked by inverted terminal repeats (ITRs), and b) a second AAV vector genome comprising a glucokinase expression cassette comprising a second promoter operably linked to a polynucleotide encoding a human glucokinase (hGck) protein, wherein the glucokinase expression cassette is flanked by ITRs, wherein the first AAV vector genome and the second AAV vector genome are in a vector ratio selected from the group consisting of 1:0.25-0.75, 1:1.75-2.25, and 1:3.75-1:4.25 (e.g., about 1:0.5, about 1:2, or about 1:4).

In some aspects, the vector ratio is about 1:0.25-0.75, 1:0.25-0.7, 1:0.25-0.65, 1:0.25-0.60, 1:0.25-0.55, or 1:0.25-0.5. In some aspects, the vector ratio is about 1:0.25-0.75, 1:0.3-0.75, 1:0.35-0.75, 1:0.40-0.75, 1:0.45-0.75, 1:0.5-0.75, 1:0.3-0.7, 1:0.3-0.65, 1:0.3-0.6, 1:0.3-0.55, 1:0.3-0.5, 1:0.35-0.7, 1:0.35-0.65, 1:0.35-0.6, 1:0.35-0.55, 1:0.35-0.5, 1:0.4-0.7, 1:0.4-0.65, 1:0.4-0.6, 1:0.4-0.55, 1:0.4-0.5, 1:0.45-0.7, 1:0.45-0.65, 1:0.45-0.6, 1:0.45-0.55, or 1:0.45-0.5. In some aspects, the vector ratio is about 1:0.25-0.75, 1:0.3-0.70, 1:0.35-0.65, 1:0.4-0.60, or 1:0.45-0.55.

In some aspects, the vector ratio is selected from the group consisting of 1:1.75-2.25, 1:1.8-2.25, 1:1.85-2.25, 1:1.9-2.25, 1:1.95-2.25, and 1:2.0-2.25. In some aspects, the vector ratio is selected from the group consisting of 1:1.75-2.25, 1:1.75-2.20, 1:1.75-2.15, 1:1.75-2.10, 1:1.75-2.05, and 1:1.75-20. In some aspects, the vector ratio is selected from the group consisting of 1:1.75-2:25, 1:1.80-2.20; 1:1.85-2.15, 1:1.90-2.10, and 1:1.95-2.05.

In some aspects, the vector ratio is selected from the group consisting of 1:3.75-1:4.25, 1:3.80-1:4.25, 1:3.85-1:4.25, 1:3.9-4.25, 1:3.95-1:4.25, and 1:4-1:4.25. In some aspects, the vector ratio is selected from the group consisting of 1:3.75-4.25, 1:3.75-4.20, 1:3.75-4.15, 1:3.75-4.10, 1:3.75-4.05, and 1:3.75-4. In some aspects, the vector ratio is selected from the group consisting of 1:3.75-4.25, 1:3.8-4.2, 1:3.85-4.15, 1:3.9-4.1, and 1:3.95-4.05.

In some aspects, the vector ratio is about 1:0.4-0.6.

In some aspects, the vector ratio is about 1:0.5.

In some aspects, the polynucleotide encoding a hIns is a modified polynucleotide (e.g., preproinsulin or proinsulin, a mutant, an analogue, or a variant thereof). In some aspects, the polynucleotide encoding a hGck is a modified polynucleotide (e.g., Gck, a mutant, an analogue, or variant thereof). In some aspects, the modifications to the coding sequence preserve the wild-type or mutant amino acid sequence for insulin and/or glucokinase. In some aspects, the encoded human Ins protein comprises a signal sequence and a proinsulin polypeptide. In some aspects, the encoded human Ins protein comprises the amino acid sequence of any of amino acids 25-110 of SEQ ID NO: 41, amino acids 25-110 of SEQ ID NO: 144, or amino acids 25-110 of SEQ ID NO: 145. In some aspects, the modified nucleic acid sequence encodes a human preproinsulin (e.g., SEQ ID NO: 41, SEQ ID NO: 144, or SEQ ID NO: 145). In some aspects, the modified nucleic acid sequence encodes a human Gck (e.g., SEQ ID NO: 82).

In some aspects, the modified nucleic acids are codon optimized. In some aspects, the codon optimization includes modifying codons in the open reading frame of the nucleic acid encoding insulin or glucokinase. In some aspects, the modified nucleic acids comprise reduced CpG content relative to the corresponding wild-type sequence and/or unmodified sequence.

In some aspects, the modified nucleic acid has reduced innate immunogenicity relative to the corresponding wild-type sequence and/or unmodified sequence. In some aspects, the modified nucleic acid has increased expression relative to the corresponding wild-type sequence and/or unmodified sequence. In some aspects, the modified nucleic acid has decreased expression relative to the corresponding wild-type sequence and/or unmodified sequence. In some aspects, the modified sequences are developed through in silico methods followed by manual sequence examination. Nucleic acids of the disclosure can be produced using molecular biology techniques, e.g., modified cDNAs encoding insulin or glucokinase can be obtained by PCR amplification or cDNA cloning techniques.

In some aspects, the nucleic acid sequences are modified to reduce CpG content, e.g., to minimize the inflammatory response through TLR9 dimerization and related pathways. In some aspects, certain CpG motifs are inhibitory or neutralizing for their inflammatory effects. In some embodiments, one or more of these motifs can be preserved. In some aspects, such CpG motifs can be introduced into a nucleic acid sequence for inhibition of the downstream effects of TLR9 dimerization.

In some aspects, the codon modifications can reduce the immunogenicity of the insulin and/or glucokinase encoding polynucleotides relative to a corresponding wild-type polynucleotide and/or unmodified polynucleotide. In some aspects, the codon modifications improve the expression of the insulin or glucokinase encoding polynucleotide relative to a corresponding wild-type and/or unmodified polynucleotide. In some aspects, the codon modifications can reduce the immunogenicity of the glucokinase encoding polynucleotides relative to a corresponding wild-type Gck polynucleotide and/or unmodified Gck polynucleotide.

The polynucleotides and modified nucleic acids of the disclosure can be present in whole cells, in a cell lysate, or in a partially purified or substantially pure form. The polynucleotides and modified nucleic acids can be isolated.

As used herein, a polynucleotide or nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art, see e.g. F. Ausubel, et al., ed. (1987) Current Protocols in Molecular Biology, Greene Publishing and Wiley Interscience, New York.

In some aspects, a polynucleotide or modified nucleic acid of the disclosure can be, for example, DNA or RNA and may or may not contain intron sequences. In some aspects, the nucleic acid can be a cDNA molecule.

### Insulin Nucleic Acids

In some aspects, the polynucleotide encoding the human insulin comprises a sequence encoding a wild-type human insulin (SEQ ID NO: 147) and/or a human Ins mutant or analogue (e.g., SEQ ID NO: 110 or SEQ ID NO: 111). In some aspects, the polynucleotide encoding the human insulin is modified relative to a wild-type (SEQ ID NO: 147) and/or an unmodified human insulin (Ins) or a human Ins mutant or analogue (e.g., SEQ ID NO: 110 or SEQ ID NO: 111). In some aspects, the polynucleotide encoding the human insulin comprises a sequence including a 5′ UTR, an ORF, and/or a 3′ UTR, e.g., corresponding to SEQ ID NO: 1 or SEQ ID NO: 127. In some aspects, the polynucleotide encoding the human insulin is modified relative to a sequence including a 5′ UTR, an ORF, and/or a 3′ UTR, e.g., corresponding to SEQ ID NO: 1 or SEQ ID NO: 127. In some aspects, the polynucleotide encoding the human insulin encodes wild-type human insulin (SEQ ID NO: 41), variants or mutants thereof (e.g., SEQ ID NO: 144 or SEQ ID NO: 145) or a functional fragment thereof.

Insulin includes two polypeptide chains, the A- and B-chains, linked together by disulfide bonds. It is first synthesized as a single polypeptide called preproinsulin. “Preproinsulin” is the primary translational product of the insulin gene. It is a peptide that is 110 amino acids in length. Preproinsulin includes a proinsulin molecule with a signal peptide attached to its N-terminus. Part of the N-terminus including the signal peptide of the preproinsulin is cleaved off, leaving the remaining amino acids as “proinsulin”. Amino acids 1-30 of the resulting cleaved sequence is the “B chain”, and here “B10” corresponds to position 34 of preproinsulin. Thus, for example, a “B10” proinsulin mutation corresponds to a H34 mutation in preproinsulin. In certain aspects, as referenced herein, “B10H” refers the wild-type histidine amino acid at the B10 position (also referenced as H34 in the wild-type preproinsulin sequence). The preproinsulin and proinsulin also include a C-peptide between the A- and B-chains. In the mature insulin protein, the C-peptide is proteolytically cleaved and the A- and B-chains are linked by disulfide bonds.

In some aspects, the polynucleotide encoding the human insulin disclosed herein encodes a preproinsulin mutant comprising one or more mutations at position(s) H34, P52, K53, R55, and/or L86 relative to the corresponding position in wild-type preproinsulin (SEQ ID NO: 41). In some aspects, the polynucleotide encoding the human insulin encodes a preproinsulin mutant comprising one or more of the following mutations H34D, H34I, H34V, P52D, K53R, R55K, and/or L86R relative to the corresponding position in SEQ ID NO: 41. In some aspects, the polynucleotide encoding the human insulin encodes a preproinsulin mutant comprising mutations H34D, H34I, H34V, P52D, K53R, R55K, and/or L86R relative to the corresponding position in SEQ ID NO: 41. In some aspects, the polynucleotide encoding the human insulin encodes a preproinsulin mutant comprising mutations P52D, K53R, R55K, and/or L86R relative to the corresponding position in SEQ ID NO: 41. In some aspects, the polynucleotide encoding the human insulin encodes an amino acids sequence at least 90%, 95%, 99% or 100% similar to an amino acid sequence selected from SEQ ID NO: 41, SEQ ID NO: 144, or SEQ ID NO: 145. In some aspects, the polynucleotide encoding the human insulin encodes an amino acids sequence at least 90%, 95%, 99% or 100% similar to an amino acid sequence selected from SEQ ID NO: 41, SEQ ID NO: 144, or SEQ ID NO: 145, wherein the amino acid sequence comprises one or more of the following mutations H34D, H34I, H34V, P52D, K53R, R55K, and/or L86R relative to the corresponding position in SEQ ID NO: 41. In some aspects, the polynucleotide encoding the human insulin encodes an amino acids sequence that does not include a H34 mutation relative to the corresponding position in SEQ ID NO: 41.

In some aspects, the polynucleotide encoding the human insulin comprises a cleavage site, e.g., a furin endoprotease cleavage site.

In some aspects, the polynucleotide encoding the human insulin comprises a nucleic acid encoding a signal peptide (e.g., a wild-type preproinsulin signal sequence, an IL-6 signal sequence, or a fibronectin signal sequence). In some aspects, the preproinsulin comprises a wild-type insulin signal sequence (e.g., MALWMRLLPLLALLALWGPDPAAA (SEQ ID NO: 165) or amino acids 1-24 of SEQ ID NO: 41). In some aspects, the signal sequence of wild-type preproinsulin is replaced with a non-insulin secretion peptide, e.g., an IL-6 signal sequence (e.g., MNSFSTSAFGPVAFSLGLLLVLPAAFPAP (SEQ ID NO: 166)) or a fibronectin signal sequence (e.g., MLRGPGPGLLLLAVQCLGTAVPSTGA (SEQ ID NO: 167)).

In some aspects, the polynucleotide encoding the human insulin encodes a human insulin comprising an amino acid modification selected from H34D, H34I, or H34V corresponding to wild-type preproinsulin amino acid positions (or a histidine (H) to aspartic acid (D), isoleucine (I) or valine (V) at position B10 of the proinsulin B-chain). In some aspects, the polynucleotide encoding the human insulin encodes a human insulin comprising an amino acid modification H34D corresponding to wild-type preproinsulin amino acid positions (or a histidine (H) to aspartic acid (D) at position B10 of the proinsulin B-chain). In some aspects, the polynucleotide encoding the human insulin encodes a human insulin comprising an amino acid modification selected from H34D, H34I, or H34V corresponding to wild-type preproinsulin amino acid positions (or a histidine (H) to aspartic acid (D), isoleucine (I) or valine (V) at position B10 of the proinsulin B-chain), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the polynucleotide encoding the human insulin encodes a human insulin comprising amino acid modifications K53R, R55K, and L86R corresponding to wild-type preproinsulin amino acid positions (or modifications corresponding to the proinsulin lysine (K) to arginine (R) at position B29, arginine (R) to lysine (K) at position C1, and leucine (L) to arginine (R) at position C32). In some aspects, the polynucleotide encoding the human insulin encodes a human insulin comprising amino acid modifications K53R, R55K, and L86R corresponding to wild-type preproinsulin amino acid positions (or modifications corresponding to the proinsulin lysine (K) to arginine (R) at position B29, arginine (R) to lysine (K) at position C1, and leucine (L) to arginine (R) at position C32), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the polynucleotide encoding the human insulin encodes a human insulin comprising amino acid modifications H34D, K53R, R55K, and L86R corresponding to wild-type preproinsulin amino acid positions (or modifications corresponding to the proinsulin a histidine (H) to aspartic acid (D) at position B10, lysine (K) to arginine (R) at position B29, arginine (R) to lysine (K) at position C1, and leucine (L) to arginine (R) at position C32). In some aspects, the modified nucleic acid encodes a human insulin comprising amino acid modifications H34D, K53R, R55K, and L86R corresponding to wild-type preproinsulin amino acid positions (or modifications corresponding to the proinsulin a histidine (H) to aspartic acid (D) at position B10, lysine (K) to arginine (R) at position B29, arginine (R) to lysine (K) at position C1, and leucine (L) to arginine (R) at position C32), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the polynucleotide encoding the human insulin encodes a human insulin comprising amino acid modifications H34I, K53R, R55K, and L86R corresponding to wild-type preproinsulin amino acid positions (or modifications corresponding to the proinsulin a histidine (H) to isoleucine (I) at position B10, lysine (K) to arginine (R) at position B29, arginine (R) to lysine (K) at position C1, and leucine (L) to arginine (R) at position C32). In some aspects, the polynucleotide encoding the human insulin encodes a human insulin comprising amino acid modifications H34I, K53R, R55K, and L86R corresponding to wild-type preproinsulin amino acid positions (or modifications corresponding to the proinsulin a histidine (H) to isoleucine (I) at position B10, lysine (K) to arginine (R) at position B29, arginine (R) to lysine (K) at position C1, and leucine (L) to arginine (R) at position C32), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the polynucleotide encoding the human insulin encodes a human insulin comprising amino acid modifications H34V, K53R, R55K, and L86R corresponding to wild-type preproinsulin amino acid positions (or modifications corresponding to the proinsulin a histidine (H) to valine (V) at position B10, lysine (K) to arginine (R) at position B29, arginine (R) to lysine (K) at position C1, and leucine (L) to arginine (R) at position C32). In some aspects, the polynucleotide encoding the human insulin encodes a human insulin comprising amino acid modifications H34V, K53R, R55K, and L86R corresponding to wild-type preproinsulin amino acid positions (or modifications corresponding to the proinsulin a histidine (H) to valine (V) at position B10, lysine (K) to arginine (R) at position B29, arginine (R) to lysine (K) at position C1, and leucine (L) to arginine (R) at position C32), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the polynucleotide encoding the human insulin encodes a human insulin comprising amino acid modifications P49D, K53R, R55K, and L86R corresponding to wild-type preproinsulin amino acid positions (or modifications corresponding to the proinsulin a proline (P) to aspartic acid (D) at position B28, lysine (K) to arginine (R) at position B29, arginine (R) to lysine (K) at position C1, and leucine (L) to arginine (R) at position C32). In some aspects, the polynucleotide encoding the human insulin encodes a human insulin comprising amino acid modifications P49D, K53R, R55K, and L86R (corresponding to wild-type preproinsulin amino acid positions), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the polynucleotide encoding the human insulin encodes a human insulin (Ins) protein (e.g., a preproinsulin or variant thereof), wherein the nucleic acid comprises: (i) a nucleotide sequence encoding a signal peptide (e.g., a wild-type preproinsulin signal sequence, an IL-6 signal sequence, or a fibronectin signal sequence) and (ii) a nucleotide sequence encoding a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid in wild-type proinsulin (or an amino acid modification at a position selected from amino acid H34, P52, K53, R55, L86, or any combination thereof relative to the corresponding amino acid in wild-type preproinsulin). In some aspects, the signal peptide is not a wild-type preproinsulin signal sequence, e.g., the wild-type preproinsulin sequence is replaced with an IL-6 signal sequence or fibronectin signal sequence). In some aspects, the polynucleotide encoding the human insulin further comprises a cleavage site (e.g., a furin cleavage site). In some aspects, the encoded human Ins protein (e.g., a preproinsulin or variant thereof) comprises an amino acid modification selected from (i) H34D, H34I, or H34V (or a histidine (H) to aspartic acid (D), isoleucine (I) or valine (V) at position B10 of the proinsulin B-chain), and/or (ii) one or more amino acid modifications at P52, K53, R55, and/or L86 relative to the wild-type preproinsulin sequence (or positions B28 and/or B29 of the proinsulin B-chain or positions C1 and/or C32 of the proinsulin C-chain). In some aspects, the one or more amino acid modifications at P52, K53, R55, and/or L86 comprise P52D, K53R, R55K, L86R, or any combination thereof (or the one or more modifications in the proinsulin B-chain or C-chain comprise a proline (P) to aspartic acid (D) at position B28 of the proinsulin B-chain, a lysine (K) to arginine (R) at position B29 of the proinsulin B-chain, arginine (R) to lysine (K) at position C1 of the proinsulin C-chain, leucine (L) to arginine (R) at position C32 of the proinsulin C-chain, or any combination thereof).

In some aspects, the polynucleotide encoding the human insulin encodes a variant or mutant human insulin protein or a functional fragment thereof. In some aspects, the human insulin protein comprises an amino acid sequence having least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to amino acids 25-110 of SEQ ID NO: 41, amino acids 25-110 of SEQ ID NO: 144, or amino acids 25-110 of SEQ ID NO: 145. In some aspects, the human insulin protein comprises an amino acid sequence having least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 41, SEQ ID NO: 144, or SEQ ID NO: 145. In some aspects, the human insulin protein comprises an insertion, a deletion, a substitution, or combinations thereof relative to wild-type human insulin. In some aspects, the human insulin protein comprises at least one substitution. In some aspects, the at least one substitution is a conservative substitution. In some aspects, the at least one substitution is a non-conservative substitution.

In some aspects, a polynucleotide of the disclosure comprises an open reading frame (ORF) encoding a human insulin comprising a nucleic acid sequence having a nucleotide sequence at least 85%, 86%, 87%, 88%, 89%, 90%, 91%, 92%, 93%, 94%, 95%, 96%, 97%, 98%, 99%, or 100% identical to nucleic acids 73-330 of any of SEQ ID NOs: 43-57, 110-116, 150-151, 154-155, and 157-159, nucleic acids 88-345 of any of SEQ ID NOs: 117-122, 152, and 156, or nucleic acids 79-336 of SEQ ID NO: 153. In some aspects, the ORF further comprises a nucleic acid sequence encoding a signal peptide.

In some aspects, a polynucleotide of the disclosure comprises an open reading frame (ORF) encoding a human insulin comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159, wherein the polynucleotide encodes a human insulin protein (e.g., SEQ ID NO: 41, SEQ ID NO: 144, or SEQ ID NO: 145) or a functional fragment thereof.

In some aspects, a polynucleotide of the disclosure comprises an open reading frame (ORF) comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 121. In some aspects, a polynucleotide of the disclosure comprises an open reading frame (ORF) comprising a nucleic acid sequence having the sequence of SEQ ID NO: 121.

In some aspects, the polynucleotide encoding the human insulin of the disclosure comprises an open reading frame comprising a nucleic acid having the sequence of SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159. In some aspects, the polynucleotide encoding the human insulin of the disclosure comprises an open reading frame comprising a nucleic acid having the sequence of SEQ ID NO: 121. In some aspects, the polynucleotide encoding the human insulin comprises an ORF sequence present or referenced in Table 1.

In some aspects, a polynucleotide of the disclosure comprises two or more ORFs. In some aspects, the two or more ORFs are operably linked. In some aspects, the ORFs are operably linked by an IRES.

In some aspects, the polynucleotide comprising an ORF encoding the human insulin of the disclosure further comprises a 5′ UTR nucleic acid sequence. In some aspects, the polynucleotide comprising an ORF encoding the human insulin of the disclosure further comprises a modified 5′ UTR nucleic acid sequence. In some aspects, the polynucleotide comprising an ORF encoding the human insulin of the disclosure further comprises a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, or SEQ ID NO: 83, SEQ ID NO: 146, or SEQ ID NO: 148.

In some aspects, the polynucleotide comprising an ORF encoding the human insulin comprises a Kozak consensus sequence (Kozak consensus or Kozak sequence). In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide comprising an ORF encoding the human insulin comprises a 5′ UTR nucleic acid sequence present or referenced in Table 1.

In some aspects, the polynucleotide comprising an ORF encoding the human insulin of the disclosure further comprises a modified 3′ UTR nucleic acid sequence. In some aspects, the polynucleotide comprising an ORF encoding the human insulin of the disclosure further comprises a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 97, SEQ ID NO: 98, SEQ ID NO: 99, SEQ ID NO: 100, SEQ ID NO: 101, SEQ ID NO: 149, or SEQ ID NO: 171. In some aspects, the 3′ UTR comprises a nucleic acid having the sequence of SEQ ID NO: 60, SEQ ID NO: 97, SEQ ID NO: 98, SEQ ID NO: 99, SEQ ID NO: 100, SEQ ID NO: 101, SEQ ID NO: 149, or SEQ ID NO: 171. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the polynucleotide comprising an ORF encoding the human insulin comprises a 3′ UTR nucleic acid sequence present or referenced in Table 1.

In some aspects, the polynucleotide comprising an ORF encoding the human insulin of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 127, SEQ ID NO: 128, SEQ ID NO: 129, SEQ ID NO: 133, SEQ ID NO: 134, SEQ ID NO: 135, SEQ ID NO: 136, SEQ ID NO 137, SEQ ID NO: 138, SEQ ID NO: 160, SEQ ID NO: 161, or SEQ ID NO: 170, wherein the nucleic acid sequence encodes a human insulin protein (e.g., SEQ ID NO: 41, SEQ ID NO: 144, or SEQ ID NO: 145) or a functional fragment thereof. In some aspects, the polynucleotide comprising an ORF encoding the human insulin of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 138. In some aspects, the polynucleotide encoding the human insulin of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 170.

In some aspects, the polynucleotide comprising an ORF encoding the human insulin of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the polynucleotide encoding the human insulin of the disclosure comprises a nucleic acid having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 127, SEQ ID NO: 128, SEQ ID NO: 129, SEQ ID NO: 133, SEQ ID NO: 134, SEQ ID NO: 135, SEQ ID NO: 136, SEQ ID NO 137, SEQ ID NO: 138, SEQ ID NO: 160, SEQ ID NO: 161, or SEQ ID NO: 170. In some aspects, the polynucleotide encoding the human insulin of the disclosure comprises a nucleic acid having the sequence of SEQ ID NO: 138. In some aspects, the polynucleotide encoding the human insulin of the disclosure comprises nucleic acids 5-957 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the polynucleotide comprising an ORF encoding the human insulin comprises a nucleic acid comprising a 5′ UTR, an ORF, and a 3′ UTR presented in Table 1.

In some aspects, the polynucleotide of the disclosure encodes a human insulin comprising a wild-type preproinsulin secretion signal peptide. In some aspects, the polynucleotide encoding the human insulin of the disclosure does not encode a wild-type preproinsulin secretion signal peptide. In some aspects, the wild-type preproinsulin is replaced by a non-insulin secretion signal. In some aspects, the polynucleotide encoding the human insulin of the disclosure encodes a human preproinsulin comprising an interleukin 6 (IL-6) secretion signal peptide. In some aspects, the polynucleotide encoding the human insulin of the disclosure encodes a human preproinsulin comprising a fibronectin secretion signal peptide.

### Glucokinase Nucleic Acids

In some aspects, the polynucleotide encoding a human glucokinase comprises a sequence encoding wild-type human glucokinase (SEQ ID NO: 82) or a functional fragment thereof. In some aspects, the polynucleotide encoding the human glucokinase comprises a modified nucleic acid sequence which encodes wild-type human glucokinase (SEQ ID NO: 82) or a functional fragment thereof. In some aspects, the polynucleotide comprising a sequence encoding the human glucokinase disclosed herein is modified relative to the wild-type and/or unmodified, e.g., including a 5′ UTR, an ORF, and/or a 3′ UTR, nucleic acid sequence, e.g., corresponding to SEQ ID NO: 19.

In some aspects, the polynucleotide of the disclosure comprises an ORF encoding the human glucokinase comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162 wherein the nucleic acid sequence encodes a human glucokinase protein (SEQ ID NO: 82) or a functional fragment thereof.

In some aspects, the polynucleotide of the disclosure comprises an ORF encoding the human glucokinase comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 68. In some aspects, the polynucleotide of the disclosure comprises an open reading frame encoding the human glucokinase comprising a nucleic acid having the sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the polynucleotide of the disclosure comprises an open reading frame encoding the human glucokinase comprising a nucleic acid having the sequence of SEQ ID NO: 68. In some aspects, the polynucleotide encoding the human glucokinase comprises an ORF sequence present or referenced in Table 2.

In some aspects, the polynucleotide comprising an ORF encoding the human glucokinase of the disclosure further comprises a modified 5′ UTR nucleic acid sequence. In some aspects, the polynucleotide comprising an ORF encoding the human glucokinase of the disclosure further comprises a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, SEQ ID NO: 83, SEQ ID NO: 146, or SEQ ID NO: 148.

In some aspects, the polynucleotide comprising an ORF encoding the human glucokinase further comprises a Kozak consensus sequence (Kozak consensus or Kozak sequence). In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide comprising an ORF encoding the human glucokinase comprises a 5′ UTR sequence present or referenced in Table 2.

In some aspects, the polynucleotide comprising an ORF encoding the human glucokinase of the disclosure further comprises a modified 3′ UTR nucleic acid sequence. In some aspects, the polynucleotide comprising an ORF encoding the human glucokinase of the disclosure further comprises a 3′ UTR comprising a nucleic acid sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 149, or SEQ ID NO: 169. In some aspects, the 3′ UTR comprises a nucleic acid having the sequence of SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 149, or SEQ ID NO: 169. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the polynucleotide comprising an ORF encoding the human glucokinase comprises a 3′ UTR sequence present or referenced in Table 2.

In some aspects, the polynucleotide encoding the human glucokinase of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, SEQ ID NO: 164, or SEQ ID NO: 168 wherein the nucleic acid sequence encodes a human glucokinase protein (e.g., SEQ ID NO: 82) or functional fragment thereof. In some aspects, the polynucleotide encoding the human glucokinase of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 168. In some aspects, the polynucleotide encoding the human glucokinase of the disclosure comprises a nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-2025 of a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the polynucleotide encoding the human glucokinase of the disclosure comprises a nucleic acid having the sequence of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, SEQ ID NO: 164, or SEQ ID NO: 168. In some aspects, the polynucleotide encoding the human glucokinase of the disclosure comprises a nucleic acid having the sequence of SEQ ID NO: 168. In some aspects, the polynucleotide encoding the human glucokinase of the disclosure comprises nucleic acids 5-2025 of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the polynucleotide encoding the human glucokinase is a nucleic acid present or referenced in Table 2.

### Expression Cassettes

Certain aspects of the disclosure are directed to a combination gene therapy, e.g., a combination AAV gene therapy for delivery of an expression cassette (or expression construct) comprising a polynucleotide encoding an insulin (Ins) protein and an expression cassette comprising a polynucleotide encoding a glucokinase (Gck) protein. In some aspects, the combination therapy includes separate administration of the Ins and Gck expression cassettes. In some aspects, the combination therapy includes administration of the insulin and glucokinase expression cassettes in a single formulation, e.g., two separate rAAV particles in the same pharmaceutical composition.

In some aspects, the combination therapy comprises an insulin expression cassette comprising a promoter operably linked to a polynucleotide encoding a human insulin protein, wherein the insulin expression cassette is flanked by inverted terminal repeats. In some aspects, the combination therapy comprises a glucokinase expression cassette comprising a promoter operably linked to a polynucleotide encoding a human glucokinase protein, wherein the glucokinase expression cassette is flanked by inverted terminal repeats. In some aspects the first and second promoter are the same or substantially the same promoter; or in some aspects the first and second promoters are different promoters that drive substantially equivalent levels of transgene expression.

An expression cassette comprising a eukaryotic promoter operably linked to a DNA of interest (e.g., a DNA encoding insulin or a DNA encoding glucokinase) can be used in the disclosure. In some aspects, the expression cassette containing the DNA sequence (or the corresponding RNA sequence), which can be used in accordance with the disclosure, can be any eukaryotic expression cassette containing the DNA or the RNA sequence of interest. For example, a plasmid or viral construct (e.g., an AAV vector) can be cleaved to provide linear DNA having ligatable termini. These termini can be bound to exogenous DNA having complementary, like ligatable termini to provide a biologically functional recombinant DNA molecule having an intact replicon and a desired phenotypic property. In some aspects, the expression cassette is capable of replication in both eukaryotic and prokaryotic hosts.

In some aspects, the exogenous DNA used in the disclosure is obtained from suitable cells, and the constructs prepared using techniques known in the art. Likewise, techniques for obtaining expression of exogenous DNA or RNA sequences in a genetically altered host cell are known in the art (see e.g., Kormal et al., Proc. Natl. Acad. Sci. USA, 84:2150-2154 (1987); Sambrook et al. Molecular Cloning: a Laboratory Manual, 2nd Ed., 1989, Cold Spring Harbor Laboratory Press, Cold Spring Harbor, N.Y.; each of which are hereby incorporated by reference with respect to methods and compositions for eukaryotic expression of a DNA of interest).

In some aspects, the DNA expression construct comprises a promoter to facilitate expression of the DNA of interest (e.g., polynucleotide encoding an insulin or a polynucleotide encoding a glucokinase) within a secretory cell. In some aspects, the promoter is a strong, eukaryotic promoter such as a promoter from cytomegalovirus (CMV), mouse mammary tumor virus (MMTV), Rous sarcoma virus (RSV), or adenovirus. Exemplary promoters include, but are not limited to the promoter from the immediate early gene of human CMV (Boshart et al., Cell 41:521-530 (1985) and the promoter from the long terminal repeat (LTR) of RSV (Gorman et al., Proc. Natl. Acad. Sci. USA 79:6777-6781 (1982)). In some aspects, the promoter is used together with an intronic sequence. In some aspects, the CMV promoter is a mini CMV promoter. Alternatively, the promoter used can be a tissue-specific promoter. In some aspects, the insulin expression cassette comprises a CMV promoter. In some aspects, the glucokinase expression cassette comprises a CMV promoter.

The expression cassettes of the disclosure can also include other components such as a marker (e.g., an antibiotic resistance gene (such as an ampicillin resistance gene) or β-galactosidase) to aid in selection of cells containing and/or expressing the construct, an origin of replication for stable replication of the construct in a bacterial cell (preferably, a high copy number origin of replication), a nuclear localization signal, or other elements which facilitate production of the DNA expression construct, the protein encoded thereby, or both.

For eukaryotic expression, the expression cassette can contain at a minimum a eukaryotic promoter operably linked to a DNA of interest (e.g., a polynucleotide encoding an insulin or a glucokinase), which is in turn operably linked to a polyadenylation sequence. The polyadenylation signal sequence can be selected from any of a variety of polyadenylation signal sequences known in the art. In some aspects, the polyadenylation signal sequence is the SV40 early polyadenylation signal sequence. In some aspects, the polyadenylation signal sequence is a growth hormone polyadenylation signal sequence (e.g., a bovine growth hormone polyA or a human growth hormone polyA). In some aspects, the glucokinase expression cassette comprises an SV40 polyadenylation signal sequence. In some aspects, the insulin expression cassette comprises a bovine orh human growth hormone polyadenylation signal sequence.

In some aspects, the expression cassette can also include one or more introns, which can increase levels of expression of the DNA of interest, particularly where the DNA of interest is a cDNA (e.g., contains no introns of the naturally-occurring sequence). Any of a variety of introns known in the art can be used (e.g., the human β-globin intron, which is inserted in the construct at a position 5′ to the DNA of interest).

The DNA of interest (e.g., a polynucleotide encoding an insulin or a polynucleotide encoding a glucokinase) can be inserted into an expression cassette so that the therapeutic molecule (e.g., a protein) is expressed as a fusion protein (e.g., a fusion protein having β-galactosidase or a portion thereof at the N-terminus and the therapeutic protein at the C-terminal portion). Production of a fusion protein can facilitate identification of transformed cells expressing the protein (e.g., by enzyme-linked immunosorbent assay (ELISA) using an antibody which binds to the fusion protein).

The vectors for delivery of the DNA of interest (e.g., the polynucleotide encoding an insulin, the polynucleotide encoding a glucokinase can be either viral or non-viral, or can be composed of naked DNA admixed with an adjuvant such as viral particles (e.g., AAV particle) or cationic lipids or liposomes. An “adjuvant” is a substance that does not by itself produce the desired effect, but acts to enhance or otherwise improve the action of the active compound. The precise vector and vector formulation used will depend upon several factors such as the cell and/or organ targeted for gene transfer.

Examples of suitable promoters include cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter, RSV promoter, and the herpes simplex virus thymidine kinase promoter. In some aspects, the promoter is a cell-specific and/or a tissue-specific promoter. In some aspects, the promoter is used together with an intronic sequence. In some aspects, the promoter is tissue specific. In some aspects, the first promoter is a CMV promoter. In some aspects, the second promoter is a CMV promoter. In some aspects, the CMV promoter is a mini CMV promoter. In some aspects, the insulin expression cassette comprises a CMV promoter. In some aspects, the glucokinase expression cassette comprises a CMV promoter. In some aspects the first and second promoter are the same or substantially the same promoter; or in some aspects the first and second promoters are different promoters that drive substantially equivalent levels of transgene expression.

In some aspects, the expression cassette comprises a promoter operably linked to polynucleotide comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159, wherein the polynucleotide encodes a human insulin protein (e.g., SEQ ID NO: 41, SEQ ID NO: 144, or SEQ ID NO: 145) or a functional fragment thereof. In some aspects, the expression cassette comprises a promoter operably linked to a polynucleotide encoding the human insulin protein comprises an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 121 or SEQ ID NO: 122. In some aspects, the polynucleotide encoding the human insulin protein of the disclosure comprises an open reading frame comprising a nucleic acid having the sequence of SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159. In some aspects, the polynucleotide encoding the human insulin protein of the disclosure comprises an open reading frame comprising a nucleic acid having the sequence of SEQ ID NO: 122. In some aspects, the polynucleotide encoding the human insulin protein of the disclosure comprises an open reading frame comprising a nucleic acid having the sequence of SEQ ID NO: 121. In some aspects, the polynucleotide encoding the human insulin protein comprises an ORF sequence present or referenced in Table 1.

In some aspects, the expression cassette comprises a polynucleotide encoding a human insulin comprising a wild-type preproinsulin secretion signal peptide. In some aspects, the polynucleotide encoding the human insulin protein of the disclosure does not encode a wild-type preproinsulin secretion signal peptide. In some aspects, the wild-type preproinsulin is replaced by a non-insulin secretion signal. In some aspects, the expression cassette comprises a polynucleotide encoding a human preproinsulin comprising an interleukin 6 (IL-6) secretion signal peptide. In some aspects, the expression cassette comprises a polynucleotide encoding a human preproinsulin comprising a fibronectin secretion signal peptide.

In some aspects, the expression cassette comprises the polynucleotide encoding the human insulin protein further comprises a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, or SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide encoding the human insulin protein comprises a 5′ UTR sequence present or referenced in Table 1.

In some aspects, the expression cassette comprises a polynucleotide encoding a human insulin protein further comprising a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 97, SEQ ID NO: 98, SEQ ID NO: 99, SEQ ID NO: 100, SEQ ID NO: 101, SEQ ID NO: 149, or SEQ ID NO: 171. In some aspects, the 3′ UTR comprises a nucleic acid having the sequence of SEQ ID NO: 60, SEQ ID NO: 97, SEQ ID NO: 98, SEQ ID NO: 99, SEQ ID NO: 100, SEQ ID NO: 101, SEQ ID NO: 149, SEQ ID NO: 171. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the polynucleotide encoding the human insulin protein comprises a 3′ UTR sequence present or referenced in Table 1.

In some aspects, the expression cassette comprises a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87 SEQ ID NO: 88, SEQ ID NO: 123, SEQ ID NO: 127, SEQ ID NO: 128, SEQ ID NO: 129, SEQ ID NO: 133, SEQ ID NO: 134, SEQ ID NO: 135, SEQ ID NO: 136, SEQ ID NO 137, SEQ ID NO: 138, SEQ ID NO: 160, SEQ ID NO: 161, or SEQ ID NO: 170, wherein the polynucleotide encodes a human insulin protein (e.g., SEQ ID NO: 41, SEQ ID NO: 144, or SEQ ID NO: 145) or a functional fragment thereof. In some aspects, the expression cassette comprises a polynucleotide encoding a human insulin protein having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 138 or SEQ ID NO: 171. In some aspects, the expression cassette comprises a polynucleotide encoding a human insulin protein having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the expression cassette comprises a polynucleotide encoding a human insulin protein having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87 SEQ ID NO: 88, SEQ ID NO: 123, SEQ ID NO: 127, SEQ ID NO: 128, SEQ ID NO: 129, SEQ ID NO: 133, SEQ ID NO: 134, SEQ ID NO: 135, SEQ ID NO: 136, SEQ ID NO 137, SEQ ID NO: 138, SEQ ID NO: 160, SEQ ID NO: 161, or SEQ ID NO: 170. In some aspects, the expression cassette comprises a polynucleotide encoding a human insulin protein having the sequence of SEQ ID NO: 138 or SEQ ID NO: 170. In some aspects, the expression cassette comprises a polynucleotide encoding a human insulin protein having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the expression cassette comprises a polynucleotide encoding a human insulin protein comprising a 5′ UTR, an ORF, and a 3′ UTR present or referenced in Table 1.

In some aspects, the expression cassette comprises a promoter operably linked to a polynucleotide encoding a human glucokinase protein sequence comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the expression cassette comprises a promoter operably linked to a polynucleotide encoding a human glucokinase protein sequence comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 68. In some aspects, the expression cassette comprises a promoter operably linked to a polynucleotide encoding a human glucokinase protein having the sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the expression cassette comprises a promoter operably linked to a polynucleotide encoding a human glucokinase protein having the sequence of SEQ ID NO: 68. In some aspects, the polynucleotide encoding a human glucokinase protein comprises an ORF sequence present or referenced in Table 2.

In some aspects, the expression cassette comprises a polynucleotide encoding a human glucokinase protein further comprising a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide encoding a human glucokinase protein comprises a 5′ UTR sequence present or referenced in Table 2.

In some aspects, the expression cassette comprises a polynucleotide encoding a human glucokinase protein further comprising a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 149, or SEQ ID NO: 169. In some aspects, the 3′ UTR comprises a nucleic acid having the sequence of SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 149, or SEQ ID NO: 169. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, or any combination thereof. In some aspects, the polynucleotide encoding a human glucokinase protein comprises a 3′ UTR sequence present or referenced in Table 2.

In some aspects, the expression cassette comprises a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, SEQ ID NO: 164, or SEQ ID NO: 168 wherein the nucleic acid sequence encodes a human glucokinase protein (e.g., SEQ ID NO: 82) or functional fragment thereof. In some aspects, the expression cassette comprises a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 168 wherein the nucleic acid sequence encodes a human glucokinase protein (e.g., SEQ ID NO: 82) or functional fragment thereof. In some aspects, the expression cassette comprises a polynucleotide encoding a human glucokinase protein having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-2025 of a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the expression cassette comprises a polynucleotide encoding a human glucokinase protein having the sequence of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, SEQ ID NO: 164, or SEQ ID NO: 168. In some aspects, the expression cassette comprises a polynucleotide encoding a human glucokinase protein comprising nucleic acids 5-2025 of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the expression cassette comprises polynucleotide encoding a human glucokinase protein comprising a 5′ UTR, an ORF, and a 3′ UTR present or referenced in Table 2.

In some aspects, the combination therapy comprises administering an insulin expression cassette comprising a polynucleotide encoding a human insulin comprising a ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159 and administering a glucokinase expression cassette comprising a polynucleotide encoding a human glucokinase comprising a ORF having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the combination therapy comprises an insulin expression cassette comprising a polynucleotide encoding a human insulin comprising a ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 121 and glucokinase expression cassette comprising a polynucleotide encoding a human glucokinase comprising a ORF having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to SEQ ID NO: 68.

In some aspects, the insulin expression cassette and the glucokinase expression cassette of the combination therapy are administered at a ratio of about 1:0.25-0.75, 1:0.25-0.7, 1:0.25-0.65, 1:0.25-0.60, 1:0.25-0.55, 1:0.25-0.5, 1:0.3-0.7, 1:0.3-0.65, 1:0.3-0.6, 1:0.3-0.55, 1:0.3-0.5, 1:0.35-0.7, 1:0.35-0.65, 1:0.35-0.6, 1:0.35-0.55, 1:0.35-0.5, 1:0.4-0.7, 1:0.4-0.65, 1:0.4-0.6, 1:0.4-0.55, 1:0.4-0.5, 1:0.45-0.7, 1:0.45-0.65, 1:0.45-0.6, 1:0.45-0.55, or 1:0.45-0.5. In some aspects, the insulin expression cassette and the glucokinase expression cassette of the combination therapy are administered at a ratio of about 1:0.25-0.75, 1:0.3-0.75, 1:0.35-0.75, 1:0.40-0.75, 1:0.45-0.75, or 1:0.5-0.75. In some aspects, the insulin expression cassette and the glucokinase expression cassette of the combination therapy are administered at a ratio of about 1:0.25-0.75, 1:0.3-0.70, 1:0.35-0.65, 1:0.4-0.60, or 1:0.45-0.55. In some aspects, the insulin expression cassette and the glucokinase expression cassette of the combination therapy are administered at a ratio of about 1:0.5, about 1:2, or about 1:4. In some aspects, the first AAV vector genome and the second AAV vector genome are in a vector ratio selected from the group consisting of 1:0.25-0.75, 1:1.75-2.25, and 1:3.75-1:4.25 (e.g., about 1:0.5, about 1:2, and about 1:4). In some aspects, the vector ratio (i.e., first AAV vector comprising the hIns expression cassette to second AAV vector comprising the hGck expression cassette) is 1:0.4-0.6. In some aspects, the vector ratio (i.e., first AAV vector comprising the hIns expression cassette to second AAV vector comprising the hGck expression cassette) is 1:0.5.

In some aspects, the polynucleotide encoding a human insulin protein encodes a human insulin comprising a wild-type preproinsulin secretion signal peptide. In some aspects, the polynucleotide encoding a human insulin protein does not encode a wild-type preproinsulin secretion signal peptide. In some aspects, the wild-type preproinsulin is replaced by a non-insulin secretion signal. In some aspects, the f polynucleotide encoding a human insulin protein encodes a human preproinsulin comprising an interleukin 6 (IL-6) secretion signal peptide. In some aspects, the polynucleotide encoding a human insulin protein encodes a human preproinsulin comprising a fibronectin secretion signal peptide.

In some aspects, the insulin expression cassette comprising a polynucleotide encoding a human insulin protein comprises a 5′ UTR, an ORF, and a 3′ UTR present or referenced in Table 1 and the glucokinase expression cassette comprising a polynucleotide encoding a human glucokinase protein comprises a 5′ UTR, an ORF, and a 3′ UTR present or referenced in Table 2.

Certain aspects of the disclosure are directed to vector, e.g., a viral vector, comprising an expression construct. In some aspects, the expression construct comprises an expression cassette. In some aspects, the expression construct further comprises a genome that is able to stabilize and remain episomal in a cell. Within the context of the disclosure, in some aspects, a cell or host cell can encompass a cell used to make the construct or a cell to which the construct is administered. In some aspects, a construct is capable of integrating into a cell's genome, e.g. through homologous recombination or otherwise. In some aspects, the expression construct is one wherein a nucleotide sequence encoding an insulin and/or a glucokinase as disclosed herein, is operably linked to a promoter as provided herein wherein the promoter is capable of directing expression of the nucleotide sequence(s) (i.e. coding sequence(s)) in a cell. In some aspects, an expression cassette as used herein comprises or consists of a nucleotide sequence encoding an insulin and/or a nucleotide sequence encoding a glucokinase, in each case the nucleotide sequence is operably linked to a promoter wherein the promoter is capable of directing expression of said nucleotide sequences. In some aspects, a viral expression construct is an expression construct that is intended to be used in gene therapy. It can be designed to comprise part of a viral genome as disclosed herein.

In some aspects, the expression construct further comprises one or more of: an ITR sequence (e.g., AAV2 ITRs), a polyA sequence (e.g., a SV40 polyadenylation signal, a bGH polyadenylation signal), and an enhancer sequence (e.g., a SV40 enhancer sequence).

In some aspects, expression constructs disclosed herein are prepared using recombinant techniques in which nucleic acid sequences encoding an insulin and/or a glucokinase are expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis).

### Delivery Vectors

The present disclosure also provides vectors comprising any of the polynucleotides, expression cassettes, or constructs described herein. In some aspects, the delivery vector is a viral vector, a non-viral vectors, a plasmid, a lipid, or a lysosome. In some aspects, the delivery vector is a viral vector. In some aspects, the viral vector is an adeno-associated virus (AAV) expression vector.

In some aspects, a polynucleotide encoding an insulin and/or a glucokinase are used in an expression construct or expression vector. The phrase “expression vector” generally refers to a nucleotide sequence that is capable of effecting expression of a gene in a host compatible with such sequences. These expression vectors can include at least suitable promoter sequences and optionally, transcription termination signals. An additional factor necessary or helpful in effecting expression can also be used as disclosed herein. A polynucleotide encoding an insulin and/or a glucokinase can be incorporated into an expression vector capable of introduction into and expression in an in vitro cell culture. In some aspects, the expression vector is suitable for replication in a prokaryotic host, such as bacteria, e.g., E. coli, or can be introduced into a cultured mammalian, plant, insect, (e.g., Sf9), yeast, fungi or other eukaryotic cell lines. In some aspects, the expression construct is suitable for expression in vivo.

In some aspects, the delivery vector comprises an insulin expression cassette comprising a promoter operably linked to a polynucleotide comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159, wherein the polynucleotide encodes a human insulin protein (e.g., SEQ ID NO: 41, SEQ ID NO: 144, or SEQ ID NO: 145) or a functional fragment thereof. In some aspects, the delivery vector comprises an insulin expression cassette comprising a promoter operably linked to a polynucleotide comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 122. In some aspects, the delivery vector comprises an insulin expression cassette comprising a promoter operably linked to a polynucleotide comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 121. In some aspects, the polynucleotide comprises an ORF having the sequence of SEQ ID NOs: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159. In some aspects, the polynucleotide comprises an ORF having the sequence of SEQ ID NO: 122. In some aspects, the polynucleotide comprises an ORF having the sequence of SEQ ID NO: 121. In some aspects, the polynucleotide encoding a human insulin protein comprises an ORF sequence present or referenced in Table 1.

In some aspects, delivery vector comprises an insulin expression cassette comprising a polynucleotide encoding a human insulin protein encodes a human insulin comprising a wild-type preproinsulin secretion signal peptide. In some aspects, delivery vector comprises an insulin expression cassette comprising a polynucleotide encoding a human insulin protein does not encode a wild-type preproinsulin secretion signal peptide. In some aspects, the wild-type preproinsulin is replaced by a non-insulin secretion signal. In some aspects, delivery vector comprises an insulin expression cassette comprising a polynucleotide encoding a human insulin protein encodes a human preproinsulin comprising an interleukin 6 (IL-6) secretion signal peptide. In some aspects, delivery vector comprises an insulin expression cassette comprising a polynucleotide encoding a human insulin protein sequence encodes a human preproinsulin comprising a fibronectin secretion signal peptide.

In some aspects, the delivery vector comprises an insulin expression cassette comprising a polynucleotide encoding a human insulin protein further comprising a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, 100% sequence identity to SEQ ID NO: 42, nucleic acids 5-329 of SEQ ID NO: 42, SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide encoding a human insulin protein comprises a 5′ UTR sequence present or referenced in Table 1.

In some aspects, the delivery vector comprises an insulin expression cassette comprising a polynucleotide encoding a human insulin protein further comprising a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 97, SEQ ID NO: 98, SEQ ID NO: 99, SEQ ID NO: 100, SEQ ID NO: 101, SEQ ID NO: 149, or SEQ ID NO: 171. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the 3′ UTR comprises a nucleic acid having the sequence of SEQ ID NO: 60, SEQ ID NO: 97, SEQ ID NO: 98, SEQ ID NO: 99, SEQ ID NO: 100, SEQ ID NO: 101, SEQ ID NO: 149, or SEQ ID NO: 171. In some aspects, the polynucleotide encoding a human insulin protein comprises a 3′ UTR sequence present or referenced in Table 1.

In some aspects, the delivery vector comprises an insulin expression cassette comprising a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 127, SEQ ID NO: 128, SEQ ID NO: 129, SEQ ID NO: 133, SEQ ID NO: 134, SEQ ID NO: 135, SEQ ID NO: 136, SEQ ID NO 137, SEQ ID NO: 138, SEQ ID NO: 160, SEQ ID NO: 161, or SEQ ID NO: 170, wherein the polynucleotide encodes a human insulin protein (e.g., SEQ ID NO: 41, SEQ ID NO: 144, or SEQ ID NO: 145) or a functional fragment thereof. In some aspects, the delivery vector comprises an insulin expression cassette comprising a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 138 or SEQ ID NO: 170. In some aspects, the delivery vector comprises an insulin expression cassette comprising a polynucleotide encoding a human insulin protein having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the delivery vector comprises an insulin expression cassette comprising a polynucleotide encoding a human insulin protein having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 127, SEQ ID NO: 128, SEQ ID NO: 129, SEQ ID NO: 134, SEQ ID NO: 135, SEQ ID NO: 136, SEQ ID NO 137, SEQ ID NO: 138, SEQ ID NO: 160, SEQ ID NO: 161, or SEQ ID NO: 170. In some aspects, the delivery vector comprises an insulin expression cassette comprising a polynucleotide encoding a human insulin protein having the sequence of SEQ ID NO: 138. In some aspects, the delivery vector comprises an insulin expression cassette comprising a polynucleotide encoding a human insulin protein having the sequence of SEQ ID NO: 170. In some aspects, the delivery vector comprises an insulin expression cassette comprising a polynucleotide encoding a human insulin protein comprising nucleic acids 5-957 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the delivery vector comprises an insulin expression cassette comprising a polynucleotide encoding a human insulin protein comprising a 5′ UTR, an ORF, and a 3′ UTR present or referenced in Table 1.

In some aspects, the delivery vector comprises a glucokinase expression cassette comprising a promoter operably linked to a polynucleotide encoding a human glucokinase protein comprising an ORF sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the delivery vector comprises a glucokinase expression cassette comprising a promoter operably linked to a polynucleotide encoding a human glucokinase protein having the sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the polynucleotide encoding a human glucokinase protein comprises an ORF sequence present or referenced in Table 2.

In some aspects, the delivery vector comprises a glucokinase expression cassette comprising a polynucleotide encoding a human glucokinase protein further comprising a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide encoding a human glucokinase protein comprises a 5′ UTR sequence present or referenced in Table 2.

In some aspects, the delivery vector comprises a glucokinase expression cassette comprising a polynucleotide encoding a human glucokinase protein further comprising a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 149, or SEQ ID NO: 169. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the 3′ UTR comprises a nucleic acid having the sequence of SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 149, or SEQ ID NO: 169. In some aspects, the polynucleotide encoding a human glucokinase protein comprises a 3′ UTR sequence present or referenced in Table 2.

In some aspects, the delivery vector comprises a glucokinase expression cassette comprising a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, SEQ ID NO: 164, or SEQ ID NO: 168, wherein the nucleic acid sequence encodes a human glucokinase protein (e.g., SEQ ID NO: 82) or functional fragment thereof. In some aspects, the delivery vector comprises a glucokinase expression cassette comprising a polynucleotide encoding a human glucokinase protein having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-2025 of a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the delivery vector comprises a glucokinase expression cassette comprising a polynucleotide encoding a human glucokinase protein having the sequence of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, SEQ ID NO: 164, or SEQ ID NO: 168. In some aspects, the delivery vector comprises a glucokinase expression cassette comprising a polynucleotide encoding a human glucokinase protein comprising nucleic acids 5-2025 of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the delivery vector comprises a glucokinase expression cassette comprising polynucleotide encoding a human glucokinase protein comprising a 5′ UTR, an ORF, and a 3′ UTR present or referenced in Table 2.

In some aspects, the delivery vectors can comprise sequences encoding a protein (e.g., insulin and/or Gck) operably linked with control or regulatory sequences, selectable markers, any fusion partners, and/or additional elements. In certain aspects, the modified nucleic acid is placed into a functional relationship with another nucleic acid sequence. The term “regulatory sequence” includes promoters, enhancers and other expression control elements (e.g., polyadenylation signals) that control the transcription or translation of the protein. Such regulatory sequences are described, for example, in Goeddel (Gene Expression Technology, Methods in Enzymology 185, Academic Press, San Diego, CA (1990)). In some aspects, the expression vectors include transcriptional and translational regulatory nucleic acid operably linked to the nucleic acid encoding the protein, and are typically appropriate to the host cell used to express the protein. In general, the transcriptional and translational regulatory sequences may include promoter sequences, ribosomal binding sites, transcriptional start and stop sequences, translational start and stop sequences, and enhancer or activator sequences. As is also known in the art, expression vectors can contain a selection gene or marker to allow the selection of transformed host cells containing the expression vector. Selection genes are known in the art and will vary with the host cell used. For example, typically the selectable marker gene confers resistance to drugs, such as G418, hygromycin or methotrexate, on a host cell into which the vector has been introduced. In some aspects, selectable marker genes include the dihydrofolate reductase (DHFR) gene (for use in dhfr-host cells with methotrexate selection/amplification) and the neo gene (for G418 selection).

In some aspects, the delivery vector is a viral vector or a gene therapy vector comprising a viral expression construct. In certain aspects, the viral vector or a gene therapy vector is a vector that is suitable for gene therapy.

In certain aspects, the delivery vector comprising the expression cassette encoding the human insulin and the delivery vector comprising the expression cassette encoding human glucokinase are delivered in a ratio selected from the group consisting of about 1:0.25-0.75, 1:1.75-2.25, and 1:3.75-4.25. In certain aspects, the delivery vector comprising the expression cassette encoding the human insulin and the delivery vector comprising the expression cassette encoding human glucokinase are delivered in a ratio selected from the group consisting of about 1:0.4-0.6, 1:1.9-2.1, and 1:3.9-4.1. In some aspects, the delivery vector comprising the expression cassette encoding the human insulin and the delivery vector comprising the expression cassette encoding human glucokinase are delivered in a ratio selected from the group consisting of about 1:0.25-0.75, 1:0.25-0.7, 1:0.25-0.65, 1:0.25-0.60, 1:0.25-0.55, 1:0.25-0.5, 1:0.3-0.7, 1:0.3-0.65, 1:0.3-0.6, 1:0.3-0.55, 1:0.3-0.5, 1:0.35-0.7, 1:0.35-0.65, 1:0.35-0.6, 1:0.35-0.55, 1:0.35-0.5, 1:0.4-0.7, 1:0.4-0.65, 1:0.4-0.6, 1:0.4-0.55, 1:0.4-0.5, 1:0.45-0.7, 1:0.45-0.65, 1:0.45-0.6, 1:0.45-0.55, and 1:0.45-0.5.

In some aspects, the delivery vector comprising the expression cassette encoding the human insulin and the delivery vector comprising the expression cassette encoding human glucokinase are delivered in a ratio selected from the group consisting of about 1:0.25-0.75, 1:0.3-0.75, 1:0.35-0.75, 1:0.40-0.75, 1:0.45-0.75, and 1:0.5-0.75. In some aspects, the delivery vector comprising the expression cassette encoding the human insulin and the delivery vector comprising the expression cassette encoding human glucokinase are delivered in a ratio selected from the group consisting of about 1:0.25-0.75, 1:0.3-0.70, 1:0.35-0.65, 1:0.4-0.60, and 1:0.45-0.55.

In certain aspects, the delivery vector comprising the expression cassette encoding the human insulin and the delivery vector comprising the expression cassette encoding human glucokinase are delivered in a ratio selected from the group consisting of about 1:0.25-0.75, 1:1.75-2.25, and 1:3.75-1:4.25 (e.g., about 1:0.5, about 1:2, and about 1:4). In some aspects, the vector ratio (i.e., AAV vector comprising the hIns expression cassette to AAV vector comprising the hGck expression cassette) is 1:0.4-0.6. In some aspects, the delivery vectors (i.e., AAV vector comprising the hIns expression cassette to AAV vector comprising the hGck expression cassette) are delivered in a ratio of 1:0.5.

In some aspects, the gene therapy vector includes an Adenoviral and Adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications. (Russell, 2000, J. Gen. Virol. 81:2573-2604; Goncalves, 2005, Virol J. 2(1):43) as indicated above. AAV vectors can result in very stable long term expression of transgene expression (up to 9 years in dog (Niemeyer et al, Blood. 2009 Jan. 22; 113(4):797-806) and up to 2 years in human (Nathwani et al, N Engl J Med. 2011 Dec. 22; 365(25): 2357-65, Simonelli et al, Mol Ther. 2010 March; 18(3):643-50. Epub 2009 Dec. 1.)). In some aspects, adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11):1049-55, Nathwani et al, N Engl J Med. 2011 Dec. 22; 365(25):2357-65, Apparailly et al, Hum Gene Ther. 2005 April; 16(4):426-34.

In some aspects, the gene therapy vector includes a retroviral vector. In some aspects, the retroviral vector is a lentiviral based expression construct. Lentiviral vectors have the ability to infect and to stably integrate into the genome of dividing and non-dividing cells (Amado and Chen, 1999 Science 285:674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2:308-16).

In some aspects, the gene therapy vector is a herpes virus vector, a polyoma virus vector or a vaccinia virus vector.

In some aspects, the gene therapy vector comprises a polynucleotide encoding an insulin and a polynucleotide encoding a glucokinase, whereby each of said polynucleotide are operably linked to the appropriate regulatory sequences. Such regulatory sequence can at least comprise a promoter sequence. Suitable promoters for expression of a nucleotide sequence encoding an insulin and/or a glucokinase from gene therapy vectors can include e.g. cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter and the herpes simplex virus thymidine kinase promoter. In some aspects, the promoter is used together with an intronic sequence. In some aspects, the CMV promoter is a mini CMV promoter.

In some aspects, the gene therapy vector includes a further nucleotide sequence coding for a further polypeptide. A further polypeptide can be a (selectable) marker polypeptide that allows for the identification, selection and/or screening for cells containing the expression construct. In some aspects, suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3^{rd} edition), Cold Spring Harbor Laboratory, Cold Spring Harbor Laboratory Press, New York.

### Non-Viral Vectors

In some aspects, a polynucleotide (e.g., polynucleotide encoding a human insulin protein or a polynucleotide encoding a human glucokinase protein) or expression cassette (e.g., insulin expression cassette or glucokinase expression cassette) of the disclosure can be administered using a non-viral vector. “Non-viral vector,” as used herein is meant to include naked DNA, chemical formulations containing naked DNA (e.g., a formulation of DNA and cationic compounds (e.g., dextran sulfate)), and naked DNA mixed with an adjuvant such as a viral particle (i.e., the DNA of interest is not contained within the viral particle, but the transforming formulation is composed of both naked DNA and viral particles (e.g., AAV particles) (see e.g., Curiel et al., Am. J. Respir. Cell Mol. Biol. 6:247-52 (1992)). Thus the “non-viral vector” can include vectors composed of DNA plus viral particles where the viral particles do not contain the DNA of interest within the viral genome.

In some aspects, a polynucleotide or expression construct of the disclosure can be complexed with polycationic substances such as poly-L-lysine or DEAC-dextran, targeting ligands, and/or DNA binding proteins (e.g., histones). DNA- or RNA-liposome complex formulations comprise a mixture of lipids which bind to genetic material (DNA or RNA) and facilitate delivery of the nucleic acid into the cell. Liposomes which can be used in accordance with the disclosure include DOPE (dioleyl phosphatidyl ethanol amine), CUDMEDA (N-(5-cholestrum-3-β-ol 3-urethanyl)-N′,N′-dimethylethylene diamine).

In some aspects, a polynucleotide or expression construct of the disclosure can also be administered as a chemical formulation of DNA or RNA coupled to a carrier molecule (e.g., an antibody or a receptor ligand) which facilitates delivery to host cells for the purpose of altering the biological properties of the host cells. The term “chemical formulations” refers to modifications of nucleic acids to allow coupling of the nucleic acid compounds to a carrier molecule such as a protein or lipid, or derivative thereof. Exemplary protein carrier molecules include antibodies specific to the target cells, i.e., molecules capable of interacting with receptors associated with a cell targeted for delivery.

### Adeno Associated Virus Vector (AAV Vector)

In some aspects, the polynucleotides (e.g., polynucleotide encoding a human insulin protein or polynucleotide encoding a human glucokinase protein) or expression cassettes (e.g., insulin expression cassette or glucokinase expression cassette) disclosed herein can be administered as a component of a packaged viral vector. In general, packaged viral vectors include a viral vector packaged in a capsid.

In some aspects, the viral vector is an AAV vector. In some aspects, an AAV vector as used herein can comprise a recombinant AAV vector (rAAV). A “rAAV vector” as used herein refers to a recombinant vector comprising part of an AAV genome encapsidated in a protein shell of capsid protein derived from an AAV serotype as disclosed herein. Part of an AAV genome can contain the inverted terminal repeats (ITR) derived from an adeno-associated virus serotype, such as AAV1, AAV2, AAV3, AAV4, AAV5, AAV6, AAV7, AAV8, AAVrh8, AAV9, AAV10, AAVrH10, AAV11, AAV12, and others. In some aspects, the ITR is derived from AAV2.

In some aspects, the combination therapy comprises an AAV vector comprising an insulin expression cassette comprising a polynucleotide encoding a human insulin protein and an AAV vector comprising a glucokinase expression cassette comprising a polynucleotide encoding a human glucokinase protein.

In some aspects, the AAV vector genome comprising an insulin expression cassette and the AAV vector genome comprising a glucokinase expression cassette are administered in a vector ratio selected from 1:0.25-0.75, 1:1.75-2.25, or 1:3.75-4.25. In some aspects, the AAV vector genome comprising an insulin expression cassette and the AAV vector genome comprising a glucokinase expression cassette are administered in a vector ratio selected from 1:0.4-0.6, 1:1.9-2.1, or 1:3.9-4.1. In some aspects, the AAV vector genome comprising an insulin expression cassette and the AAV vector genome comprising a glucokinase expression cassette are administered in a ratio of about 1:0.25-0.75, 1:0.25-0.7, 1:0.25-0.65, 1:0.25-0.60, 1:0.25-0.55, 1:0.25-0.5, 1:0.3-0.7, 1:0.3-0.65, 1:0.3-0.6, 1:0.3-0.55, 1:0.3-0.5, 1:0.35-0.7, 1:0.35-0.65, 1:0.35-0.6, 1:0.35-0.55, 1:0.35-0.5, 1:0.4-0.7, 1:0.4-0.65, 1:0.4-0.6, 1:0.4-0.55, 1:0.4-0.5, 1:0.45-0.7, 1:0.45-0.65, 1:0.45-0.6, 1:0.45-0.55, or 1:0.45-0.5. In some aspects, the AAV vector genome comprising an insulin expression cassette and the AAV vector genome comprising a glucokinase expression cassette are administered in a ratio of about 1:0.25-0.75, 1:0.3-0.75, 1:0.35-0.75, 1:0.40-0.75, 1:0.45-0.75, or 1:0.5-0.75. In some aspects, the AAV vector genome comprising an insulin expression cassette and the AAV vector genome comprising a glucokinase expression cassette are administered in a ratio of about 1:0.25-0.75, 1:0.3-0.70, 1:0.35-0.65, 1:0.4-0.60, or 1:0.45-0.55. In some aspects, the first AAV vector genome and the second AAV vector genome are in a vector ratio selected from the group consisting of 1:0.25-0.75, 1:1.75-2.25, and 1:3.75-1:4.25 (e.g., about 1:0.5, about 1:2, and about 1:4). In some aspects, the vector ratio is 1:0.4-0.6. In some aspects, the vector ratio is 1:0.5.

In some aspects, the AAV vector genome comprising an insulin expression cassette and the AAV vector genome comprising a glucokinase expression cassette are administered in a ratio of about 1:0.5, 1:2, or 1:4. In some aspects, the AAV vector genome comprising an insulin expression cassette and the AAV vector genome comprising a glucokinase expression cassette are administered in a ratio of 1:0.4-0.6 (e.g., about 1:0.5).

Typically, a vector genome requires the use of flanking 5′ and a 3′ ITR sequences to allow for efficient packaging of the vector genome into the rAAV capsid. In some aspects, the rAAV genome present in a rAAV vector comprises at least the nucleotide sequences of the inverted terminal repeat regions (ITR) of one of the AAV serotypes (e.g., of serotype AAV2 as disclosed earlier herein), or nucleotide sequences substantially identical thereto, and a modified nucleic acid sequence encoding an insulin and/or a glucokinase under control of a suitable regulatory element (e.g., a promoter), wherein the regulatory element and modified nucleic acid sequence(s) are inserted between the two ITRs.

The complete genome of several AAV serotypes and corresponding ITR has been sequenced (Chiorini et al. 1999, J. of Virology Vol. 73, No. 2, p 1309-1319). They can be either cloned or made by chemical synthesis as known in the art, using for example an oligonucleotide synthesizer as supplied e.g. by Applied Biosystems Inc. (Fosters, Calif., USA) or by standard molecular biology techniques. The ITRs can be cloned from the AAV viral genome or excised from a vector comprising the AAV ITRs. The ITR nucleotide sequences can be either ligated at either end to the nucleotide sequence encoding one or more therapeutic proteins using standard molecular biology techniques, or the wild type AAV sequence between the ITRs can be replaced with the desired nucleotide sequence.

The viral capsid component of the packaged viral vectors can be a parvovirus capsid, e.g., AAV Cap and/or chimeric capsids. Examples of suitable parvovirus viral capsid components are capsid components from the family Parvoviridae, such as an autonomous parvovirus or a Dependovirus. For example, the viral capsid may be an AAV capsid (e.g., AAV1, AAV2, AAV3, AAV4, AAV5, AAV6, AAV7, AAV8, AAVrH8 AAV9, AAV10, AAVrH10, AAV11 or AAV12 capsid; one skilled in the art would know there are likely other variants not yet identified that perform the same or similar function), or may include components from two or more AAV capsids. A full complement of AAV Cap proteins includes VP1, VP2, and VP3. The ORF comprising nucleotide sequences encoding AAV VP capsid proteins can comprise less than a full complement AAV Cap proteins or the full complement of AAV Cap proteins can be provided. In some aspects, the viral capsid is an AAV1 capsid. In some aspects the serotype of the viral vector delivering the insulin expression cassette is the same as the serotype of the viral vector delivering the glucokinase expression cassette.

One or more of the AAV Cap proteins can be a chimeric protein, including amino acid sequences AAV Caps from two or more viruses, preferably two or more AAVs. For example, the chimeric virus capsid can include an AAV1 Cap protein or subunit and at least one AAV2 Cap or subunit. In some aspects, the rAAV genome as present in a rAAV vector does not comprise any nucleotide sequences encoding viral proteins, such as the rep (replication) or cap (capsid) genes of AAV. This rAAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

In some aspects, the rAAV genome as present in said rAAV vector further comprises a promoter sequence operably linked to the polynucleotide encoding an insulin and/or a glucokinase. In some aspects, the promoter sequences are promoters which confer expression in muscle cells and/or muscle tissues. Examples of such promoters include a CMV and a RSV promoters as disclosed herein. In some aspects, the promoter is a CMV promoter. In some aspects, the promoter is used together with an intronic sequence. In some aspects, the CMV promoter is a mini CMV promoter.

In some aspects, suitable 3′ untranslated sequence can also be operably linked to the polynucleotide encoding an insulin or a glucokinase. Suitable 3′ untranslated regions can be those naturally associated with the nucleotide sequence or can be derived from different genes, such as for example the human growth hormone (hGH) or bovine growth hormone (bGH) 3′ untranslated region (e.g., hGH or bGH polyadenylation signal, SV40 polyadenylation signal, SV40 polyadenylation signal and enhancer sequence).

In some aspects, additional nucleotide sequences can be operably linked to the polynucleotides encoding an insulin and/or a glucokinase, such as nucleotide sequences encoding signal sequences, nuclear localization signals, expression enhancers, and the like.

Except as otherwise indicated, methods known to those skilled in the art may be used for the construction of recombinant parvovirus and AAV (rAAV) constructs, packaging vectors expressing the parvovirus Rep and/or Cap sequences, and transiently and stably transacted packaging cells. Such techniques are known to those skilled in the art. See, e.g., SAMBROOK et al., MOLECULAR CLONING: A LABORATORY MANUAL 2nd Ed. (Cold Spring Harbor, N.Y., 1989); AUSUBEL et al., CURRENT PROTOCOLS IN MOLECULAR BIOLOGY (Green Publishing Associates, Inc. and John Wiley Sons, Inc., New York).

Certain aspects of the disclosure are directed to a method of administering a combination therapy comprising a rAAV vector comprising a insulin expression cassette comprising a polynucleotide encoding a human insulin protein and a rAAV vector comprising a glucokinase expression cassette comprising a polynucleotide encoding a human glucokinase protein.

In some aspects, the rAAV vector genome comprising an insulin expression cassette and the rAAV vector genome comprising a glucokinase expression cassette are administered in a ratio selected from 1:0.25-0.75, 1:1.75-2.25, or 1:3.75-4.25. In some aspects, the rAAV vector genome comprising an insulin expression cassette and the rAAV vector genome comprising a glucokinase expression cassette are administered in a ratio selected from 1:0.4-0.6, 1:1.9-2.1, or 1:3.9-4.1.

In some aspects, the rAAV vector genome comprising an insulin expression cassette and the rAAV vector genome comprising a glucokinase expression cassette are administered in a ratio of about 1:0.25-0.75, 1:0.25-0.7, 1:0.25-0.65, 1:0.25-0.60, 1:0.25-0.55, 1:0.25-0.5, 1:0.3-0.7, 1:0.3-0.65, 1:0.3-0.6, 1:0.3-0.55, 1:0.3-0.5, 1:0.35-0.7, 1:0.35-0.65, 1:0.35-0.6, 1:0.35-0.55, 1:0.35-0.5, 1:0.4-0.7, 1:0.4-0.65, 1:0.4-0.6, 1:0.4-0.55, 1:0.4-0.5, 1:0.45-0.7, 1:0.45-0.65, 1:0.45-0.6, 1:0.45-0.55, or 1:0.45-0.5. In some aspects, the rAAV vector genome comprising an insulin expression cassette and the rAAV vector genome comprising a glucokinase expression cassette are administered in a ratio of about 1:0.25-0.75, 1:0.3-0.75, 1:0.35-0.75, 1:0.40-0.75, 1:0.45-0.75, or 1:0.5-0.75. In some aspects, the rAAV vector genome comprising an insulin expression cassette and the rAAV vector genome comprising a glucokinase expression cassette are administered in a ratio of about 1:0.25-0.75, 1:0.3-0.70, 1:0.35-0.65, 1:0.4-0.60, or 1:0.45-0.55. In some aspects, the first AAV vector genome and the second AAV vector genome are in a vector ratio selected from the group consisting of 1:0.25-0.75, 1:1.75-2.25, and 1:3.75-1:4.25 (e.g., about 1:0.5, about 1:2, and about 1:4). In some aspects, the vector ratio is 1:0.4-0.6. In some aspects, the vector ratio is about 1:0.5. In some aspects, the rAAV vector genome comprising an insulin expression cassette and the rAAV vector genome comprising a glucokinase expression cassette are administered in a ratio of about 1:0.5, 1:2, or 1:4. In some aspects, the rAAV vector genome comprising an insulin expression cassette and the rAAV vector genome comprising a glucokinase expression cassette are administered in a ratio of about 1:0.5.

### Host Cells

In some aspects, the present disclosure also provides host cells comprising the polynucleotides expression cassettes, vectors, or expression constructs disclosed herein. In some aspects, the host cell is a mammalian cell.

A construct prepared for introduction into a particular host can include a replication system recognized by the host, an intended DNA segment encoding a desired polypeptide, and transcriptional and translational initiation and termination regulatory sequences operably linked to the polypeptide-encoding segment. The term “operably linked” has already been defined herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognized by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for the polypeptide encoding segment can be employed. In most cases, the replication system is only functional in the cell that is used to make the vector (bacterial cell as E. Coli). Most plasmids and vectors do not replicate in the cells infected with the vector. Examples of workable combinations of cell lines and expression vectors are described in Sambrook and Russell (2001, supra) and in Metzger et al. (1988) Nature 334:31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media.

The methods of introducing exogenous nucleic acid into host cells are well known in the art, and will vary with the host cell used. Techniques include but are not limited to dextran-mediated transfection, calcium phosphate precipitation, calcium chloride treatment, polyethylenimine mediated transfection, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

Host cells may be yeast, e.g. S. cerevisiae, e.g., insect cells, e.g., Sf9 cells, mammalian cells, e.g., CHO cells, and bacterial cells, e.g., E. coli. A cell may thus be a prokaryotic or eukaryotic host cell. A cell may be a cell that is suitable for culture in liquid or on solid media. Alternatively, a host cell is a cell that is part of a multicellular organism such as a transgenic plant or animal. In some aspects, the host cell is a mammalian cell.

In some aspects, methods of introducing the viral vectors comprising the polynucleotides disclosed herein into a cellular host for replication and packaging can be employed, including but not limited to, electroporation, calcium phosphate precipitation, microinjection, cationic or anionic liposomes, and liposomes in combination with a nuclear localization signal. In embodiments wherein the viral vector functions are provided by transfection using a virus vector; standard methods for producing viral infection may be used.

In some aspects, packaging functions can include genes for viral vector replication and packaging. Thus, for example, the packaging functions may include, as needed, functions necessary for viral gene expression, viral vector replication, rescue of the viral vector from the integrated state, viral gene expression, and packaging of the viral vector into a viral particle. The packaging functions can be supplied together or separately to the packaging cell using a genetic construct such as a plasmid or an amplicon. The packaging functions can exist extrachromosomally within the packaging cell, or can be integrated into the cell's chromosomal DNA. Examples include genes encoding AAV Rep and Cap proteins.

In some aspects, helper functions can include helper virus elements needed for establishing active infection of the packaging cell, which is required to initiate packaging of the viral vector. Examples include functions derived from adenovirus, baculovirus and/or herpes virus sufficient to result in packaging of the viral vector. For example, adenovirus helper functions will typically include adenovirus components E1a, E1b, E2a, E4, and VA RNA. The packaging functions can be supplied by infection of the packaging cell with the required virus. The packaging functions can be supplied together or separately to the packaging cell using a genetic construct such as a plasmid or an amplicon. The packaging functions can exist extrachromosomally within the packaging cell, or can be integrated into the cell's chromosomal DNA.

Any suitable helper virus functions may be employed. For example, where the packaging cells are insect cells, baculovirus can serve as a helper virus. Herpes virus can also be used as a helper virus in AAV packaging methods.

Any method of introducing the nucleotide sequence carrying the helper functions into a cellular host for replication and packaging can be employed, including but not limited to, electroporation, calcium phosphate precipitation, microinjection, cationic or anionic liposomes, and liposomes in combination with a nuclear localization signal. In embodiments wherein the helper functions are provided by transfection using a virus vector or infection using a helper virus; standard methods for producing viral infection may be used.

Any suitable permissive or packaging cell known in the art can be employed in the production of the packaged viral vector. Mammalian cells or insect cells are preferred. Examples of cells useful for the production of packaging cells in the practice of the invention include, for example, human cell lines or primate cells, such as VERO, WI38, MRC5, A549, 293 cells, B-50 or any other HeLa cells, HepG2, Saos-2, HuH7, and HT1080 cell lines.

In some aspects, the cell lines for use as packaging cells are insect cell lines. Any insect cell which allows for replication of AAV and which can be maintained in culture can be used in accordance with the present invention. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, NJ (1995); O'Reilly et al., Baculovirus Expression Vectors: A Laboratory Manual, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059.

During production, the packaging cells can include one or more viral vector functions along with helper functions and packaging functions sufficient to result in replication and packaging of the viral vector. These various functions can be supplied together or separately to the packaging cell using a genetic construct such as a plasmid or an amplicon, and they can exist extrachromosomally within the cell line or integrated into the cell's chromosomes.

The cells can be supplied with any one or more of the functions already incorporated, e.g., a cell line with one or more vector functions incorporated extrachromosomally or integrated into the cell's chromosomal DNA, a cell line with one or more packaging functions incorporated extrachromosomally or integrated into the cell's chromosomal DNA, or a cell line with helper functions incorporated extrachromosomally or integrated into the cell's chromosomal DNA.

### Pharmaceutical Compositions

In some aspects, the present disclosure also provides pharmaceutical compositions comprising the polynucleotides, expression cassettes, vectors, or expression constructs disclosed herein. In some aspects there is provided a composition comprising an expression cassette or a delivery vector (e.g., a viral vector packaged in an AAV capsid) comprising a polynucleotide encoding an insulin or glucokinase as disclosed herein. In some aspects, a composition is a gene therapy composition. In some aspects, the composition is a pharmaceutical composition said pharmaceutical composition comprising a pharmaceutically acceptable carrier, adjuvant, diluents, solubilizer, filler, preservative and/or excipient.

Such pharmaceutically acceptable carrier, filler, preservative, solubilizer, diluent and/or excipient may for instance be found in Remington: The Science and Practice of Pharmacy, 20th Edition. Baltimore, Md.: Lippincott Williams & Wilkins, 2000.

In some aspects, the composition is for use as a medicament. In some aspects, the medicament is used for preventing, reducing or ameliorating the symptoms of, delaying, curing, reverting and/or treating a diabetes. In some aspects, diabetes can be Diabetes Type 1, Diabetes Type 2 or Monogenic Diabetes. In some aspects, the subject treated is a mammal, e.g. cats, rodent, (mice, rats, gerbils, guinea pigs, mice or rats), dogs, or human beings. In some aspects, the diabetes is diabetes mellitus type 1 (T1DM). In some aspects, the diabetes is diabetes mellitus type 2 (T2DM).

In some aspects, the polynucleotide, expression cassette, expression construct, delivery vector and/or composition is used for preventing, reducing or ameliorating the symptoms of, delaying, reverting, curing and/or treating a diabetes, when said the polynucleotides, expression cassettes, expression constructs, delivery vector and/or composition is able to exhibit an anti-diabetes effect. An anti-diabetes effect can be reached when glucose disposal in blood is increased and/or when glucose tolerance is improved. This can be assessed using techniques known to the skilled person. In this context, “increase” (respectively “improvement”) means at least a detectable increase (respectively a detectable improvement) using an assay known to the skilled person or using assays as carried out in the experimental part.

An anti-diabetes effect can also be observed when the progression of a typical symptom (i.e. insulitis, beta cell loss) has been slowed down as assessed by a physician. A decrease of a typical symptom associated with diabetes can mean a slowdown in progression of symptom development or a complete disappearance of symptoms. Symptoms, and also a decrease in symptoms, can be assessed using a variety of methods, to a large extent the same methods as used in diagnosis of diabetes, including clinical examination and routine laboratory tests. Such methods include both macroscopic and microscopic methods, as well as molecular methods, biochemical, immunohistochemical and others.

A medicament as defined herein (polynucleotide, expression cassette, expression construct, delivery vector, composition, etc.) is preferably able to alleviate one symptom or one characteristic of a patient or of a cell, tissue or organ of said diabetes patient if after at least one week, one month, six month, one year or more of treatment using the polynucleotide, expression cassette, viral expression construct, viral vector, or composition disclosed herein, said symptom or characteristic is decreased or no longer detectable.

A polynucleotide, expression cassette, expression construct, delivery vector, or composition as disclosed herein for use in preventing, reducing or ameliorating the symptoms of, delaying, reverting, curing and/or treating a diabetes can be suitable for administration to a cell, tissue and/or an organ in vivo of individuals affected by or at risk of developing a diabetes, and may be administered in vivo, ex vivo or in vitro. Said combination and/or composition can be directly or indirectly administrated to a cell, tissue and/or an organ in vivo of an individual affected by or at risk of developing a diabetes, and may be administered directly or indirectly in vivo, ex vivo or in vitro. In some aspects, the administration mode is intramuscular.

In some aspects, the polynucleotide, expression cassette, expression construct, delivery vector, or composition as disclosed herein can be directly or indirectly administered using suitable means known in the art. In some aspects, the polynucleotide, expression cassette, expression construct, delivery vector, or composition as disclosed herein can be delivered as is to an individual, a cell, tissue or organ of said individual. Depending on the disease or condition, a cell, tissue or organ of said individual may be as earlier defined herein. In some aspects, polynucleotide, expression cassette, expression construct, delivery vector, or composition as disclosed herein is dissolved in a solution that is compatible with the delivery method. For intravenous, subcutaneous, intramuscular, intrathecal, intraarticular and/or intraventricular administration the solution may be a physiological salt solution. In some aspects, administration is intramuscular administration. In some aspects, intramuscular administration is carried out using a multineedle. In some aspects, a therapeutically effective dose of the polynucleotide, expression cassette, expression construct, the vector, or the composition as described herein is administered in a single and unique dose hence avoiding repeated periodical administration. In some aspects, the single dose is administered to muscle tissue. In some aspects, the single dose is administered to skeletal muscle tissue. In some aspects, the single dose comprise multiple injections (e.g., two, three, four, or five) to one or more muscles (e.g., multiple muscle groups).

In some aspects, a compound can be present in a composition of the invention. Said compound can help in delivery of the polynucleotide, expression cassette, or composition comprising the same. In some aspects, the compound is a compound capable of forming complexes, nanoparticles, micelles, liposomes that deliver each constituent as defined herein, complexed or trapped in a vesicle or liposome through a cell membrane, or combinations thereof. Many of these compounds are known in the art. In some aspects, the further compound is polyethylenimine (PEI), or similar cationic polymers, including polypropyleneimine or polyethylenimine copolymers (PECs) and derivatives, synthetic amphiphiles (SAINT-18), Lipofectin™, DOTAP, or combinations thereof.

### Methods of Use

The present disclosure also provides a method for preventing, reducing or ameliorating the symptoms of, delaying, reverting, curing and/or treating diabetes comprising administering to a subject in need thereof a combination therapy comprising the polynucleotides, the expression cassettes, the delivery vectors, or expression constructs disclosed herein. In some aspects, the diabetes can be T1DM. In some aspects, the diabetes can be T2DM. In some aspects, the method is a gene therapy. In certain aspects, the methods of the disclosure comprise administration (e.g., intramuscular administration) a combination therapy comprising the polynucleotides, the expression cassettes, a delivery vectors, or expression construct disclosed herein to a cell, tissue, or subject in need thereof. In certain aspects, the methods comprise administration of a combination therapy comprising (i) a polynucleotide encoding a human insulin protein, an insulin expression cassette, a delivery vectors, or expression construct encoding a human insulin (Ins) protein (e.g., a preproinsulin or variant thereof) and/or (ii) a polynucleotide encoding a human glucokinase protein, a glucokinase expression cassette, a delivery vectors, or expression construct comprising a nucleic acid encoding a human glucokinase (Gck) protein. In certain aspects, the administration of (i) and (ii) is simultaneous or sequential.

In some aspects, (i) the polynucleotide encoding a human insulin protein, the insulin expression cassette, the delivery vector or expression construct encoding a human insulin (Ins) protein (e.g., a preproinsulin or variant thereof) and/or (ii) the polynucleotide encoding a human glucokinase protein, the glucokinase expression cassette, the delivery vector or expression construct comprising a nucleic acid encoding a human glucokinase (Gck) protein are administered in a ratio selected from the group consisting of about 1:0.25-0.75, 1:0.25-0.7, 1:0.25-0.65, 1:0.25-0.60, 1:0.25-0.55, 1:0.25-0.5, 1:0.3-0.7, 1:0.3-0.65, 1:0.3-0.6, 1:0.3-0.55, 1:0.3-0.5, 1:0.35-0.7, 1:0.35-0.65, 1:0.35-0.6, 1:0.35-0.55, 1:0.35-0.5, 1:0.4-0.7, 1:0.4-0.65, 1:0.4-0.6, 1:0.4-0.55, 1:0.4-0.5, 1:0.45-0.7, 1:0.45-0.65, 1:0.45-0.6, 1:0.45-0.55, and 1:0.45-0.5.

In some aspects, (i) the polynucleotide encoding a human insulin protein, the insulin expression cassette, the delivery vector or expression construct encoding a human insulin (Ins) protein (e.g., a preproinsulin or variant thereof) and/or (ii) the polynucleotide encoding a human glucokinase protein, the glucokinase expression cassette, the delivery vector or expression construct comprising a nucleic acid encoding a human glucokinase (Gck) protein are administered in a ratio selected from the group consisting of about 1:0.25-0.75, 1:0.3-0.75, 1:0.35-0.75, 1:0.40-0.75, 1:0.45-0.75, and 1:0.5-0.75.

In some aspects, (i) the polynucleotide encoding a human insulin protein, the insulin expression cassette, the delivery vector or expression construct encoding a human insulin (Ins) protein (e.g., a preproinsulin or variant thereof) and/or (ii) the polynucleotide encoding a human glucokinase protein, the glucokinase expression cassette, the delivery vector or expression construct comprising a nucleic acid encoding a human glucokinase (Gck) protein are administered in a ratio selected from the group consisting of about 1:0.25-0.75, 1:0.3-0.70, 1:0.35-0.65, 1:0.4-0.60, and 1:0.45-0.55.

In some aspects, (i) the polynucleotide encoding a human insulin protein, the insulin expression cassette, the delivery vector or expression construct encoding a human insulin (Ins) protein (e.g., a preproinsulin or variant thereof) and/or (ii) the polynucleotide encoding a human glucokinase protein, the glucokinase expression cassette, the delivery vector or expression construct comprising a nucleic acid encoding a human glucokinase (Gck) protein are administered in a ratio selected from the group consisting of 1:0.25-0.75, 1:1.75-2.25, and 1:3.75-1:4.25 (e.g., about 1:0.5, about 1:2, and about 1:4).

Certain aspects of the disclosure are directed methods of use comprising administering combination therapy a polynucleotide encoding a human insulin (Ins) protein (e.g., a preproinsulin or variant thereof) comprising (i) a nucleotide sequence encoding a signal peptide, optionally wherein the signal peptide is not a wild-type preproinsulin signal sequence, and (ii) a nucleotide sequence encoding a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid position in wild-type proinsulin, and optionally the polynucleotide further comprises a cleavage site. In some aspects, the signal peptide is a wild-type preproinsulin signal sequence, an IL-6 signal sequence, or a fibronectin signal sequence. In some aspects, the cleavage site between the signal sequence and the circulating protein is a furin cleavage site.

Certain aspects of the disclosure are directed to methods of use comprising administering a combination therapy comprising a polynucleotide comprising a nucleic acid encoding a human insulin (Ins) protein, wherein the nucleic acid comprises an open reading frame (ORF) comprising: (i) a nucleotide sequence encoding a signal peptide and (ii) a nucleotide sequence at least 85%, 86%, 87%, 88%, 89%, 90%, 91%, 92%, 93%, 94%, 95%, 96%, 97%, 98%, 99%, or 100% identical to nucleic acids 73-330 of any of SEQ ID NOs: 43-57, 110-116, 150-151, 154-155, and 157-159, nucleic acids 88-345 of any of SEQ ID NOs: 117-122, 152, and 156, or nucleic acids 79-336 of SEQ ID NO: 153. In some aspects, the encoded human Ins protein comprises (i) a signal peptide (e.g., a wild-type preproinsulin signal sequence, an IL-6 signal sequence, or a fibronectin signal sequence) and (ii) amino acids 25-110 of SEQ ID NO: 41, amino acids 25-110 of SEQ ID NO: 144, or amino acids 25-110 of SEQ ID NO: 145. In some aspects, the encoded human insulin protein further comprises a cleavage site (e.g., a furin cleavage site).

Certain aspects of the disclosure are directed to a method of use comprising administering combination therapy comprising a polynucleotide comprising a nucleic acid encoding a human insulin (Ins) protein, wherein the nucleic acid comprises an open reading frame (ORF) comprising: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 43-57, 110-122, or 150-159. In some aspects, the encoded human Ins protein comprises a signal sequence and a proinsulin polypeptide. In some aspects, the encoded human Ins protein comprises the amino acid sequence of any of amino acids 25-110 of SEQ ID NO: 41, amino acids 25-110 of SEQ ID NO: 144, or amino acids 25-110 of SEQ ID NO: 145. In some aspects, the encoded human Ins protein is a preproinsulin. In some aspects, the encoded human Ins protein comprises the amino acid sequence of SEQ ID NO: 41, SEQ ID NO: 144, or SEQ ID NO: 145. In some aspects, the polynucleotide or nucleic acid sequence further comprises a 5′ UTR and/or a 3′ UTR. In some aspects, the polynucleotide or nucleic acid comprises: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 1-16, 84-88, 123, 127-129, 133-138, 160-161, or 170.

Certain aspects of the disclosure are directed to a method of use comprising administering a combination therapy comprising a polynucleotide comprising a nucleic acid encoding a human insulin (Ins) protein (e.g., a preproinsulin or variant thereof), wherein the nucleic acid comprises: (i) a nucleotide sequence encoding a signal peptide (e.g., a wild-type preproinsulin signal sequence, an IL-6 signal sequence, or a fibronectin signal sequence) and (ii) a nucleotide sequence encoding a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid in wild-type proinsulin (or an amino acid modification at a position selected from amino acid H34, P52, K53, R55, L86, or any combination thereof relative to the corresponding amino acid in wild-type preproinsulin). In some aspects, the signal peptide is not a wild-type preproinsulin signal sequence (e.g., the wild-type preproinsulin sequence is replaced with an IL-6 signal sequence or fibronectin signal sequence). In some aspects, the proinsulin polypeptide comprises the amino acid sequence of any of amino acids 25-110 of SEQ ID NO: 41, amino acids 25-110 of SEQ ID NO: 144, or amino acids 25-110 of SEQ ID NO: 145. In some aspects, the polynucleotide further comprises a cleavage site (e.g., a furin cleavage site).

Certain aspects of the disclosure are directed to a method of use comprising administering combination therapy comprising a polynucleotide comprising a nucleic acid encoding a human glucokinase (Gck) protein, wherein the nucleic acid comprises an ORF comprising: a nucleotide sequence at least 85%, 86%, 87%, 88%, 89%, 90%, 91%, 92%, 93%, 94%, 95%, 96%, 97%, 98%, 99%, or 100% identical to any of nucleic acids 1-1398 of any of SEQ ID NO: 61-80 or 162; or SEQ ID NO: 61-80 and 162. In some aspects, the encoded human Gck protein comprises the amino acid sequence of SEQ ID NO: 82. In some aspects, the polynucleotide or nucleic acid sequence encoding a Gck protein further comprises a 5′ UTR and/or a 3′ UTR. In some aspects, the nucleic acid further comprises a 5′ UTR comprising a nucleotide sequence at least 85%, 90%, 95%, 99%, or 100% identical to SEQ ID NO: 42, 5-329 of SEQ ID NO: 42, SEQ ID NO: 83, SEQ ID NO: 146, or SEQ ID NO: 148. In some aspects, the nucleic acid further comprises a 3′ UTR comprising a nucleotide sequence at least 85%, 90%, 95%, 99%, or 100% identical to SEQ ID NO: 60, SEQ ID NO: 97, SEQ ID NO: 98, SEQ ID NO: 99, SEQ ID NO: 100, SEQ ID NO: 101, SEQ ID NO: 149, or SEQ ID NO: 169. In some aspects, the polynucleotide or nucleic acid comprises: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 20-39 and 89-96, 163-164, or 168. In some aspects, the nucleic acid is operably linked to a promoter (e.g., a eukaryotic promoter). Certain aspects of the disclosure are directed to an expression cassette comprising a polynucleotide of the disclosure and a heterologous expression control sequence operably linked to the nucleic acid sequence. In some aspects, the nucleic acid is operably linked to a polyadenylation (polyA) element.

Certain aspects of the disclosure are directed to a method of use comprising administering combination therapy comprising a polynucleotide comprising a nucleic acid encoding a human insulin (Ins) protein, wherein the nucleic acid comprises an open reading frame (ORF) comprising: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 43-57, 110-122, or 150-159 and a polynucleotide comprising a nucleic acid encoding a human glucokinase (Gck) protein, wherein the nucleic acid comprises an ORF comprising: a nucleotide sequence at least 85%, 86%, 87%, 88%, 89%, 90%, 91%, 92%, 93%, 94%, 95%, 96%, 97%, 98%, 99%, or 100% identical to any of nucleic acids 1-1398 of any of SEQ ID NO: 61-80 or 162; or SEQ ID NO: 61-80 and 162.

Certain aspects of the disclosure are directed to a method of use comprising administering combination therapy comprising a polynucleotide or nucleic acid encoding a human insulin protein wherein the polynucleotide comprises a sequence at least 85%, 86%, 87%, 88%, 89%, 90%, 91%, 92%, 93%, 94%, 95%, 96%, 97%, 98%, 99%, or 100% identical to any of SEQ ID NOs: 1-16, 84-88, 123, 127-129, 133-138, 160-161, or 170 and polynucleotide or nucleic acid encoding a human glucokinase protein wherein the polynucleotide comprises a sequence at least 85%, 86%, 87%, 88%, 89%, 90%, 91%, 92%, 93%, 94%, 95%, 96%, 97%, 98%, 99%, or 100% identical to any of SEQ ID NOs: 20-39 and 89-96, 163-164, and 168.

Certain aspects of the disclosure are directed to methods of use comprising administering a combination therapy comprising a vector (e.g., viral vector, a non-viral vector, a plasmid, a lipid, or a lysosome) comprising a polynucleotide or an expression cassette of the disclosure. In some aspects, the vector is an adeno-associated virus (AAV) vector or a Lentivirus vector.

Certain aspects of the disclosure are directed to a method of administering a combination therapy comprising an AAV vector comprising an insulin expression cassette comprising a polynucleotide encoding a human insulin protein and a AAV vector comprising a glucokinase expression cassette comprising a polynucleotide encoding a human glucokinase protein. In some aspects, the serotype of the AAV vector delivering the insulin expression cassette is the same as the serotype of the AAV vector delivering the glucokinase expression cassette. In some aspects, the serotype of the AAV vector delivering the insulin expression cassette is different from the serotype of the AAV vector delivering the glucokinase expression cassette.

In some aspects, the AAV vector genome comprising an insulin expression cassette and the AAV vector genome comprising a glucokinase expression cassette are administered in a vector ratio selected from 1:0.25-0.75, 1:1.75-2.25, or 1:3.75-4.25. In some aspects, the AAV vector genome comprising an insulin expression cassette and the AAV vector genome comprising a glucokinase expression cassette are administered in a vector ratio selected from 1:0.4-0.6, 1:1.9-2.1, or 1:3.9-4.1. In some aspects, the AAV vector genome comprising an insulin expression cassette and the AAV vector genome comprising a glucokinase expression cassette are administered in a vector ratio of about 1:0.25-0.75, 1:0.25-0.7, 1:0.25-0.65, 1:0.25-0.60, 1:0.25-0.55, or 1:0.25-0.5. In some aspects, the AAV vector genome comprising an insulin expression cassette and the AAV vector genome comprising a glucokinase expression cassette are administered in a vector ratio of about 1:0.25-0.75, 1:0.3-0.75, 1:0.35-0.75, 1:0.40-0.75, 1:0.45-0.75, or 1:0.5-0.75. In some aspects, the AAV vector genome comprising an insulin expression cassette and the AAV vector genome comprising a glucokinase expression cassette are administered in a vector ratio of about 1:0.25-0.75, 1:0.3-0.70, 1:0.35-0.65, 1:0.4-0.60, or 1:0.45-0.55. In some aspects, the first AAV vector genome and the second AAV vector genome are in a vector ratio selected from the group consisting of 1:0.25-0.75, 1:1.75-2.25, and 1:3.75-1:4.25 (e.g., about 1:0.5, about 1:2, and about 1:4). In some aspects, the vector ratio is 1:0.4-0.6. In some aspects, the vector ratio is 1:0.5. In some aspects, the AAV vector genome comprising an insulin expression cassette and the AAV vector genome comprising a glucokinase expression cassette are administered in a vector ratio of about 1:0.5, 1:2, or 1:4. In some aspects, the AAV vector genome comprising an insulin expression cassette and the AAV vector genome comprising a glucokinase expression cassette are administered in a vector ratio of about 1:0.5.

Certain aspects of the disclosure are directed to a method of administering combination therapy comprising a recombinant AAV (rAAV) particle, comprising an AAV capsid and a vector genome comprising the polynucleotide or the expression cassette of the disclosure. Certain aspects of the disclosure are directed to a method of administering a combination therapy comprising a recombinant AAV (rAAV) particle comprising a insulin expression cassette comprising a polynucleotide encoding a human insulin protein and a rAAV particle comprising a glucokinase expression cassette comprising a polynucleotide encoding a human glucokinase protein. In some aspects, the AAV serotype is selected from the group consisting of AAV1, AAV2, AAV3, AAV4, AAV5, AAV6, AAV7, AAV8, AAV8, AAVrh9, AAV9, AAVrh10, AAV10, AAV11, and AAV12.

In some aspects, the rAAV vector genome comprising an insulin expression cassette and the rAAV vector genome comprising a glucokinase expression cassette are administered in a ratio selected from 1:0.25-0.75, 1:1.75-2.25, or 1:3.75-4.25. In some aspects, the rAAV vector genome comprising an insulin expression cassette and the rAAV vector genome comprising a glucokinase expression cassette are administered in a ratio selected from 1:0.4-0.6, 1:1.9-2.1, or 1:3.9-4.1.

In some aspects, the rAAV vector genome comprising an insulin expression cassette and the rAAV vector genome comprising a glucokinase expression cassette are administered in a ratio selected from about 1:0.25-0.75, 1:0.25-0.7, 1:0.25-0.65, 1:0.25-0.60, 1:0.25-0.55, or 1:0.25-0.5. In some aspects, the rAAV vector genome comprising an insulin expression cassette and the rAAV vector genome comprising a glucokinase expression cassette are administered in a ratio selected from about 1:0.25-0.75, 1:0.3-0.75, 1:0.35-0.75, 1:0.40-0.75, 1:0.45-0.75, or 1:0.5-0.75. In some aspects, the rAAV vector genome comprising an insulin expression cassette and the rAAV vector genome comprising a glucokinase expression cassette are administered in a ratio selected from about 1:0.25-0.75, 1:0.3-0.70, 1:0.35-0.65, 1:0.4-0.60, or 1:0.45-0.55.

In some aspects, the rAAV vector genome comprising an insulin expression cassette and the rAAV vector genome comprising a glucokinase expression cassette are administered in a ratio selected from 1:0.25-0.75, 1:1.75-2.25, or 1:3.75-1:4.25 (e.g., about 1:0.5, about 1:2, and about 1:4). In some aspects, the rAAV vector genome comprising an insulin expression cassette and the rAAV vector genome comprising a glucokinase expression cassette are administered in a ratio of about 1:0.5.

In some aspects, the combination therapy is prepared as a dosage form for administration to a subject by combining a composition comprising a AAV vector comprising an insulin expression cassette comprising a polynucleotide encoding a human insulin protein disclosed herein and a composition comprising a AAV vector comprising a glucokinase expression cassette comprising a polynucleotide encoding a human glucokinase protein disclosed herein, wherein each composition in an amount sufficient to provide a vector ratio selected from the group consisting of 1:0.25-0.75, 1:1.75-2.25, and 1:3.75-4.25. In some aspects, the vector ratio is selected from 1:0.4-0.6, 1:1.9-2.1, or 1:3.9-4.1. In some aspects, the ratio is about 1:0.25-0.75, 1:0.3-0.75, 1:0.35-0.75, 1:0.40-0.75, 1:0.45-0.75, 1:0.5-0.75, 1:0.3-0.7, 1:0.3-0.65, 1:0.3-0.6, 1:0.3-0.55, 1:0.3-0.5, 1:0.35-0.7, 1:0.35-0.65, 1:0.35-0.6, 1:0.35-0.55, 1:0.35-0.5, 1:0.4-0.7, 1:0.4-0.65, 1:0.4-0.6, 1:0.4-0.55, 1:0.4-0.5, 1:0.45-0.7, 1:0.45-0.65, 1:0.45-0.6, 1:0.45-0.55, or 1:0.45-0.5. In some aspects, the vector ratio is about 1:0.5, about 1:2, and about 1:4. In some aspects, the vector ratio is 1:0.4-0.6. In some aspects, the vector ratio is about 1:0.5. In some aspects, the combination therapy comprises a mixture of an AAV vector comprising an insulin expression cassette and an AAV vector comprising a glucokinase expression cassette in the same pharmaceutical formulation (e.g., with a vector ratio of 1:0.25-0.75, e.g., about 1:0.5). In some aspects, the combination therapy comprises an AAV vector comprising an insulin expression cassette and an AAV vector comprising a glucokinase expression cassette in separate pharmaceutical formulations administered simultaneously or sequentially according to a ratio disclosed herein.

Certain advantages for the gene therapy methods disclosed herein include the potential for administration of the polynucleotides, the expression cassettes, the delivery vectors, or expression constructs disclosed herein that provides the therapeutic gene expression through the lifetime of the diabetic subject. WO 2012/007458 discloses the generation of two viral vectors, one expressing the insulin gene and one expressing the glucokinase gene as a treatment of diabetes. Furthermore, WO 2016/110518 discloses single-vector gene constructs comprising insulin and glucokinase genes. In certain aspects, the present disclosure provides improved nucleic acid sequences, expression constructs, and/or delivery vectors for diabetes treatment or prevention having increased expression of insulin and glucokinase, decreased adverse immune reaction, and/or allowing for administration of a lower dose of viral vector.

In some aspects, the methods of the disclosure alleviates or reduces one or more symptom(s) of diabetes in an individual, in a cell, tissue or organ of said individual or alleviates or reduces one or more characteristic(s) or symptom(s) of a cell, tissue or organ of said individual, the method comprising administering to said individual one or more of the polynucleotides, expression cassettes, vectors, or expression constructs disclosed herein.

Treatment recommendations for adults with diabetes generally target a HbA1c <7.0% without significant hypoglycemia. In some aspects, the ‘normal range’ for HbA1c is <7.0%, e.g., <6.5%, <6.0%, <5.7%, e.g., between about 5.0% and about 6.5%. Most marketed products lower HbA1c between 0.5% and 1.50%. In some aspects, the methods of the disclosure normalize HbA1c levels in a treated diabetic subject to HbA1c levels in a non-diabetic subject, e.g., within 8 weeks. In some aspects, the methods of the disclosure allow for reduction and/or regulation of glycated blood hemoglobin (HbA1c) levels in the subject. In some aspects, the HbA1c levels in a subject after treatment are <7.0% (e.g., <6.5%, <6.0%, <5.7%, e.g., between 5.0% and 6.5%), e.g., within 8 weeks after treatment.

Insulin plays a central role in the regulation of lipid metabolism in liver, adipose, and gut (Vergès B. Insulin sensitivity and lipids. Diabetes Metab. 2001 April; 27(2 Pt 2):223-7. PMID: 11452214.). In uncontrolled Type 1 diabetes, patients are unable to utilize glucose, requiring an alternative fuel source. In adipose tissue, insulin inhibits hormone-sensitive lipase normally promoting storage of triglycerides in the adipocytes and reducing release of free fatty acids from adipose tissue in the circulation. When circulating insulin levels are low, there is a profound reduction of lipoprotein catabolism (Taskinen M R. Lipoprotein lipase in diabetes. Diabetes Metab Rev. 3:551-570. 1987 doi: 10.1002/dmr.5610030208. 1987). Lipolysis, resulting in an increased level of circulating triglyceride-rich lipoproteins (chylomicrons, VLDLs), occurs leading to hypertriglyceridemia. In some aspects, the methods of the disclosure reduce the level of a triglyceride-rich lipoprotein (e.g., chylomicrons or VLDLs) in a subject (e.g., a subject suffering from diabetes), in a cell, tissue or organ of said subject, the method comprising administering to the subject one or more of the polynucleotides, expression cassettes, vectors, or expression constructs disclosed herein.

In the liver, ketone bodies (β-hydroxybutyrate (β-HB) and acetoacetate (AcAc)) are produced by the β-oxidation of fatty acids. During fasting or dietary carbohydrate restriction, ketones serve as a source of alternative energy in glucose limiting conditions and can provide up to 80% of the brain's energy requirements. While useful in the short term, a chronic elevation of circulating ketones can produce unwanted effects in the brain, kidney, liver, and microvasculature (Kanikarla-Marie P, Jain S K. Hyperketonemia and ketosis increase the risk of complications in type 1 diabetes. Free Radic Biol Med. 95:268-277, 2016. doi: 10.1016/j.freeradbiomed.2016.03.020) and a resulting ketoacidosis which can be fatal. In some aspects, the methods of the disclosure reduce the level of a ketones in a subject (e.g., a subject suffering from diabetes), in a cell, tissue or organ of said subject, the method comprising administering to the subject one or more of the polynucleotides, expression cassettes, vectors, or expression constructs disclosed herein

In some aspects, the methods of the disclosure provide (i) reduction and/or regulation of glycated blood hemoglobin (HbA1c) levels in the subject; (ii) reduction in circulating ketones in the subject, (iii) reduction in triglycerides in the subject, or (iv) any combination thereof.

In some aspects, the method or use is performed in vitro, for instance using a cell culture. In some aspects, the method or use is performed in vivo. In some aspects, a polynucleotide, an expression cassette, a delivery vectors, or expression construct disclosed herein is combined with an additional compound known to be used for treating diabetes in an individual. In some aspects, the method further comprises administering recombinant insulin, e.g., via regular injections.

In some aspects, the method disclosed herein is not repeated. In some aspects, the method disclosed herein is repeated each year or each 2, 3, 4, 5, 6, 7, 8, 9, or 10 years.

In some aspects, the method comprises administering a therapeutically effective dose of the combination therapy comprising the polynucleotides, expression constructs, the vectors, or the compositions as described herein, wherein the administration is a single, e.g., avoiding repeated periodical administration. In some aspects, the single dose is administered to muscle tissue. In some aspects, the single dose is administered to skeletal muscle tissue. In some aspects, the single dose comprise multiple injections (e.g., two, three, four, or five) to one or more muscles (e.g., multiple muscle groups).

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The breadth and scope of the present invention should not be limited by any of the above-described exemplary embodiments, but should be defined in accordance with the following claims and their equivalents.

Having described the present invention, the same will be explained in greater detail in the following examples, which are included herein for illustration purposes only, and which are not intended to be limiting to the invention.

### EXAMPLES

### Example 1: Insulin Nucleic Acids

The following human insulin nucleic acid sequences (shown in Table 1) corresponding to SEQ ID Nos: 1-16, 84-88, 123, 127-129, 133-138, 160-161, and 170 were designed in silico. 5′UTR sequences (SEQ ID NO: 42, SEQ ID NO: 83, SEQ ID NO: 146, or SEQ ID NO: 148) are in BOLD, ORF sequences are underlined (SEQ ID NOs: 43-57, 110-122), and 3′ UTR sequences are italicized (SEQ ID NO: 60, SEQ ID NO: 97, SEQ ID NO: 98, SEQ ID NO: 99, SEQ ID NO: 100, SEQ ID NO: 101, or SEQ ID NO: 149). The 5′UTRs having the sequence of SEQ ID NO: 42 was further modified to remove the CTAG at positions 1-4. Accordingly, in certain constructs the 5′UTR included nucleic acids 5-329 of SEQ ID NO: 42.

TABLE 1
Nucleic Acid Sequences Encoding Human Insulin

| SEQ ID | | |
|---|---|---|
| NOs | Name | Sequence |
| Full: 1 | Unmodified | AAGCTTTATTGCGGTAGTTTATCACAGTTAAATTGCTAACGCAGTCAG |
| 5′ UTR: 148 | hIns- | TGCTTCTGACACAACAGTCTCGAACTTAAGCTGCAGTGACTCTCTTAA |
| ORF: 110 | 1 (B10D) | GGTAGCCTTGCAGAAGTTGGTCGTGAGGCACTGGGCAGGTAAGTATC |
| 3′ UTR: 149 | | AAGGTTACAAGACAGGTTTAAGGAGACCAATAGAAACTGGGCTTGTC |
| | | GAGACAGAGAAGACTCTTGCGTTTCTGATAGGCACCTATTGGTCTTA |
| | | CTGACATCCACTTTGCCTTTCTCTCCACAGGTGTCCACTCCCAGTTCA |
| | | ATTACAGCTCTTAAGGCTAGAGTACTTAATACGACTCACTATAGGCTA |
| | | GCCTCGAGAATTCTGCCATGGCCCTGTGGATGCGCCTCCTGCCCCTGCT |
| | | GGCGCTGCTGGCCCTCTGGGGACCTGACCCAGCCGCAGCCTTTGTGAACC |
| | | AACACCTGTGCGGCTCAGATCTGGTGGAAGCTCTCTACCTAGTGTGCGGG |
| | | GAACGAGGCTTCTTCTACACACCCAGGACCAAGCGGGAGGCAGAGGACC |
| | | TGCAGGTGGGGCAGGTGGAGCTGGGCGGGGGCCCTGGTGCAGGCAGCCT |
| | | GCAGCCCTTGGCCCTGGAGGGGTCGCGACAGAAGCGTGGCATTGTGGAA |
| | | CAATGCTGTACCAGCATCTGCTCCCTCTACCAGCTGGAGAACTACTGCAA |
| | | CTAGACGCAGCTGCTAGCTAATCGATACCGTCGACCTCGAGGAATTCACGCGT |
| | | GGTACCTCTAGAGTCGACCCGGGCGGCCGCTTCCCTTTAGTGAGGGTTAATG |
| | | CTTCGAGCAGACATGATAAGATACATTGATGAGTTTGGACAAACCACAACTAGA |
| | | ATGCAGTGAAAAAAATGCTTTATTTGTGAAATTTGTGATGCTATTGCTTTATTTGT |
| | | AACCATTATAAGCTGCAATAAACAAGTTAACAACAACAATTGCATTCATTTTATG |
| | | TTTCAGGTTCAGGGGGAGATGTGGGAGGTTTTTTAAAGCAAGTAAAACCTCTAC |
| | | AAATGTGGTAAAATCCGATAAGGGACTAGAGCAGAAGCTTTTG |
| Full: 2 | Modified | [CTAG]AAGCTTTATTGTGGTAGTTTATCACAGTTAAATTGCTAAGGCA |
| 5′UTR: 42 | hIns-2 | GTCAGTGCTTCTGACACAACAGTCTCCAACTTAAGCTGCAGTGACTC |
| ORF: 43 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCACTGCTGGCCCTCTGG |
| | | GGACCTGACCCAGCAGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGGGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGTCCAGACAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 3 | Modified | [CTAG]AAGCTTTATTGTGGTAGTTTATCACAGTTAAATTGCTAAGGCA |
| 5′UTR: 42 | hIns-3 | GTCAGTGCTTCTGACACAACAGTCTCCAACTTAAGCTGCAGTGACTC |
| ORF: 44 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGGCTCCTGCCCCTGCTGGCCCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGGGGCTTCTTCTACA |
| | | CACCCAGGACCAAGAGAGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGAGCAGGCAGAAGAGGGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 4 | Modified | [CTAG]AAGCTTTATTGTGGTAGTTTATCACAGTTAAATTGCTAAGGCA |
| 5′UTR: 42 | hIns-4 | GTCAGTGCTTCTGACACAACAGTCTCCAACTTAAGCTGCAGTGACTC |
| ORF: 45 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGGCTCCTGCCCCTGCTGGCTCTGCTGGCCCTCTGG |
| | | GGACCTGACCCAGCAGCAGCCTTTGTGAACCAACACCTGTGTGGCTCAGA |
| | | TCTGGTGGAAGCTCTCTACCTAGTGTGTGGGGAAAGGGGCTTCTTCTACA |
| | | CACCCAGGACCAAGAGAGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGAGCAGGCAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 5 | Modified | [CTAG]AAGCTTTATTGTGGTAGTTTATCACAGTTAAATTGCTAAGGCA |
| 5′UTR: 42 | hIns-5 | GTCAGTGCTTCTGACACAACAGTCTCCAACTTAAGCTGCAGTGACTC |
| ORF: 46 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTGCTCCCCCTCCTGGCTTTGCTGGCCCTGTGGG |
| | | GACCAGATCCTGCAGCTGCCTTTGTGAATCAGCACCTGTGTGGCTCAGAT |
| | | CTGGTTGAGGCACTCTACCTGGTGTGTGGGGAGAGAGGCTTCTTTTACAC |
| | | ACCAAGGACCAAGAGGGAGGCAGAGGATCTGCAGGTAGGTCAGGTGGA |
| | | GCTGGGAGGTGGCCCTGGAGCAGGATCCCTGCAGCCCTTGGCACTGGAG |
| | | GGCTCCAGACAGAAGAGGGGCATTGTGGAACAGTGTTGCACTAGCATCT |
| | | GCAGCCTGTACCAGCTGGAGAACTACTGCAACTAAAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 6 | Modified | [CTAG]AAGCTTTATTGTGGTAGTTTATCACAGTTAAATTGCTAAGGCA |
| 5′UTR: 42 | hIns-6 | GTCAGTGCTTCTGACACAACAGTCTCCAACTTAAGCTGCAGTGACTC |
| ORF: 47 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGCCTCCTGCCCCTGCTGGCGCTGCTGGCCCTCTGG |
| | | GGACCTGACCCAGCCGCAGCCTTTGTGAACCAACACCTGTGCGGCTCAGA |
| | | TCTGGTGGAAGCTCTCTACCTAGTGTGCGGGGAAAGAGGCTTCTTCTACA |
| | | CACCCAGGACCAAGCGGGAGGCAGAGGACCTGCAGGTGGGGCAGGTGG |
| | | AGCTGGGCGGGGGCCCTGGTGCAGGCAGCCTGCAGCCCTTGGCCCTGGA |
| | | GGGGTCGCGACAGAAGCGGGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 7 | Modified | [CTAG]AAGCTTTATTGTGGTAGTTTATCACAGTTAAATTGCTAAGGCA |
| 5′UTR: 42 | hIns-7 | GTCAGTGCTTCTGACACAACAGTCTCCAACTTAAGCTGCAGTGACTC |
| ORF: 48 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGCCTCCTGCCCCTGCTGGCGCTGCTGGCCCTCTGG |
| | | GGACCTGACCCAGCCGCAGCCTTTGTGAACCAACACCTGTGCGGCTCAGA |
| | | TCTGGTGGAAGCTCTCTACCTAGTGTGCGGGGAAAGAGGCTTCTTCTACA |
| | | CACCCAGGACCAAGCGGGAGGCAGAGGACCTGCAGGTGGGGCAGGTGG |
| | | AGCTGGGCGGGGGCCCTGGTGCAGGCAGCCTGCAGCCCTTGGCCCTGGA |
| | | GGGGTCGCGACAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 8 | Modified | [CTAG]AAGCTTTATTGTGGTAGTTTATCACAGTTAAATTGCTAAGGCA |
| 5′UTR: 42 | hIns-8 | GTCAGTGCTTCTGACACAACAGTCTCCAACTTAAGCTGCAGTGACTC |
| ORF: 49 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCTCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGGGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGTCTAGACAGAAGAGGGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 9 | Modified | [CTAG]AAGCTTTATTGTGGTAGTTTATCACAGTTAAATTGCTAAGGCA |
| 5′UTR: 42 | hIns-9 | GTCAGTGCTTCTGACACAACAGTCTCCAACTTAAGCTGCAGTGACTC |
| ORF: 50 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCCCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGAGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGAGCAGACAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 10 | Modified | [CTAG]AAGCTTTATTGTGGTAGTTTATCACAGTTAAATTGCTAAGGCA |
| 5′UTR: 42 | hIns-10 | GTCAGTGCTTCTGACACAACAGTCTCCAACTTAAGCTGCAGTGACTC |
| ORF: 51 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCTCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGGGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGAGTAGACAGAAGAGGGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 11 | Modified | [CTAG]AAGCTTTATTGTGGTAGTTTATCACAGTTAAATTGCTAAGGCA |
| 5′UTR: 42 | hIns-11 | GTCAGTGCTTCTGACACAACAGTCTCCAACTTAAGCTGCAGTGACTC |
| ORF: 52 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCTCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGAGAGGCAGAGGACCTGCAGGTGGGGCAGGTGG |
| | | AGCTGGGTGGGGGCCCTGGTGCAGGCAGCCTGCAGCCCTTGGCCCTGGA |
| | | GGGGTCTAGACAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 12 | Modified | [CTAG]AAGCTTTATTGTGGTAGTTTATCACAGTTAAATTGCTAAGGCA |
| 5′UTR: 42 | hIns-12 | GTCAGTGCTTCTGACACAACAGTCTCCAACTTAAGCTGCAGTGACTC |
| ORF: 53 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGGCTCTTGCCCCTTCTGGCTTTACTGGCACTATGGG |
| | | GCCCTGACCCAGCGGCCGCTTTCGTGAACCAGCACCTCTGCGGAAGCGAT |
| | | CTGGTCGAGGCCTTGTACCTGGTTTGTGGGGAAAGAGGTTTTTTCTATACC |
| | | CCGAGGACAAAGCGCGAGGCAGAAGACCTTCAAGTGGGCCAGGTAGAGC |
| | | TCGGAGGGGGCCCCGGTGCCGGATCCCTGCAGCCTCTGGCTCTCGAAGGG |
| | | TCTCGACAAAAACGTGGCATCGTGGAGCAGTGCTGTACTTCAATTTGCAG |
| | | TTTGTACCAGCTTGAGAATTATTGTAACTGAAGCCAGCTGCCTTCCCTTTAC |
| | | TGTGGGTTAATGCTTGCAGCAGACATGATAAGATACATTGATGAGTTTGGACAA |
| | | ACCACAACTAGAATGCAGTGAAAAAAATGCTTTATTTGTGAAATTTGTGATGCTA |
| | | TTGCTTTATTTGTAACCATTATAAGCTGCAATAAACAAGTTAACAACAACAATTG |
| | | CATTCATTTTATGTTTCAGGTTCAGGGGGAGATGTGGGAGGTTTTTTAAAGCAA |
| | | GTAAAACCTCTACAAATGTGGTAAAATCCGATAAGGGACTAGAGCAGAAGCTTT |
| | | TG |
| Full: 13 | Modified | [CTAG]AAGCTTTATTGTGGTAGTTTATCACAGTTAAATTGCTAAGGCA |
| 5′UTR: 42 | hIns-13 | GTCAGTGCTTCTGACACAACAGTCTCCAACTTAAGCTGCAGTGACTC |
| ORF: 54 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGGCTCTTGCCCCTTCTGGCTTTACTGGCACTATGGG |
| | | GCCCTGACCCAGCGGCCGCTTTTGTGAACCAGCACCTCTGCGGATCTGAT |
| | | CTGGTTGAGGCCTTGTACCTGGTTTGTGGGGAAAGAGGTTTTTTCTATACC |
| | | CCAAGGACAAAGCGCGAGGCAGAAGACCTTCAAGTGGGCCAGGTAGAGC |
| | | TGGGAGGGGGCCCGGGTGCAGGATCCCTGCAGCCTCTGGCTCTAGAAGG |
| | | GTCTAGACAAAAAAGGGGCATAGTGGAGCAGTGCTGTACTTCAATTTGCA |
| | | GTTTGTACCAGCTTGAGAATTATTGTAACTGAAGCCAGCTGCCTTCCCTTTA |
| | | CTGTGGGTTAATGCTTGCAGCAGACATGATAAGATACATTGATGAGTTTGGACA |
| | | AACCACAACTAGAATGCAGTGAAAAAAATGCTTTATTTGTGAAATTTGTGATGCT |
| | | ATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 14 | Modified | [CTAG]AAGCTTTATTGTGGTAGTTTATCACAGTTAAATTGCTAAGGCA |
| 5′UTR: 42 | hIns-14 | GTCAGTGCTTCTGACACAACAGTCTCCAACTTAAGCTGCAGTGACTC |
| ORF: 55 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGGCTCTTGCCCCTTCTGGCTTTACTGGCACTATGGG |
| | | GCCCTGACCCAGCGGCCGCTTTTGTGAACCAGCACCTCTGCGGATCTGAT |
| | | CTGGTGGAGGCCTTGTACCTGGTTTGTGGGGAAAGAGGTTTTTTCTATAC |
| | | CCCCAGGACAAAGCGCGAGGCAGAAGACCTTCAAGTGGGCCAGGTAGAG |
| | | CTGGGAGGGGGCCCGGGTGCAGGATCCCTGCAGCCTCTGGCTCTGGAAG |
| | | GGTCTAGACAAAAAAGGGGCATAGTGGAGCAGTGCTGTACTTCAATTTGC |
| | | AGTTTGTACCAGCTTGAGAATTATTGTAACTGAAGCCAGCTGCCTTCCCTTT |
| | | ACTGTGGGTTAATGCTTGCAGCAGACATGATAAGATACATTGATGAGTTTGGAC |
| | | AAACCACAACTAGAATGCAGTGAAAAAAATGCTTTATTTGTGAAATTTGTGATGC |
| | | TATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 15 | Modified | [CTAG]AAGCTTTATTGTGGTAGTTTATCACAGTTAAATTGCTAAGGCA |
| 5′UTR: 42 | hIns-15 | GTCAGTGCTTCTGACACAACAGTCTCCAACTTAAGCTGCAGTGACTC |
| ORF: 56 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCTTGCCCCTTCTGGCTTTACTGGCACTATGGG |
| | | GCCCTGACCCAGCAGCTGCTTTTGTGAACCAGCACCTCTGTGGATCTGAT |
| | | CTGGTTGAGGCCTTGTACCTGGTTTGTGGGGAAAGAGGTTTTTTCTATACC |
| | | CCAAGGACAAAGAGGGAGGCAGAAGACCTTCAAGTGGGCCAGGTAGAG |
| | | CTGGGAGGGGGCCCTGGTGCAGGATCCCTGCAGCCTCTGGCTCTAGAAGG |
| | | GTCTAGACAAAAAAGGGGCATAGTGGAGCAGTGCTGTACTTCAATTTGCA |
| | | GTTTGTACCAGCTTGAGAATTATTGTAACTGAAGCCAGCTGCCTTCCCTTTA |
| | | CTGTGGGTTAATGCTTGCAGCAGACATGATAAGATACATTGATGAGTTTGGACA |
| | | AACCACAACTAGAATGCAGTGAAAAAAATGCTTTATTTGTGAAATTTGTGATGCT |
| | | ATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 16 | Modified | [CTAG]AAGCTTTATTGTGGTAGTTTATCACAGTTAAATTGCTAAGGCA |
| 5′UTR: 42 | hIns-16 | GTCAGTGCTTCTGACACAACAGTCTCCAACTTAAGCTGCAGTGACTC |
| ORF: 57 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCTTGCCCCTTCTGGCTTTACTGGCACTATGGG |
| | | GCCCTGACCCAGCAGCTGCTTTTGTGAACCAGCACCTCTGTGGATCTGAT |
| | | CTGGTGGAGGCCTTGTACCTGGTTTGTGGGGAAAGAGGTTTTTTCTATAC |
| | | CCCCAGGACAAAGAGAGAGGCAGAAGACCTTCAAGTGGGCCAGGTAGAG |
| | | CTGGGAGGGGGCCCAGGTGCAGGATCCCTGCAGCCTCTGGCTCTGGAAG |
| | | GGTCTAGACAAAAAAGAGGCATAGTGGAGCAGTGCTGTACTTCAATTTGC |
| | | AGTTTGTACCAGCTTGAGAATTATTGTAACTGAAGCCAGCTGCCTTCCCTTT |
| | | ACTGTGGGTTAATGCTTGCAGCAGACATGATAAGATACATTGATGAGTTTGGAC |
| | | AAACCACAACTAGAATGCAGTGAAAAAAATGCTTTATTTGTGAAATTTGTGATGC |
| | | TATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 84 | Modified | AAGCTTTATTGCGGTAGTTTATCACAGTTAAATTGCTAACGCAGTCAG |
| 5′UTR: 83 | hIns-17 | TGCTTCTGACACAACAGTCTCGAACTTAAGCTGCAGTGACTCTCTTAA |
| ORF: 43 | | GGTAGCCTTGCAGAAGTTGGTCGTGAGGCACTGGGCAGGTAAGTATC |
| 3′UTR: 97 | | AAGGTTACAAGACAGGTTTAAGGAGACCAATAGAAACTGGGCTTGTC |
| | | GAGACAGAGAAGACTCTTGCGTTTCTGATAGGCACCTATTGGTCTTA |
| | | CTGACATCCACTTTGCCTTTCTCTCCACAGGTGTCCACTCCCAGTTCA |
| | | ATTACAGCTCTTAAGGCTAGAGTCCTGCAGAATTGCCACCATGGCCCT |
| | | GTGGATGAGACTCCTGCCCCTGCTGGCACTGCTGGCCCTCTGGGGACCTG |
| | | ACCCAGCAGCAGCCTTTGTGAACCAACACCTGTGTGGCTCAGATCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCC |
| | | AGACAGAAGAGAGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATAAGGGACTAGAGCAGGGATCCAAGCTT |
| Full: 85 | Modified | AAGCTTTATTGTGGTAGTTTATCACAGTTAAATTGCTAAGGCAGTCAG |
| 5′UTR: | hIns-18 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 43 | | CTGACATCCACTTTGCCTTTCTCTCCACAGGTGTCCACTCCCAGTTCA |
| 3′UTR: 98 | | ATTACAGCTCTTAAGGCTAGAGTCCTGCAGAATTGCCACCATGGCCCT |
| | | GTGGATGAGACTCCTGCCCCTGCTGGCACTGCTGGCCCTCTGGGGACCTG |
| | | ACCCAGCAGCAGCCTTTGTGAACCAACACCTGTGTGGCTCAGATCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCC |
| | | AGACAGAAGAGAGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATAAGGGACTAGAGCAGGAATTCAAGCTT |
| Full: 86 | Modified | AAGCTTTATTGTGGTAGTTTATCACAGTTAAATTGCTAAGGCAGTCAG |
| 5′UTR: | hIns-19 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 46 | | CTGACATCCACTTTGCCTTTCTCTCCACAGGTGTCCACTCCCAGTTCA |
| 3′UTR: 99 | | ATTACAGCTCTTAAGGCTAGAGTCCTGCAGAATTGCCACCATGGCCCT |
| | | GTGGATGAGACTGCTCCCCCTCCTGGCTTTGCTGGCCCTGTGGGGACCAG |
| | | ATCCTGCAGCTGCCTTTGTGAATCAGCACCTGTGTGGCTCAGATCTGGTTG |
| | | AGGCACTCTACCTGGTGTGTGGGGAGAGAGGCTTCTTTTACACACCAAGG |
| | | ACCAAGAGGGAGGCAGAGGATCTGCAGGTAGGTCAGGTGGAGCTGGGAG |
| | | GTGGCCCTGGAGCAGGATCCCTGCAGCCCTTGGCACTGGAGGGCTCCAGA |
| | | CAGAAGAGGGGCATTGTGGAACAGTGTTGCACTAGCATCTGCAGCCTGTA |
| | | CCAGCTGGAGAACTACTGCAACTAAAGCCAGCTGCCTTCCCTTTACTGTGG |
| | | GTTAATGCTTGCAGCAGACATGATAAGATACATTGATGAGTTTGGACAAACCAC |
| | | AACTAGAATGCAGTGAAAAAAATGCTTTATTTGTGAAATTTGTGATGCTATTGCT |
| | | TTATTTGTAACCATTATAAGCTGCAATAAACAAGTTAACAACAACAATTGCATTC |
| | | ATTTTATGTTTCAGGTTCAGGGGGAGATGTGGGAGGTTTTTTAAAGCAAGTAAA |
| | | ACCTCTACAAATGTGGTAAAATAAGGGACTAGAGCAGCATATGAAGCTT |
| Full: 87 | Modified | AAGCTTTATTGTGGTAGTTTATCACAGTTAAATTGCTAAGGCAGTCAG |
| 5′UTR: | hIns-20 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 47 | | CTGACATCCACTTTGCCTTTCTCTCCACAGGTGTCCACTCCCAGTTCA |
| 3′UTR: 100 | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCAGATCTGGTG |
| | | GAAGCTCTCTACCTAGTGTGCGGGGAAAGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGGGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATAAGGGACTAGAGCAGGATATCAAGCTT |
| Full: 88 | Modified | AAGCTTTATTGTGGTAGTTTATCACAGTTAAATTGCTAAGGCAGTCAG |
| 5′UTR: | hIns-21 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 49 | | CTGACATCCACTTTGCCTTTCTCTCCACAGGTGTCCACTCCCAGTTCA |
| 3′UTR: 101 | | ATTACAGCTCTTAAGGCTAGAGTCCTGCAGAATTGCCACCATGGCCCT |
| | | GTGGATGAGACTCCTGCCCCTGCTGGCTCTGCTGGCCCTCTGGGGACCTG |
| | | ACCCAGCTGCAGCCTTTGTGAACCAACACCTGTGTGGCTCAGATCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCT |
| | | AGACAGAAGAGGGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATAAGGGACTAGAGCAGACTAGTAAGCTT |
| Full: 123 | Modified | AAGCTTTATTGTGGTAGTTTATCACAGTTAAATTGCTAAGGCAGTCAG |
| 5′ UTR: | hIns-22 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 110 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCAGATCTGGTG |
| | | GAAGCTCTCTACCTAGTGTGCGGGGAACGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGTGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATCCGATAAGGGACTAGAGCAGAAGCTTTTG |
| Full: 127 | Unmodified | AAGCTTTATTGTGGTAGTTTATCACAGTTAAATTGCTAAGGCAGTCAG |
| 5′ UTR: | hIns- | TGCTTCTGACACAACAGTCTCCAACTTAAGCTGCAGTGACTCTCTTAA |
| nucleic | 2 (B10H) | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 111 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCACACCTGGTG |
| | | GAAGCTCTCTACCTAGTGTGCGGGGAACGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGTGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATCCGATAAGGGACTAGAGCAGAAGCTTTTG |
| Full: 128 | Modified | AAGCTTTATTGTGGTAGTTTATCACAGTTAAATTGCTAAGGCAGTCAG |
| 5′ UTR: | hIns-27 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 112 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCACACCTGGTG |
| | | GAAGCTCTCTACCTAGTGTGCGGGGAAAGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGGGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATCCGATAAGGGACTAGAGCAGAAGCTTTTG |
| Full: 129 | Modified | AAGCTTTATTGTGGTAGTTTATCACAGTTAAATTGCTAAGGCAGTCAG |
| 5′ UTR: | hIns-28 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 113 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGGCCCT |
| | | GTGGATGAGACTCCTGCCCCTGCTGGCTCTGCTGGCCCTCTGGGGACCTG |
| | | ACCCAGCTGCAGCCTTTGTGAACCAACACCTGTGTGGCTCACACCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCT |
| | | AGACAGAAGAGGGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATCCGATAAGGGACTAGAGCAGAAGCTTTTG |
| Full: 133 | Modified | AAGCTTTATTGTGGTAGTTTATCACAGTTAAATTGCTAAGGCAGTCAG |
| 5′ UTR: | hIns-32 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 117 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGCGCCTTCGGCCCCGTGGCTTTCTCTCTGGGACTGCTGC |
| | | TGGTGCTGCCTGCCGCTTTCCCTGCCCCTTTTGTGAACCAACACCTGTGCG |
| | | GCTCAGATCTGGTGGAAGCTCTCTACCTAGTGTGCGGGGAACGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGTGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 134 | Modified | AAGCTTTATTGTGGTAGTTTATCACAGTTAAATTGCTAAGGCAGTCAG |
| 5′ UTR: | hIns-33 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 118 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGCGCCTTTGGCCCAGTGGCTTTCTCTCTGGGACTGCTGC |
| | | TGGTGCTGCCTGCCGCTTTCCCTGCCCCTTTTGTGAACCAACACCTGTGCG |
| | | GCTCAGATCTGGTGGAAGCTCTCTACCTAGTGTGCGGGGAAAGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGGGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 135 | Modified | AAGCTTTATTGTGGTAGTTTATCACAGTTAAATTGCTAAGGCAGTCAG |
| 5′ UTR: | hIns-34 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 119 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGTGCCTTTGGCCCAGTGGCTTTCTCTCTGGGACTGCTGC |
| | | TGGTGCTGCCTGCAGCTTTCCCTGCCCCTTTTGTGAACCAACACCTGTGTG |
| | | GCTCAGATCTGGTGGAAGCTCTCTACCTAGTGTGTGGGGAAAGAGGCTTC |
| | | TTCTACACACCCAGGACCAAGAGAGAGGCAGAGGACCTGCAGGTGGGGC |
| | | AGGTGGAGCTGGGAGGGGGCCCTGGTGCAGGCAGCCTGCAGCCCTTGGC |
| | | CCTGGAGGGGTCCAGGCAGAAGAGAGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 136 | Modified | AAGCTTTATTGTGGTAGTTTATCACAGTTAAATTGCTAAGGCAGTCAG |
| 5′ UTR: | hIns-35 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 120 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGAACA |
| 3′ UTR: 60 | | GCTTCAGCACAAGCGCCTTCGGCCCCGTGGCTTTCTCTCTGGGACTGCTGC |
| | | TGGTGCTGCCTGCCGCTTTCCCTGCCCCTTTTGTGAACCAACACCTGTGCG |
| | | GCTCACACCTGGTGGAAGCTCTCTACCTAGTGTGCGGGGAACGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGTGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 137 | Modified | AAGCTTTATTGTGGTAGTTTATCACAGTTAAATTGCTAAGGCAGTCAG |
| 5′ UTR: | hIns-36 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 121 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGCGCCTTTGGCCCAGTGGCTTTCTCTCTGGGACTGCTGC |
| | | TGGTGCTGCCTGCCGCTTTCCCTGCCCCTTTTGTGAACCAACACCTGTGCG |
| | | GCTCACACCTGGTGGAAGCTCTCTACCTAGTGTGCGGGGAAAGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGGGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 138 | Modified | AAGCTTTATTGTGGTAGTTTATCACAGTTAAATTGCTAAGGCAGTCAG |
| 5′ UTR: | hIns-37 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 122 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGTGCCTTTGGCCCAGTGGCTTTCTCTCTGGGACTGCTGC |
| | | TGGTGCTGCCTGCAGCTTTCCCTGCCCCTTTTGTGAACCAACACCTGTGTG |
| | | GCTCACACCTGGTGGAAGCTCTCTACCTAGTGTGTGGGGAAAGAGGCTTC |
| | | TTCTACACACCCAGGACCAAGAGAGAGGCAGAGGACCTGCAGGTGGGGC |
| | | AGGTGGAGCTGGGAGGGGGCCCTGGTGCAGGCAGCCTGCAGCCCTTGGC |
| | | CCTGGAGGGGTCCAGGCAGAAGAGAGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 160 | Unmodified | AAGCTTTATTGCGGTAGTTTATCACAGTTAAATTGCTAACGCAGTCAG |
| 5′ UTR: 148 | hIns 2 | TGCTTCTGACACAACAGTCTCGAACTTAAGCTGCAGTGACTCTCTTAA |
| ORF: 110 | (B10D) | GGTAGCCTTGCAGAAGTTGGTCGTGAGGCACTGGGCAGGTAAGTATC |
| | | AAGGTTACAAGACAGGTTTAAGGAGACCAATAGAAACTGGGCTTGTC |
| | | GAGACAGAGAAGACTCTTGCGTTTCTGATAGGCACCTATTGGTCTTA |
| | | CTGACATCCACTTTGCCTTTCTCTCCACAGGTGTCCACTCCCAGTTCA |
| | | ATTACAGCTCTTAAGGCTAGAGTACTTAATACGACTCACTATAGGCTA |
| | | GCCTCGAGAATTCTGCCATGGCCCTGTGGATGCGCCTCCTGCCCCTGCT |
| | | GGCGCTGCTGGCCCTCTGGGGACCTGACCCAGCCGCAGCCTTTGTGAACC |
| | | AACACCTGTGCGGCTCAGATCTGGTGGAAGCTCTCTACCTAGTGTGCGGG |
| | | GAACGAGGCTTCTTCTACACACCCAGGACCAAGCGGGAGGCAGAGGACC |
| | | TGCAGGTGGGGCAGGTGGAGCTGGGCGGGGGCCCTGGTGCAGGCAGCCT |
| | | GCAGCCCTTGGCCCTGGAGGGGTCGCGACAGAAGCGTGGCATTGTGGAA |
| | | CAATGCTGTACCAGCATCTGCTCCCTCTACCAGCTGGAGAACTACTGCAA |
| | | CTAG |
| Full: 161 | Unmodified | [CTAG]AAGCTTTATTGTGGTAGTTTATCACAGTTAAATTGCTAAGGCA |
| 5′ UTR: 42 | hIns 3 | GTCAGTGCTTCTGACACAACAGTCTCCAACTTAAGCTGCAGTGACTC |
| ORF-110 | (B10D) | TCTTAAGGTAGCCTTGCAGAAGTTGGTCCTGAGGCACTGGGCAGGTA |
| 3′ UTR-60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGCCTCCTGCCCCTGCTGGCGCTGCTGGCCCTCTGG |
| | | GGACCTGACCCAGCCGCAGCCTTTGTGAACCAACACCTGTGCGGCTCAGA |
| | | TCTGGTGGAAGCTCTCTACCTAGTGTGCGGGGAACGAGGCTTCTTCTACA |
| | | CACCCAGGACCAAGCGGGAGGCAGAGGACCTGCAGGTGGGGCAGGTGG |
| | | AGCTGGGCGGGGGCCCTGGTGCAGGCAGCCTGCAGCCCTTGGCCCTGGA |
| | | GGGGTCGCGACAGAAGCGTGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 170 | Modified | AAGCTTTATTGTGGTAGTTTATCACAGTTAAATTGCTAAGGCAGTCAG |
| 5′ UTR: | hIns 41 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 121 | | CTGACATCCACTTTGCCTTTCTCTCCACAGGTGTCCACTCCCAGTTCA |
| 3′ UTR: 171 | | ATTACAGCTCTTAAGGCTAGAGTCCTGCAGAATTGCCACCATGAACA |
| | | GCTTCAGCACAAGCGCCTTTGGCCCAGTGGCTTTCTCTCTGGGACTGCTGC |
| | | TGGTGCTGCCTGCCGCTTTCCCTGCCCCTTTTGTGAACCAACACCTGTGCG |
| | | GCTCACACCTGGTGGAAGCTCTCTACCTAGTGTGCGGGGAAAGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGGGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCATGGCTACGTA |

In some aspects, the nucleic acids include codon optimized and CpG reduced sequences relative to wild-type and/or unmodified human insulin nucleic acid sequences (e.g., SEQ ID NO: 1). The modified nucleic acids were chemically synthesized, prepared in expression cassettes including a CMV promoter, and cloned into an expression plasmid. The modified sequences were confirmed by Sanger sequencing.

### Example 2: Gck Nucleic Acids

The following human glucokinase (Gck) nucleic acid sequences (shown in Table 2) corresponding to SEQ ID Nos: 20-39 89-96, 163-164, and 168 were designed in silico. 5′UTR sequences (SEQ ID NO: 42 or SEQ ID NO: 83) are in BOLD, ORF sequences are underlined (SEQ ID NOs: 61-80 and 162), and 3′ UTR are italicized (SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109). The 5′UTRs having the sequence of SEQ ID NO: 42 was further modified to remove the CTAG at positions 1-4. Accordingly, in certain constructs the 5′UTR included nucleic acids 5-329 of SEQ ID NO: 42.

TABLE 2
Nucleic Acid Sequences Encoding Glucokinase

| SEQ ID | | |
|---|---|---|
| NOs | Name | Sequence |
| Full: 19 | Wildtype | AAGCTTTATTGCGGTAGTTTATCACAGTTAAATTGCTAACGCAGTCA |
| 5′ UTR: 148 | hGck | GTGCTTCTGACACAACAGTCTCGAACTTAAGCTGCAGTGACTCTCTT |
| ORF: 162 | | AAGGTAGCCTTGCAGAAGTTGGTCGTGAGGCACTGGGCAGGTAAGT |
| 3′ UTR: 149 | | ATCAAGGTTACAAGACAGGTTTAAGGAGACCAATAGAAACTGGGCT |
| | | TGTCGAGACAGAGAAGACTCTTGCGTTTCTGATAGGCACCTATTGGT |
| | | CTTACTGACATCCACTTTGCCTTTCTCTCCACAGGTGTCCACTCCCA |
| | | GTTCAATTACAGCTCTTAAGGCTAGAGTACTTAATACGACTCACTAT |
| | | AGGCTAGCCTCGAGAATTCTGCCATGGCGATGGATGTCACAAGGAGC |
| | | CAGGCCCAGACAGCCTTGACTCTGGTAGAGCAGATCCTGGCAGAGTTCC |
| | | AGCTGCAGGAGGAGGACCTGAAGAAGGTGATGAGACGGATGCAGAAGG |
| | | AGATGGACCGCGGCCTGAGGCTGGAGACCCATGAAGAGGCCAGTGTGA |
| | | AGATGCTGCCCACCTACGTGCGCTCCACCCCAGAAGGCTCAGAAGTCGG |
| | | GGACTTCCTCTCCCTGGACCTGGGTGGCACTAACTTCAGGGTGATGCTGG |
| | | TGAAGGTGGGAGAAGGTGAGGAGGGGCAGTGGAGCGTGAAGACCAAAC |
| | | ACCAGATGTACTCCATCCCCGAGGACGCCATGACCGGCACTGCTGAGAT |
| | | GCTCTTCGACTACATCTCTGAGTGCATCTCCGACTTCCTGGACAAGCATC |
| | | AGATGAAACACAAGAAGCTGCCCCTGGGCTTCACCTTCTCCTTTCCTGTG |
| | | AGGCACGAAGACATCGATAAGGGCATCCTTCTCAACTGGACCAAGGGCT |
| | | TCAAGGCCTCAGGAGCAGAAGGGAACAATGTCGTGGGGCTTCTGCGAG |
| | | ACGCTATCAAACGGAGAGGGGACTTTGAAATGGATGTGGTGGCAATGGT |
| | | GAATGACACGGTGGCCACGATGATCTCCTGCTACTACGAAGACCATCAG |
| | | TGCGAGGTCGGCATGATCGTGGGCACGGGCTGCAATGCCTGCTACATGG |
| | | AGGAGATGCAGAATGTGGAGCTGGTGGAGGGGGACGAGGGCCGCATGT |
| | | GCGTCAATACCGAGTGGGGCGCCTTCGGGGACTCCGGCGAGCTGGACGA |
| | | GTTCCTGCTGGAGTATGACCGCCTGGTGGACGAGAGCTCTGCAAACCCC |
| | | GGTCAGCAGCTGTATGAGAAGCTCATAGGTGGCAAGTACATGGGCGAGC |
| | | TGGTGCGGCTTGTGCTGCTCAGGCTCGTGGACGAAAACCTGCTCTTCCAC |
| | | GGGGAGGCCTCCGAGCAGCTGCGCACACGCGGAGCCTTCGAGACGCGCT |
| | | TCGTGTCGCAGGTGGAGAGCGACACGGGCGACCGCAAGCAGATCTACA |
| | | ACATCCTGAGCACGCTGGGGCTGCGACCCTCGACCACCGACTGCGACAT |
| | | CGTGCGCCGCGCCTGCGAGAGCGTGTCTACGCGCGCTGCGCACATGTGC |
| | | TCGGCGGGGCTGGCGGGCGTCATCAACCGCATGCGCGAGAGCCGCAGC |
| | | GAGGACGTAATGCGCATCACTGTGGGCGTGGATGGCTCCGTGTACAAGC |
| | | TGCACCCCAGCTTCAAGGAGCGGTTCCATGCCAGCGTGCGCAGGCTGAC |
| | | GCCCAGCTGCGAGATCACCTTCATCGAGTCGGAGGAGGGCAGTGGCCGG |
| | | GGCGCGGCCCTGGTCTCGGCGGTGGCCTGTAAGAAGGCCTGTATGCTGG |
| | | GCCAGTGAACGCAGCTGCTAGCTAATCGATACCGTCGACCTCGAGGAATTCA |
| | | CGCGTGGTACCTCTAGAGTCGACCCGGGCGGCCGCTTCCCTTTAGTGAGGG |
| | | TTAATGCTTCGAGCAGACATGATAAGATACATTGATGAGTTTGGACAAACCAC |
| | | AACTAGAATGCAGTGAAAAAAATGCTTTATTTGTGAAATTTGTGATGCTATTGC |
| | | TTTATTTGTAACCATTATAAGCTGCAATAAACAAGTTAACAACAACAATTGCATT |
| | | CATTTTATGTTTCAGGTTCAGGGGGAGATGTGGGAGGTTTTTTAAAGCAAGTA |
| | | AAACCTCTACAAATGTGGTAAAATCCGATAAGGGACTAGAGCAGAAGCTTTTG |
| Full: 20 | Modified | [CTAG]AAGCTTTATTGTGGTAGTTTATCACAGTTAAATTGCTAAGGC |
| 5′UTR: 42 | hGck-2 | AGTCAGTGCTTCTGACACAACAGTCTCCAACTTAAGCTGCAGTGACT |
| ORF: 61 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGGATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGGAGAGGG |
| | | GACTTTGAAATGGATGTGGTGGCAATGGTGAATGACACTGTGGCCACCA |
| | | TGATCTCCTGCTACTATGAAGACCATCAGTGTGAGGTGGGCATGATTGT |
| | | GGGCACAGGCTGCAATGCCTGCTACATGGAGGAGATGCAGAATGTGGA |
| | | GCTGGTGGAGGGGGATGAGGGCAGGATGTGTGTCAATACAGAGTGGGG |
| | | AGCCTTTGGGGACTCTGGGGAGCTGGATGAGTTCCTGCTGGAGTATGAC |
| | | AGGCTGGTGGATGAGAGCTCAGCAAACCCTGGTCAGCAGCTGTATGAGA |
| | | AGCTCATAGGTGGCAAGTACATGGGAGAGCTGGTGAGGCTTGTGCTGCT |
| | | CAGGCTGGTGGATGAAAACCTGCTCTTCCATGGGGAGGCCTCTGAGCAG |
| | | CTGAGGACAAGGGGAGCCTTTGAGACCAGGTTTGTGTCCCAGGTGGAGA |
| | | GTGACACAGGAGACAGGAAGCAGATCTACAACATCCTGAGCACCCTGG |
| | | GGCTGAGACCCTCCACCACAGACTGTGACATTGTGAGGAGGGCTTGTGA |
| | | GAGTGTGTCTACCAGGGCTGCCCACATGTGCTCTGCTGGGCTGGCTGGG |
| | | GTCATCAACAGGATGAGGGAGAGCAGGAGTGAGGATGTAATGAGGATC |
| | | ACTGTGGGAGTGGATGGCTCTGTGTACAAGCTGCACCCCTCATTCAAGG |
| | | AGAGGTTCCATGCCAGTGTGAGGAGGCTGACCCCCAGCTGTGAGATCAC |
| | | CTTTATTGAGTCTGAGGAGGGCAGTGGCAGGGGAGCAGCCCTGGTCTCT |
| | | GCTGTGGCCTGTAAGAAGGCCTGTATGCTGGGCCAGTGAAGCCAGCTGC |
| | | CTTCCCTTTACTGTGGGTTAATGCTTGCAGCAGACATGATAAGATACATTGATG |
| | | AGTTTGGACAAACCACAACTAGAATGCAGTGAAAAAAATGCTTTATTTGTGAAA |
| | | TTTGTGATGCTATTGCTTTATTTGTAACCATTATAAGCTGCAATAAACAAGTTAA |
| | | CAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTA |
| | | GAGCAGAAGCTTTTG |
| Full: 21 | Modified | [CTAG]AAGCTTTATTGTGGTAGTTTATCACAGTTAAATTGCTAAGGC |
| 5′UTR: 42 | hGck-3 | AGTCAGTGCTTCTGACACAACAGTCTCCAACTTAAGCTGCAGTGACT |
| ORF: 62 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGTCTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGGGATGCTATCAAAAGGAGAGGG |
| | | GACTTTGAAATGGATGTGGTGGCAATGGTGAATGACACAGTGGCCACAA |
| | | TGATCTCCTGCTACTATGAAGACCATCAGTGTGAGGTGGGCATGATTGT |
| | | GGGCACAGGCTGCAATGCCTGCTACATGGAGGAGATGCAGAATGTGGA |
| | | GCTGGTGGAGGGGGATGAGGGCAGAATGTGTGTCAATACAGAGTGGGG |
| | | AGCCTTTGGGGACTCTGGGGAGCTGGATGAGTTCCTGCTGGAGTATGAC |
| | | AGACTGGTGGATGAGAGCTCAGCAAACCCTGGTCAGCAGCTGTATGAGA |
| | | AGCTCATAGGTGGCAAGTACATGGGAGAGCTGGTGAGACTTGTGCTGCT |
| | | CAGGCTGGTGGATGAAAACCTGCTCTTCCATGGGGAGGCCTCTGAGCAG |
| | | CTGAGAACAAGAGGAGCCTTTGAGACCAGATTTGTGTCCCAGGTGGAGT |
| | | CTGACACAGGAGACAGGAAGCAGATCTACAACATCCTGAGCACCCTGG |
| | | GGCTGAGGCCCTCCACCACAGACTGTGACATTGTGAGAAGAGCCTGTGA |
| | | GTCTGTGTCTACCAGGGCTGCCCACATGTGCTCTGCTGGGCTGGCTGGGG |
| | | TCATCAACAGAATGAGGGAGAGCAGGTCTGAGGATGTAATGAGAATCA |
| | | CTGTGGGAGTGGATGGCTCTGTGTACAAGCTGCACCCCTCATTCAAGGA |
| | | GAGATTCCATGCCTCTGTGAGAAGGCTGACCCCCAGCTGTGAGATCACC |
| | | TTTATTGAGTCTGAGGAGGGCAGTGGCAGAGGAGCTGCCCTGGTCTCTG |
| | | CTGTGGCCTGTAAGAAGGCCTGTATGCTGGGCCAGTGAAGCCAGCTGCCT |
| | | TCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAGAAGCTTTTG |
| Full: 22 | Modified | [CTAG]AAGCTTTATTGTGGTAGTTTATCACAGTTAAATTGCTAAGGC |
| 5′UTR: 42 | hGck-4 | AGTCAGTGCTTCTGACACAACAGTCTCCAACTTAAGCTGCAGTGACT |
| ORF: 63 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACTGGCACTGCTGAGATGCTCTTTGACTACATCTCTGAG |
| | | TGCATCTCAGACTTCCTGGACAAGCATCAGATGAAACACAAGAAGCTGC |
| | | CCCTGGGCTTCACCTTCTCCTTTCCTGTGAGGCATGAAGACATTGATAAG |
| | | GGCATCCTTCTCAACTGGACCAAGGGCTTCAAGGCCTCAGGAGCAGAAG |
| | | GGAACAATGTTGTGGGGCTTCTGAGGGATGCTATCAAAAGGAGAGGGG |
| | | ACTTTGAAATGGATGTGGTGGCAATGGTGAATGACACTGTGGCCACTAT |
| | | GATCTCCTGCTACTATGAAGACCATCAGTGTGAGGTAGGCATGATTGTG |
| | | GGCACTGGCTGCAATGCCTGCTACATGGAGGAGATGCAGAATGTGGAGC |
| | | TGGTGGAGGGGGATGAGGGCAGAATGTGTGTCAATACTGAGTGGGGTG |
| | | CCTTTGGGGACTCAGGGGAGCTGGATGAGTTCCTGCTGGAGTATGACAG |
| | | ACTGGTGGATGAGAGCTCTGCAAACCCTGGTCAGCAGCTGTATGAGAAG |
| | | CTCATAGGTGGCAAGTACATGGGAGAGCTGGTGAGACTTGTGCTGCTCA |
| | | GGCTGGTGGATGAAAACCTGCTCTTCCATGGGGAGGCCTCTGAGCAGCT |
| | | GAGAACAAGAGGAGCCTTTGAGACCAGATTTGTGTCCCAGGTGGAGTCT |
| | | GACACTGGTGACAGGAAGCAGATCTACAACATCCTGAGCACACTGGGGC |
| | | TGAGGCCCTCCACCACAGACTGTGACATTGTGAGAAGAGCTTGTGAGAG |
| | | TGTGTCTACCAGGGCTGCTCACATGTGCAGTGCTGGGCTGGCTGGGGTC |
| | | ATCAACAGAATGAGGGAGAGCAGGTCTGAGGATGTAATGAGAATCACT |
| | | GTGGGTGTGGATGGCAGTGTGTACAAGCTGCACCCCTCATTCAAGGAGA |
| | | GATTCCATGCCAGTGTGAGAAGGCTGACCCCCAGCTGTGAGATCACCTT |
| | | CATTGAGTCTGAGGAGGGCAGTGGCAGGGGTGCAGCCCTGGTCAGTGCA |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 23 | Modified | [CTAG]AAGCTTTATTGTGGTAGTTTATCACAGTTAAATTGCTAAGGC |
| 5′UTR: 42 | hGck-5 | AGTCAGTGCTTCTGACACAACAGTCTCCAACTTAAGCTGCAGTGACT |
| ORF: 64 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGTCTGTGAAGACCAAACACCAGATGTACTCCATCCCTG |
| | | AGGATGCCATGACTGGCACTGCTGAGATGCTCTTTGACTACATCTCTGAG |
| | | TGCATCTCAGACTTCCTGGACAAGCATCAGATGAAACACAAGAAGCTGC |
| | | CCCTGGGCTTCACCTTCTCCTTTCCTGTGAGGCATGAAGACATAGATAAG |
| | | GGCATCCTTCTCAACTGGACCAAGGGCTTCAAGGCCTCAGGAGCAGAAG |
| | | GGAACAATGTGGTGGGGCTTCTGAGAGATGCTATCAAAAGGAGAGGGG |
| | | ACTTTGAAATGGATGTGGTGGCAATGGTGAATGACACTGTGGCCACTAT |
| | | GATCTCCTGCTACTATGAAGACCATCAGTGTGAGGTGGGCATGATAGTG |
| | | GGCACTGGCTGCAATGCCTGCTACATGGAGGAGATGCAGAATGTGGAGC |
| | | TGGTGGAGGGGGATGAGGGCAGGATGTGTGTCAATACTGAGTGGGGAG |
| | | CCTTTGGGGACTCTGGGGAGCTGGATGAGTTCCTGCTGGAGTATGACAG |
| | | GCTGGTGGATGAGAGCTCAGCAAACCCTGGTCAGCAGCTGTATGAGAAG |
| | | CTCATAGGTGGCAAGTACATGGGGGAGCTGGTGAGGCTTGTGCTGCTCA |
| | | GGCTGGTGGATGAAAACCTGCTCTTCCATGGGGAGGCCTCTGAGCAGCT |
| | | GAGAACAAGGGGAGCCTTTGAGACCAGGTTTGTGTCCCAGGTGGAGTCT |
| | | GACACTGGGGACAGGAAGCAGATCTACAACATCCTGAGCACCCTGGGG |
| | | CTGAGGCCCTCCACCACTGACTGTGACATAGTGAGGAGAGCCTGTGAGT |
| | | CTGTGTCTACCAGGGCTGCCCACATGTGCTCTGCTGGGCTGGCAGGGGT |
| | | CATCAACAGGATGAGGGAGAGCAGATCTGAGGATGTAATGAGGATCAC |
| | | TGTGGGTGTGGATGGCTCTGTGTACAAGCTGCACCCCTCATTCAAGGAG |
| | | AGGTTCCATGCCTCTGTGAGGAGGCTGACCCCCAGCTGTGAGATCACCT |
| | | TTATAGAGTCTGAGGAGGGCAGTGGCAGAGGGGCAGCCCTGGTCTCTGC |
| | | AGTGGCCTGTAAGAAGGCCTGTATGCTGGGCCAGTGAAGCCAGCTGCCTT |
| | | CCCTTTACTGTGGGTTAATGCTTGCAGCAGACATGATAAGATACATTGATGAG |
| | | TTTGGACAAACCACAACTAGAATGCAGTGAAAAAAATGCTTTATTTGTGAAATT |
| | | TGTGATGCTATTGCTTTATTTGTAACCATTATAAGCTGCAATAAACAAGTTAACA |
| | | ACAACAATTGCATTCATTTTATGTTTCAGGTTCAGGGGGAGATGTGGGAGGTT |
| | | TTTTAAAGCAAGTAAAACCTCTACAAATGTGGTAAAATCCGATAAGGGACTAGA |
| | | GCAGAAGCTTTTG |
| Full: 24 | Modified | [CTAG]AAGCTTTATTGTGGTAGTTTATCACAGTTAAATTGCTAAGGC |
| 5′UTR: 42 | hGck-6 | AGTCAGTGCTTCTGACACAACAGTCTCCAACTTAAGCTGCAGTGACT |
| ORF: 65 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGGATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGGAGAGGG |
| | | GACTTTGAAATGGATGTGGTGGCAATGGTGAATGACACAGTGGCCACCA |
| | | TGATCTCCTGCTACTATGAAGACCATCAGTGTGAGGTGGGCATGATAGT |
| | | GGGCACAGGCTGCAATGCCTGCTACATGGAGGAGATGCAGAATGTGGA |
| | | GCTGGTGGAGGGGGATGAGGGCAGGATGTGTGTCAATACTGAGTGGGG |
| | | AGCCTTTGGGGACTCTGGGGAGCTGGATGAGTTCCTGCTGGAGTATGAC |
| | | AGGCTGGTGGATGAGAGCTCAGCAAACCCTGGTCAGCAGCTGTATGAGA |
| | | AGCTCATAGGTGGCAAGTACATGGGGGAGCTGGTGAGGCTTGTGCTGCT |
| | | CAGGCTGGTGGATGAAAACCTGCTCTTCCATGGGGAGGCCTCAGAGCAG |
| | | CTGAGAACAAGGGGAGCCTTTGAGACCAGGTTTGTGTCCCAGGTGGAGA |
| | | GTGACACAGGTGACAGGAAGCAGATCTACAACATCCTGAGCACCCTGGG |
| | | GCTGAGACCCTCCACCACAGACTGTGACATTGTGAGGAGGGCCTGTGAG |
| | | AGTGTGTCTACCAGGGCTGCCCACATGTGCTCTGCTGGGCTGGCAGGAG |
| | | TCATCAACAGGATGAGGGAGAGCAGGAGTGAGGATGTAATGAGGATCA |
| | | CTGTGGGAGTGGATGGCTCTGTGTACAAGCTGCACCCCTCATTCAAGGA |
| | | GAGGTTCCATGCCAGTGTGAGAAGGCTGACCCCCAGCTGTGAGATCACC |
| | | TTTATAGAGTCTGAGGAGGGCAGTGGCAGGGGAGCAGCCCTGGTCTCTG |
| | | CTGTGGCCTGTAAGAAGGCCTGTATGCTGGGCCAGTGAAGCCAGCTGCCT |
| | | TCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAGAAGCTTTTG |
| Full: 25 | Modified | [CTAG]AAGCTTTATTGTGGTAGTTTATCACAGTTAAATTGCTAAGGC |
| 5′UTR: 42 | hGck-7 | AGTCAGTGCTTCTGACACAACAGTCTCCAACTTAAGCTGCAGTGACT |
| ORF: 66 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGGATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGGAGAGGG |
| | | GACTTTGAAATGGATGTGGTGGCAATGGTGAATGACACTGTGGCCACAA |
| | | TGATCTCCTGCTACTATGAAGACCATCAGTGTGAGGTGGGCATGATAGT |
| | | GGGCACAGGCTGCAATGCCTGCTACATGGAGGAGATGCAGAATGTGGA |
| | | GCTGGTGGAGGGGGATGAGGGCAGAATGTGTGTCAATACAGAGTGGGG |
| | | AGCCTTTGGGGACTCTGGGGAGCTGGATGAGTTCCTGCTGGAGTATGAC |
| | | AGACTGGTGGATGAGAGCTCAGCAAACCCTGGTCAGCAGCTGTATGAGA |
| | | AGCTCATAGGTGGCAAGTACATGGGAGAGCTGGTGAGGCTTGTGCTGCT |
| | | CAGGCTGGTGGATGAAAACCTGCTCTTCCATGGGGAGGCCTCTGAGCAG |
| | | CTGAGGACAAGAGGAGCCTTTGAGACCAGATTTGTGTCACAGGTGGAGT |
| | | CTGACACTGGAGACAGAAAGCAGATCTACAACATCCTGAGCACACTGGG |
| | | GCTGAGGCCCTCCACCACTGACTGTGACATAGTGAGGAGAGCTTGTGAG |
| | | AGTGTGTCTACCAGAGCTGCCCACATGTGCTCTGCTGGGCTGGCTGGGG |
| | | TCATCAACAGGATGAGAGAGAGCAGGAGTGAGGATGTAATGAGAATCA |
| | | CTGTGGGGGTGGATGGCTCTGTGTACAAGCTGCACCCCTCATTCAAGGA |
| | | GAGGTTCCATGCCTCTGTGAGGAGGCTGACCCCCAGCTGTGAGATCACC |
| | | TTTATTGAGTCTGAGGAGGGCAGTGGAAGGGGAGCAGCCCTGGTCTCTG |
| | | CAGTGGCCTGTAAGAAGGCCTGTATGCTGGGCCAGTGAAGCCAGCTGCC |
| | | TTCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAGAAGCTTTTG |
| Full: 26 | Modified | [CTAG]AAGCTTTATTGTGGTAGTTTATCACAGTTAAATTGCTAAGGC |
| 5′UTR: 42 | hGck-8 | AGTCAGTGCTTCTGACACAACAGTCTCCAACTTAAGCTGCAGTGACT |
| ORF: 67 | | CTCTTAAGGTAGCCTTGCAGAAGTTGGTCCTGAGGCACTGGGCAGG |
| 3′UTR: 59 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGGATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGGAGAGGG |
| | | GACTTTGAAATGGATGTGGTGGCAATGGTGAATGACACAGTGGCCACTA |
| | | TGATCTCCTGCTACTATGAAGACCATCAGTGTGAGGTGGGCATGATTGT |
| | | GGGCACTGGCTGCAATGCCTGCTACATGGAGGAGATGCAGAATGTGGAG |
| | | CTGGTGGAGGGGGATGAGGGCAGGATGTGTGTCAATACTGAGTGGGGA |
| | | GCCTTTGGGGACTCTGGGGAGCTGGATGAGTTCCTGCTGGAGTATGACA |
| | | GGCTGGTGGATGAGAGCTCAGCAAACCCTGGTCAGCAGCTGTATGAGAA |
| | | GCTCATAGGTGGCAAGTACATGGGAGAGCTGGTGAGACTTGTGCTGCTC |
| | | AGGCTGGTGGATGAAAACCTGCTCTTCCATGGGGAGGCCTCAGAGCAGC |
| | | TGAGGACAAGGGGAGCCTTTGAGACCAGGTTTGTGTCCCAGGTGGAGAG |
| | | TGACACTGGAGACAGGAAGCAGATCTACAACATCCTGAGCACCCTGGGG |
| | | CTGAGGCCCTCTACCACAGACTGTGACATAGTGAGAAGGGCCTGTGAGT |
| | | CTGTGTCTACCAGGGCTGCCCACATGTGCTCTGCTGGGCTGGCTGGAGTC |
| | | ATCAACAGAATGAGGGAGAGCAGAAGTGAGGATGTAATGAGGATCACT |
| | | GTGGGAGTGGATGGCTCTGTGTACAAGCTGCACCCCTCATTCAAGGAGA |
| | | GGTTCCATGCCAGTGTGAGGAGGCTGACCCCCAGCTGTGAGATCACCTT |
| | | TATTGAGTCTGAGGAGGGCAGTGGCAGGGGAGCTGCCCTGGTCTCTGCT |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 27 | Modified | [CTAG]AAGCTTTATTGTGGTAGTTTATCACAGTTAAATTGCTAAGGC |
| 5′UTR: 42 | hGck-9 | AGTCAGTGCTTCTGACACAACAGTCTCCAACTTAAGCTGCAGTGACT |
| ORF: 68 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACCGCGGCCTGAGGC |
| | | TGGAGACCCATGAAGAGGCCAGTGTGAAGATGCTGCCCACCTATGTGCG |
| | | CTCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCAGACTTCCTGGACAAGCATCAGATGAAACACAAGAAGCTG |
| | | CCCCTGGGCTTCACCTTCTCCTTTCCTGTGAGGCATGAAGACATTGATAA |
| | | GGGCATCCTTCTCAACTGGACCAAGGGCTTCAAGGCCTCAGGAGCAGAA |
| | | GGGAACAATGTTGTGGGGCTTCTGAGAGATGCTATCAAAAGAAGAGGG |
| | | GACTTTGAAATGGATGTGGTGGCAATGGTGAATGACACAGTGGCCACTA |
| | | TGATCTCCTGCTACTATGAAGACCATCAGTGTGAGGTTGGCATGATTGTG |
| | | GGCACAGGCTGCAATGCCTGCTACATGGAGGAGATGCAGAATGTGGAG |
| | | CTGGTGGAGGGGGATGAGGGCCGCATGTGTGTCAATACTGAGTGGGGCG |
| | | CCTTTGGGGACTCTGGAGAGCTGGATGAGTTCCTGCTGGAGTATGACCG |
| | | CCTGGTGGATGAGAGCTCAGCAAACCCAGGTCAGCAGCTGTATGAGAAG |
| | | CTCATAGGTGGCAAGTACATGGGAGAGCTGGTGCGGCTTGTGCTGCTCA |
| | | GGTTGGTGGATGAAAACCTGCTCTTCCATGGGGAGGCCAGTGAGCAGCT |
| | | GCGCACACGCGGAGCCTTTGAGACGCGCTTTGTGTCACAAGTGGAGTCT |
| | | GACACAGGAGACCGCAAGCAGATCTACAACATCCTGAGCACACTGGGG |
| | | CTGCGGCCCTCCACCACTGACTGTGACATTGTGCGCCGCGCCTGTGAGA |
| | | GTGTGTCTACGCGCGCTGCGCACATGTGCTCAGCGGGGCTGGCGGGAGT |
| | | CATCAACCGCATGCGCGAGAGCCGCTCTGAGGATGTAATGCGCATCACT |
| | | GTGGGAGTGGATGGCAGTGTGTACAAGCTGCACCCCAGCTTCAAGGAGC |
| | | GGTTCCATGCCTCAGTGCGCAGGCTGACCCCCAGCTGTGAGATCACCTTT |
| | | ATTGAGTCTGAGGAGGGCAGTGGCAGAGGCGCGGCCCTGGTCAGCGCG |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 28 | Modified | [CTAG]AAGCTTTATTGTGGTAGTTTATCACAGTTAAATTGCTAAGGC |
| 5′UTR: 42 | hGck-10 | AGTCAGTGCTTCTGACACAACAGTCTCCAACTTAAGCTGCAGTGACT |
| ORF: 69 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACCGCGGCCTGAGGC |
| | | TGGAGACCCATGAAGAGGCCAGTGTGAAGATGCTGCCCACCTATGTGCG |
| | | CTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCAGACTTCCTGGACAAGCATCAGATGAAACACAAGAAGCTG |
| | | CCCCTGGGCTTCACCTTCTCCTTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGCGGGATGCTATCAAAAGAAGAGGG |
| | | GACTTTGAAATGGATGTGGTGGCAATGGTGAATGACACAGTGGCCACCA |
| | | TGATCTCCTGCTACTATGAAGACCATCAGTGTGAGGTGGGCATGATTGT |
| | | GGGCACTGGCTGCAATGCCTGCTACATGGAGGAGATGCAGAATGTGGAG |
| | | CTGGTGGAGGGGGATGAGGGCCGCATGTGTGTCAATACAGAGTGGGGC |
| | | GCCTTTGGGGACAGCGGAGAGCTGGATGAGTTCCTGCTGGAGTATGACC |
| | | GCCTGGTGGATGAGAGCTCAGCAAACCCAGGTCAGCAGCTGTATGAGAA |
| | | GCTCATAGGTGGCAAGTACATGGGAGAGCTGGTGCGGCTTGTGCTGCTC |
| | | AGGCTGGTGGATGAAAACCTGCTCTTCCATGGGGAGGCCTCTGAGCAGC |
| | | TGCGCACACGCGGAGCCTTTGAGACGCGCTTTGTGAGCCAGGTGGAGAG |
| | | TGACACAGGAGACCGCAAGCAGATCTACAACATCCTGAGCACCCTGGGG |
| | | CTGCGGCCCAGCACCACTGACTGTGACATTGTGCGCCGCGCCTGTGAGT |
| | | CAGTGTCTACGCGCGCTGCGCACATGTGCAGCGCGGGGCTGGCGGGAGT |
| | | CATCAACCGCATGCGCGAGAGCCGCTCTGAGGATGTAATGCGCATCACT |
| | | GTGGGAGTGGATGGCAGTGTGTACAAGCTGCACCCCAGCTTCAAGGAGC |
| | | GGTTCCATGCCTCAGTGCGCAGGCTGACCCCCAGCTGTGAGATCACCTTT |
| | | ATTGAGTCTGAGGAGGGCAGTGGCAGAGGCGCGGCCCTGGTCAGCGCG |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 29 | Modified | [CTAG]AAGCTTTATTGTGGTAGTTTATCACAGTTAAATTGCTAAGGC |
| 5′UTR: 42 | hGck-11 | AGTCAGTGCTTCTGACACAACAGTCTCCAACTTAAGCTGCAGTGACT |
| ORF: 70 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACCGCGGCCTGAGGC |
| | | TGGAGACCCATGAAGAGGCCAGTGTGAAGATGCTGCCCACCTATGTGCG |
| | | CTCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCG |
| | | GAGGATGCCATGACAGGCACTGCTGAGATGCTCTTTGACTACATCTCTG |
| | | AGTGCATCTCAGACTTCCTGGACAAGCATCAGATGAAACACAAGAAGCT |
| | | GCCCCTGGGCTTCACCTTCTCCTTTCCTGTGAGGCATGAAGACATTGATA |
| | | AGGGCATCCTTCTCAACTGGACCAAGGGCTTCAAGGCCTCAGGAGCAGA |
| | | AGGGAACAATGTTGTGGGGCTTCTGAGAGATGCTATCAAAAGAAGAGG |
| | | GGACTTTGAAATGGATGTGGTGGCAATGGTGAATGACACAGTGGCCACT |
| | | ATGATCTCCTGCTACTATGAAGACCATCAGTGTGAGGTAGGCATGATAG |
| | | TGGGCACAGGCTGCAATGCCTGCTACATGGAGGAGATGCAGAATGTGGA |
| | | GCTGGTGGAGGGGGATGAGGGCCGCATGTGTGTCAATACTGAGTGGGGC |
| | | GCCTTTGGGGACAGTGGAGAGCTGGATGAGTTCCTGCTGGAGTATGACC |
| | | GCCTGGTGGATGAGAGCTCTGCAAACCCAGGTCAGCAGCTGTATGAGAA |
| | | GCTCATAGGTGGCAAGTACATGGGAGAGCTGGTGCGGCTTGTGCTGCTC |
| | | AGGCTAGTGGATGAAAACCTGCTCTTCCATGGGGAGGCCTCAGAGCAGC |
| | | TGCGCACACGCGGAGCCTTTGAGACGCGCTTTGTGTCTCAAGTGGAGAG |
| | | TGACACAGGTGACCGCAAGCAGATCTACAACATCCTGAGCACTCTGGGG |
| | | CTGCGGCCCTCAACCACAGACTGTGACATTGTGCGCCGCGCCTGTGAGT |
| | | CTGTGTCTACGCGCGCTGCGCACATGTGCAGTGCGGGGCTGGCGGGAGT |
| | | CATCAACCGCATGCGCGAGAGCCGCTCAGAGGATGTAATGCGCATCACT |
| | | GTGGGTGTGGATGGCTCTGTGTACAAGCTGCACCCCAGCTTCAAGGAGC |
| | | GGTTCCATGCCAGTGTGCGCAGGCTGACTCCCAGCTGTGAGATCACCTTT |
| | | ATAGAGTCAGAGGAGGGCAGTGGCAGAGGCGCGGCCCTGGTCTCCGCG |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 30 | Modified | [CTAG]AAGCTTTATTGTGGTAGTTTATCACAGTTAAATTGCTAAGGC |
| 5′UTR: 42 | hGck-12 | AGTCAGTGCTTCTGACACAACAGTCTCCAACTTAAGCTGCAGTGACT |
| ORF: 71 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACCGCGGCCTGAGGC |
| | | TGGAGACCCATGAAGAGGCCAGTGTGAAGATGCTGCCCACCTATGTGCG |
| | | CTCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGTCTGTGAAGACCAAACACCAGATGTACTCCATCCCTG |
| | | AGGATGCCATGACTGGCACTGCTGAGATGCTCTTTGACTACATCTCTGAG |
| | | TGCATCTCTGACTTCCTGGACAAGCATCAGATGAAACACAAGAAGCTGC |
| | | CCCTGGGCTTCACCTTCTCATTTCCTGTGAGGCATGAAGACATTGATAAG |
| | | GGCATCCTTCTCAACTGGACCAAGGGCTTCAAGGCCTCAGGAGCAGAAG |
| | | GGAACAATGTTGTGGGGCTTCTGAGAGATGCTATCAAAAGAAGAGGGG |
| | | ACTTTGAAATGGATGTGGTGGCAATGGTGAATGACACTGTGGCCACTAT |
| | | GATCTCCTGCTACTATGAAGACCATCAGTGTGAGGTTGGCATGATTGTG |
| | | GGCACTGGCTGCAATGCCTGCTACATGGAGGAGATGCAGAATGTGGAGC |
| | | TGGTGGAGGGGGATGAGGGCCGCATGTGTGTCAATACTGAGTGGGGCGC |
| | | CTTTGGGGACTCTGGTGAGCTGGATGAGTTCCTGCTGGAGTATGACCGC |
| | | CTGGTGGATGAGAGCTCTGCAAACCCTGGTCAGCAGCTGTATGAGAAGC |
| | | TCATAGGTGGCAAGTACATGGGTGAGCTGGTGCGGCTTGTGCTGCTCAG |
| | | GTTAGTGGATGAAAACCTGCTCTTCCATGGGGAGGCCTCTGAGCAGCTG |
| | | CGCACACGCGGAGCCTTTGAGACGCGCTTTGTGTCTCAAGTGGAGTCTG |
| | | ACACTGGTGACCGCAAGCAGATCTACAACATCCTGAGCACTCTGGGGCT |
| | | GAGACCCTCTACCACTGACTGTGACATTGTGCGCCGCGCCTGTGAGTCTG |
| | | TGTCTACGCGCGCTGCGCACATGTGCTCTGCGGGGCTGGCGGGTGTCAT |
| | | CAACCGCATGCGCGAGAGCCGCTCTGAGGATGTAATGCGCATCACTGTG |
| | | GGTGTGGATGGCTCTGTGTACAAGCTGCACCCCTCATTCAAGGAGCGGT |
| | | TCCATGCCTCTGTGCGCAGGCTGACTCCCAGCTGTGAGATCACCTTTATT |
| | | GAGTCTGAGGAGGGCAGTGGCAGAGGCGCGGCCCTGGTCTCTGCGGTGG |
| | | CCTGTAAGAAGGCCTGTATGCTGGGCCAGTGAAGCCAGCTGCCTTCCCTT |
| | | TACTGTGGGTTAATGCTTGCAGCAGACATGATAAGATACATTGATGAGTTTGG |
| | | ACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATCCGATAAGGGACTAGAGCAGA |
| | | AGCTTTTG |
| Full: 31 | Modified | [CTAG]AAGCTTTATTGTGGTAGTTTATCACAGTTAAATTGCTAAGGC |
| 5′UTR: 42 | hGck-13 | AGTCAGTGCTTCTGACACAACAGTCTCCAACTTAAGCTGCAGTGACT |
| ORF: 72 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGAGGCCTGAGGC |
| | | TGGAGACCCATGAAGAGGCCAGTGTGAAGATGCTGCCCACCTATGTGAG |
| | | ATCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCAGACTTCCTGGACAAGCATCAGATGAAACACAAGAAGCTG |
| | | CCCCTGGGCTTCACCTTCTCCTTTCCTGTGAGGCATGAAGACATTGATAA |
| | | GGGCATCCTTCTCAACTGGACCAAGGGCTTCAAGGCCTCAGGAGCAGAA |
| | | GGGAACAATGTTGTGGGGCTTCTGAGAGATGCTATCAAAAGAAGAGGG |
| | | GACTTTGAAATGGATGTGGTGGCAATGGTGAATGACACAGTGGCCACTA |
| | | TGATCTCCTGCTACTATGAAGACCATCAGTGTGAGGTTGGCATGATTGTG |
| | | GGCACAGGCTGCAATGCCTGCTACATGGAGGAGATGCAGAATGTGGAG |
| | | CTGGTGGAGGGGGATGAGGGCAGGATGTGTGTCAATACTGAGTGGGGA |
| | | GCCTTTGGGGACTCTGGAGAGCTGGATGAGTTCCTGCTGGAGTATGACA |
| | | GACTGGTGGATGAGAGCTCAGCAAACCCAGGTCAGCAGCTGTATGAGA |
| | | AGCTCATAGGTGGCAAGTACATGGGAGAGCTGGTGAGGCTTGTGCTGCT |
| | | CAGGTTGGTGGATGAAAACCTGCTCTTCCATGGGGAGGCCAGTGAGCAG |
| | | CTGAGAACAAGGGGAGCCTTTGAGACAAGATTTGTGTCACAGGTGGAGT |
| | | CTGACACAGGAGACAGGAAGCAGATCTACAACATCCTGAGCACCCTGG |
| | | GGCTGAGACCCTCCACCACTGACTGTGACATTGTGAGGAGAGCCTGTGA |
| | | GAGTGTGTCTACCAGGGCTGCTCACATGTGCTCAGCAGGGCTGGCTGGA |
| | | GTCATCAACAGAATGAGGGAGAGCAGATCTGAGGATGTAATGAGGATC |
| | | ACTGTGGGTGTGGATGGCAGTGTGTACAAGCTGCACCCCAGCTTCAAGG |
| | | AGAGATTCCATGCCTCAGTGAGGAGGCTGACCCCCAGCTGTGAGATCAC |
| | | CTTCATTGAGTCTGAGGAGGGCAGTGGCAGAGGAGCAGCCCTGGTCTCT |
| | | GCTGTGGCCTGTAAGAAGGCCTGTATGCTGGGCCAGTGAAGCCAGCTGC |
| | | CTTCCCTTTACTGTGGGTTAATGCTTGCAGCAGACATGATAAGATACATTGATG |
| | | AGTTTGGACAAACCACAACTAGAATGCAGTGAAAAAAATGCTTTATTTGTGAAA |
| | | TTTGTGATGCTATTGCTTTATTTGTAACCATTATAAGCTGCAATAAACAAGTTAA |
| | | CAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTA |
| | | GAGCAGAAGCTTTTG |
| Full: 32 | Modified | [CTAG]AAGCTTTATTGTGGTAGTTTATCACAGTTAAATTGCTAAGGC |
| 5′UTR: 42 | hGck-14 | AGTCAGTGCTTCTGACACAACAGTCTCCAACTTAAGCTGCAGTGACT |
| ORF: 73 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGAGGCCTGAGGC |
| | | TGGAGACCCATGAAGAGGCCAGTGTGAAGATGCTGCCCACCTATGTGAG |
| | | ATCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCAGACTTCCTGGACAAGCATCAGATGAAACACAAGAAGCTG |
| | | CCCCTGGGCTTCACCTTCTCCTTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGAAGAGGG |
| | | GACTTTGAAATGGATGTGGTGGCAATGGTGAATGACACAGTGGCCACCA |
| | | TGATCTCCTGCTACTATGAAGACCATCAGTGTGAGGTGGGCATGATTGT |
| | | GGGCACTGGCTGCAATGCCTGCTACATGGAGGAGATGCAGAATGTGGAG |
| | | CTGGTGGAGGGGGATGAGGGCAGAATGTGTGTCAATACAGAGTGGGGA |
| | | GCCTTTGGGGACTCTGGAGAGCTGGATGAGTTCCTGCTGGAGTATGACA |
| | | GACTGGTGGATGAGAGCTCAGCAAACCCAGGTCAGCAGCTGTATGAGA |
| | | AGCTCATAGGTGGCAAGTACATGGGAGAGCTGGTGAGACTTGTGCTGCT |
| | | CAGGCTGGTGGATGAAAACCTGCTCTTCCATGGGGAGGCCAGTGAGCAG |
| | | CTGAGAACAAGAGGAGCCTTTGAGACCAGATTTGTGAGCCAGGTGGAGT |
| | | CAGACACAGGAGACAGAAAGCAGATCTACAACATCCTGAGCACCCTGG |
| | | GGCTGAGACCCAGCACCACTGACTGTGACATTGTGAGAAGAGCCTGTGA |
| | | GTCTGTGTCTACCAGAGCTGCCCACATGTGCAGTGCTGGGCTGGCTGGA |
| | | GTCATCAACAGAATGAGAGAGAGCAGATCAGAGGATGTAATGAGAATC |
| | | ACTGTGGGTGTGGATGGCTCTGTGTACAAGCTGCACCCCAGCTTCAAGG |
| | | AGAGATTCCATGCCAGTGTGAGAAGGCTGACCCCCAGCTGTGAGATCAC |
| | | CTTTATTGAGTCAGAGGAGGGCAGTGGCAGAGGAGCAGCCCTGGTCTCT |
| | | GCTGTGGCCTGTAAGAAGGCCTGTATGCTGGGCCAGTGAAGCCAGCTGC |
| | | CTTCCCTTTACTGTGGGTTAATGCTTGCAGCAGACATGATAAGATACATTGATG |
| | | AGTTTGGACAAACCACAACTAGAATGCAGTGAAAAAAATGCTTTATTTGTGAAA |
| | | TTTGTGATGCTATTGCTTTATTTGTAACCATTATAAGCTGCAATAAACAAGTTAA |
| | | CAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTA |
| | | GAGCAGAAGCTTTTG |
| Full: 33 | Modified | [CTAG]AAGCTTTATTGTGGTAGTTTATCACAGTTAAATTGCTAAGGC |
| 5′UTR: 42 | hGck-15 | AGTCAGTGCTTCTGACACAACAGTCTCCAACTTAAGCTGCAGTGACT |
| ORF: 74 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGAGGCCTGAGGC |
| | | TGGAGACCCATGAAGAGGCCAGTGTGAAGATGCTGCCCACCTATGTGAG |
| | | ATCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCA |
| | | GAGGATGCCATGACAGGCACTGCTGAGATGCTCTTTGACTACATCTCTG |
| | | AGTGCATCTCAGACTTCCTGGACAAGCATCAGATGAAACACAAGAAGCT |
| | | GCCCCTGGGCTTCACCTTCTCCTTTCCTGTGAGGCATGAAGACATTGATA |
| | | AGGGCATCCTTCTCAACTGGACCAAGGGCTTCAAGGCCTCAGGAGCAGA |
| | | AGGGAACAATGTTGTGGGGCTTCTGAGAGATGCTATCAAAAGAAGAGG |
| | | GGACTTTGAAATGGATGTGGTGGCAATGGTGAATGACACAGTGGCCACT |
| | | ATGATCTCCTGCTACTATGAAGACCATCAGTGTGAGGTAGGCATGATAG |
| | | TGGGCACAGGCTGCAATGCCTGCTACATGGAGGAGATGCAGAATGTGGA |
| | | GCTGGTGGAGGGGGATGAGGGCAGGATGTGTGTCAATACTGAGTGGGG |
| | | AGCCTTTGGGGACAGTGGAGAGCTGGATGAGTTCCTGCTGGAGTATGAC |
| | | AGACTGGTGGATGAGAGCTCTGCAAACCCTGGTCAGCAGCTGTATGAGA |
| | | AGCTCATAGGTGGCAAGTACATGGGTGAGCTGGTGAGGCTTGTGCTGCT |
| | | CAGGCTAGTGGATGAAAACCTGCTCTTCCATGGGGAGGCCTCAGAGCAG |
| | | CTGAGAACAAGGGGAGCCTTTGAGACAAGATTTGTGTCTCAGGTGGAGA |
| | | GTGACACTGGAGACAGGAAGCAGATCTACAACATCCTGAGCACACTGG |
| | | GGCTGAGACCCTCAACCACTGACTGTGACATTGTGAGGAGAGCCTGTGA |
| | | GTCTGTGTCTACAAGGGCTGCACACATGTGCAGTGCTGGGCTGGCAGGT |
| | | GTCATCAACAGAATGAGGGAGAGCAGATCAGAGGATGTAATGAGGATC |
| | | ACTGTGGGAGTGGATGGCTCTGTGTACAAGCTGCACCCCAGCTTCAAGG |
| | | AGAGATTCCATGCCAGTGTGAGGAGGCTGACCCCCAGCTGTGAGATCAC |
| | | CTTTATAGAGTCAGAGGAGGGCAGTGGCAGAGGTGCTGCCCTGGTCTCT |
| | | GCAGTGGCCTGTAAGAAGGCCTGTATGCTGGGCCAGTGAAGCCAGCTGC |
| | | CTTCCCTTTACTGTGGGTTAATGCTTGCAGCAGACATGATAAGATACATTGATG |
| | | AGTTTGGACAAACCACAACTAGAATGCAGTGAAAAAAATGCTTTATTTGTGAAA |
| | | TTTGTGATGCTATTGCTTTATTTGTAACCATTATAAGCTGCAATAAACAAGTTAA |
| | | CAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTA |
| | | GAGCAGAAGCTTTTG |
| Full: 34 | Modified | [CTAG]AAGCTTTATTGTGGTAGTTTATCACAGTTAAATTGCTAAGGC |
| 5′UTR: 42 | hGck-16 | AGTCAGTGCTTCTGACACAACAGTCTCCAACTTAAGCTGCAGTGACT |
| ORF: 75 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGAGGCCTGAGGC |
| | | TGGAGACCCATGAAGAGGCCAGTGTGAAGATGCTGCCCACCTATGTGAG |
| | | ATCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGTCTGTGAAGACCAAACACCAGATGTACTCCATCCCTG |
| | | AGGATGCCATGACTGGCACTGCTGAGATGCTCTTTGACTACATCTCTGAG |
| | | TGCATCTCTGACTTCCTGGACAAGCATCAGATGAAACACAAGAAGCTGC |
| | | CCCTGGGCTTCACCTTCTCATTTCCTGTGAGGCATGAAGACATTGATAAG |
| | | GGCATCCTTCTCAACTGGACCAAGGGCTTCAAGGCCTCAGGAGCAGAAG |
| | | GGAACAATGTTGTGGGGCTTCTGAGAGATGCTATCAAAAGAAGAGGGG |
| | | ACTTTGAAATGGATGTGGTGGCAATGGTGAATGACACTGTGGCCACTAT |
| | | GATCTCCTGCTACTATGAAGACCATCAGTGTGAGGTTGGCATGATTGTG |
| | | GGCACTGGCTGCAATGCCTGCTACATGGAGGAGATGCAGAATGTGGAGC |
| | | TGGTGGAGGGGGATGAGGGCAGAATGTGTGTCAATACTGAGTGGGGTG |
| | | CCTTTGGGGACTCTGGTGAGCTGGATGAGTTCCTGCTGGAGTATGACAG |
| | | ACTGGTGGATGAGAGCTCTGCAAACCCTGGTCAGCAGCTGTATGAGAAG |
| | | CTCATAGGTGGCAAGTACATGGGTGAGCTGGTGAGACTTGTGCTGCTCA |
| | | GGTTAGTGGATGAAAACCTGCTCTTCCATGGGGAGGCCTCTGAGCAGCT |
| | | GAGAACAAGAGGAGCCTTTGAGACTAGATTTGTGTCTCAGGTGGAGTCT |
| | | GACACTGGTGACAGAAAGCAGATCTACAACATCCTGAGCACTCTGGGGC |
| | | TGAGACCCTCTACCACTGACTGTGACATTGTGAGAAGAGCCTGTGAGTC |
| | | TGTGTCTACTAGAGCTGCTCACATGTGCTCTGCTGGGCTGGCTGGTGTCA |
| | | TCAACAGAATGAGAGAGAGCAGATCTGAGGATGTAATGAGAATCACTG |
| | | TGGGTGTGGATGGCTCTGTGTACAAGCTGCACCCCTCATTCAAGGAGAG |
| | | ATTCCATGCCTCTGTGAGAAGGCTGACTCCCAGCTGTGAGATCACCTTCA |
| | | TTGAGTCTGAGGAGGGCAGTGGCAGAGGTGCTGCCCTGGTCTCTGCTGT |
| | | GGCCTGTAAGAAGGCCTGTATGCTGGGCCAGTGAAGCCAGCTGCCTTCCC |
| | | TTTACTGTGGGTTAATGCTTGCAGCAGACATGATAAGATACATTGATGAGTTTG |
| | | GACAAACCACAACTAGAATGCAGTGAAAAAAATGCTTTATTTGTGAAATTTGTG |
| | | ATGCTATTGCTTTATTTGTAACCATTATAAGCTGCAATAAACAAGTTAACAACAA |
| | | CAATTGCATTCATTTTATGTTTCAGGTTCAGGGGGAGATGTGGGAGGTTTTTTA |
| | | AAGCAAGTAAAACCTCTACAAATGTGGTAAAATCCGATAAGGGACTAGAGCAG |
| | | AAGCTTTTG |
| Full: 35 | Modified | [CTAG]AAGCTTTATTGTGGTAGTTTATCACAGTTAAATTGCTAAGGC |
| 5′UTR: 42 | hGck-17 | AGTCAGTGCTTCTGACACAACAGTCTCCAACTTAAGCTGCAGTGACT |
| ORF: 76 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGACGTGACCCGGAGCCAGGCTCAAACAGCACTGAC |
| | | TCTCGTCGAGCAGATCTTGGCGGAATTCCAGCTTCAAGAGGAAGATCTG |
| | | AAGAAAGTTATGAGAAGGATGCAGAAGGAGATGGACCGCGGCTTACGA |
| | | CTGGAAACGCACGAGGAGGCCTCCGTGAAAATGCTACCCACCTACGTGC |
| | | GTTCTACACCTGAAGGATCAGAGGTGGGGGATTTTCTCAGTCTGGACTT |
| | | GGGTGGCACTAACTTCCGGGTCATGCTGGTGAAGGTTGGAGAAGGGGAG |
| | | GAAGGCCAGTGGTCGGTGAAAACCAAGCATCAGATGTATAGCATTCCAG |
| | | AGGATGCTATGACAGGCACCGCCGAGATGCTTTTTGACTACATATCCGA |
| | | ATGCATCTCTGATTTCCTCGACAAGCACCAAATGAAACATAAGAAACTG |
| | | CCGCTGGGATTTACTTTCTCATTTCCCGTGAGACACGAGGATATTGACAA |
| | | GGGGATCCTCTTGAATTGGACGAAAGGCTTCAAGGCAAGTGGCGCCGAA |
| | | GGAAACAATGTAGTGGGGCTTCTGAGGGATGCTATTAAGCGCCGGGGTG |
| | | ACTTTGAGATGGACGTTGTGGCAATGGTCAACGATACAGTGGCCACAAT |
| | | GATCAGCTGTTATTACGAGGACCATCAGTGCGAAGTCGGCATGATAGTG |
| | | GGAACTGGGTGTAATGCGTGCTATATGGAGGAAATGCAGAACGTTGAGT |
| | | TAGTGGAAGGCGATGAGGGTAGAATGTGTGTAAATACCGAGTGGGGAG |
| | | CTTTCGGGGACTCCGGCGAACTGGATGAGTTCCTACTCGAATACGACAG |
| | | GCTGGTGGATGAGTCTAGCGCCAACCCTGGCCAGCAATTGTATGAAAAA |
| | | CTGATCGGAGGGAAGTACATGGGTGAGCTTGTCCGCCTCGTTCTGCTGC |
| | | GATTAGTGGACGAGAATCTCCTGTTTCACGGCGAAGCATCCGAGCAGTT |
| | | GCGTACACGGGGAGCCTTCGAAACCAGATTTGTCTCACAAGTGGAGAGT |
| | | GATACTGGGGACAGGAAACAGATTTACAACATCCTTAGCACACTGGGCC |
| | | TACGCCCATCTACGACCGATTGCGACATTGTGCGGCGAGCTTGTGAGTC |
| | | CGTTTCGACTAGAGCCGCACACATGTGCTCAGCTGGTCTCGCCGGAGTA |
| | | ATCAATAGGATGCGCGAAAGCCGGAGTGAGGACGTGATGAGAATAACA |
| | | GTCGGCGTGGATGGGTCTGTCTATAAGCTGCATCCCTCCTTCAAGGAAA |
| | | GGTTTCACGCGAGCGTGCGCCGTCTGACCCCTTCATGTGAGATTACTTTC |
| | | ATCGAATCCGAGGAGGGCAGTGGACGGGGGGCAGCCTTGGTTTCTGCTG |
| | | TGGCCTGCAAAAAGGCATGCATGCTTGGCCAATGAAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCA |
| | | GAAGCTTTTG |
| Full: 36 | Modified | [CTAG]AAGCTTTATTGTGGTAGTTTATCACAGTTAAATTGCTAAGGC |
| 5′UTR: 42 | hGck-18 | AGTCAGTGCTTCTGACACAACAGTCTCCAACTTAAGCTGCAGTGACT |
| ORF: 77 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTGACCAGAAGCCAGGCTCAAACAGCACTGAC |
| | | TCTGGTAGAGCAGATCTTGGCGGAATTCCAGCTTCAAGAGGAAGATCTG |
| | | AAGAAAGTTATGAGAAGGATGCAGAAGGAGATGGACCGCGGCTTAAGG |
| | | CTGGAAACCCATGAGGAGGCCAGTGTGAAAATGCTACCCACCTATGTAA |
| | | GATCTACACCTGAAGGATCAGAGGTGGGGGATTTTCTCAGTCTGGACTT |
| | | GGGTGGCACTAACTTCAGGGTCATGCTGGTGAAGGTTGGAGAAGGGGA |
| | | GGAAGGCCAGTGGTCAGTGAAAACCAAGCATCAGATGTATAGCATTCCA |
| | | GAGGATGCTATGACAGGTACCGCTGAGATGCTTTTTGACTACATATCTG |
| | | AATGCATCTCTGATTTCCTAGACAAGCACCAAATGAAACATAAGAAACT |
| | | GCCGCTGGGATTTACTTTCTCATTTCCTGTGAGACATGAGGATATTGACA |
| | | AGGGGATCCTCTTGAATTGGACAAAAGGCTTCAAGGCAAGTGGCGCAGA |
| | | AGGAAACAATGTAGTGGGGCTTCTGAGGGATGCTATTAAGCGCAGAGGT |
| | | GACTTTGAGATGGATGTTGTGGCAATGGTCAATGATACAGTGGCCACAA |
| | | TGATCAGCTGTTATTATGAGGACCATCAGTGTGAAGTGGGCATGATAGT |
| | | GGGAACTGGGTGTAATGCCTGCTATATGGAGGAAATGCAGAATGTTGAG |
| | | TTAGTGGAAGGTGATGAGGGTAGAATGTGTGTAAATACAGAGTGGGGA |
| | | GCTTTTGGGGACAGCGGAGAACTGGATGAGTTCCTATTAGAATATGACA |
| | | GGCTGGTGGATGAGTCTAGCGCCAACCCTGGCCAGCAATTGTATGAAAA |
| | | ACTGATTGGAGGGAAGTACATGGGTGAGCTTGTCCGCCTTGTTCTGCTGC |
| | | GGTTAGTGGATGAGAATCTCCTGTTTCATGGGGAAGCAAGTGAGCAGTT |
| | | GCGGACAAGGGGAGCCTTTGAAACCAGATTTGTCTCACAAGTGGAGAGT |
| | | GATACTGGGGACAGGAAACAGATTTACAACATCCTTAGCACACTGGGCC |
| | | TAAGACCATCTACCACTGATTGTGACATTGTGCGGCGGGCTTGTGAGTC |
| | | AGTTTCCACTAGAGCCGCACACATGTGCTCAGCTGGTTTGGCTGGAGTA |
| | | ATCAATAGGATGCGCGAAAGCAGGAGTGAGGATGTGATGAGAATAACA |
| | | GTTGGTGTGGATGGGTCTGTCTATAAGCTGCATCCCTCCTTCAAGGAAAG |
| | | GTTTCACGCGTCTGTGCGCAGACTGACCCCTTCATGTGAGATTACTTTCA |
| | | TAGAATCTGAGGAGGGCAGTGGAAGGGGGGCAGCCTTGGTTTCTGCTGT |
| | | GGCCTGCAAAAAGGCATGCATGCTTGGCCAATGAAGCCAGCTGCCTTCCC |
| | | TTTACTGTGGGTTAATGCTTGCAGCAGACATGATAAGATACATTGATGAGTTTG |
| | | GACAAACCACAACTAGAATGCAGTGAAAAAAATGCTTTATTTGTGAAATTTGTG |
| | | ATGCTATTGCTTTATTTGTAACCATTATAAGCTGCAATAAACAAGTTAACAACAA |
| | | CAATTGCATTCATTTTATGTTTCAGGTTCAGGGGGAGATGTGGGAGGTTTTTTA |
| | | AAGCAAGTAAAACCTCTACAAATGTGGTAAAATCCGATAAGGGACTAGAGCAG |
| | | AAGCTTTTG |
| Full: 37 | Modified | [CTAG]AAGCTTTATTGTGGTAGTTTATCACAGTTAAATTGCTAAGGC |
| 5′UTR: 42 | hGck-19 | AGTCAGTGCTTCTGACACAACAGTCTCCAACTTAAGCTGCAGTGACT |
| ORF: 78 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTGACCAGAAGCCAGGCTCAAACAGCACTGAC |
| | | TCTGGTGGAGCAGATCTTGGCGGAATTCCAGCTTCAAGAGGAAGATCTG |
| | | AAGAAAGTTATGAGAAGGATGCAGAAGGAGATGGACCGCGGCTTAAGG |
| | | CTGGAAACCCATGAGGAGGCCAGTGTGAAAATGCTACCCACCTATGTAA |
| | | GATCTACACCTGAAGGATCAGAGGTGGGGGATTTTCTCAGTCTGGACTT |
| | | GGGTGGCACTAACTTCAGGGTCATGCTGGTGAAGGTTGGAGAAGGGGA |
| | | GGAAGGCCAGTGGTCAGTGAAAACCAAGCATCAGATGTATAGCATTCCA |
| | | GAGGATGCTATGACAGGTACCGCTGAGATGCTTTTTGACTACATATCTG |
| | | AATGCATCTCTGATTTCCTGGACAAGCACCAAATGAAACATAAGAAACT |
| | | GCCGCTGGGATTTACTTTCTCATTTCCTGTGAGACATGAGGATATTGACA |
| | | AGGGGATCCTCTTGAATTGGACCAAAGGCTTCAAGGCAAGTGGCGCAGA |
| | | AGGAAACAATGTAGTGGGGCTTCTGAGGGATGCTATTAAGCGCAGAGGT |
| | | GACTTTGAGATGGATGTTGTGGCAATGGTCAATGATACAGTGGCCACAA |
| | | TGATCAGCTGTTATTATGAGGACCATCAGTGTGAAGTGGGCATGATAGT |
| | | GGGAACTGGGTGTAATGCCTGCTATATGGAGGAAATGCAGAATGTTGAG |
| | | TTAGTGGAAGGTGATGAGGGTAGAATGTGTGTAAATACAGAGTGGGGA |
| | | GCTTTTGGGGACAGCGGAGAACTGGATGAGTTCCTACTGGAATATGACA |
| | | GGCTGGTGGATGAGTCTAGCGCCAACCCTGGCCAGCAATTGTATGAAAA |
| | | ACTGATTGGAGGGAAGTACATGGGTGAGCTTGTCCGCCTGGTTCTGCTG |
| | | CGGTTAGTGGATGAGAATCTCCTGTTTCATGGGGAAGCAAGTGAGCAGT |
| | | TGCGGACAAGGGGAGCCTTTGAAACCAGATTTGTCTCACAAGTGGAGAG |
| | | TGATACTGGGGACAGGAAACAGATTTACAACATCCTTAGCACACTGGGC |
| | | CTAAGACCATCTACCACAGATTGTGACATTGTGCGGCGGGCTTGTGAGT |
| | | CAGTTAGCACTAGAGCCGCACACATGTGCTCAGCTGGTCTGGCTGGAGT |
| | | AATCAATAGGATGCGCGAAAGCAGGAGTGAGGATGTGATGAGAATAAC |
| | | AGTGGGTGTGGATGGGTCTGTCTATAAGCTGCATCCCTCCTTCAAGGAA |
| | | AGGTTTCACGCGTCTGTGCGCAGACTGACCCCTTCATGTGAGATTACTTT |
| | | CATAGAATCTGAGGAGGGCAGTGGAAGGGGGGCAGCCTTGGTTTCTGCT |
| | | GTGGCCTGCAAAAAGGCATGCATGCTTGGCCAATGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 38 | Modified | [CTAG]AAGCTTTATTGTGGTAGTTTATCACAGTTAAATTGCTAAGGC |
| 5′UTR: 42 | hGck-20 | AGTCAGTGCTTCTGACACAACAGTCTCCAACTTAAGCTGCAGTGACT |
| ORF: 79 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTGACCAGAAGCCAGGCTCAAACAGCACTGAC |
| | | TCTGGTAGAGCAGATCTTGGCTGAATTCCAGCTTCAAGAGGAAGATCTG |
| | | AAGAAAGTTATGAGAAGGATGCAGAAGGAGATGGACAGGGGCTTAAGA |
| | | CTGGAAACCCATGAGGAGGCCAGTGTGAAAATGCTACCCACCTATGTAA |
| | | GGTCTACACCTGAAGGATCAGAGGTGGGGGATTTTCTCAGTCTGGACTT |
| | | GGGTGGCACTAACTTCAGAGTCATGCTGGTGAAGGTTGGAGAAGGGGA |
| | | GGAAGGCCAGTGGTCAGTGAAAACCAAGCATCAGATGTATAGCATTCCA |
| | | GAGGATGCTATGACAGGCACAGCAGAGATGCTTTTTGACTACATATCTG |
| | | AATGCATCTCTGATTTCCTAGACAAGCACCAAATGAAACATAAGAAACT |
| | | GCCACTGGGATTTACTTTCTCATTTCCTGTGAGACATGAGGATATTGACA |
| | | AGGGGATCCTCTTGAATTGGACCAAAGGCTTCAAGGCAAGTGGTGCTGA |
| | | AGGAAACAATGTAGTGGGGCTTCTGAGGGATGCTATTAAGAGGAGAGG |
| | | TGACTTTGAGATGGATGTTGTGGCAATGGTCAATGATACAGTGGCCACA |
| | | ATGATCAGCTGTTATTATGAGGACCATCAGTGTGAAGTGGGCATGATAG |
| | | TGGGAACTGGGTGTAATGCCTGCTATATGGAGGAAATGCAGAATGTTGA |
| | | GTTAGTGGAAGGAGATGAGGGTAGAATGTGTGTAAATACAGAGTGGGG |
| | | AGCTTTTGGGGACAGTGGGGAACTGGATGAGTTCCTATTAGAATATGAC |
| | | AGGCTGGTGGATGAGTCTTCAGCCAACCCTGGCCAGCAATTGTATGAAA |
| | | AACTGATTGGAGGGAAGTACATGGGTGAGCTTGTCAGGCTTGTTCTGCT |
| | | GAGATTAGTGGATGAGAATCTCCTGTTTCATGGTGAAGCATCTGAGCAG |
| | | TTGAGGACAAGAGGAGCCTTTGAAACCAGATTTGTCTCACAAGTGGAGA |
| | | GTGATACTGGGGACAGGAAACAGATTTACAACATCCTTAGCACACTGGG |
| | | CCTAAGGCCATCTACCACTGATTGTGACATTGTGAGAAGGGCTTGTGAG |
| | | AGTGTTTCCACTAGAGCAGCACACATGTGCTCAGCTGGTTTGGCTGGAG |
| | | TAATCAATAGGATGAGAGAAAGCAGGAGTGAGGATGTGATGAGAATAA |
| | | CAGTTGGAGTGGATGGGTCTGTCTATAAGCTGCATCCCTCCTTCAAGGA |
| | | AAGGTTTCATGCCTCTGTGAGAAGGCTGACCCCTTCATGTGAGATTACTT |
| | | TCATAGAATCAGAGGAGGGCAGTGGAAGAGGGGCAGCCTTGGTTTCTGC |
| | | TGTGGCCTGCAAAAAGGCATGCATGCTTGGCCAATGAAGCCAGCTGCCTT |
| | | CCCTTTACTGTGGGTTAATGCTTGCAGCAGACATGATAAGATACATTGATGAG |
| | | TTTGGACAAACCACAACTAGAATGCAGTGAAAAAAATGCTTTATTTGTGAAATT |
| | | TGTGATGCTATTGCTTTATTTGTAACCATTATAAGCTGCAATAAACAAGTTAACA |
| | | ACAACAATTGCATTCATTTTATGTTTCAGGTTCAGGGGGAGATGTGGGAGGTT |
| | | TTTTAAAGCAAGTAAAACCTCTACAAATGTGGTAAAATCCGATAAGGGACTAGA |
| | | GCAGAAGCTTTTG |
| Full: 39 | Modified | [CTAG]AAGCTTTATTGTGGTAGTTTATCACAGTTAAATTGCTAAGGC |
| 5′UTR: 42 | hGck-21 | AGTCAGTGCTTCTGACACAACAGTCTCCAACTTAAGCTGCAGTGACT |
| ORF: 80 | | CTCTTAAGGTAGCCTTGCAGAAGTTGGTCCTGAGGCACTGGGCAGG |
| 3′UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTGACCAGAAGCCAGGCTCAAACAGCACTGAC |
| | | TCTGGTGGAGCAGATCTTGGCTGAATTCCAGCTTCAAGAGGAAGATCTG |
| | | AAGAAAGTTATGAGAAGGATGCAGAAGGAGATGGACAGAGGCTTAAGA |
| | | CTGGAAACCCATGAGGAGGCCAGTGTGAAAATGCTACCCACCTATGTAA |
| | | GATCTACACCTGAAGGATCAGAGGTGGGGGATTTTCTCAGTCTGGACTT |
| | | GGGTGGCACTAACTTCAGAGTCATGCTGGTGAAGGTTGGAGAAGGGGA |
| | | GGAAGGCCAGTGGTCAGTGAAAACCAAGCATCAGATGTATAGCATTCCA |
| | | GAGGATGCTATGACAGGCACAGCAGAGATGCTTTTTGACTACATATCTG |
| | | AATGCATCTCTGATTTCCTGGACAAGCACCAAATGAAACATAAGAAACT |
| | | GCCCCTGGGATTTACTTTCTCATTTCCTGTGAGACATGAGGATATTGACA |
| | | AGGGGATCCTCTTGAATTGGACCAAAGGCTTCAAGGCAAGTGGTGCTGA |
| | | AGGAAACAATGTAGTGGGGCTTCTGAGGGATGCTATTAAGAGAAGAGG |
| | | TGACTTTGAGATGGATGTTGTGGCAATGGTCAATGATACAGTGGCCACA |
| | | ATGATCAGCTGTTATTATGAGGACCATCAGTGTGAAGTGGGCATGATAG |
| | | TGGGAACTGGGTGTAATGCCTGCTATATGGAGGAAATGCAGAATGTTGA |
| | | GTTAGTGGAAGGAGATGAGGGTAGAATGTGTGTAAATACAGAGTGGGG |
| | | AGCTTTTGGGGACAGTGGGGAACTGGATGAGTTCCTACTGGAATATGAC |
| | | AGGCTGGTGGATGAGTCTTCAGCCAACCCTGGCCAGCAATTGTATGAAA |
| | | AACTGATTGGAGGGAAGTACATGGGTGAGCTTGTCAGACTGGTTCTGCT |
| | | GAGATTAGTGGATGAGAATCTCCTGTTTCATGGTGAAGCATCTGAGCAG |
| | | TTGAGAACAAGAGGAGCCTTTGAAACCAGATTTGTCTCACAAGTGGAGA |
| | | GTGATACTGGGGACAGGAAACAGATTTACAACATCCTTAGCACACTGGG |
| | | CCTAAGACCATCTACCACTGATTGTGACATTGTGAGAAGAGCTTGTGAG |
| | | AGTGTTAGCACTAGAGCAGCACACATGTGCTCAGCTGGTCTGGCTGGAG |
| | | TAATCAATAGGATGAGAGAAAGCAGAAGTGAGGATGTGATGAGAATAA |
| | | CAGTGGGAGTGGATGGGTCTGTCTATAAGCTGCATCCCTCCTTCAAGGA |
| | | AAGGTTTCATGCCTCTGTGAGAAGACTGACCCCTTCATGTGAGATTACTT |
| | | TCATAGAATCAGAGGAGGGCAGTGGAAGAGGGGCAGCCTTGGTTTCTGC |
| | | TGTGGCCTGCAAAAAGGCATGCATGCTTGGCCAATGAAGCCAGCTGCCTT |
| | | CCCTTTACTGTGGGTTAATGCTTGCAGCAGACATGATAAGATACATTGATGAG |
| | | TTTGGACAAACCACAACTAGAATGCAGTGAAAAAAATGCTTTATTTGTGAAATT |
| | | TGTGATGCTATTGCTTTATTTGTAACCATTATAAGCTGCAATAAACAAGTTAACA |
| | | ACAACAATTGCATTCATTTTATGTTTCAGGTTCAGGGGGAGATGTGGGAGGTT |
| | | TTTTAAAGCAAGTAAAACCTCTACAAATGTGGTAAAATCCGATAAGGGACTAGA |
| | | GCAGAAGCTTTTG |
| Full: 89 | Modified | AAGCTTTATTGCGGTAGTTTATCACAGTTAAATTGCTAACGCAGTCA |
| 5′UTR: 83 | hGck-22 | GTGCTTCTGACACAACAGTCTCGAACTTAAGCTGCAGTGACTCTCTT |
| ORF: 61 | | AAGGTAGCCTTGCAGAAGTTGGTCGTGAGGCACTGGGCAGGTAAGT |
| 3′UTR: 102 | | ATCAAGGTTACAAGACAGGTTTAAGGAGACCAATAGAAACTGGGCT |
| | | TGTCGAGACAGAGAAGACTCTTGCGTTTCTGATAGGCACCTATTGGT |
| | | CTTACTGACATCCACTTTGCCTTTCTCTCCACAGGTGTCCACTCCCA |
| | | GTTCAATTACAGCTCTTAAGGCTAGAGTCCTGCAGAATTGCCACCAT |
| | | GGCCATGGATGTCACAAGGAGCCAGGCCCAGACAGCCTTGACTCTGGTA |
| | | GAGCAGATCCTGGCAGAGTTCCAGCTGCAGGAGGAGGACCTGAAGAAG |
| | | GTGATGAGAAGGATGCAGAAGGAGATGGACAGGGGCCTGAGGCTGGAG |
| | | ACCCATGAAGAGGCCAGTGTGAAGATGCTGCCCACCTATGTGAGGTCCA |
| | | CCCCAGAAGGCTCAGAAGTGGGGGACTTCCTCTCCCTGGACCTGGGTGG |
| | | CACTAACTTCAGGGTGATGCTGGTGAAGGTGGGAGAAGGTGAGGAGGG |
| | | GCAGTGGAGTGTGAAGACCAAACACCAGATGTACTCCATCCCTGAGGAT |
| | | GCCATGACAGGCACTGCTGAGATGCTCTTTGACTACATCTCTGAGTGCAT |
| | | CTCTGACTTCCTGGACAAGCATCAGATGAAACACAAGAAGCTGCCCCTG |
| | | GGCTTCACCTTCTCATTTCCTGTGAGGCATGAAGACATTGATAAGGGCAT |
| | | CCTTCTCAACTGGACCAAGGGCTTCAAGGCCTCAGGAGCAGAAGGGAAC |
| | | AATGTGGTGGGGCTTCTGAGAGATGCTATCAAAAGGAGAGGGGACTTTG |
| | | AAATGGATGTGGTGGCAATGGTGAATGACACTGTGGCCACCATGATCTC |
| | | CTGCTACTATGAAGACCATCAGTGTGAGGTGGGCATGATTGTGGGCACA |
| | | GGCTGCAATGCCTGCTACATGGAGGAGATGCAGAATGTGGAGCTGGTGG |
| | | AGGGGGATGAGGGCAGGATGTGTGTCAATACAGAGTGGGGAGCCTTTG |
| | | GGGACTCTGGGGAGCTGGATGAGTTCCTGCTGGAGTATGACAGGCTGGT |
| | | GGATGAGAGCTCAGCAAACCCTGGTCAGCAGCTGTATGAGAAGCTCATA |
| | | GGTGGCAAGTACATGGGAGAGCTGGTGAGGCTTGTGCTGCTCAGGCTGG |
| | | TGGATGAAAACCTGCTCTTCCATGGGGAGGCCTCTGAGCAGCTGAGGAC |
| | | AAGGGGAGCCTTTGAGACCAGGTTTGTGTCCCAGGTGGAGAGTGACACA |
| | | GGAGACAGGAAGCAGATCTACAACATCCTGAGCACCCTGGGGCTGAGA |
| | | CCCTCCACCACAGACTGTGACATTGTGAGGAGGGCTTGTGAGAGTGTGT |
| | | CTACCAGGGCTGCCCACATGTGCTCTGCTGGGCTGGCTGGGGTCATCAA |
| | | CAGGATGAGGGAGAGCAGGAGTGAGGATGTAATGAGGATCACTGTGGG |
| | | AGTGGATGGCTCTGTGTACAAGCTGCACCCCTCATTCAAGGAGAGGTTC |
| | | CATGCCAGTGTGAGGAGGCTGACCCCCAGCTGTGAGATCACCTTTATTG |
| | | AGTCTGAGGAGGGCAGTGGCAGGGGAGCAGCCCTGGTCTCTGCTGTGGC |
| | | CTGTAAGAAGGCCTGTATGCTGGGCCAGTGAAGCCAGCTGCCTTCCCTTT |
| | | ACTGTGGGTTAATGCTTGCAGCAGACATGATAAGATACATTGATGAGTTTGGA |
| | | CAAACCACAACTAGAATGCAGTGAAAAAAATGCTTTATTTGTGAAATTTGTGAT |
| | | GCTATTGCTTTATTTGTAACCATTATAAGCTGCAATAAACAAGTTAACAACAACA |
| | | ATTGCATTCATTTTATGTTTCAGGTTCAGGGGGAGATGTGGGAGGTTTTTTAAA |
| | | GCAAGTAAAACCTCTACAAATGTGGTAAAATAAGGGACTAGAGCAGTCTAGAA |
| | | AGCTT |
| Full: 90 | Modified | AAGCTTTATTGTGGTAGTTTATCACAGTTAAATTGCTAAGGCAGTCA |
| 5′UTR: | hGck-23 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 61 | | TCTTACTGACATCCACTTTGCCTTTCTCTCCACAGGTGTCCACTCCC |
| 3′UTR: 103 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGGATGCAGAAGGAGATGGACAGGGGCCTGAGGCTGGA |
| | | GACCCATGAAGAGGCCAGTGTGAAGATGCTGCCCACCTATGTGAGGTCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCTGACTTCCTGGACAAGCATCAGATGAAACACAAGAAGCTGCCCCT |
| | | GGGCTTCACCTTCTCATTTCCTGTGAGGCATGAAGACATTGATAAGGGC |
| | | ATCCTTCTCAACTGGACCAAGGGCTTCAAGGCCTCAGGAGCAGAAGGGA |
| | | ACAATGTGGTGGGGCTTCTGAGAGATGCTATCAAAAGGAGAGGGGACTT |
| | | TGAAATGGATGTGGTGGCAATGGTGAATGACACTGTGGCCACCATGATC |
| | | TCCTGCTACTATGAAGACCATCAGTGTGAGGTGGGCATGATTGTGGGCA |
| | | CAGGCTGCAATGCCTGCTACATGGAGGAGATGCAGAATGTGGAGCTGGT |
| | | GGAGGGGGATGAGGGCAGGATGTGTGTCAATACAGAGTGGGGAGCCTT |
| | | TGGGGACTCTGGGGAGCTGGATGAGTTCCTGCTGGAGTATGACAGGCTG |
| | | GTGGATGAGAGCTCAGCAAACCCTGGTCAGCAGCTGTATGAGAAGCTCA |
| | | TAGGTGGCAAGTACATGGGAGAGCTGGTGAGGCTTGTGCTGCTCAGGCT |
| | | GGTGGATGAAAACCTGCTCTTCCATGGGGAGGCCTCTGAGCAGCTGAGG |
| | | ACAAGGGGAGCCTTTGAGACCAGGTTTGTGTCCCAGGTGGAGAGTGACA |
| | | CAGGAGACAGGAAGCAGATCTACAACATCCTGAGCACCCTGGGGCTGA |
| | | GACCCTCCACCACAGACTGTGACATTGTGAGGAGGGCTTGTGAGAGTGT |
| | | GTCTACCAGGGCTGCCCACATGTGCTCTGCTGGGCTGGCTGGGGTCATC |
| | | AACAGGATGAGGGAGAGCAGGAGTGAGGATGTAATGAGGATCACTGTG |
| | | GGAGTGGATGGCTCTGTGTACAAGCTGCACCCCTCATTCAAGGAGAGGT |
| | | TCCATGCCAGTGTGAGGAGGCTGACCCCCAGCTGTGAGATCACCTTTATT |
| | | GAGTCTGAGGAGGGCAGTGGCAGGGGAGCAGCCCTGGTCTCTGCTGTGG |
| | | CCTGTAAGAAGGCCTGTATGCTGGGCCAGTGAAGCCAGCTGCCTTCCCTT |
| | | TACTGTGGGTTAATGCTTGCAGCAGACATGATAAGATACATTGATGAGTTTGG |
| | | ACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATAAGGGACTAGAGCAGGCTAGC |
| | | AAGCTT |
| Full: 91 | Modified | AAGCTTTATTGTGGTAGTTTATCACAGTTAAATTGCTAAGGCAGTCA |
| 5′UTR: | hGck-24 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 65 | | TCTTACTGACATCCACTTTGCCTTTCTCTCCACAGGTGTCCACTCCC |
| 3′UTR: 104 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGGATGCAGAAGGAGATGGACAGGGGCCTGAGGCTGGA |
| | | GACCCATGAAGAGGCCAGTGTGAAGATGCTGCCCACCTATGTGAGGTCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCTGACTTCCTGGACAAGCATCAGATGAAACACAAGAAGCTGCCCCT |
| | | GGGCTTCACCTTCTCATTTCCTGTGAGGCATGAAGACATTGATAAGGGC |
| | | ATCCTTCTCAACTGGACCAAGGGCTTCAAGGCCTCAGGAGCAGAAGGGA |
| | | ACAATGTGGTGGGGCTTCTGAGAGATGCTATCAAAAGGAGAGGGGACTT |
| | | TGAAATGGATGTGGTGGCAATGGTGAATGACACAGTGGCCACCATGATC |
| | | TCCTGCTACTATGAAGACCATCAGTGTGAGGTGGGCATGATAGTGGGCA |
| | | CAGGCTGCAATGCCTGCTACATGGAGGAGATGCAGAATGTGGAGCTGGT |
| | | GGAGGGGGATGAGGGCAGGATGTGTGTCAATACTGAGTGGGGAGCCTTT |
| | | GGGGACTCTGGGGAGCTGGATGAGTTCCTGCTGGAGTATGACAGGCTGG |
| | | TGGATGAGAGCTCAGCAAACCCTGGTCAGCAGCTGTATGAGAAGCTCAT |
| | | AGGTGGCAAGTACATGGGGGAGCTGGTGAGGCTTGTGCTGCTCAGGCTG |
| | | GTGGATGAAAACCTGCTCTTCCATGGGGAGGCCTCAGAGCAGCTGAGAA |
| | | CAAGGGGAGCCTTTGAGACCAGGTTTGTGTCCCAGGTGGAGAGTGACAC |
| | | AGGTGACAGGAAGCAGATCTACAACATCCTGAGCACCCTGGGGCTGAG |
| | | ACCCTCCACCACAGACTGTGACATTGTGAGGAGGGCCTGTGAGAGTGTG |
| | | TCTACCAGGGCTGCCCACATGTGCTCTGCTGGGCTGGCAGGAGTCATCA |
| | | ACAGGATGAGGGAGAGCAGGAGTGAGGATGTAATGAGGATCACTGTGG |
| | | GAGTGGATGGCTCTGTGTACAAGCTGCACCCCTCATTCAAGGAGAGGTT |
| | | CCATGCCAGTGTGAGAAGGCTGACCCCCAGCTGTGAGATCACCTTTATA |
| | | GAGTCTGAGGAGGGCAGTGGCAGGGGAGCAGCCCTGGTCTCTGCTGTGG |
| | | CCTGTAAGAAGGCCTGTATGCTGGGCCAGTGAAGCCAGCTGCCTTCCCTT |
| | | TACTGTGGGTTAATGCTTGCAGCAGACATGATAAGATACATTGATGAGTTTGG |
| | | ACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATAAGGGACTAGAGCAGATTAAT |
| | | AAGCTT |
| Full: 92 | Modified | AAGCTTTATTGTGGTAGTTTATCACAGTTAAATTGCTAAGGCAGTCA |
| 5′UTR: | hGck-25 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 67 | | TCTTACTGACATCCACTTTGCCTTTCTCTCCACAGGTGTCCACTCCC |
| 3′UTR: 105 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGGATGCAGAAGGAGATGGACAGGGGCCTGAGGCTGGA |
| | | GACCCATGAAGAGGCCAGTGTGAAGATGCTGCCCACCTATGTGAGGTCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCTGACTTCCTGGACAAGCATCAGATGAAACACAAGAAGCTGCCCCT |
| | | GGGCTTCACCTTCTCATTTCCTGTGAGGCATGAAGACATTGATAAGGGC |
| | | ATCCTTCTCAACTGGACCAAGGGCTTCAAGGCCTCAGGAGCAGAAGGGA |
| | | ACAATGTGGTGGGGCTTCTGAGAGATGCTATCAAAAGGAGAGGGGACTT |
| | | TGAAATGGATGTGGTGGCAATGGTGAATGACACAGTGGCCACTATGATC |
| | | TCCTGCTACTATGAAGACCATCAGTGTGAGGTGGGCATGATTGTGGGCA |
| | | CTGGCTGCAATGCCTGCTACATGGAGGAGATGCAGAATGTGGAGCTGGT |
| | | GGAGGGGGATGAGGGCAGGATGTGTGTCAATACTGAGTGGGGAGCCTTT |
| | | GGGGACTCTGGGGAGCTGGATGAGTTCCTGCTGGAGTATGACAGGCTGG |
| | | TGGATGAGAGCTCAGCAAACCCTGGTCAGCAGCTGTATGAGAAGCTCAT |
| | | AGGTGGCAAGTACATGGGAGAGCTGGTGAGACTTGTGCTGCTCAGGCTG |
| | | GTGGATGAAAACCTGCTCTTCCATGGGGAGGCCTCAGAGCAGCTGAGGA |
| | | CAAGGGGAGCCTTTGAGACCAGGTTTGTGTCCCAGGTGGAGAGTGACAC |
| | | TGGAGACAGGAAGCAGATCTACAACATCCTGAGCACCCTGGGGCTGAG |
| | | GCCCTCTACCACAGACTGTGACATAGTGAGAAGGGCCTGTGAGTCTGTG |
| | | TCTACCAGGGCTGCCCACATGTGCTCTGCTGGGCTGGCTGGAGTCATCA |
| | | ACAGAATGAGGGAGAGCAGAAGTGAGGATGTAATGAGGATCACTGTGG |
| | | GAGTGGATGGCTCTGTGTACAAGCTGCACCCCTCATTCAAGGAGAGGTT |
| | | CCATGCCAGTGTGAGGAGGCTGACCCCCAGCTGTGAGATCACCTTTATT |
| | | GAGTCTGAGGAGGGCAGTGGCAGGGGAGCTGCCCTGGTCTCTGCTGTGG |
| | | CCTGTAAGAAGGCCTGTATGCTGGGCCAGTGAAGCCAGCTGCCTTCCCTT |
| | | TACTGTGGGTTAATGCTTGCAGCAGACATGATAAGATACATTGATGAGTTTGG |
| | | ACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATAAGGGACTAGAGCAGATGCAT |
| | | AAGCTT |
| Full: 93 | Modified | AAGCTTTATTGTGGTAGTTTATCACAGTTAAATTGCTAAGGCAGTCA |
| 5′UTR: | hGck-26 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 68 | | TCTTACTGACATCCACTTTGCCTTTCTCTCCACAGGTGTCCACTCCC |
| 3′UTR: 106 | | AGTTCAATTACAGCTCTTAAGGCTAGAGTCCTGCAGAATTGCCACCA |
| | | TGGCTATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGAATGCAGAAGGAGATGGACCGCGGCCTGAGGCTGGA |
| | | GACCCATGAAGAGGCCAGTGTGAAGATGCTGCCCACCTATGTGCGCTCC |
| | | ACCCCAGAAGGCTCAGAAGTTGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCAGACTTCCTGGACAAGCATCAGATGAAACACAAGAAGCTGCCCCT |
| | | GGGCTTCACCTTCTCCTTTCCTGTGAGGCATGAAGACATTGATAAGGGCA |
| | | TCCTTCTCAACTGGACCAAGGGCTTCAAGGCCTCAGGAGCAGAAGGGAA |
| | | CAATGTTGTGGGGCTTCTGAGAGATGCTATCAAAAGAAGAGGGGACTTT |
| | | GAAATGGATGTGGTGGCAATGGTGAATGACACAGTGGCCACTATGATCT |
| | | CCTGCTACTATGAAGACCATCAGTGTGAGGTTGGCATGATTGTGGGCAC |
| | | AGGCTGCAATGCCTGCTACATGGAGGAGATGCAGAATGTGGAGCTGGTG |
| | | GAGGGGGATGAGGGCCGCATGTGTGTCAATACTGAGTGGGGCGCCTTTG |
| | | GGGACTCTGGAGAGCTGGATGAGTTCCTGCTGGAGTATGACCGCCTGGT |
| | | GGATGAGAGCTCAGCAAACCCAGGTCAGCAGCTGTATGAGAAGCTCATA |
| | | GGTGGCAAGTACATGGGAGAGCTGGTGCGGCTTGTGCTGCTCAGGTTGG |
| | | TGGATGAAAACCTGCTCTTCCATGGGGAGGCCAGTGAGCAGCTGCGCAC |
| | | ACGCGGAGCCTTTGAGACGCGCTTTGTGTCACAAGTGGAGTCTGACACA |
| | | GGAGACCGCAAGCAGATCTACAACATCCTGAGCACACTGGGGCTGCGGC |
| | | CCTCCACCACTGACTGTGACATTGTGCGCCGCGCCTGTGAGAGTGTGTCT |
| | | ACGCGCGCTGCGCACATGTGCTCAGCGGGGCTGGCGGGAGTCATCAACC |
| | | GCATGCGCGAGAGCCGCTCTGAGGATGTAATGCGCATCACTGTGGGAGT |
| | | GGATGGCAGTGTGTACAAGCTGCACCCCAGCTTCAAGGAGCGGTTCCAT |
| | | GCCTCAGTGCGCAGGCTGACCCCCAGCTGTGAGATCACCTTTATTGAGTC |
| | | TGAGGAGGGCAGTGGCAGAGGCGCGGCCCTGGTCAGCGCGGTGGCCTG |
| | | TAAGAAGGCCTGTATGCTGGGCCAGTGAAGCCAGCTGCCTTCCCTTTACTG |
| | | TGGGTTAATGCTTGCAGCAGACATGATAAGATACATTGATGAGTTTGGACAAA |
| | | CCACAACTAGAATGCAGTGAAAAAAATGCTTTATTTGTGAAATTTGTGATGCTA |
| | | TTGCTTTATTTGTAACCATTATAAGCTGCAATAAACAAGTTAACAACAACAATTG |
| | | CATTCATTTTATGTTTCAGGTTCAGGGGGAGATGTGGGAGGTTTTTTAAAGCA |
| | | AGTAAAACCTCTACAAATGTGGTAAAATAAGGGACTAGAGCAGAGTACTAAGC |
| | | TT |
| Full: 94 | Modified | AAGCTTTATTGTGGTAGTTTATCACAGTTAAATTGCTAAGGCAGTCA |
| 5′UTR: | hGck-27 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 69 | | TCTTACTGACATCCACTTTGCCTTTCTCTCCACAGGTGTCCACTCCC |
| 3′UTR: 107 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGAATGCAGAAGGAGATGGACCGCGGCCTGAGGCTGGA |
| | | GACCCATGAAGAGGCCAGTGTGAAGATGCTGCCCACCTATGTGCGCTCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCAGACTTCCTGGACAAGCATCAGATGAAACACAAGAAGCTGCCCCT |
| | | GGGCTTCACCTTCTCCTTTCCTGTGAGGCATGAAGACATTGATAAGGGCA |
| | | TCCTTCTCAACTGGACCAAGGGCTTCAAGGCCTCAGGAGCAGAAGGGAA |
| | | CAATGTGGTGGGGCTTCTGCGGGATGCTATCAAAAGAAGAGGGGACTTT |
| | | GAAATGGATGTGGTGGCAATGGTGAATGACACAGTGGCCACCATGATCT |
| | | CCTGCTACTATGAAGACCATCAGTGTGAGGTGGGCATGATTGTGGGCAC |
| | | TGGCTGCAATGCCTGCTACATGGAGGAGATGCAGAATGTGGAGCTGGTG |
| | | GAGGGGGATGAGGGCCGCATGTGTGTCAATACAGAGTGGGGCGCCTTTG |
| | | GGGACAGCGGAGAGCTGGATGAGTTCCTGCTGGAGTATGACCGCCTGGT |
| | | GGATGAGAGCTCAGCAAACCCAGGTCAGCAGCTGTATGAGAAGCTCATA |
| | | GGTGGCAAGTACATGGGAGAGCTGGTGCGGCTTGTGCTGCTCAGGCTGG |
| | | TGGATGAAAACCTGCTCTTCCATGGGGAGGCCTCTGAGCAGCTGCGCAC |
| | | ACGCGGAGCCTTTGAGACGCGCTTTGTGAGCCAGGTGGAGAGTGACACA |
| | | GGAGACCGCAAGCAGATCTACAACATCCTGAGCACCCTGGGGCTGCGGC |
| | | CCAGCACCACTGACTGTGACATTGTGCGCCGCGCCTGTGAGTCAGTGTCT |
| | | ACGCGCGCTGCGCACATGTGCAGCGCGGGGCTGGCGGGAGTCATCAACC |
| | | GCATGCGCGAGAGCCGCTCTGAGGATGTAATGCGCATCACTGTGGGAGT |
| | | GGATGGCAGTGTGTACAAGCTGCACCCCAGCTTCAAGGAGCGGTTCCAT |
| | | GCCTCAGTGCGCAGGCTGACCCCCAGCTGTGAGATCACCTTTATTGAGTC |
| | | TGAGGAGGGCAGTGGCAGAGGCGCGGCCCTGGTCAGCGCGGTGGCCTG |
| | | TAAGAAGGCCTGTATGCTGGGCCAGTGAAGCCAGCTGCCTTCCCTTTACTG |
| | | TGGGTTAATGCTTGCAGCAGACATGATAAGATACATTGATGAGTTTGGACAAA |
| | | CCACAACTAGAATGCAGTGAAAAAAATGCTTTATTTGTGAAATTTGTGATGCTA |
| | | TTGCTTTATTTGTAACCATTATAAGCTGCAATAAACAAGTTAACAACAACAATTG |
| | | CATTCATTTTATGTTTCAGGTTCAGGGGGAGATGTGGGAGGTTTTTTAAAGCA |
| | | AGTAAAACCTCTACAAATGTGGTAAAATAAGGGACTAGAGCAGGGTACCAAGC |
| | | TT |
| Full: 95 | Modified | AAGCTTTATTGTGGTAGTTTATCACAGTTAAATTGCTAAGGCAGTCA |
| 5′UTR: | hGck-28 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 72 | | TCTTACTGACATCCACTTTGCCTTTCTCTCCACAGGTGTCCACTCCC |
| 3′UTR: 108 | | AGTTCAATTACAGCTCTTAAGGCTAGAGTCCTGCAGAATTGCCACCA |
| | | TGGCTATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGAATGCAGAAGGAGATGGACAGAGGCCTGAGGCTGGA |
| | | GACCCATGAAGAGGCCAGTGTGAAGATGCTGCCCACCTATGTGAGATCC |
| | | ACCCCAGAAGGCTCAGAAGTTGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCAGACTTCCTGGACAAGCATCAGATGAAACACAAGAAGCTGCCCCT |
| | | GGGCTTCACCTTCTCCTTTCCTGTGAGGCATGAAGACATTGATAAGGGCA |
| | | TCCTTCTCAACTGGACCAAGGGCTTCAAGGCCTCAGGAGCAGAAGGGAA |
| | | CAATGTTGTGGGGCTTCTGAGAGATGCTATCAAAAGAAGAGGGGACTTT |
| | | GAAATGGATGTGGTGGCAATGGTGAATGACACAGTGGCCACTATGATCT |
| | | CCTGCTACTATGAAGACCATCAGTGTGAGGTTGGCATGATTGTGGGCAC |
| | | AGGCTGCAATGCCTGCTACATGGAGGAGATGCAGAATGTGGAGCTGGTG |
| | | GAGGGGGATGAGGGCAGGATGTGTGTCAATACTGAGTGGGGAGCCTTTG |
| | | GGGACTCTGGAGAGCTGGATGAGTTCCTGCTGGAGTATGACAGACTGGT |
| | | GGATGAGAGCTCAGCAAACCCAGGTCAGCAGCTGTATGAGAAGCTCATA |
| | | GGTGGCAAGTACATGGGAGAGCTGGTGAGGCTTGTGCTGCTCAGGTTGG |
| | | TGGATGAAAACCTGCTCTTCCATGGGGAGGCCAGTGAGCAGCTGAGAAC |
| | | AAGGGGAGCCTTTGAGACAAGATTTGTGTCACAGGTGGAGTCTGACACA |
| | | GGAGACAGGAAGCAGATCTACAACATCCTGAGCACCCTGGGGCTGAGA |
| | | CCCTCCACCACTGACTGTGACATTGTGAGGAGAGCCTGTGAGAGTGTGT |
| | | CTACCAGGGCTGCTCACATGTGCTCAGCAGGGCTGGCTGGAGTCATCAA |
| | | CAGAATGAGGGAGAGCAGATCTGAGGATGTAATGAGGATCACTGTGGG |
| | | TGTGGATGGCAGTGTGTACAAGCTGCACCCCAGCTTCAAGGAGAGATTC |
| | | CATGCCTCAGTGAGGAGGCTGACCCCCAGCTGTGAGATCACCTTCATTG |
| | | AGTCTGAGGAGGGCAGTGGCAGAGGAGCAGCCCTGGTCTCTGCTGTGGC |
| | | CTGTAAGAAGGCCTGTATGCTGGGCCAGTGAAGCCAGCTGCCTTCCCTTT |
| | | ACTGTGGGTTAATGCTTGCAGCAGACATGATAAGATACATTGATGAGTTTGGA |
| | | CAAACCACAACTAGAATGCAGTGAAAAAAATGCTTTATTTGTGAAATTTGTGAT |
| | | GCTATTGCTTTATTTGTAACCATTATAAGCTGCAATAAACAAGTTAACAACAACA |
| | | ATTGCATTCATTTTATGTTTCAGGTTCAGGGGGAGATGTGGGAGGTTTTTTAAA |
| | | GCAAGTAAAACCTCTACAAATGTGGTAAAATAAGGGACTAGAGCAGAATATTA |
| | | AGCTT |
| Full: 96 | Modified | AAGCTTTATTGTGGTAGTTTATCACAGTTAAATTGCTAAGGCAGTCA |
| 5′UTR: | hGck-29 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 73 | | TCTTACTGACATCCACTTTGCCTTTCTCTCCACAGGTGTCCACTCCC |
| 3′UTR: 109 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGAATGCAGAAGGAGATGGACAGAGGCCTGAGGCTGGA |
| | | GACCCATGAAGAGGCCAGTGTGAAGATGCTGCCCACCTATGTGAGATCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCAGACTTCCTGGACAAGCATCAGATGAAACACAAGAAGCTGCCCCT |
| | | GGGCTTCACCTTCTCCTTTCCTGTGAGGCATGAAGACATTGATAAGGGCA |
| | | TCCTTCTCAACTGGACCAAGGGCTTCAAGGCCTCAGGAGCAGAAGGGAA |
| | | CAATGTGGTGGGGCTTCTGAGAGATGCTATCAAAAGAAGAGGGGACTTT |
| | | GAAATGGATGTGGTGGCAATGGTGAATGACACAGTGGCCACCATGATCT |
| | | CCTGCTACTATGAAGACCATCAGTGTGAGGTGGGCATGATTGTGGGCAC |
| | | TGGCTGCAATGCCTGCTACATGGAGGAGATGCAGAATGTGGAGCTGGTG |
| | | GAGGGGGATGAGGGCAGAATGTGTGTCAATACAGAGTGGGGAGCCTTT |
| | | GGGGACTCTGGAGAGCTGGATGAGTTCCTGCTGGAGTATGACAGACTGG |
| | | TGGATGAGAGCTCAGCAAACCCAGGTCAGCAGCTGTATGAGAAGCTCAT |
| | | AGGTGGCAAGTACATGGGAGAGCTGGTGAGACTTGTGCTGCTCAGGCTG |
| | | GTGGATGAAAACCTGCTCTTCCATGGGGAGGCCAGTGAGCAGCTGAGAA |
| | | CAAGAGGAGCCTTTGAGACCAGATTTGTGAGCCAGGTGGAGTCAGACAC |
| | | AGGAGACAGAAAGCAGATCTACAACATCCTGAGCACCCTGGGGCTGAG |
| | | ACCCAGCACCACTGACTGTGACATTGTGAGAAGAGCCTGTGAGTCTGTG |
| | | TCTACCAGAGCTGCCCACATGTGCAGTGCTGGGCTGGCTGGAGTCATCA |
| | | ACAGAATGAGAGAGAGCAGATCAGAGGATGTAATGAGAATCACTGTGG |
| | | GTGTGGATGGCTCTGTGTACAAGCTGCACCCCAGCTTCAAGGAGAGATT |
| | | CCATGCCAGTGTGAGAAGGCTGACCCCCAGCTGTGAGATCACCTTTATT |
| | | GAGTCAGAGGAGGGCAGTGGCAGAGGAGCAGCCCTGGTCTCTGCTGTG |
| | | GCCTGTAAGAAGGCCTGTATGCTGGGCCAGTGAAGCCAGCTGCCTTCCCT |
| | | TTACTGTGGGTTAATGCTTGCAGCAGACATGATAAGATACATTGATGAGTTTG |
| | | GACAAACCACAACTAGAATGCAGTGAAAAAAATGCTTTATTTGTGAAATTTGTG |
| | | ATGCTATTGCTTTATTTGTAACCATTATAAGCTGCAATAAACAAGTTAACAACAA |
| | | CAATTGCATTCATTTTATGTTTCAGGTTCAGGGGGAGATGTGGGAGGTTTTTTA |
| | | AAGCAAGTAAAACCTCTACAAATGTGGTAAAATAAGGGACTAGAGCAGTTAAT |
| | | TAAGCTT |
| Full: 163 | Un- | CTAGAAGCTTTATTGTGGTAGTTTATCACAGTTAAATTGCTAAGGCA |
| 5′ UTR: 42 | modified | GTCAGTGCTTCTGACACAACAGTCTCCAACTTAAGCTGCAGTGACTC |
| ORF: 162 | hGck_2 | TCTTAAGGTAGCCTTGCAGAAGTTGGTCCTGAGGCACTGGGCAGGT |
| 3′ UTR: | | AAGTATCAAGGTTACAAGACAGGTTTAAGGAGACCAATAGAAACTG |
| nucleotides | | GGCTTGTGGAGACAGAGAAGACTCTTGGCTTTCTGATAGGCACCTA |
| 1-285 of | | TTGGTCTTACTGACATCCACTTTGCCTTTCTCTCCACAGGTGTCCAC |
| SEQ ID | | TCCCAGTTCAATTACAGCTCTTAAGGCTAGAGTCCTGCAGAATTGCC |
| NO: 60 | | ACCATGGCGATGGATGTCACAAGGAGCCAGGCCCAGACAGCCTTGACTC |
| | | TGGTAGAGCAGATCCTGGCAGAGTTCCAGCTGCAGGAGGAGGACCTGA |
| | | AGAAGGTGATGAGACGGATGCAGAAGGAGATGGACCGCGGCCTGAGGC |
| | | TGGAGACCCATGAAGAGGCCAGTGTGAAGATGCTGCCCACCTACGTGCG |
| | | CTCCACCCCAGAAGGCTCAGAAGTCGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGCGTGAAGACCAAACACCAGATGTACTCCATCCCC |
| | | GAGGACGCCATGACCGGCACTGCTGAGATGCTCTTCGACTACATCTCTG |
| | | AGTGCATCTCCGACTTCCTGGACAAGCATCAGATGAAACACAAGAAGCT |
| | | GCCCCTGGGCTTCACCTTCTCCTTTCCTGTGAGGCACGAAGACATCGATA |
| | | AGGGCATCCTTCTCAACTGGACCAAGGGCTTCAAGGCCTCAGGAGCAGA |
| | | AGGGAACAATGTCGTGGGGCTTCTGCGAGACGCTATCAAACGGAGAGG |
| | | GGACTTTGAAATGGATGTGGTGGCAATGGTGAATGACACGGTGGCCACG |
| | | ATGATCTCCTGCTACTACGAAGACCATCAGTGCGAGGTCGGCATGATCG |
| | | TGGGCACGGGCTGCAATGCCTGCTACATGGAGGAGATGCAGAATGTGGA |
| | | GCTGGTGGAGGGGGACGAGGGCCGCATGTGCGTCAATACCGAGTGGGG |
| | | CGCCTTCGGGGACTCCGGCGAGCTGGACGAGTTCCTGCTGGAGTATGAC |
| | | CGCCTGGTGGACGAGAGCTCTGCAAACCCCGGTCAGCAGCTGTATGAGA |
| | | AGCTCATAGGTGGCAAGTACATGGGCGAGCTGGTGCGGCTTGTGCTGCT |
| | | CAGGCTCGTGGACGAAAACCTGCTCTTCCACGGGGAGGCCTCCGAGCAG |
| | | CTGCGCACACGCGGAGCCTTCGAGACGCGCTTCGTGTCGCAGGTGGAGA |
| | | GCGACACGGGCGACCGCAAGCAGATCTACAACATCCTGAGCACGCTGG |
| | | GGCTGCGACCCTCGACCACCGACTGCGACATCGTGCGCCGCGCCTGCGA |
| | | GAGCGTGTCTACGCGCGCTGCGCACATGTGCTCGGCGGGGCTGGCGGGC |
| | | GTCATCAACCGCATGCGCGAGAGCCGCAGCGAGGACGTAATGCGCATCA |
| | | CTGTGGGCGTGGATGGCTCCGTGTACAAGCTGCACCCCAGCTTCAAGGA |
| | | GCGGTTCCATGCCAGCGTGCGCAGGCTGACGCCCAGCTGCGAGATCACC |
| | | TTCATCGAGTCGGAGGAGGGCAGTGGCCGGGGCGCGGCCCTGGTCTCGG |
| | | CGGTGGCCTGTAAGAAGGCCTGTATGCTGGGCCAGTGAAGCCAGCTGCC |
| | | TTCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAT |
| Full: 164 | Un- | CTAGAAGCTTTATTGTGGTAGTTTATCACAGTTAAATTGCTAAGGCA |
| 5′ UTR: 42 | modified | GTCAGTGCTTCTGACACAACAGTCTCCAACTTAAGCTGCAGTGACTC |
| ORF: 162 | hGck_3 | TCTTAAGGTAGCCTTGCAGAAGTTGGTCCTGAGGCACTGGGCAGGT |
| 3′ UTR: 60 | | AAGTATCAAGGTTACAAGACAGGTTTAAGGAGACCAATAGAAACTG |
| | | GGCTTGTGGAGACAGAGAAGACTCTTGGCTTTCTGATAGGCACCTA |
| | | TTGGTCTTACTGACATCCACTTTGCCTTTCTCTCCACAGGTGTCCAC |
| | | TCCCAGTTCAATTACAGCTCTTAAGGCTAGAGTCCTGCAGAATTGCC |
| | | ACCATGGCGATGGATGTCACAAGGAGCCAGGCCCAGACAGCCTTGACTC |
| | | TGGTAGAGCAGATCCTGGCAGAGTTCCAGCTGCAGGAGGAGGACCTGA |
| | | AGAAGGTGATGAGACGGATGCAGAAGGAGATGGACCGCGGCCTGAGGC |
| | | TGGAGACCCATGAAGAGGCCAGTGTGAAGATGCTGCCCACCTACGTGCG |
| | | CTCCACCCCAGAAGGCTCAGAAGTCGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGCGTGAAGACCAAACACCAGATGTACTCCATCCCC |
| | | GAGGACGCCATGACCGGCACTGCTGAGATGCTCTTCGACTACATCTCTG |
| | | AGTGCATCTCCGACTTCCTGGACAAGCATCAGATGAAACACAAGAAGCT |
| | | GCCCCTGGGCTTCACCTTCTCCTTTCCTGTGAGGCACGAAGACATCGATA |
| | | AGGGCATCCTTCTCAACTGGACCAAGGGCTTCAAGGCCTCAGGAGCAGA |
| | | AGGGAACAATGTCGTGGGGCTTCTGCGAGACGCTATCAAACGGAGAGG |
| | | GGACTTTGAAATGGATGTGGTGGCAATGGTGAATGACACGGTGGCCACG |
| | | ATGATCTCCTGCTACTACGAAGACCATCAGTGCGAGGTCGGCATGATCG |
| | | TGGGCACGGGCTGCAATGCCTGCTACATGGAGGAGATGCAGAATGTGGA |
| | | GCTGGTGGAGGGGGACGAGGGCCGCATGTGCGTCAATACCGAGTGGGG |
| | | CGCCTTCGGGGACTCCGGCGAGCTGGACGAGTTCCTGCTGGAGTATGAC |
| | | CGCCTGGTGGACGAGAGCTCTGCAAACCCCGGTCAGCAGCTGTATGAGA |
| | | AGCTCATAGGTGGCAAGTACATGGGCGAGCTGGTGCGGCTTGTGCTGCT |
| | | CAGGCTCGTGGACGAAAACCTGCTCTTCCACGGGGAGGCCTCCGAGCAG |
| | | CTGCGCACACGCGGAGCCTTCGAGACGCGCTTCGTGTCGCAGGTGGAGA |
| | | GCGACACGGGCGACCGCAAGCAGATCTACAACATCCTGAGCACGCTGG |
| | | GGCTGCGACCCTCGACCACCGACTGCGACATCGTGCGCCGCGCCTGCGA |
| | | GAGCGTGTCTACGCGCGCTGCGCACATGTGCTCGGCGGGGCTGGCGGGC |
| | | GTCATCAACCGCATGCGCGAGAGCCGCAGCGAGGACGTAATGCGCATCA |
| | | CTGTGGGCGTGGATGGCTCCGTGTACAAGCTGCACCCCAGCTTCAAGGA |
| | | GCGGTTCCATGCCAGCGTGCGCAGGCTGACGCCCAGCTGCGAGATCACC |
| | | TTCATCGAGTCGGAGGAGGGCAGTGGCCGGGGCGCGGCCCTGGTCTCGG |
| | | CGGTGGCCTGTAAGAAGGCCTGTATGCTGGGCCAGTGAAGCCAGCTGCC |
| | | TTCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAGAAGCTTTTG |
| Full: 168 | hGck | [CTAG]AAGCTTTATTGTGGTAGTTTATCACAGTTAAATTGCTAAGGC |
| 5′ UTR: 42 | | AGTCAGTGCTTCTGACACAACAGTCTCCAACTTAAGCTGCAGTGACT |
| ORF: 68 | | CTCTTAAGGTAGCCTTGCAGAAGTTGGTCCTGAGGCACTGGGCAGG |
| 3′ UTR: 169 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACCGCGGCCTGAGGC |
| | | TGGAGACCCATGAAGAGGCCAGTGTGAAGATGCTGCCCACCTATGTGCG |
| | | CTCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCAGACTTCCTGGACAAGCATCAGATGAAACACAAGAAGCTG |
| | | CCCCTGGGCTTCACCTTCTCCTTTCCTGTGAGGCATGAAGACATTGATAA |
| | | GGGCATCCTTCTCAACTGGACCAAGGGCTTCAAGGCCTCAGGAGCAGAA |
| | | GGGAACAATGTTGTGGGGCTTCTGAGAGATGCTATCAAAAGAAGAGGG |
| | | GACTTTGAAATGGATGTGGTGGCAATGGTGAATGACACAGTGGCCACTA |
| | | TGATCTCCTGCTACTATGAAGACCATCAGTGTGAGGTTGGCATGATTGTG |
| | | GGCACAGGCTGCAATGCCTGCTACATGGAGGAGATGCAGAATGTGGAG |
| | | CTGGTGGAGGGGGATGAGGGCCGCATGTGTGTCAATACTGAGTGGGGCG |
| | | CCTTTGGGGACTCTGGAGAGCTGGATGAGTTCCTGCTGGAGTATGACCG |
| | | CCTGGTGGATGAGAGCTCAGCAAACCCAGGTCAGCAGCTGTATGAGAAG |
| | | CTCATAGGTGGCAAGTACATGGGAGAGCTGGTGCGGCTTGTGCTGCTCA |
| | | GGTTGGTGGATGAAAACCTGCTCTTCCATGGGGAGGCCAGTGAGCAGCT |
| | | GCGCACACGCGGAGCCTTTGAGACGCGCTTTGTGTCACAAGTGGAGTCT |
| | | GACACAGGAGACCGCAAGCAGATCTACAACATCCTGAGCACACTGGGG |
| | | CTGCGGCCCTCCACCACTGACTGTGACATTGTGCGCCGCGCCTGTGAGA |
| | | GTGTGTCTACGCGCGCTGCGCACATGTGCTCAGCGGGGCTGGCGGGAGT |
| | | CATCAACCGCATGCGCGAGAGCCGCTCTGAGGATGTAATGCGCATCACT |
| | | GTGGGAGTGGATGGCAGTGTGTACAAGCTGCACCCCAGCTTCAAGGAGC |
| | | GGTTCCATGCCTCAGTGCGCAGGCTGACCCCCAGCTGTGAGATCACCTTT |
| | | ATTGAGTCTGAGGAGGGCAGTGGCAGAGGCGCGGCCCTGGTCAGCGCG |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATCCGATAAGGGACTAGAGCATG |
| | | GCTACGTA |

In some aspects, the nucleic acids include codon optimized and CpG reduced sequences relative to wild-type and/or unmodified human Gck nucleic acid sequences (e.g., SEQ ID NO: 19). The modified nucleic acids were chemically synthesized, prepared in expression cassettes including a CMV promoter, and cloned into an expression plasmid. The modified sequences were confirmed by Sanger sequencing.

### Example 3: In Vivo Evaluation of Insulin and Glucokinase Combination Therapy in Diabetic Mice

4-wk old C57Bl/6J mice were administered either streptozotocin (STZ) in a Na-Citrate buffer (40 mg/kg) or buffer alone on five consecutive days. Diabetes developed in STZ mice over the next 4 weeks. Prior to AAV injection, body weight, fed glucose and circulating insulin were determined and the mice blocked and sorted into groups such that there were no significant differences within or between treatment groups.

Stock samples of AAV1-hINS (2.20E13 vg/ml) and AAV1-hGCK (2.58E13 vg/ml) were thawed on ice. AAV1-hINS and AAV1-hGCK stocks were diluted in Dulbecco's PBS+0.001% Pluronic F68 to reach the doses listed in Table 3 in a final volume of 30 μl. Doeses in vg/kg were calculated based on a weight of 25 g per mouse.

Intramuscular (IM) injections of a low-, mid-, or high-dose of AAV1-hINS/AAV1-hGCK at a 1:1 ratio were administered bilaterally in the quadriceps, gastrocnemius and tibialis anterior muscles. hINS:hGCK ratio was studied using a 1:1, 1:0.5, 1:2 and 1:4 ratio based on the mid-dose of hINS (1.2E11 vg). Fed glucose and lactate were measured weekly; fasted glucose and circulating insulin levels were determined on weeks 4 and 8. An OGTT was performed in fasted animals at wk 8, and HbA1c measured at the end of the study. Muscles were collected and mRNA expression, protein expression and enzymatic activity (GCK) of vector-mediated INS and GCK were determined.

TABLE 3
AAV1-hINS and AAV1-hGCK Doses

| | AAV1-hINS | AAV1-hGCK | |
|---|---|---|---|
| | Dose | Dose | Total Dose |
| Low-dose | 9.0E10 vg | 9.0E10 vg | 1.8E11 vg |
| | (3.6E12 vg/kg) | (3.6E12 vg/kg) | (7.2E12 vg/kg) |
| Mid-dose | 1.2E11 vg | 1.2E11 vg | 2.4E11 vg |
| | (4.8E12 vg/kg) | (4.8E12 vg/kg) | (9.6E12 vg/kg) |
| High-dose | 2.4E11 vg | 2.4E11 vg | 4.8E11 vg |
| | (9.6E12 vg/kg) | (9.6E12 vg/kg) | (1.9E13 vg/kg) |
| 1:1 | 1.2E11 vg | 1.2E11 vg | 2.4E11 vg |
| | (4.8E12 vg/kg) | (4.8E12 vg/kg) | (9.6E12 vg/kg) |
| 1:0.5 | 1.2E11 vg | 6.0E10 vg | 1.8E11 vg |
| | (4.8E12 vg/kg) | (2.4E12 vg/kg) | (7.2E12 vg/kg) |
| 1:2 | 1.2E11 vg | 2.4E11 vg | 3.6E11 vg |
| | (4.8E12 vg/kg) | (9.6E12 vg/kg) | (1.4E13 vg/kg) |
| 1:4 | 1.2E11 vg | 4.8E11 vg | 6.0E11 vg |
| | (4.8E12 vg/kg) | (1.9E13 vg/kg) | (2.4E13 vg/kg) |

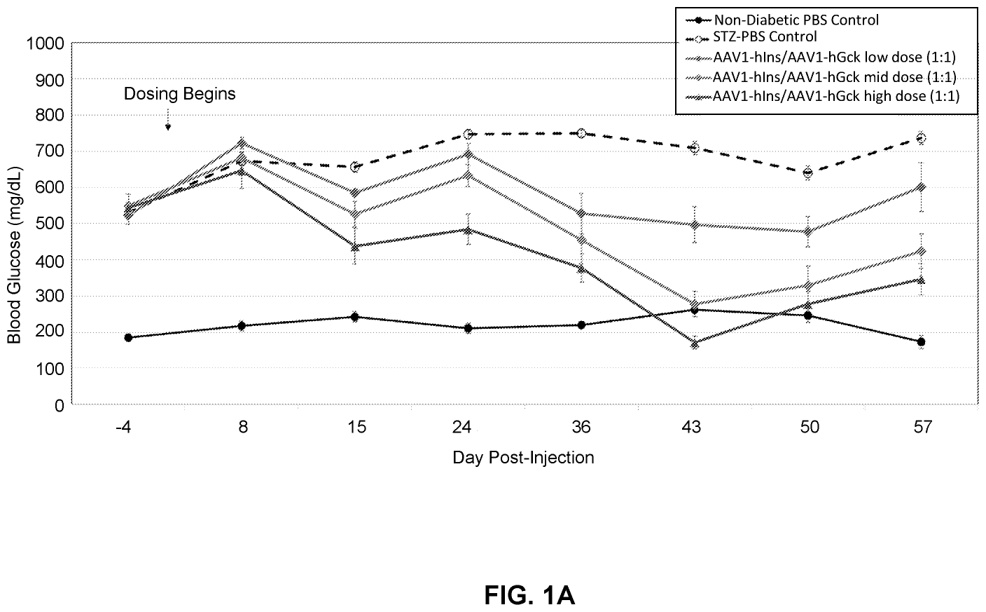

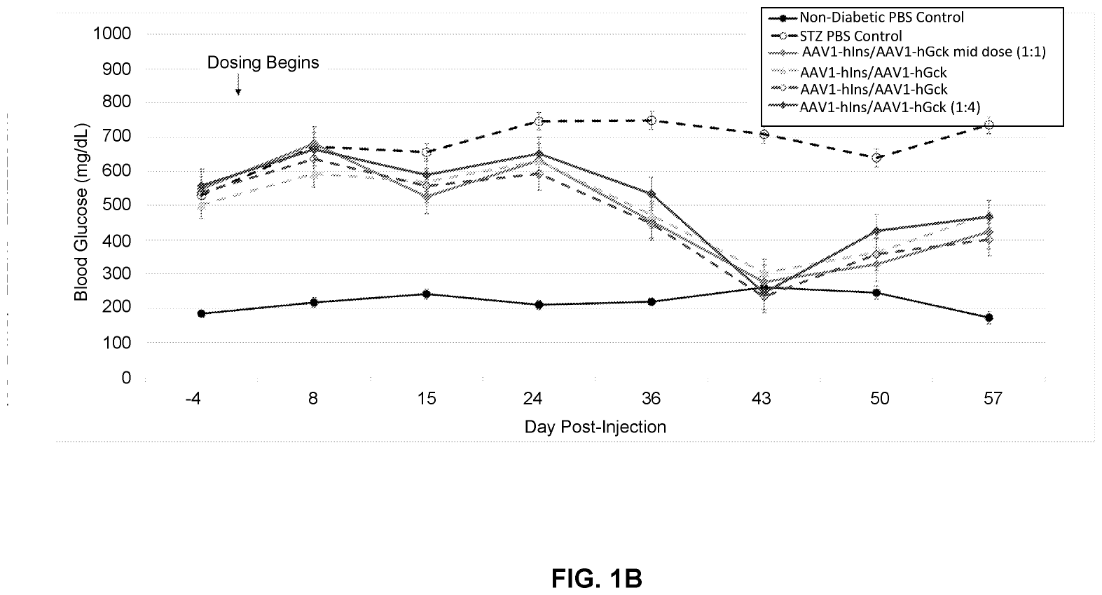

Treatment with a 1:1 mixture of AAV1-hINS and AAV1-hGCK produced significant, dose related improvement in glycemic control in the STZ inducted diabetic animals. Fed Blood Glucose (BG) was measured weekly. Administration of the combination of AAV1-hINS and AAV1-hGck demonstrated a decrease in blood glucose (BG) over 57 days (FIG. 1A). At the highest dose (4.8E11 vg), AAV1-hINS and AAV1-hGck (1:1) significantly reduced BG compared to STZ-Control mice by Day 15 and normalized BG to the level of Non-Diabetic PBS Control by Day 42. The response of the intermediate dose (2.4 E11 vg) was offset by ˜1 wk, but also significantly reduced BG to a level comparable to the non-Diabetic PBS Control mice (p<0.001 compared to STZ-PBS Controls).

The mid-dose of hINS was used as a base to test ratios of doses on blood glucose (FIG. 1B). Ratios of 1:0.5, 1:2 and 1:4 ratio of hINS:hGCK were compared to the 1:1 ratio (2.4E11 vg total). Similar blood glucose levels were observed for the tested ratios.

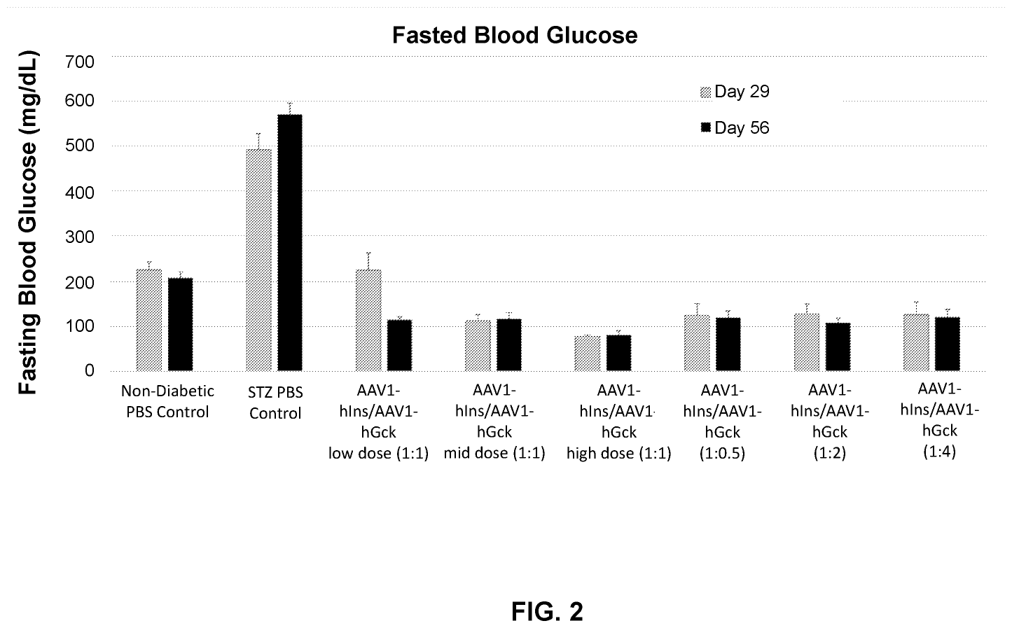

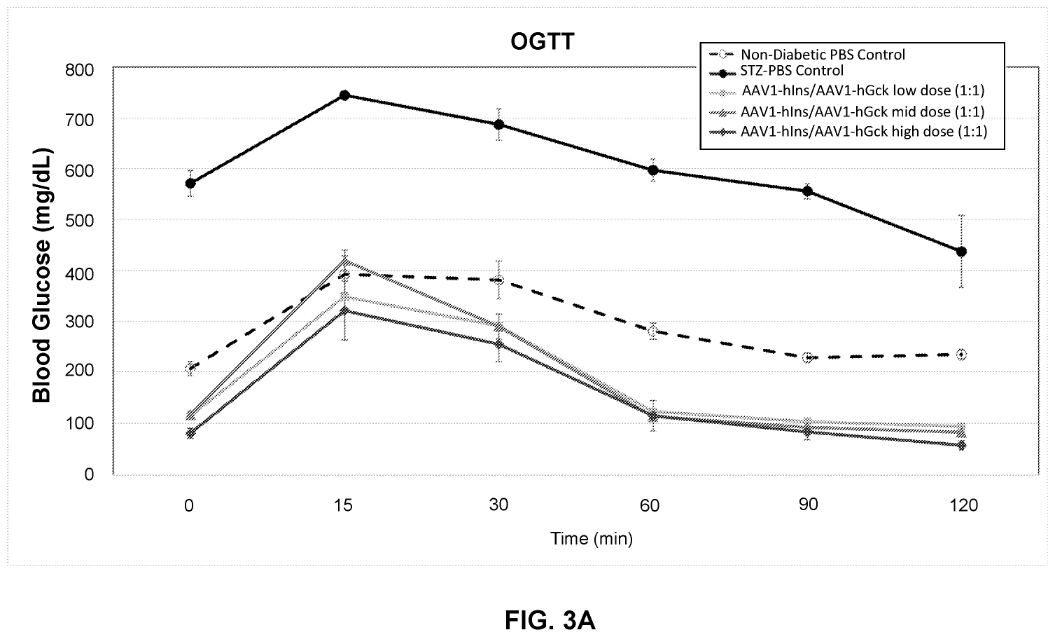

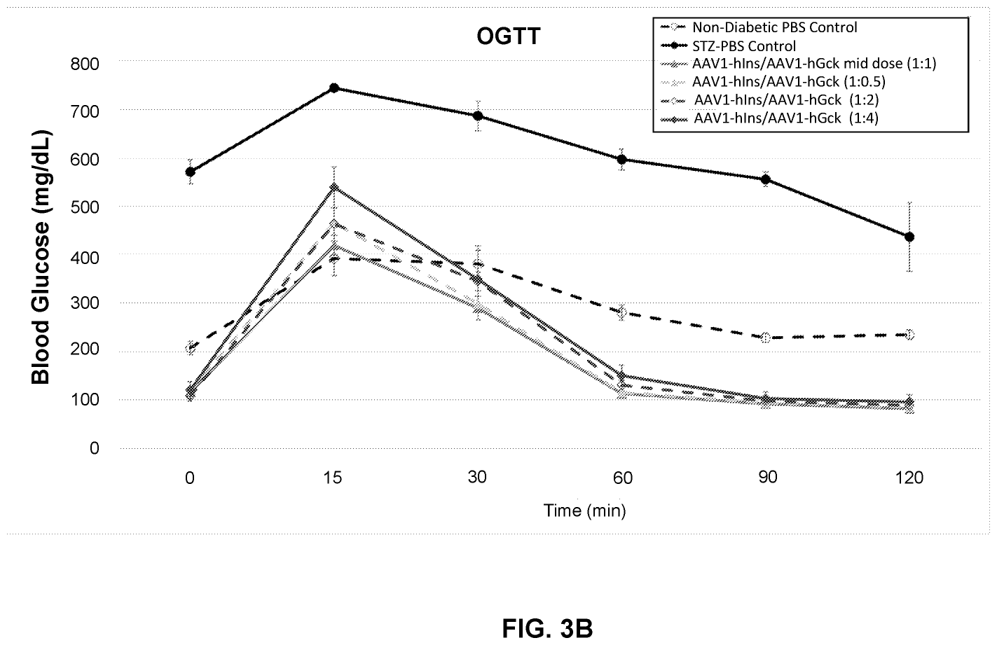

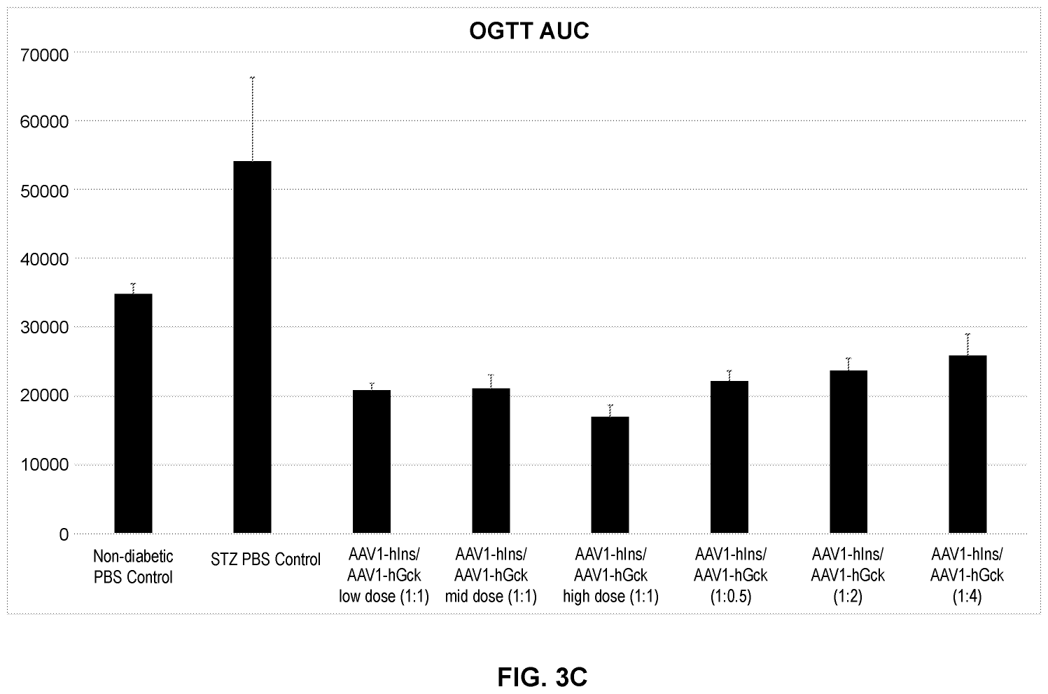

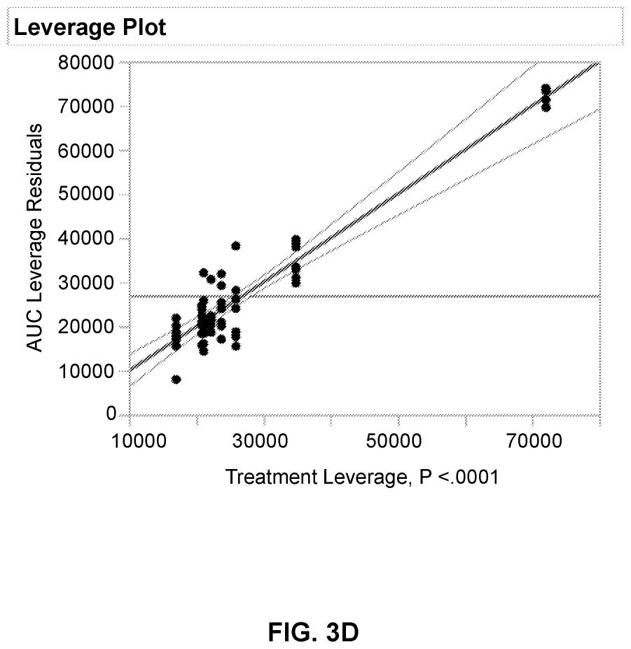

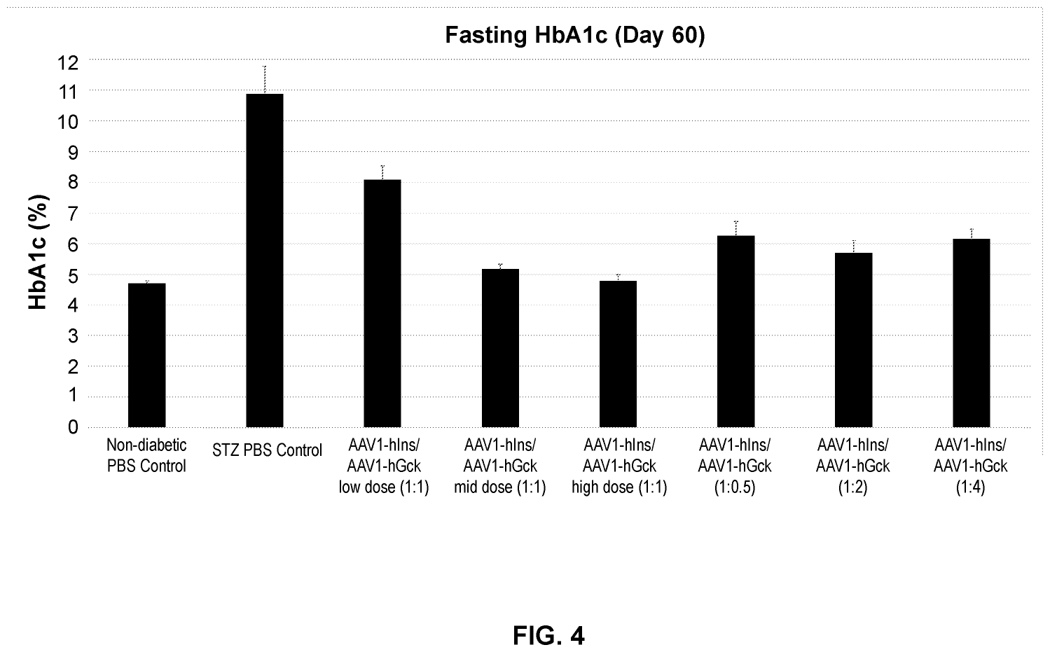

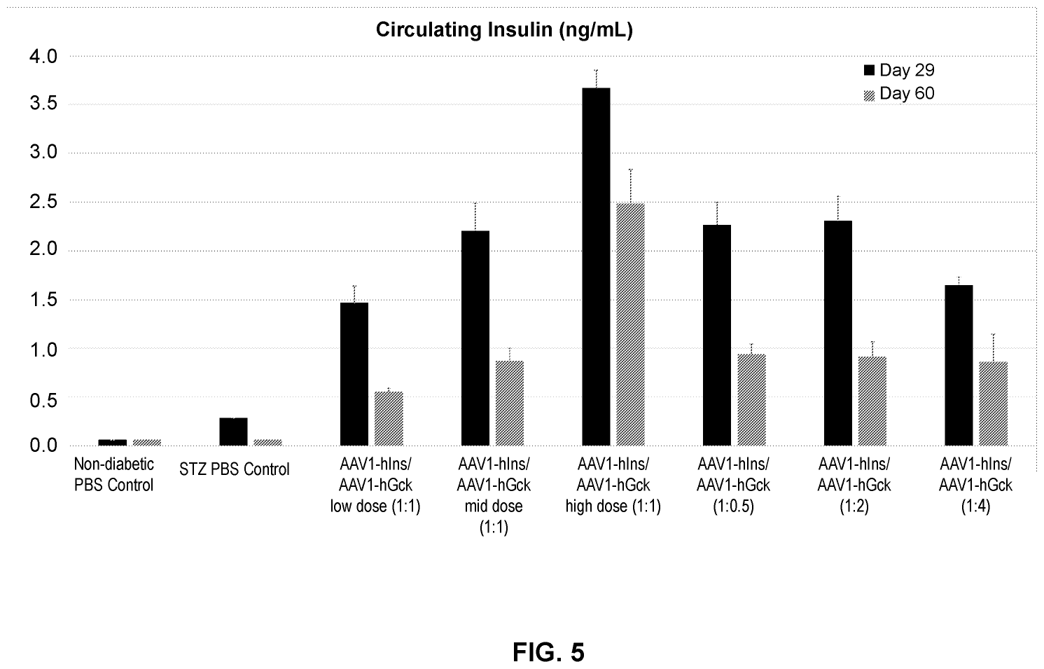

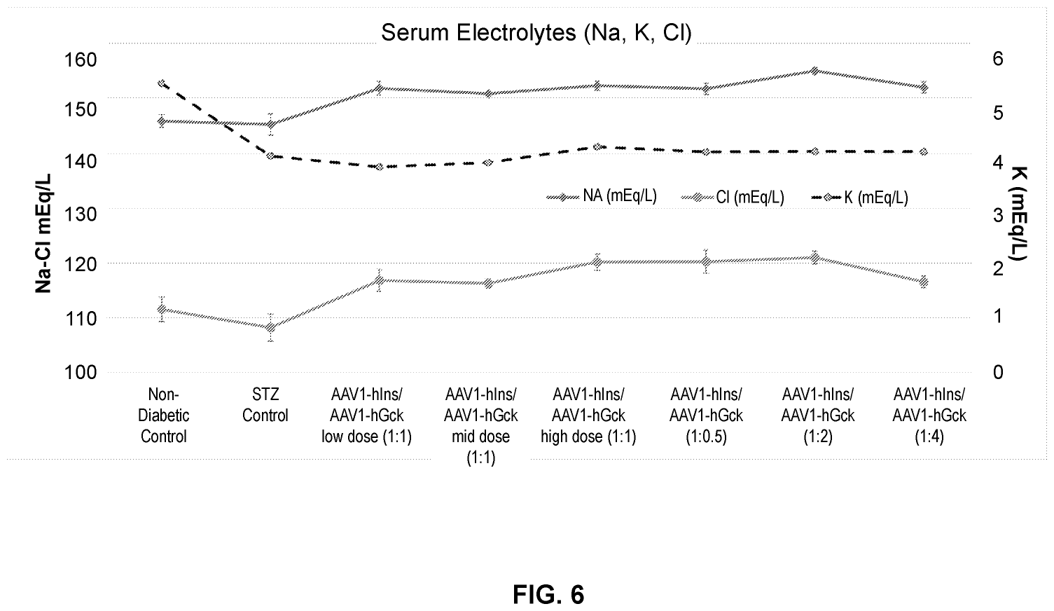

Fasted blood glucose was restored at or below the level of non-STZ controls (FIG. 2). Glucose disposal measured by the Area Under the Curve (AUC) of an oral glucose tolerance test (OGTT) was improved (FIGS. 3A-3D). HbA1c was significantly reduced (>2%) in all treatments (FIG. 4). No significant changes in blood lactic acid production or episodes of hypoglycemia were observed. Circulating insulin levels were higher in all treatments compared to the non-diabetic and STZ treated mice (FIG. 5). Electrolyte levels were similar between all mice (FIG. 6 and Table 3).

TABLE 4
Electrolyte Levels in Mice from All Treatment Groups

| Electrolyte | mEq/L (Mean ± SE) | N= |
|---|---|---|
| Sodium | 140 ± 1.03 | 114 |
| Chloride | 110 ± 0.80 | 153 |
| Potassium | 6.1 ± 0.10 | 133 |

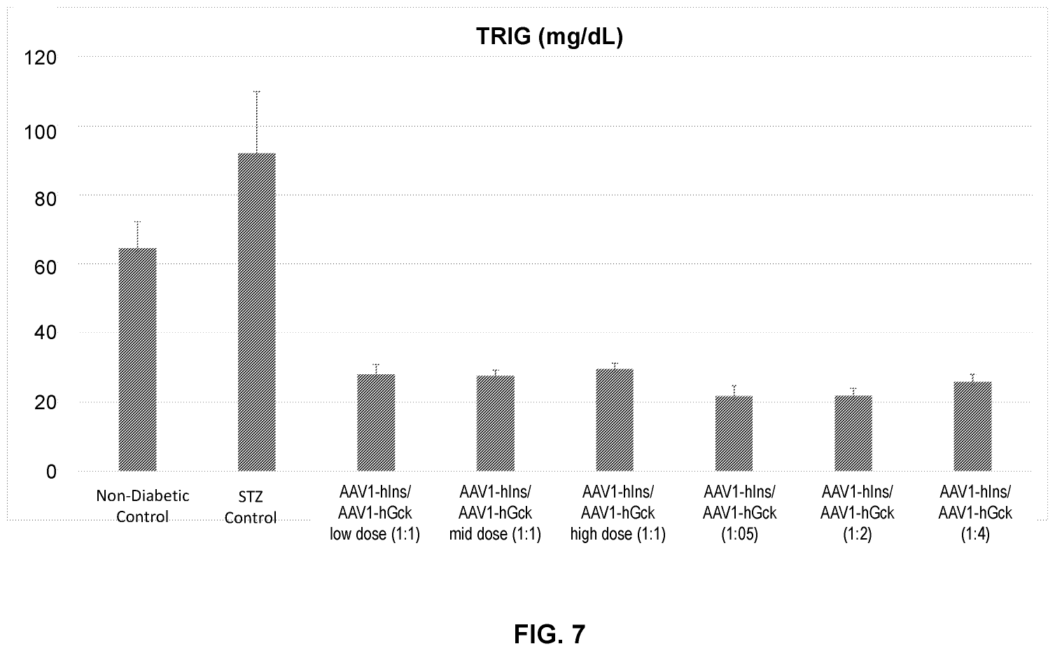

T1D patients with poor glycemic control often present with elevated plasma triglycerides and low-density lipoprotein (LDL) cholesterol. T1D associated nephropathy also results in increased triglycerides and LDL cholesterol, as well as a lower level of high density lipoprotein (HDL) cholesterol. When glycemic control is regained, plasma triglycerides and LDL cholesterol return to normal or slightly decreased, while HDL cholesterol is normal or slightly increased. Triglyceride levels were lower in the treated mice compared to the non-diabetic and STZ control mice (FIG. 7).

Overall, results from this study demonstrate that IM administration of AAV1-hINS/AAV1-hGCK effectively normalize hyperglycemia in an STZ-induced mouse model of T1D. Administration of the combination therapy at the vector ratio of 1:0.5 showed efficacy in the animal model that was comparable to treatments with more GCK, both relative to the dose of INS, and in terms of absolute amount of virus delivered. Furthermore, this investigational therapy has the potential to provide safe, durable maintenance of glycemic control in T1D without the need to employ additional therapeutic agents.

### Example 4: In Vivo Evaluation of Insulin and Glucokinase Combination Therapy in Non-Human Primates

### 4.1 Blood Glucose and Exogenous Insulin Requirements

Cohort 1 included one female (NHP-1) and one male (NHP-2) cynomolgus macaque dosed with a 1:0.5 ratio of AAV1-Ins:AAV1-Gck. Each animal received 28 injections distributed bilaterally to the quadriceps (16 injections) and biceps (12 injections) with a total dose of 1.9E+13 vg/animal of AAV1-Ins and 9.6E+12 vg/animal of AAV1-Gck.

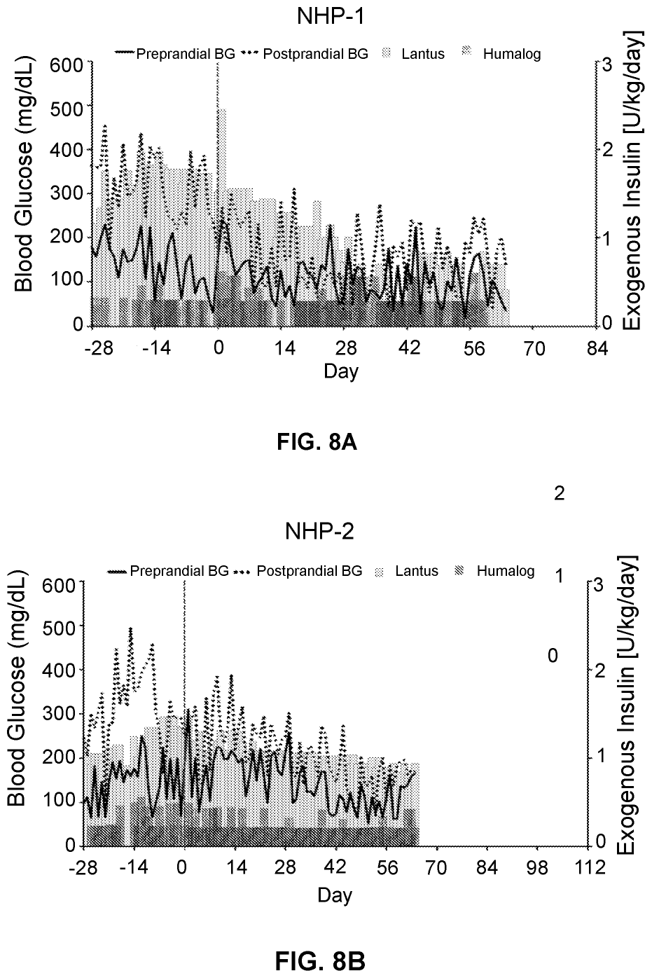

Daily measurements of pre- and post-prandial blood glucose (mg/dL) and the amount of exogenous insulin administered (U/kg/day) to maintain a target glucose level are shown in FIGS. 8A-8B. Compared to baseline (7-day average prior to AAV administration), 30% less exogenous insulin was required to maintain glucose control in NHP-1. At day 60, 56% less was required to achieve the same effect. A similar effect was seen in NHP-2 with a 19% and 25% reduction of exogenous insulin at day 30 and 60, respectively. Previously published data in STZ treated NHPs (Graham, Xenotransplantation 20:5-17, 2013) demonstrated that over this same time frame an untreated STZ-control animal will require more than a 25% increase in exogenous insulin compared to baseline to maintain glucose control.

### 4.2 Circulating C-Peptide Levels and HbA1C

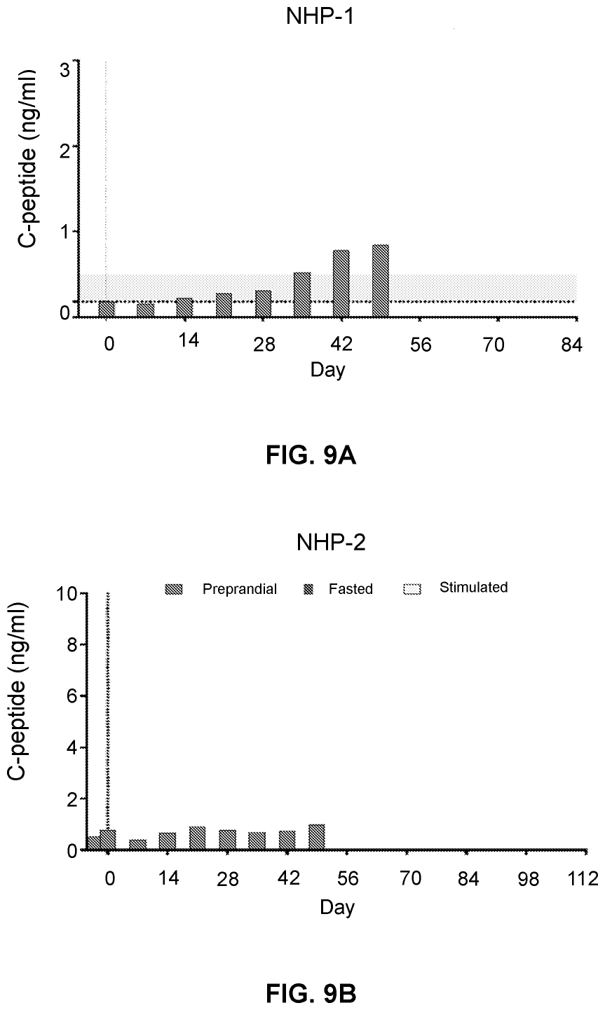

Blood samples were analyzed for circulating human c-peptide weekly. HbA1c was determined on days 0, 28 and 56. FIGS. 9A-9B demonstrate that there was increase in the concentration of human c-peptide (ng/mL) in NHP-1 over the sixty-day observation period. There was a small and variable change in circulating c-peptide observed in NHP-2 over the same period.

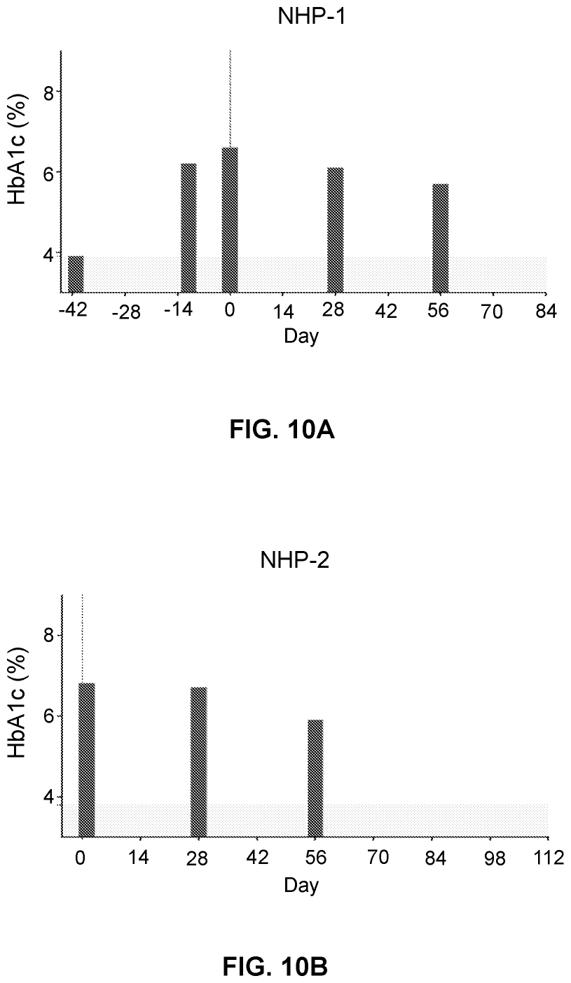

FIGS. 10A-10B illustrate the changes in glycated hemoglobin (HbA1c) over the 60-day observation period. HbA1c is a form of hemoglobin (Hb) that is chemically linked to a sugar. This measurement served as an integrated signal of post-prandial glucose exposure over time with elevated levels indicating poor glycemic control, which has been associated with cardiovascular disease, nephropathy, neuropathy, and retinopathy.

In humans, HbA1c is assumed to be a three-month average of blood glucose levels (see, e.g., Sherwani S I, Khan H A, Ekhzaimy A, Masood A, Sakharkar M K. Significance of HbA1c Test in Diagnosis and Prognosis of Diabetic Patients. Biomark Insights. 2016 Jul. 3; 11:95-104. doi: 10.4137/BMI.S38440. PMID: 27398023; PMCID: PMC4933534.). The lifespan of a red blood cell in cynomolgus NHPs is much less than that of humans and trends can be observed much more rapidly (see, e.g., Glomski, Chester A., Alessandra Pica, and Jessica F. Greene. Erythrocytes of the rhesus and cynomolgus monkeys. CRC Press, 2015). Approximately six weeks after intravenous administration of streptozotocin (STZ), HbA1c levels were determined to be 6.6% and 6.8% in NHP-1 and NHP-2 respectively. Within four weeks of treatment with AAV1-Ins+AAV1-Gck, HbA1c was reduced to 6.1% in NHP-1, and was further reduced (5.7%) at day 56. NHP-2 followed a similar trend reducing HbA1c to 5.9% at day 56. Both NHP-1 and NHP-2 had about a 1% decrease in HbA1c levels over the 60 days of observation.

### 4.3 Intravenous Glucose Tolerance Test (IVGTT)

The intravenous glucose tolerance test (IVGTT) is used more often than oral glucose tolerance test (OGTTs) in NHPs, since most monkeys are not trained to take oral dosing or repeated blood samplings and since inter-animal differences in intestinal glucose absorption may complicate interpretation.

Glucose disposal is estimated from the elimination rate, which allows a glucose elimination constant (Kg) to be calculated. The glucose elimination after intravenous glucose administration follows an exponential function. After logarithmic transformation of the data, the elimination becomes linear. Kg is calculated as the slope for the logarithmic transformation of the individual glucose values and is calculated from the formula kg=(0.693×100)/t_{1/2} where t_{1/2} is the half-time of glucose elimination (in minutes). The unit for kg is percentage of glucose decay per minute. The slopes of the glucose elimination curve show a strong linear correlation with first and second phase glucose disposal. However, STZ destroys the β-cells, thus eliminating the first phase response making the slope primarily a second phase event.

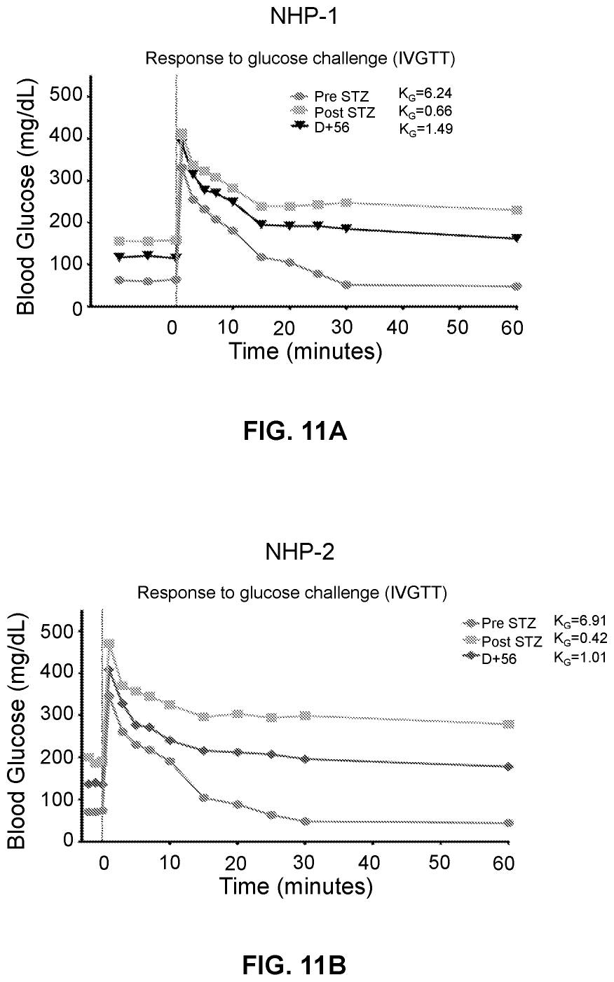

FIGS. 11A-11B depict the IVGTT results of NHP-1 and NHP-2 prior to STZ administration, days before IM administration of AAV1-Ins+AAV1-Gck and 56 days post AAV dosing. Prior to administration of STZ, the Kg of NHP-1 and NHP-2 was calculated to be 6.24 and 6.91 respectively. Thirty days after STZ administration, Kg fell significantly to 0.66 and 0.42. At day 56, Kg significantly improved in both animals to 1.49 and 1.01. Historical data in untreated STZ animals show a progressive decline in Kg over time. The improved glucose elimination rate in the NHPs administered AAV1-Ins+AAV1-Gck suggests improved insulin function, reversing the effect of STZ.

What is claimed is:

1. A combination therapy comprising:
(a) a first AAV vector genome comprising an insulin expression cassette comprising a first promoter operably linked to a polynucleotide encoding a human insulin (hIns) protein, wherein the insulin expression cassette is flanked by inverted terminal repeats (ITRs); and
(b) a second AAV vector genome comprising a glucokinase expression cassette comprising a second promoter operably linked to a polynucleotide encoding a human glucokinase (hGck) protein, wherein the glucokinase expression cassette is flanked by inverted terminal repeats (ITRs);
wherein the first AAV vector genome and the second AAV vector genome are in a ratio selected from the group consisting of 1:0.25-0.75, 1:1.75-2.25, and 1:3.75-4.25.

2. A method of treating or ameliorating the symptoms associated with diabetes in a subject in need thereof, comprising administering a combination therapy to the subject comprising:
(a) a first AAV vector genome comprising an insulin expression cassette comprising a first promoter operably linked to a polynucleotide encoding a human insulin (hIns) protein, wherein the insulin expression cassette is flanked by inverted terminal repeats (ITRs); and
(b) a second AAV vector genome comprising a glucokinase expression cassette comprising a second promoter operably linked to a polynucleotide encoding a human glucokinase (hGck) protein, wherein the glucokinase expression cassette is flanked by inverted terminal repeats (ITRs);
wherein the first AAV vector genome and the second AAV vector genome are administered at a ratio selected from the group consisting of 1:0.25-0.75, 1:1.75-2.25, and 1:3.75-4.25.

3. The combination therapy or method of claim 1 or 2, wherein the ratio is selected from the group consisting of 1:0.4-0.6, 1:1.9-2.1, and 1:3.9-4.1.

4. The combination therapy or method of any one of claims 1-3, wherein the ratio is selected from the group consisting of 1:0.4-0.60 and 1:0.45-0.55.

5. The combination therapy or method of anyone of claims 1-4, wherein the ratio is selected from the group consisting of about 1:0.5, about 1:2, and about 1:4.

6. The combination therapy or method of anyone of claims 1-5, wherein the ratio is about 1:0.5.

7. The combination therapy or method of any one of claims 1-6, wherein:
(a) the polynucleotide encoding the hIns protein comprises an open reading frame (ORF) comprising: a nucleotide sequence at least 85%, 90%, 95%, 99%, or 100% identical to any one of: (i) nucleic acids 73-330 of any of SEQ ID NOs: 43-57, 110-116, 150-151, 154-155 or 157-159, nucleic acids 88-345 of any of SEQ ID NOs: 117-122, 152 or 156, or nucleic acids 79-336 of SEQ ID NO: 153; or (ii) SEQ ID NO: 43-57, SEQ ID NO: 110-122, or SEQ ID NO: 150-159; and/or
(b) the polynucleotide encoding the human glucokinase hGck protein, comprises an ORF comprising (i) a nucleotide sequence at least 85%, 90%, 95%, 99%, or 100% identical to a sequence selected from any of (a) nucleic acids 1-1398 of any of SEQ ID NO: 61-80 or 162; or (ii) SEQ ID NO: 61-80 and 162.

8. The combination therapy or method of any one of claims 1-7, wherein the hIns protein comprises the amino acid sequence of any of amino acids 25-110 of SEQ ID NO: 41, amino acids 25-110 of SEQ ID NO: 144, amino acids 25-110 of SEQ ID NO: 145, SEQ ID NO: 41, SEQ ID NO: 144, or SEQ ID NO: 145.

9. The combination therapy or method of any one of claims 1-8, wherein the hIns protein comprises a signal peptide.

10. The combination therapy or method of claim 9, wherein the signal peptide is a wild-type preproinsulin signal sequence, an IL-6 signal sequence, a fibronectin signal sequence, or a non-wild-type preproinsulin signal sequence.

11. The combination therapy or method of any one of claim 9 or 10, wherein the signal peptide comprises amino acids 25-110 of SEQ ID NO: 41, amino acids 25-110 of SEQ ID NO: 144, or amino acids 25-110 of SEQ ID NO: 145.

12. The combination therapy or method of claim 9, wherein the signal peptide is a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid position in wild-type proinsulin.

13. The combination therapy or method of any one of claims 1-12, wherein the hIns protein further comprises a cleavage site.

14. The combination therapy or method of any one of claims 1-13, wherein the polynucleotide encoding the hIns protein further comprises a 5′ UTR comprising a nucleotide sequence at least 85%, 90%, 95%, 99%, or 100% identical to nucleic acids 5-329 of SEQ ID NO: 42.

15. The combination therapy or method of any one of claims 1-14, wherein the polynucleotide encoding the hIns protein further comprises a 5′ UTR comprising a nucleotide sequence at least 85%, 90%, 95%, 99%, or 100% identical to SEQ ID NO: 42, SEQ ID NO: 83, SEQ ID NO: 146, or SEQ ID NO: 148.

16. The combination therapy or method of any one of claims 1-15, wherein the polynucleotide encoding the hIns protein further comprises a 3′ UTR comprising a nucleotide sequence at least 85%, 90%, 95%, 99%, or 100% identical to SEQ ID NO: 60, SEQ ID NO: 97, SEQ ID NO: 98, SEQ ID NO: 99, SEQ ID NO: 100, or SEQ ID NO: 101, SEQ ID NO: 149, or SEQ ID NO: 171.

17. The combination therapy or method of any one of claims 1-16, wherein the encoded hGck protein comprises the amino acid sequence of SEQ ID NO: 82.

18. The combination therapy or method of any one of claims 1-17, wherein the polynucleotide encoding the hGck protein further comprises a 5′ UTR comprising a nucleotide sequence at least 85%, 90%, 95%, 99%, or 100% identical to nucleic acids 5-329 of SEQ ID NO: 42.

19. The combination therapy or method of any one of claims 1-17, wherein the polynucleotide encoding the hGck protein further comprises a 5′ UTR comprising a nucleotide sequence at least 85%, 90%, 95%, 99%, or 100% identical to SEQ ID NO: 42, SEQ ID NO: 83, SEQ ID NO: 146, or SEQ ID NO: 148.

20. The combination therapy or method of any one of claims 1-17, wherein the polynucleotide encoding the hGck protein further comprises a 3′ UTR comprising a nucleotide sequence at least 85%, 90%, 95%, 99%, or 100% identical to SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 149 or SEQ ID NO: 169.

21. The combination therapy or method of any one of claims 1-20, wherein first the promoter is a eukaryotic promoter.

22. The combination therapy or method of claim 21, wherein the first promoter is a CMV promoter.

23. The combination therapy or method of any one of claims 1-22, wherein the second promoter is a eukaryotic promoter.

24. The combination therapy or method of claim 23, wherein the second promoter is a CMV promoter.

25. The combination therapy or method of any one of claims 1-24, wherein the insulin expression cassette comprises a polyadenylation (polyA) element.

26. The combination therapy or method of any one of claims 1-24, wherein the glucokinase expression cassette comprises a polyadenylation (polyA) element.

27. The combination therapy or method of any one of claims 1-26 comprising a first recombinant AAV (rAAV) particle comprises the first AAV vector genome.

28. The combination therapy or method of any one of claims 1-27 comprising a second recombinant AAV (rAAV) particle comprises the second vector genome.

29. The combination therapy or method of claim 27 or 28, wherein the AAV serotype is selected from the group consisting of AAV1, AAV2, AAV3, AAV4, AAV5, AAV6, AAV7, AAV8, AAVrh8, AAVrh9, AAV9, AAVrh10, AAV10, AAV11, and AAV12.

30. The combination therapy or method of any one of claims 27-29, wherein the first rAAV particle and the second rAAV particle are formulated in a single composition.

31. A method of treating or ameliorating the symptoms associated with diabetes in a subject in need thereof, comprising delivering to the subject a therapeutically effective amount of the combination therapy of any one of claims 1 and 3-29, thereby treating diabetes in the subject.

32. A method of producing human Ins protein and human Gck protein in a subject in need thereof and/or treating or ameliorating the symptoms associate with diabetes in a subject in need thereof comprising administering to the subject the combination therapy of any one of claims 1 and 3-29, thereby producing human Ins protein and human Gck protein and/or treating diabetes in the subject.

33. The method of any one of claim 2-29 or 31-32, wherein the diabetes is diabetes mellitus type 1 (T1DM) or diabetes mellitus type 2 (T2DM).

34. The method of claim 33, wherein the diabetes is diabetes mellitus type 1 (T1DM).

35. The method of claim 33, wherein the diabetes is diabetes mellitus type 2 (T2DM).

36. The method of any one of claim 2-29 or 31-35, wherein the first AAV vector genome of and the second AAV vector genome are administered simultaneously or sequentially.

37. The method of any one of claim 27-29 or 31-36, wherein the first rAAV particle of and the second rAAV particle are administered simultaneously or sequentially.

38. The combination therapy or method of any of the previous claims, wherein the delivery and/or administration is intramuscular.

39. The combination therapy or method of any of the previous claims, wherein (i) glycated blood hemoglobin (HbA1c) levels are reduced and/or regulated in the subject; (ii) circulating ketones are reduced in the subject, (iii) triglycerides are reduced in the subject, or (iv) any combination thereof.

40. A composition comprising:
(a) a first recombinant AAV (rAAV) particle comprising a first AAV vector genome comprising an insulin expression cassette comprising a first promoter operably linked to a polynucleotide encoding a human insulin (hIns) protein, wherein the insulin expression cassette is flanked by inverted terminal repeats (ITRs); and
(b) a second recombinant AAV (rAAV) particle comprising a second AAV vector genome comprising a glucokinase expression cassette comprising a second promoter operably linked to a polynucleotide encoding a human glucokinase (hGck) protein, wherein the glucokinase expression cassette is flanked by inverted terminal repeats (ITRs);
wherein the first rAAV particle and the second rAAV particle are in a ratio selected from the group consisting of 1:0.25-0.75, 1:1.75-2.25, and 1:3.75-4.25.

41. The composition of claim 40, wherein the ratio is selected from the group consisting of 1:0.4-0.6, 1:1.9-2.1, and 1:3.9-4.1.

42. The composition of any one of claims 40-41, wherein the ratio is selected from the group consisting of 1:0.4-0.60 and 1:0.45-0.55

43. The composition of claim 40 or 41, wherein the ratio is selected from the group consisting of about 1:0.5, about 1:2, and about 1:4.

44. The composition of anyone of claims 40-43, wherein the ratio is about 1:0.5.


SEQUENCE LISTING (171 sequences)

SEQ ID NO: 1 - DNA, 1046 bp, synthetic construct
  source 1..1046: mol_type = other DNA; note = Unmodified hIns 1 (B10D)
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagctgctagctaatcgataccgtcgacctcgaggaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 2 - DNA, 957 bp, synthetic construct
  source 1..957: mol_type = other DNA; note = Modified hIns - 2
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggcactgctggccctctggggacctgacccagcagcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtccagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 3 - DNA, 957 bp, synthetic construct
  source 1..957: mol_type = other DNA; note = Modified hIns - 3
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgaggctcctgcccctgctggccctgctggccctctggggacctgacccagctgcagcctttgtgaaccaacacctgtgtggctcagatctggtggaagctctctacctagtgtgtggggaaaggggcttcttctacacacccaggaccaagagagaggcagaggacctgcaggtggggcaggtggagctgggagggggccctggtgcaggcagcctgcagcccttggccctggaggggagcaggcagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 4 - DNA, 957 bp, synthetic construct
  source 1..957: mol_type = other DNA; note = Modified hIns - 4
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgaggctcctgcccctgctggctctgctggccctctggggacctgacccagcagcagcctttgtgaaccaacacctgtgtggctcagatctggtggaagctctctacctagtgtgtggggaaaggggcttcttctacacacccaggaccaagagagaggcagaggacctgcaggtggggcaggtggagctgggagggggccctggtgcaggcagcctgcagcccttggccctggaggggagcaggcagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 5 - DNA, 957 bp, synthetic construct
  source 1..957: mol_type = other DNA; note = Modified hIns - 5
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactgctccccctcctggctttgctggccctgtggggaccagatcctgcagctgcctttgtgaatcagcacctgtgtggctcagatctggttgaggcactctacctggtgtgtggggagagaggcttcttttacacaccaaggaccaagagggaggcagaggatctgcaggtaggtcaggtggagctgggaggtggccctggagcaggatccctgcagcccttggcactggagggctccagacagaagaggggcattgtggaacagtgttgcactagcatctgcagcctgtaccagctggagaactactgcaactaaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 6 - DNA, 957 bp, synthetic construct
  source 1..957: mol_type = other DNA; note = Modified hIns - 6
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 7 - DNA, 957 bp, synthetic construct
  source 1..957: mol_type = other DNA; note = Modified hIns - 7
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 8 - DNA, 957 bp, synthetic construct
  source 1..957: mol_type = other DNA; note = Modified hIns -8
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 9 - DNA, 957 bp, synthetic construct
  source 1..957: mol_type = other DNA; note = Modified hIns - 9
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggccctgctggccctctggggacctgacccagctgcagcctttgtgaaccaacacctgtgtggctcagatctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggagcagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 10 - DNA, 957 bp, synthetic construct
  source 1..957: mol_type = other DNA; note = Modified hIns - 10
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggagtagacagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 11 - DNA, 957 bp, synthetic construct
  source 1..957: mol_type = other DNA; note = Modified hIns - 11
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagagaggcagaggacctgcaggtggggcaggtggagctgggtgggggccctggtgcaggcagcctgcagcccttggccctggaggggtctagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 12 - DNA, 957 bp, synthetic construct
  source 1..957: mol_type = other DNA; note = Modified hIns -12
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcggctcttgccccttctggctttactggcactatggggccctgacccagcggccgctttcgtgaaccagcacctctgcggaagcgatctggtcgaggccttgtacctggtttgtggggaaagaggttttttctataccccgaggacaaagcgcgaggcagaagaccttcaagtgggccaggtagagctcggagggggccccggtgccggatccctgcagcctctggctctcgaagggtctcgacaaaaacgtggcatcgtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 13 - DNA, 957 bp, synthetic construct
  source 1..957: mol_type = other DNA; note = Modified hIns 13
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcggctcttgccccttctggctttactggcactatggggccctgacccagcggccgcttttgtgaaccagcacctctgcggatctgatctggttgaggccttgtacctggtttgtggggaaagaggttttttctataccccaaggacaaagcgcgaggcagaagaccttcaagtgggccaggtagagctgggagggggcccgggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 14 - DNA, 957 bp, synthetic construct
  source 1..957: mol_type = other DNA; note = Modified hIns - 14
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcggctcttgccccttctggctttactggcactatggggccctgacccagcggccgcttttgtgaaccagcacctctgcggatctgatctggtggaggccttgtacctggtttgtggggaaagaggttttttctatacccccaggacaaagcgcgaggcagaagaccttcaagtgggccaggtagagctgggagggggcccgggtgcaggatccctgcagcctctggctctggaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 15 - DNA, 957 bp, synthetic construct
  source 1..957: mol_type = other DNA; note = Modified hIns - 15
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcttgccccttctggctttactggcactatggggccctgacccagcagctgcttttgtgaaccagcacctctgtggatctgatctggttgaggccttgtacctggtttgtggggaaagaggttttttctataccccaaggacaaagagggaggcagaagaccttcaagtgggccaggtagagctgggagggggccctggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 16 - DNA, 957 bp, synthetic construct
  source 1..957: mol_type = other DNA; note = Modified hIns - 16
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcttgccccttctggctttactggcactatggggccctgacccagcagctgcttttgtgaaccagcacctctgtggatctgatctggtggaggccttgtacctggtttgtggggaaagaggttttttctatacccccaggacaaagagagaggcagaagaccttcaagtgggccaggtagagctgggagggggcccaggtgcaggatccctgcagcctctggctctggaagggtctagacaaaaaagaggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 17-18 - skipped (2 entries)

SEQ ID NO: 19 - DNA, 2114 bp, synthetic construct
  source 1..2114: mol_type = other DNA; note = Wildtype hGck
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgaacgcagctgctagctaatcgataccgtcgacctcgaggaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 20 - DNA, 2025 bp, synthetic construct
  source 1..2025: mol_type = other DNA; note = Modified hGck - 2
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccaccatgatctcctgctactatgaagaccatcagtgtgaggtgggcatgattgtgggcacaggctgcaatgcctgctacatggaggagatgcagaatgtggagctggtggagggggatgagggcaggatgtgtgtcaatacagagtggggagcctttggggactctggggagctggatgagttcctgctggagtatgacaggctggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaaggggagcctttgagaccaggtttgtgtcccaggtggagagtgacacaggagacaggaagcagatctacaacatcctgagcaccctggggctgagaccctccaccacagactgtgacattgtgaggagggcttgtgagagtgtgtctaccagggctgcccacatgtgctctgctgggctggctggggtcatcaacaggatgagggagagcaggagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 21 - DNA, 2025 bp, synthetic construct
  source 1..2025: mol_type = other DNA; note = Modified hGck-3
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggtctgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagggatgctatcaaaaggagaggggactttgaaatggatgtggtggcaatggtgaatgacacagtggccacaatgatctcctgctactatgaagaccatcagtgtgaggtgggcatgattgtgggcacaggctgcaatgcctgctacatggaggagatgcagaatgtggagctggtggagggggatgagggcagaatgtgtgtcaatacagagtggggagcctttggggactctggggagctggatgagttcctgctggagtatgacagactggtggatgagagctcagcaaaccctggtcagcagctgtatgagaagctcataggtggcaagtacatgggagagctggtgagacttgtgctgctcaggctggtggatgaaaacctgctcttccatggggaggcctctgagcagctgagaacaagaggagcctttgagaccagatttgtgtcccaggtggagtctgacacaggagacaggaagcagatctacaacatcctgagcaccctggggctgaggccctccaccacagactgtgacattgtgagaagagcctgtgagtctgtgtctaccagggctgcccacatgtgctctgctgggctggctggggtcatcaacagaatgagggagagcaggtctgaggatgtaatgagaatcactgtgggagtggatggctctgtgtacaagctgcacccctcattcaaggagagattccatgcctctgtgagaaggctgacccccagctgtgagatcacctttattgagtctgaggagggcagtggcagaggagctgccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 22 - DNA, 2025 bp, synthetic construct
  source 1..2025: mol_type = other DNA; note = Modified hGck-4
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgactggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagggatgctatcaaaaggagaggggactttgaaatggatgtggtggcaatggtgaatgacactgtggccactatgatctcctgctactatgaagaccatcagtgtgaggtaggcatgattgtgggcactggctgcaatgcctgctacatggaggagatgcagaatgtggagctggtggagggggatgagggcagaatgtgtgtcaatactgagtggggtgcctttggggactcaggggagctggatgagttcctgctggagtatgacagactggtggatgagagctctgcaaaccctggtcagcagctgtatgagaagctcataggtggcaagtacatgggagagctggtgagacttgtgctgctcaggctggtggatgaaaacctgctcttccatggggaggcctctgagcagctgagaacaagaggagcctttgagaccagatttgtgtcccaggtggagtctgacactggtgacaggaagcagatctacaacatcctgagcacactggggctgaggccctccaccacagactgtgacattgtgagaagagcttgtgagagtgtgtctaccagggctgctcacatgtgcagtgctgggctggctggggtcatcaacagaatgagggagagcaggtctgaggatgtaatgagaatcactgtgggtgtggatggcagtgtgtacaagctgcacccctcattcaaggagagattccatgccagtgtgagaaggctgacccccagctgtgagatcaccttcattgagtctgaggagggcagtggcaggggtgcagccctggtcagtgcagtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 23 - DNA, 2025 bp, synthetic construct
  source 1..2025: mol_type = other DNA; note = Modified hGck-5
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctcagacttcctggacaagcatcagatgaaacacaagaagctgcccctgggcttcaccttctcctttcctgtgaggcatgaagacatagataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccactatgatctcctgctactatgaagaccatcagtgtgaggtgggcatgatagtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctctgagcagctgagaacaaggggagcctttgagaccaggtttgtgtcccaggtggagtctgacactggggacaggaagcagatctacaacatcctgagcaccctggggctgaggccctccaccactgactgtgacatagtgaggagagcctgtgagtctgtgtctaccagggctgcccacatgtgctctgctgggctggcaggggtcatcaacaggatgagggagagcagatctgaggatgtaatgaggatcactgtgggtgtggatggctctgtgtacaagctgcacccctcattcaaggagaggttccatgcctctgtgaggaggctgacccccagctgtgagatcacctttatagagtctgaggagggcagtggcagaggggcagccctggtctctgcagtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 24 - DNA, 2025 bp, synthetic construct
  source 1..2025: mol_type = other DNA; note = Modified hGck-6
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccaccatgatctcctgctactatgaagaccatcagtgtgaggtgggcatgatagtgggcacaggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctcagagcagctgagaacaaggggagcctttgagaccaggtttgtgtcccaggtggagagtgacacaggtgacaggaagcagatctacaacatcctgagcaccctggggctgagaccctccaccacagactgtgacattgtgaggagggcctgtgagagtgtgtctaccagggctgcccacatgtgctctgctgggctggcaggagtcatcaacaggatgagggagagcaggagtgaggatgtaatgaggatcactgtgggagtggatggctctgtgtacaagctgcacccctcattcaaggagaggttccatgccagtgtgagaaggctgacccccagctgtgagatcacctttatagagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 25 - DNA, 2025 bp, synthetic construct
  source 1..2025: mol_type = other DNA; note = Modified hGck-7
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccacaatgatctcctgctactatgaagaccatcagtgtgaggtgggcatgatagtgggcacaggctgcaatgcctgctacatggaggagatgcagaatgtggagctggtggagggggatgagggcagaatgtgtgtcaatacagagtggggagcctttggggactctggggagctggatgagttcctgctggagtatgacagactggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaagaggagcctttgagaccagatttgtgtcacaggtggagtctgacactggagacagaaagcagatctacaacatcctgagcacactggggctgaggccctccaccactgactgtgacatagtgaggagagcttgtgagagtgtgtctaccagagctgcccacatgtgctctgctgggctggctggggtcatcaacaggatgagagagagcaggagtgaggatgtaatgagaatcactgtgggggtggatggctctgtgtacaagctgcacccctcattcaaggagaggttccatgcctctgtgaggaggctgacccccagctgtgagatcacctttattgagtctgaggagggcagtggaaggggagcagccctggtctctgcagtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 26 - DNA, 2025 bp, synthetic construct
  source 1..2025: mol_type = other DNA; note = Modified hGck-8
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccactatgatctcctgctactatgaagaccatcagtgtgaggtgggcatgattgtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggagagctggtgagacttgtgctgctcaggctggtggatgaaaacctgctcttccatggggaggcctcagagcagctgaggacaaggggagcctttgagaccaggtttgtgtcccaggtggagagtgacactggagacaggaagcagatctacaacatcctgagcaccctggggctgaggccctctaccacagactgtgacatagtgagaagggcctgtgagtctgtgtctaccagggctgcccacatgtgctctgctgggctggctggagtcatcaacagaatgagggagagcagaagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagctgccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 27 - DNA, 2025 bp, synthetic construct
  source 1..2025: mol_type = other DNA; note = Modified hGck-9
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggccgcatgtgtgtcaatactgagtggggcgcctttggggactctggagagctggatgagttcctgctggagtatgaccgcctggtggatgagagctcagcaaacccaggtcagcagctgtatgagaagctcataggtggcaagtacatgggagagctggtgcggcttgtgctgctcaggttggtggatgaaaacctgctcttccatggggaggccagtgagcagctgcgcacacgcggagcctttgagacgcgctttgtgtcacaagtggagtctgacacaggagaccgcaagcagatctacaacatcctgagcacactggggctgcggccctccaccactgactgtgacattgtgcgccgcgcctgtgagagtgtgtctacgcgcgctgcgcacatgtgctcagcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 28 - DNA, 2025 bp, synthetic construct
  source 1..2025: mol_type = other DNA; note = Modified hGck-10
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgcgggatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggccgcatgtgtgtcaatacagagtggggcgcctttggggacagcggagagctggatgagttcctgctggagtatgaccgcctggtggatgagagctcagcaaacccaggtcagcagctgtatgagaagctcataggtggcaagtacatgggagagctggtgcggcttgtgctgctcaggctggtggatgaaaacctgctcttccatggggaggcctctgagcagctgcgcacacgcggagcctttgagacgcgctttgtgagccaggtggagagtgacacaggagaccgcaagcagatctacaacatcctgagcaccctggggctgcggcccagcaccactgactgtgacattgtgcgccgcgcctgtgagtcagtgtctacgcgcgctgcgcacatgtgcagcgcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 29 - DNA, 2025 bp, synthetic construct
  source 1..2025: mol_type = other DNA; note = Modified hGck-11
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatcccggaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggtaggcatgatagtgggcacaggctgcaatgcctgctacatggaggagatgcagaatgtggagctggtggagggggatgagggccgcatgtgtgtcaatactgagtggggcgcctttggggacagtggagagctggatgagttcctgctggagtatgaccgcctggtggatgagagctctgcaaacccaggtcagcagctgtatgagaagctcataggtggcaagtacatgggagagctggtgcggcttgtgctgctcaggctagtggatgaaaacctgctcttccatggggaggcctcagagcagctgcgcacacgcggagcctttgagacgcgctttgtgtctcaagtggagagtgacacaggtgaccgcaagcagatctacaacatcctgagcactctggggctgcggccctcaaccacagactgtgacattgtgcgccgcgcctgtgagtctgtgtctacgcgcgctgcgcacatgtgcagtgcggggctggcgggagtcatcaaccgcatgcgcgagagccgctcagaggatgtaatgcgcatcactgtgggtgtggatggctctgtgtacaagctgcaccccagcttcaaggagcggttccatgccagtgtgcgcaggctgactcccagctgtgagatcacctttatagagtcagaggagggcagtggcagaggcgcggccctggtctccgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 30 - DNA, 2025 bp, synthetic construct
  source 1..2025: mol_type = other DNA; note = Modified hGck-12
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacactgtggccactatgatctcctgctactatgaagaccatcagtgtgaggttggcatgattgtgggcactggctgcaatgcctgctacatggaggagatgcagaatgtggagctggtggagggggatgagggccgcatgtgtgtcaatactgagtggggcgcctttggggactctggtgagctggatgagttcctgctggagtatgaccgcctggtggatgagagctctgcaaaccctggtcagcagctgtatgagaagctcataggtggcaagtacatgggtgagctggtgcggcttgtgctgctcaggttagtggatgaaaacctgctcttccatggggaggcctctgagcagctgcgcacacgcggagcctttgagacgcgctttgtgtctcaagtggagtctgacactggtgaccgcaagcagatctacaacatcctgagcactctggggctgagaccctctaccactgactgtgacattgtgcgccgcgcctgtgagtctgtgtctacgcgcgctgcgcacatgtgctctgcggggctggcgggtgtcatcaaccgcatgcgcgagagccgctctgaggatgtaatgcgcatcactgtgggtgtggatggctctgtgtacaagctgcacccctcattcaaggagcggttccatgcctctgtgcgcaggctgactcccagctgtgagatcacctttattgagtctgaggagggcagtggcagaggcgcggccctggtctctgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 31 - DNA, 2025 bp, synthetic construct
  source 1..2025: mol_type = other DNA; note = Modified hGck-13
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggcaggatgtgtgtcaatactgagtggggagcctttggggactctggagagctggatgagttcctgctggagtatgacagactggtggatgagagctcagcaaacccaggtcagcagctgtatgagaagctcataggtggcaagtacatgggagagctggtgaggcttgtgctgctcaggttggtggatgaaaacctgctcttccatggggaggccagtgagcagctgagaacaaggggagcctttgagacaagatttgtgtcacaggtggagtctgacacaggagacaggaagcagatctacaacatcctgagcaccctggggctgagaccctccaccactgactgtgacattgtgaggagagcctgtgagagtgtgtctaccagggctgctcacatgtgctcagcagggctggctggagtcatcaacagaatgagggagagcagatctgaggatgtaatgaggatcactgtgggtgtggatggcagtgtgtacaagctgcaccccagcttcaaggagagattccatgcctcagtgaggaggctgacccccagctgtgagatcaccttcattgagtctgaggagggcagtggcagaggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 32 - DNA, 2025 bp, synthetic construct
  source 1..2025: mol_type = other DNA; note = Modified hGck-14
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgagagatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggcagaatgtgtgtcaatacagagtggggagcctttggggactctggagagctggatgagttcctgctggagtatgacagactggtggatgagagctcagcaaacccaggtcagcagctgtatgagaagctcataggtggcaagtacatgggagagctggtgagacttgtgctgctcaggctggtggatgaaaacctgctcttccatggggaggccagtgagcagctgagaacaagaggagcctttgagaccagatttgtgagccaggtggagtcagacacaggagacagaaagcagatctacaacatcctgagcaccctggggctgagacccagcaccactgactgtgacattgtgagaagagcctgtgagtctgtgtctaccagagctgcccacatgtgcagtgctgggctggctggagtcatcaacagaatgagagagagcagatcagaggatgtaatgagaatcactgtgggtgtggatggctctgtgtacaagctgcaccccagcttcaaggagagattccatgccagtgtgagaaggctgacccccagctgtgagatcacctttattgagtcagaggagggcagtggcagaggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 33 - DNA, 2025 bp, synthetic construct
  source 1..2025: mol_type = other DNA; note = Modified hGck-15
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatcccagaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggtaggcatgatagtgggcacaggctgcaatgcctgctacatggaggagatgcagaatgtggagctggtggagggggatgagggcaggatgtgtgtcaatactgagtggggagcctttggggacagtggagagctggatgagttcctgctggagtatgacagactggtggatgagagctctgcaaaccctggtcagcagctgtatgagaagctcataggtggcaagtacatgggtgagctggtgaggcttgtgctgctcaggctagtggatgaaaacctgctcttccatggggaggcctcagagcagctgagaacaaggggagcctttgagacaagatttgtgtctcaggtggagagtgacactggagacaggaagcagatctacaacatcctgagcacactggggctgagaccctcaaccactgactgtgacattgtgaggagagcctgtgagtctgtgtctacaagggctgcacacatgtgcagtgctgggctggcaggtgtcatcaacagaatgagggagagcagatcagaggatgtaatgaggatcactgtgggagtggatggctctgtgtacaagctgcaccccagcttcaaggagagattccatgccagtgtgaggaggctgacccccagctgtgagatcacctttatagagtcagaggagggcagtggcagaggtgctgccctggtctctgcagtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 34 - DNA, 2025 bp, synthetic construct
  source 1..2025: mol_type = other DNA; note = Modified hGck-16
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacactgtggccactatgatctcctgctactatgaagaccatcagtgtgaggttggcatgattgtgggcactggctgcaatgcctgctacatggaggagatgcagaatgtggagctggtggagggggatgagggcagaatgtgtgtcaatactgagtggggtgcctttggggactctggtgagctggatgagttcctgctggagtatgacagactggtggatgagagctctgcaaaccctggtcagcagctgtatgagaagctcataggtggcaagtacatgggtgagctggtgagacttgtgctgctcaggttagtggatgaaaacctgctcttccatggggaggcctctgagcagctgagaacaagaggagcctttgagactagatttgtgtctcaggtggagtctgacactggtgacagaaagcagatctacaacatcctgagcactctggggctgagaccctctaccactgactgtgacattgtgagaagagcctgtgagtctgtgtctactagagctgctcacatgtgctctgctgggctggctggtgtcatcaacagaatgagagagagcagatctgaggatgtaatgagaatcactgtgggtgtggatggctctgtgtacaagctgcacccctcattcaaggagagattccatgcctctgtgagaaggctgactcccagctgtgagatcaccttcattgagtctgaggagggcagtggcagaggtgctgccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 35 - DNA, 2025 bp, synthetic construct
  source 1..2025: mol_type = other DNA; note = Modified hGck-17
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggacgtgacccggagccaggctcaaacagcactgactctcgtcgagcagatcttggcggaattccagcttcaagaggaagatctgaagaaagttatgagaaggatgcagaaggagatggaccgcggcttacgactggaaacgcacgaggaggcctccgtgaaaatgctacccacctacgtgcgttctacacctgaaggatcagaggtgggggattttctcagtctggacttgggtggcactaacttccgggtcatgctggtgaaggttggagaaggggaggaaggccagtggtcggtgaaaaccaagcatcagatgtatagcattccagaggatgctatgacaggcaccgccgagatgctttttgactacatatccgaatgcatctctgatttcctcgacaagcaccaaatgaaacataagaaactgccgctgggatttactttctcatttcccgtgagacacgaggatattgacaaggggatcctcttgaattggacgaaaggcttcaaggcaagtggcgccgaaggaaacaatgtagtggggcttctgagggatgctattaagcgccggggtgactttgagatggacgttgtggcaatggtcaacgatacagtggccacaatgatcagctgttattacgaggaccatcagtgcgaagtcggcatgatagtgggaactgggtgtaatgcgtgctatatggaggaaatgcagaacgttgagttagtggaaggcgatgagggtagaatgtgtgtaaataccgagtggggagctttcggggactccggcgaactggatgagttcctactcgaatacgacaggctggtggatgagtctagcgccaaccctggccagcaattgtatgaaaaactgatcggagggaagtacatgggtgagcttgtccgcctcgttctgctgcgattagtggacgagaatctcctgtttcacggcgaagcatccgagcagttgcgtacacggggagccttcgaaaccagatttgtctcacaagtggagagtgatactggggacaggaaacagatttacaacatccttagcacactgggcctacgcccatctacgaccgattgcgacattgtgcggcgagcttgtgagtccgtttcgactagagccgcacacatgtgctcagctggtctcgccggagtaatcaataggatgcgcgaaagccggagtgaggacgtgatgagaataacagtcggcgtggatgggtctgtctataagctgcatccctccttcaaggaaaggtttcacgcgagcgtgcgccgtctgaccccttcatgtgagattactttcatcgaatccgaggagggcagtggacggggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 36 - DNA, 2025 bp, synthetic construct
  source 1..2025: mol_type = other DNA; note = Modified hGck-18
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtgaccagaagccaggctcaaacagcactgactctggtagagcagatcttggcggaattccagcttcaagaggaagatctgaagaaagttatgagaaggatgcagaaggagatggaccgcggcttaaggctggaaacccatgaggaggccagtgtgaaaatgctacccacctatgtaagatctacacctgaaggatcagaggtgggggattttctcagtctggacttgggtggcactaacttcagggtcatgctggtgaaggttggagaaggggaggaaggccagtggtcagtgaaaaccaagcatcagatgtatagcattccagaggatgctatgacaggtaccgctgagatgctttttgactacatatctgaatgcatctctgatttcctagacaagcaccaaatgaaacataagaaactgccgctgggatttactttctcatttcctgtgagacatgaggatattgacaaggggatcctcttgaattggacaaaaggcttcaaggcaagtggcgcagaaggaaacaatgtagtggggcttctgagggatgctattaagcgcagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggtgatgagggtagaatgtgtgtaaatacagagtggggagcttttggggacagcggagaactggatgagttcctattagaatatgacaggctggtggatgagtctagcgccaaccctggccagcaattgtatgaaaaactgattggagggaagtacatgggtgagcttgtccgccttgttctgctgcggttagtggatgagaatctcctgtttcatggggaagcaagtgagcagttgcggacaaggggagcctttgaaaccagatttgtctcacaagtggagagtgatactggggacaggaaacagatttacaacatccttagcacactgggcctaagaccatctaccactgattgtgacattgtgcggcgggcttgtgagtcagtttccactagagccgcacacatgtgctcagctggtttggctggagtaatcaataggatgcgcgaaagcaggagtgaggatgtgatgagaataacagttggtgtggatgggtctgtctataagctgcatccctccttcaaggaaaggtttcacgcgtctgtgcgcagactgaccccttcatgtgagattactttcatagaatctgaggagggcagtggaaggggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 37 - DNA, 2025 bp, synthetic construct
  source 1..2025: mol_type = other DNA; note = Modified hGck-19
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtgaccagaagccaggctcaaacagcactgactctggtggagcagatcttggcggaattccagcttcaagaggaagatctgaagaaagttatgagaaggatgcagaaggagatggaccgcggcttaaggctggaaacccatgaggaggccagtgtgaaaatgctacccacctatgtaagatctacacctgaaggatcagaggtgggggattttctcagtctggacttgggtggcactaacttcagggtcatgctggtgaaggttggagaaggggaggaaggccagtggtcagtgaaaaccaagcatcagatgtatagcattccagaggatgctatgacaggtaccgctgagatgctttttgactacatatctgaatgcatctctgatttcctggacaagcaccaaatgaaacataagaaactgccgctgggatttactttctcatttcctgtgagacatgaggatattgacaaggggatcctcttgaattggaccaaaggcttcaaggcaagtggcgcagaaggaaacaatgtagtggggcttctgagggatgctattaagcgcagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggtgatgagggtagaatgtgtgtaaatacagagtggggagcttttggggacagcggagaactggatgagttcctactggaatatgacaggctggtggatgagtctagcgccaaccctggccagcaattgtatgaaaaactgattggagggaagtacatgggtgagcttgtccgcctggttctgctgcggttagtggatgagaatctcctgtttcatggggaagcaagtgagcagttgcggacaaggggagcctttgaaaccagatttgtctcacaagtggagagtgatactggggacaggaaacagatttacaacatccttagcacactgggcctaagaccatctaccacagattgtgacattgtgcggcgggcttgtgagtcagttagcactagagccgcacacatgtgctcagctggtctggctggagtaatcaataggatgcgcgaaagcaggagtgaggatgtgatgagaataacagtgggtgtggatgggtctgtctataagctgcatccctccttcaaggaaaggtttcacgcgtctgtgcgcagactgaccccttcatgtgagattactttcatagaatctgaggagggcagtggaaggggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 38 - DNA, 2025 bp, synthetic construct
  source 1..2025: mol_type = other DNA; note = Modified hGck-20
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtgaccagaagccaggctcaaacagcactgactctggtagagcagatcttggctgaattccagcttcaagaggaagatctgaagaaagttatgagaaggatgcagaaggagatggacaggggcttaagactggaaacccatgaggaggccagtgtgaaaatgctacccacctatgtaaggtctacacctgaaggatcagaggtgggggattttctcagtctggacttgggtggcactaacttcagagtcatgctggtgaaggttggagaaggggaggaaggccagtggtcagtgaaaaccaagcatcagatgtatagcattccagaggatgctatgacaggcacagcagagatgctttttgactacatatctgaatgcatctctgatttcctagacaagcaccaaatgaaacataagaaactgccactgggatttactttctcatttcctgtgagacatgaggatattgacaaggggatcctcttgaattggaccaaaggcttcaaggcaagtggtgctgaaggaaacaatgtagtggggcttctgagggatgctattaagaggagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggagatgagggtagaatgtgtgtaaatacagagtggggagcttttggggacagtggggaactggatgagttcctattagaatatgacaggctggtggatgagtcttcagccaaccctggccagcaattgtatgaaaaactgattggagggaagtacatgggtgagcttgtcaggcttgttctgctgagattagtggatgagaatctcctgtttcatggtgaagcatctgagcagttgaggacaagaggagcctttgaaaccagatttgtctcacaagtggagagtgatactggggacaggaaacagatttacaacatccttagcacactgggcctaaggccatctaccactgattgtgacattgtgagaagggcttgtgagagtgtttccactagagcagcacacatgtgctcagctggtttggctggagtaatcaataggatgagagaaagcaggagtgaggatgtgatgagaataacagttggagtggatgggtctgtctataagctgcatccctccttcaaggaaaggtttcatgcctctgtgagaaggctgaccccttcatgtgagattactttcatagaatcagaggagggcagtggaagaggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 39 - DNA, 2025 bp, synthetic construct
  source 1..2025: mol_type = other DNA; note = Modified hGck-21
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtgaccagaagccaggctcaaacagcactgactctggtggagcagatcttggctgaattccagcttcaagaggaagatctgaagaaagttatgagaaggatgcagaaggagatggacagaggcttaagactggaaacccatgaggaggccagtgtgaaaatgctacccacctatgtaagatctacacctgaaggatcagaggtgggggattttctcagtctggacttgggtggcactaacttcagagtcatgctggtgaaggttggagaaggggaggaaggccagtggtcagtgaaaaccaagcatcagatgtatagcattccagaggatgctatgacaggcacagcagagatgctttttgactacatatctgaatgcatctctgatttcctggacaagcaccaaatgaaacataagaaactgcccctgggatttactttctcatttcctgtgagacatgaggatattgacaaggggatcctcttgaattggaccaaaggcttcaaggcaagtggtgctgaaggaaacaatgtagtggggcttctgagggatgctattaagagaagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggagatgagggtagaatgtgtgtaaatacagagtggggagcttttggggacagtggggaactggatgagttcctactggaatatgacaggctggtggatgagtcttcagccaaccctggccagcaattgtatgaaaaactgattggagggaagtacatgggtgagcttgtcagactggttctgctgagattagtggatgagaatctcctgtttcatggtgaagcatctgagcagttgagaacaagaggagcctttgaaaccagatttgtctcacaagtggagagtgatactggggacaggaaacagatttacaacatccttagcacactgggcctaagaccatctaccactgattgtgacattgtgagaagagcttgtgagagtgttagcactagagcagcacacatgtgctcagctggtctggctggagtaatcaataggatgagagaaagcagaagtgaggatgtgatgagaataacagtgggagtggatgggtctgtctataagctgcatccctccttcaaggaaaggtttcatgcctctgtgagaagactgaccccttcatgtgagattactttcatagaatcagaggagggcagtggaagaggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 40 - skipped

SEQ ID NO: 41 - protein, 110 aa, synthetic construct
  source 1..110: note = wild type insulin aa
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 42 - DNA, 329 bp, synthetic construct
  source 1..329: mol_type = other DNA; note = 5' UTR
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccacc

SEQ ID NO: 43 - DNA, 333 bp, synthetic construct
  source 1..333: mol_type = other DNA; note = ORF
atggccctgtggatgagactcctgcccctgctggcactgctggccctctggggacctgacccagcagcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtccagacagaagagaggcattgtggaacaatgctgtaccagcatctgctccctctaccagctggagaactactgcaactag

SEQ ID NO: 44 - DNA, 333 bp, synthetic construct
  source 1..333: mol_type = other DNA; note = ORF
atggccctgtggatgaggctcctgcccctgctggccctgctggccctctggggacctgacccagctgcagcctttgtgaaccaacacctgtgtggctcagatctggtggaagctctctacctagtgtgtggggaaaggggcttcttctacacacccaggaccaagagagaggcagaggacctgcaggtggggcaggtggagctgggagggggccctggtgcaggcagcctgcagcccttggccctggaggggagcaggcagaagaggggcattgtggaacaatgctgtaccagcatctgctccctctaccagctggagaactactgcaactag

SEQ ID NO: 45 - DNA, 333 bp, synthetic construct
  source 1..333: mol_type = other DNA; note = ORF
atggccctgtggatgaggctcctgcccctgctggctctgctggccctctggggacctgacccagcagcagcctttgtgaaccaacacctgtgtggctcagatctggtggaagctctctacctagtgtgtggggaaaggggcttcttctacacacccaggaccaagagagaggcagaggacctgcaggtggggcaggtggagctgggagggggccctggtgcaggcagcctgcagcccttggccctggaggggagcaggcagaagagaggcattgtggaacaatgctgtaccagcatctgctccctctaccagctggagaactactgcaactag

SEQ ID NO: 46 - DNA, 333 bp, synthetic construct
  source 1..333: mol_type = other DNA; note = ORF
atggccctgtggatgagactgctccccctcctggctttgctggccctgtggggaccagatcctgcagctgcctttgtgaatcagcacctgtgtggctcagatctggttgaggcactctacctggtgtgtggggagagaggcttcttttacacaccaaggaccaagagggaggcagaggatctgcaggtaggtcaggtggagctgggaggtggccctggagcaggatccctgcagcccttggcactggagggctccagacagaagaggggcattgtggaacagtgttgcactagcatctgcagcctgtaccagctggagaactactgcaactaa

SEQ ID NO: 47 - DNA, 333 bp, synthetic construct
  source 1..333: mol_type = other DNA; note = ORF
atggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactag

SEQ ID NO: 48 - DNA, 333 bp, synthetic construct
  source 1..333: mol_type = other DNA; note = ORF
atggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagagaggcattgtggaacaatgctgtaccagcatctgctccctctaccagctggagaactactgcaactag

SEQ ID NO: 49 - DNA, 333 bp, synthetic construct
  source 1..333: mol_type = other DNA; note = ORF
atggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactag

SEQ ID NO: 50 - DNA, 333 bp, synthetic construct
  source 1..333: mol_type = other DNA; note = ORF
atggccctgtggatgagactcctgcccctgctggccctgctggccctctggggacctgacccagctgcagcctttgtgaaccaacacctgtgtggctcagatctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggagcagacagaagagaggcattgtggaacaatgctgtaccagcatctgctccctctaccagctggagaactactgcaactag

SEQ ID NO: 51 - DNA, 333 bp, synthetic construct
  source 1..333: mol_type = other DNA; note = ORF
atggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggagtagacagaagaggggcattgtggaacaatgctgtaccagcatctgctccctctaccagctggagaactactgcaactag

SEQ ID NO: 52 - DNA, 333 bp, synthetic construct
  source 1..333: mol_type = other DNA; note = ORF
atggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagagaggcagaggacctgcaggtggggcaggtggagctgggtgggggccctggtgcaggcagcctgcagcccttggccctggaggggtctagacagaagagaggcattgtggaacaatgctgtaccagcatctgctccctctaccagctggagaactactgcaactag

SEQ ID NO: 53 - DNA, 333 bp, synthetic construct
  source 1..333: mol_type = other DNA; note = ORF
atggccctgtggatgcggctcttgccccttctggctttactggcactatggggccctgacccagcggccgctttcgtgaaccagcacctctgcggaagcgatctggtcgaggccttgtacctggtttgtggggaaagaggttttttctataccccgaggacaaagcgcgaggcagaagaccttcaagtgggccaggtagagctcggagggggccccggtgccggatccctgcagcctctggctctcgaagggtctcgacaaaaacgtggcatcgtggagcagtgctgtacttcaatttgcagtttgtaccagcttgagaattattgtaactga

SEQ ID NO: 54 - DNA, 333 bp, synthetic construct
  source 1..333: mol_type = other DNA; note = ORF
atggccctgtggatgcggctcttgccccttctggctttactggcactatggggccctgacccagcggccgcttttgtgaaccagcacctctgcggatctgatctggttgaggccttgtacctggtttgtggggaaagaggttttttctataccccaaggacaaagcgcgaggcagaagaccttcaagtgggccaggtagagctgggagggggcccgggtgcaggatccctgcagcctctggctctagaagggtctagacaaaaaaggggcatagtggagcagtgctgtacttcaatttgcagtttgtaccagcttgagaattattgtaactga

SEQ ID NO: 55 - DNA, 333 bp, synthetic construct
  source 1..333: mol_type = other DNA; note = ORF
atggccctgtggatgcggctcttgccccttctggctttactggcactatggggccctgacccagcggccgcttttgtgaaccagcacctctgcggatctgatctggtggaggccttgtacctggtttgtggggaaagaggttttttctatacccccaggacaaagcgcgaggcagaagaccttcaagtgggccaggtagagctgggagggggcccgggtgcaggatccctgcagcctctggctctggaagggtctagacaaaaaaggggcatagtggagcagtgctgtacttcaatttgcagtttgtaccagcttgagaattattgtaactga

SEQ ID NO: 56 - DNA, 333 bp, synthetic construct
  source 1..333: mol_type = other DNA; note = ORF
atggccctgtggatgagactcttgccccttctggctttactggcactatggggccctgacccagcagctgcttttgtgaaccagcacctctgtggatctgatctggttgaggccttgtacctggtttgtggggaaagaggttttttctataccccaaggacaaagagggaggcagaagaccttcaagtgggccaggtagagctgggagggggccctggtgcaggatccctgcagcctctggctctagaagggtctagacaaaaaaggggcatagtggagcagtgctgtacttcaatttgcagtttgtaccagcttgagaattattgtaactga

SEQ ID NO: 57 - DNA, 333 bp, synthetic construct
  source 1..333: mol_type = other DNA; note = ORF
atggccctgtggatgagactcttgccccttctggctttactggcactatggggccctgacccagcagctgcttttgtgaaccagcacctctgtggatctgatctggtggaggccttgtacctggtttgtggggaaagaggttttttctatacccccaggacaaagagagaggcagaagaccttcaagtgggccaggtagagctgggagggggcccaggtgcaggatccctgcagcctctggctctggaagggtctagacaaaaaagaggcatagtggagcagtgctgtacttcaatttgcagtttgtaccagcttgagaattattgtaactga

SEQ ID NO: 58 - skipped

SEQ ID NO: 59 - DNA, 295 bp, synthetic construct
  source 1..295: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 60 - DNA, 295 bp, synthetic construct; identical to SEQ ID NO: 59
  source 1..295: mol_type = other DNA; note = 3' UTR

SEQ ID NO: 61 - DNA, 1401 bp, synthetic construct
  source 1..1401: mol_type = other DNA; note = ORF
atggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccaccatgatctcctgctactatgaagaccatcagtgtgaggtgggcatgattgtgggcacaggctgcaatgcctgctacatggaggagatgcagaatgtggagctggtggagggggatgagggcaggatgtgtgtcaatacagagtggggagcctttggggactctggggagctggatgagttcctgctggagtatgacaggctggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaaggggagcctttgagaccaggtttgtgtcccaggtggagagtgacacaggagacaggaagcagatctacaacatcctgagcaccctggggctgagaccctccaccacagactgtgacattgtgaggagggcttgtgagagtgtgtctaccagggctgcccacatgtgctctgctgggctggctggggtcatcaacaggatgagggagagcaggagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagcagccctggtctctgctgtggcctgtaagaaggcctgtatgctgggccagtga

SEQ ID NO: 62 - DNA, 1401 bp, synthetic construct
  source 1..1401: mol_type = other DNA; note = ORF
atggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggtctgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagggatgctatcaaaaggagaggggactttgaaatggatgtggtggcaatggtgaatgacacagtggccacaatgatctcctgctactatgaagaccatcagtgtgaggtgggcatgattgtgggcacaggctgcaatgcctgctacatggaggagatgcagaatgtggagctggtggagggggatgagggcagaatgtgtgtcaatacagagtggggagcctttggggactctggggagctggatgagttcctgctggagtatgacagactggtggatgagagctcagcaaaccctggtcagcagctgtatgagaagctcataggtggcaagtacatgggagagctggtgagacttgtgctgctcaggctggtggatgaaaacctgctcttccatggggaggcctctgagcagctgagaacaagaggagcctttgagaccagatttgtgtcccaggtggagtctgacacaggagacaggaagcagatctacaacatcctgagcaccctggggctgaggccctccaccacagactgtgacattgtgagaagagcctgtgagtctgtgtctaccagggctgcccacatgtgctctgctgggctggctggggtcatcaacagaatgagggagagcaggtctgaggatgtaatgagaatcactgtgggagtggatggctctgtgtacaagctgcacccctcattcaaggagagattccatgcctctgtgagaaggctgacccccagctgtgagatcacctttattgagtctgaggagggcagtggcagaggagctgccctggtctctgctgtggcctgtaagaaggcctgtatgctgggccagtga

SEQ ID NO: 63 - DNA, 1401 bp, synthetic construct
  source 1..1401: mol_type = other DNA; note = ORF
atggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgactggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagggatgctatcaaaaggagaggggactttgaaatggatgtggtggcaatggtgaatgacactgtggccactatgatctcctgctactatgaagaccatcagtgtgaggtaggcatgattgtgggcactggctgcaatgcctgctacatggaggagatgcagaatgtggagctggtggagggggatgagggcagaatgtgtgtcaatactgagtggggtgcctttggggactcaggggagctggatgagttcctgctggagtatgacagactggtggatgagagctctgcaaaccctggtcagcagctgtatgagaagctcataggtggcaagtacatgggagagctggtgagacttgtgctgctcaggctggtggatgaaaacctgctcttccatggggaggcctctgagcagctgagaacaagaggagcctttgagaccagatttgtgtcccaggtggagtctgacactggtgacaggaagcagatctacaacatcctgagcacactggggctgaggccctccaccacagactgtgacattgtgagaagagcttgtgagagtgtgtctaccagggctgctcacatgtgcagtgctgggctggctggggtcatcaacagaatgagggagagcaggtctgaggatgtaatgagaatcactgtgggtgtggatggcagtgtgtacaagctgcacccctcattcaaggagagattccatgccagtgtgagaaggctgacccccagctgtgagatcaccttcattgagtctgaggagggcagtggcaggggtgcagccctggtcagtgcagtggcctgtaagaaggcctgtatgctgggccagtga

SEQ ID NO: 64 - DNA, 1401 bp, synthetic construct
  source 1..1401: mol_type = other DNA; note = ORF
atggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctcagacttcctggacaagcatcagatgaaacacaagaagctgcccctgggcttcaccttctcctttcctgtgaggcatgaagacatagataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccactatgatctcctgctactatgaagaccatcagtgtgaggtgggcatgatagtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctctgagcagctgagaacaaggggagcctttgagaccaggtttgtgtcccaggtggagtctgacactggggacaggaagcagatctacaacatcctgagcaccctggggctgaggccctccaccactgactgtgacatagtgaggagagcctgtgagtctgtgtctaccagggctgcccacatgtgctctgctgggctggcaggggtcatcaacaggatgagggagagcagatctgaggatgtaatgaggatcactgtgggtgtggatggctctgtgtacaagctgcacccctcattcaaggagaggttccatgcctctgtgaggaggctgacccccagctgtgagatcacctttatagagtctgaggagggcagtggcagaggggcagccctggtctctgcagtggcctgtaagaaggcctgtatgctgggccagtga

SEQ ID NO: 65 - DNA, 1401 bp, synthetic construct
  source 1..1401: mol_type = other DNA; note = ORF
atggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccaccatgatctcctgctactatgaagaccatcagtgtgaggtgggcatgatagtgggcacaggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctcagagcagctgagaacaaggggagcctttgagaccaggtttgtgtcccaggtggagagtgacacaggtgacaggaagcagatctacaacatcctgagcaccctggggctgagaccctccaccacagactgtgacattgtgaggagggcctgtgagagtgtgtctaccagggctgcccacatgtgctctgctgggctggcaggagtcatcaacaggatgagggagagcaggagtgaggatgtaatgaggatcactgtgggagtggatggctctgtgtacaagctgcacccctcattcaaggagaggttccatgccagtgtgagaaggctgacccccagctgtgagatcacctttatagagtctgaggagggcagtggcaggggagcagccctggtctctgctgtggcctgtaagaaggcctgtatgctgggccagtga

SEQ ID NO: 66 - DNA, 1401 bp, synthetic construct
  source 1..1401: mol_type = other DNA; note = ORF
atggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccacaatgatctcctgctactatgaagaccatcagtgtgaggtgggcatgatagtgggcacaggctgcaatgcctgctacatggaggagatgcagaatgtggagctggtggagggggatgagggcagaatgtgtgtcaatacagagtggggagcctttggggactctggggagctggatgagttcctgctggagtatgacagactggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaagaggagcctttgagaccagatttgtgtcacaggtggagtctgacactggagacagaaagcagatctacaacatcctgagcacactggggctgaggccctccaccactgactgtgacatagtgaggagagcttgtgagagtgtgtctaccagagctgcccacatgtgctctgctgggctggctggggtcatcaacaggatgagagagagcaggagtgaggatgtaatgagaatcactgtgggggtggatggctctgtgtacaagctgcacccctcattcaaggagaggttccatgcctctgtgaggaggctgacccccagctgtgagatcacctttattgagtctgaggagggcagtggaaggggagcagccctggtctctgcagtggcctgtaagaaggcctgtatgctgggccagtga

SEQ ID NO: 67 - DNA, 1401 bp, synthetic construct
  source 1..1401: mol_type = other DNA; note = ORF
atggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccactatgatctcctgctactatgaagaccatcagtgtgaggtgggcatgattgtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggagagctggtgagacttgtgctgctcaggctggtggatgaaaacctgctcttccatggggaggcctcagagcagctgaggacaaggggagcctttgagaccaggtttgtgtcccaggtggagagtgacactggagacaggaagcagatctacaacatcctgagcaccctggggctgaggccctctaccacagactgtgacatagtgagaagggcctgtgagtctgtgtctaccagggctgcccacatgtgctctgctgggctggctggagtcatcaacagaatgagggagagcagaagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagctgccctggtctctgctgtggcctgtaagaaggcctgtatgctgggccagtga

SEQ ID NO: 68 - DNA, 1401 bp, synthetic construct
  source 1..1401: mol_type = other DNA; note = ORF
atggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggccgcatgtgtgtcaatactgagtggggcgcctttggggactctggagagctggatgagttcctgctggagtatgaccgcctggtggatgagagctcagcaaacccaggtcagcagctgtatgagaagctcataggtggcaagtacatgggagagctggtgcggcttgtgctgctcaggttggtggatgaaaacctgctcttccatggggaggccagtgagcagctgcgcacacgcggagcctttgagacgcgctttgtgtcacaagtggagtctgacacaggagaccgcaagcagatctacaacatcctgagcacactggggctgcggccctccaccactgactgtgacattgtgcgccgcgcctgtgagagtgtgtctacgcgcgctgcgcacatgtgctcagcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtga

SEQ ID NO: 69 - DNA, 1401 bp, synthetic construct
  source 1..1401: mol_type = other DNA; note = ORF
atggccatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgcgggatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggccgcatgtgtgtcaatacagagtggggcgcctttggggacagcggagagctggatgagttcctgctggagtatgaccgcctggtggatgagagctcagcaaacccaggtcagcagctgtatgagaagctcataggtggcaagtacatgggagagctggtgcggcttgtgctgctcaggctggtggatgaaaacctgctcttccatggggaggcctctgagcagctgcgcacacgcggagcctttgagacgcgctttgtgagccaggtggagagtgacacaggagaccgcaagcagatctacaacatcctgagcaccctggggctgcggcccagcaccactgactgtgacattgtgcgccgcgcctgtgagtcagtgtctacgcgcgctgcgcacatgtgcagcgcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtga

SEQ ID NO: 70 - DNA, 1401 bp, synthetic construct
  source 1..1401: mol_type = other DNA; note = ORF
atggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatcccggaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggtaggcatgatagtgggcacaggctgcaatgcctgctacatggaggagatgcagaatgtggagctggtggagggggatgagggccgcatgtgtgtcaatactgagtggggcgcctttggggacagtggagagctggatgagttcctgctggagtatgaccgcctggtggatgagagctctgcaaacccaggtcagcagctgtatgagaagctcataggtggcaagtacatgggagagctggtgcggcttgtgctgctcaggctagtggatgaaaacctgctcttccatggggaggcctcagagcagctgcgcacacgcggagcctttgagacgcgctttgtgtctcaagtggagagtgacacaggtgaccgcaagcagatctacaacatcctgagcactctggggctgcggccctcaaccacagactgtgacattgtgcgccgcgcctgtgagtctgtgtctacgcgcgctgcgcacatgtgcagtgcggggctggcgggagtcatcaaccgcatgcgcgagagccgctcagaggatgtaatgcgcatcactgtgggtgtggatggctctgtgtacaagctgcaccccagcttcaaggagcggttccatgccagtgtgcgcaggctgactcccagctgtgagatcacctttatagagtcagaggagggcagtggcagaggcgcggccctggtctccgcggtggcctgtaagaaggcctgtatgctgggccagtga

SEQ ID NO: 71 - DNA, 1401 bp, synthetic construct
  source 1..1401: mol_type = other DNA; note = ORF
atggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacactgtggccactatgatctcctgctactatgaagaccatcagtgtgaggttggcatgattgtgggcactggctgcaatgcctgctacatggaggagatgcagaatgtggagctggtggagggggatgagggccgcatgtgtgtcaatactgagtggggcgcctttggggactctggtgagctggatgagttcctgctggagtatgaccgcctggtggatgagagctctgcaaaccctggtcagcagctgtatgagaagctcataggtggcaagtacatgggtgagctggtgcggcttgtgctgctcaggttagtggatgaaaacctgctcttccatggggaggcctctgagcagctgcgcacacgcggagcctttgagacgcgctttgtgtctcaagtggagtctgacactggtgaccgcaagcagatctacaacatcctgagcactctggggctgagaccctctaccactgactgtgacattgtgcgccgcgcctgtgagtctgtgtctacgcgcgctgcgcacatgtgctctgcggggctggcgggtgtcatcaaccgcatgcgcgagagccgctctgaggatgtaatgcgcatcactgtgggtgtggatggctctgtgtacaagctgcacccctcattcaaggagcggttccatgcctctgtgcgcaggctgactcccagctgtgagatcacctttattgagtctgaggagggcagtggcagaggcgcggccctggtctctgcggtggcctgtaagaaggcctgtatgctgggccagtga

SEQ ID NO: 72 - DNA, 1401 bp, synthetic construct
  source 1..1401: mol_type = other DNA; note = ORF
atggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggcaggatgtgtgtcaatactgagtggggagcctttggggactctggagagctggatgagttcctgctggagtatgacagactggtggatgagagctcagcaaacccaggtcagcagctgtatgagaagctcataggtggcaagtacatgggagagctggtgaggcttgtgctgctcaggttggtggatgaaaacctgctcttccatggggaggccagtgagcagctgagaacaaggggagcctttgagacaagatttgtgtcacaggtggagtctgacacaggagacaggaagcagatctacaacatcctgagcaccctggggctgagaccctccaccactgactgtgacattgtgaggagagcctgtgagagtgtgtctaccagggctgctcacatgtgctcagcagggctggctggagtcatcaacagaatgagggagagcagatctgaggatgtaatgaggatcactgtgggtgtggatggcagtgtgtacaagctgcaccccagcttcaaggagagattccatgcctcagtgaggaggctgacccccagctgtgagatcaccttcattgagtctgaggagggcagtggcagaggagcagccctggtctctgctgtggcctgtaagaaggcctgtatgctgggccagtga

SEQ ID NO: 73 - DNA, 1401 bp, synthetic construct
  source 1..1401: mol_type = other DNA; note = ORF
atggccatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgagagatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggcagaatgtgtgtcaatacagagtggggagcctttggggactctggagagctggatgagttcctgctggagtatgacagactggtggatgagagctcagcaaacccaggtcagcagctgtatgagaagctcataggtggcaagtacatgggagagctggtgagacttgtgctgctcaggctggtggatgaaaacctgctcttccatggggaggccagtgagcagctgagaacaagaggagcctttgagaccagatttgtgagccaggtggagtcagacacaggagacagaaagcagatctacaacatcctgagcaccctggggctgagacccagcaccactgactgtgacattgtgagaagagcctgtgagtctgtgtctaccagagctgcccacatgtgcagtgctgggctggctggagtcatcaacagaatgagagagagcagatcagaggatgtaatgagaatcactgtgggtgtggatggctctgtgtacaagctgcaccccagcttcaaggagagattccatgccagtgtgagaaggctgacccccagctgtgagatcacctttattgagtcagaggagggcagtggcagaggagcagccctggtctctgctgtggcctgtaagaaggcctgtatgctgggccagtga

SEQ ID NO: 74 - DNA, 1401 bp, synthetic construct
  source 1..1401: mol_type = other DNA; note = ORF
atggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatcccagaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggtaggcatgatagtgggcacaggctgcaatgcctgctacatggaggagatgcagaatgtggagctggtggagggggatgagggcaggatgtgtgtcaatactgagtggggagcctttggggacagtggagagctggatgagttcctgctggagtatgacagactggtggatgagagctctgcaaaccctggtcagcagctgtatgagaagctcataggtggcaagtacatgggtgagctggtgaggcttgtgctgctcaggctagtggatgaaaacctgctcttccatggggaggcctcagagcagctgagaacaaggggagcctttgagacaagatttgtgtctcaggtggagagtgacactggagacaggaagcagatctacaacatcctgagcacactggggctgagaccctcaaccactgactgtgacattgtgaggagagcctgtgagtctgtgtctacaagggctgcacacatgtgcagtgctgggctggcaggtgtcatcaacagaatgagggagagcagatcagaggatgtaatgaggatcactgtgggagtggatggctctgtgtacaagctgcaccccagcttcaaggagagattccatgccagtgtgaggaggctgacccccagctgtgagatcacctttatagagtcagaggagggcagtggcagaggtgctgccctggtctctgcagtggcctgtaagaaggcctgtatgctgggccagtga

SEQ ID NO: 75 - DNA, 1401 bp, synthetic construct
  source 1..1401: mol_type = other DNA; note = ORF
atggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacactgtggccactatgatctcctgctactatgaagaccatcagtgtgaggttggcatgattgtgggcactggctgcaatgcctgctacatggaggagatgcagaatgtggagctggtggagggggatgagggcagaatgtgtgtcaatactgagtggggtgcctttggggactctggtgagctggatgagttcctgctggagtatgacagactggtggatgagagctctgcaaaccctggtcagcagctgtatgagaagctcataggtggcaagtacatgggtgagctggtgagacttgtgctgctcaggttagtggatgaaaacctgctcttccatggggaggcctctgagcagctgagaacaagaggagcctttgagactagatttgtgtctcaggtggagtctgacactggtgacagaaagcagatctacaacatcctgagcactctggggctgagaccctctaccactgactgtgacattgtgagaagagcctgtgagtctgtgtctactagagctgctcacatgtgctctgctgggctggctggtgtcatcaacagaatgagagagagcagatctgaggatgtaatgagaatcactgtgggtgtggatggctctgtgtacaagctgcacccctcattcaaggagagattccatgcctctgtgagaaggctgactcccagctgtgagatcaccttcattgagtctgaggagggcagtggcagaggtgctgccctggtctctgctgtggcctgtaagaaggcctgtatgctgggccagtga

SEQ ID NO: 76 - DNA, 1401 bp, synthetic construct
  source 1..1401: mol_type = other DNA; note = ORF
atggccatggacgtgacccggagccaggctcaaacagcactgactctcgtcgagcagatcttggcggaattccagcttcaagaggaagatctgaagaaagttatgagaaggatgcagaaggagatggaccgcggcttacgactggaaacgcacgaggaggcctccgtgaaaatgctacccacctacgtgcgttctacacctgaaggatcagaggtgggggattttctcagtctggacttgggtggcactaacttccgggtcatgctggtgaaggttggagaaggggaggaaggccagtggtcggtgaaaaccaagcatcagatgtatagcattccagaggatgctatgacaggcaccgccgagatgctttttgactacatatccgaatgcatctctgatttcctcgacaagcaccaaatgaaacataagaaactgccgctgggatttactttctcatttcccgtgagacacgaggatattgacaaggggatcctcttgaattggacgaaaggcttcaaggcaagtggcgccgaaggaaacaatgtagtggggcttctgagggatgctattaagcgccggggtgactttgagatggacgttgtggcaatggtcaacgatacagtggccacaatgatcagctgttattacgaggaccatcagtgcgaagtcggcatgatagtgggaactgggtgtaatgcgtgctatatggaggaaatgcagaacgttgagttagtggaaggcgatgagggtagaatgtgtgtaaataccgagtggggagctttcggggactccggcgaactggatgagttcctactcgaatacgacaggctggtggatgagtctagcgccaaccctggccagcaattgtatgaaaaactgatcggagggaagtacatgggtgagcttgtccgcctcgttctgctgcgattagtggacgagaatctcctgtttcacggcgaagcatccgagcagttgcgtacacggggagccttcgaaaccagatttgtctcacaagtggagagtgatactggggacaggaaacagatttacaacatccttagcacactgggcctacgcccatctacgaccgattgcgacattgtgcggcgagcttgtgagtccgtttcgactagagccgcacacatgtgctcagctggtctcgccggagtaatcaataggatgcgcgaaagccggagtgaggacgtgatgagaataacagtcggcgtggatgggtctgtctataagctgcatccctccttcaaggaaaggtttcacgcgagcgtgcgccgtctgaccccttcatgtgagattactttcatcgaatccgaggagggcagtggacggggggcagccttggtttctgctgtggcctgcaaaaaggcatgcatgcttggccaatga

SEQ ID NO: 77 - DNA, 1401 bp, synthetic construct
  source 1..1401: mol_type = other DNA; note = ORF
atggccatggatgtgaccagaagccaggctcaaacagcactgactctggtagagcagatcttggcggaattccagcttcaagaggaagatctgaagaaagttatgagaaggatgcagaaggagatggaccgcggcttaaggctggaaacccatgaggaggccagtgtgaaaatgctacccacctatgtaagatctacacctgaaggatcagaggtgggggattttctcagtctggacttgggtggcactaacttcagggtcatgctggtgaaggttggagaaggggaggaaggccagtggtcagtgaaaaccaagcatcagatgtatagcattccagaggatgctatgacaggtaccgctgagatgctttttgactacatatctgaatgcatctctgatttcctagacaagcaccaaatgaaacataagaaactgccgctgggatttactttctcatttcctgtgagacatgaggatattgacaaggggatcctcttgaattggacaaaaggcttcaaggcaagtggcgcagaaggaaacaatgtagtggggcttctgagggatgctattaagcgcagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggtgatgagggtagaatgtgtgtaaatacagagtggggagcttttggggacagcggagaactggatgagttcctattagaatatgacaggctggtggatgagtctagcgccaaccctggccagcaattgtatgaaaaactgattggagggaagtacatgggtgagcttgtccgccttgttctgctgcggttagtggatgagaatctcctgtttcatggggaagcaagtgagcagttgcggacaaggggagcctttgaaaccagatttgtctcacaagtggagagtgatactggggacaggaaacagatttacaacatccttagcacactgggcctaagaccatctaccactgattgtgacattgtgcggcgggcttgtgagtcagtttccactagagccgcacacatgtgctcagctggtttggctggagtaatcaataggatgcgcgaaagcaggagtgaggatgtgatgagaataacagttggtgtggatgggtctgtctataagctgcatccctccttcaaggaaaggtttcacgcgtctgtgcgcagactgaccccttcatgtgagattactttcatagaatctgaggagggcagtggaaggggggcagccttggtttctgctgtggcctgcaaaaaggcatgcatgcttggccaatga

SEQ ID NO: 78 - DNA, 1401 bp, synthetic construct
  source 1..1401: mol_type = other DNA; note = ORF
atggccatggatgtgaccagaagccaggctcaaacagcactgactctggtggagcagatcttggcggaattccagcttcaagaggaagatctgaagaaagttatgagaaggatgcagaaggagatggaccgcggcttaaggctggaaacccatgaggaggccagtgtgaaaatgctacccacctatgtaagatctacacctgaaggatcagaggtgggggattttctcagtctggacttgggtggcactaacttcagggtcatgctggtgaaggttggagaaggggaggaaggccagtggtcagtgaaaaccaagcatcagatgtatagcattccagaggatgctatgacaggtaccgctgagatgctttttgactacatatctgaatgcatctctgatttcctggacaagcaccaaatgaaacataagaaactgccgctgggatttactttctcatttcctgtgagacatgaggatattgacaaggggatcctcttgaattggaccaaaggcttcaaggcaagtggcgcagaaggaaacaatgtagtggggcttctgagggatgctattaagcgcagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggtgatgagggtagaatgtgtgtaaatacagagtggggagcttttggggacagcggagaactggatgagttcctactggaatatgacaggctggtggatgagtctagcgccaaccctggccagcaattgtatgaaaaactgattggagggaagtacatgggtgagcttgtccgcctggttctgctgcggttagtggatgagaatctcctgtttcatggggaagcaagtgagcagttgcggacaaggggagcctttgaaaccagatttgtctcacaagtggagagtgatactggggacaggaaacagatttacaacatccttagcacactgggcctaagaccatctaccacagattgtgacattgtgcggcgggcttgtgagtcagttagcactagagccgcacacatgtgctcagctggtctggctggagtaatcaataggatgcgcgaaagcaggagtgaggatgtgatgagaataacagtgggtgtggatgggtctgtctataagctgcatccctccttcaaggaaaggtttcacgcgtctgtgcgcagactgaccccttcatgtgagattactttcatagaatctgaggagggcagtggaaggggggcagccttggtttctgctgtggcctgcaaaaaggcatgcatgcttggccaatga

SEQ ID NO: 79 - DNA, 1401 bp, synthetic construct
  source 1..1401: mol_type = other DNA; note = ORF
atggccatggatgtgaccagaagccaggctcaaacagcactgactctggtagagcagatcttggctgaattccagcttcaagaggaagatctgaagaaagttatgagaaggatgcagaaggagatggacaggggcttaagactggaaacccatgaggaggccagtgtgaaaatgctacccacctatgtaaggtctacacctgaaggatcagaggtgggggattttctcagtctggacttgggtggcactaacttcagagtcatgctggtgaaggttggagaaggggaggaaggccagtggtcagtgaaaaccaagcatcagatgtatagcattccagaggatgctatgacaggcacagcagagatgctttttgactacatatctgaatgcatctctgatttcctagacaagcaccaaatgaaacataagaaactgccactgggatttactttctcatttcctgtgagacatgaggatattgacaaggggatcctcttgaattggaccaaaggcttcaaggcaagtggtgctgaaggaaacaatgtagtggggcttctgagggatgctattaagaggagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggagatgagggtagaatgtgtgtaaatacagagtggggagcttttggggacagtggggaactggatgagttcctattagaatatgacaggctggtggatgagtcttcagccaaccctggccagcaattgtatgaaaaactgattggagggaagtacatgggtgagcttgtcaggcttgttctgctgagattagtggatgagaatctcctgtttcatggtgaagcatctgagcagttgaggacaagaggagcctttgaaaccagatttgtctcacaagtggagagtgatactggggacaggaaacagatttacaacatccttagcacactgggcctaaggccatctaccactgattgtgacattgtgagaagggcttgtgagagtgtttccactagagcagcacacatgtgctcagctggtttggctggagtaatcaataggatgagagaaagcaggagtgaggatgtgatgagaataacagttggagtggatgggtctgtctataagctgcatccctccttcaaggaaaggtttcatgcctctgtgagaaggctgaccccttcatgtgagattactttcatagaatcagaggagggcagtggaagaggggcagccttggtttctgctgtggcctgcaaaaaggcatgcatgcttggccaatga

SEQ ID NO: 80 - DNA, 1401 bp, synthetic construct
  source 1..1401: mol_type = other DNA; note = ORF
atggccatggatgtgaccagaagccaggctcaaacagcactgactctggtggagcagatcttggctgaattccagcttcaagaggaagatctgaagaaagttatgagaaggatgcagaaggagatggacagaggcttaagactggaaacccatgaggaggccagtgtgaaaatgctacccacctatgtaagatctacacctgaaggatcagaggtgggggattttctcagtctggacttgggtggcactaacttcagagtcatgctggtgaaggttggagaaggggaggaaggccagtggtcagtgaaaaccaagcatcagatgtatagcattccagaggatgctatgacaggcacagcagagatgctttttgactacatatctgaatgcatctctgatttcctggacaagcaccaaatgaaacataagaaactgcccctgggatttactttctcatttcctgtgagacatgaggatattgacaaggggatcctcttgaattggaccaaaggcttcaaggcaagtggtgctgaaggaaacaatgtagtggggcttctgagggatgctattaagagaagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggagatgagggtagaatgtgtgtaaatacagagtggggagcttttggggacagtggggaactggatgagttcctactggaatatgacaggctggtggatgagtcttcagccaaccctggccagcaattgtatgaaaaactgattggagggaagtacatgggtgagcttgtcagactggttctgctgagattagtggatgagaatctcctgtttcatggtgaagcatctgagcagttgagaacaagaggagcctttgaaaccagatttgtctcacaagtggagagtgatactggggacaggaaacagatttacaacatccttagcacactgggcctaagaccatctaccactgattgtgacattgtgagaagagcttgtgagagtgttagcactagagcagcacacatgtgctcagctggtctggctggagtaatcaataggatgagagaaagcagaagtgaggatgtgatgagaataacagtgggagtggatgggtctgtctataagctgcatccctccttcaaggaaaggtttcatgcctctgtgagaagactgaccccttcatgtgagattactttcatagaatcagaggagggcagtggaagaggggcagccttggtttctgctgtggcctgcaaaaaggcatgcatgcttggccaatga

SEQ ID NO: 81 - skipped

SEQ ID NO: 82 - protein, 465 aa, synthetic construct [sequence omitted]

SEQ ID NO: 83 - DNA, 325 bp, synthetic construct
  source 1..325: mol_type = other DNA; note = 5' UTR
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtcctgcagaattgccacc

SEQ ID NO: 84 - DNA, 951 bp, synthetic construct
  source 1..951: mol_type = other DNA; note = Modified hIns 17
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtcctgcagaattgccaccatggccctgtggatgagactcctgcccctgctggcactgctggccctctggggacctgacccagcagcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtccagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagggatccaagctt

SEQ ID NO: 85 - DNA, 951 bp, synthetic construct
  source 1..951: mol_type = other DNA; note = Modified hIns 18
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggcactgctggccctctggggacctgacccagcagcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtccagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggaattcaagctt

SEQ ID NO: 86 - DNA, 951 bp, synthetic construct
  source 1..951: mol_type = other DNA; note = Modified hIns 19
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactgctccccctcctggctttgctggccctgtggggaccagatcctgcagctgcctttgtgaatcagcacctgtgtggctcagatctggttgaggcactctacctggtgtgtggggagagaggcttcttttacacaccaaggaccaagagggaggcagaggatctgcaggtaggtcaggtggagctgggaggtggccctggagcaggatccctgcagcccttggcactggagggctccagacagaagaggggcattgtggaacagtgttgcactagcatctgcagcctgtaccagctggagaactactgcaactaaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagcatatgaagctt

SEQ ID NO: 87 - DNA, 951 bp, synthetic construct
  source 1..951: mol_type = other DNA; note = Modified hIns 20
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggatatcaagctt

SEQ ID NO: 88 - DNA, 951 bp, synthetic construct
  source 1..951: mol_type = other DNA; note = Modified hIns 21
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagactagtaagctt

SEQ ID NO: 89 - DNA, 2019 bp, synthetic construct
  source 1..2019: mol_type = other DNA; note = Modified hGck-22
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccaccatgatctcctgctactatgaagaccatcagtgtgaggtgggcatgattgtgggcacaggctgcaatgcctgctacatggaggagatgcagaatgtggagctggtggagggggatgagggcaggatgtgtgtcaatacagagtggggagcctttggggactctggggagctggatgagttcctgctggagtatgacaggctggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaaggggagcctttgagaccaggtttgtgtcccaggtggagagtgacacaggagacaggaagcagatctacaacatcctgagcaccctggggctgagaccctccaccacagactgtgacattgtgaggagggcttgtgagagtgtgtctaccagggctgcccacatgtgctctgctgggctggctggggtcatcaacaggatgagggagagcaggagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagtctagaaagctt

SEQ ID NO: 90 - DNA, 2019 bp, synthetic construct
  source 1..2019: mol_type = other DNA; note = Modified hGck-23
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccaccatgatctcctgctactatgaagaccatcagtgtgaggtgggcatgattgtgggcacaggctgcaatgcctgctacatggaggagatgcagaatgtggagctggtggagggggatgagggcaggatgtgtgtcaatacagagtggggagcctttggggactctggggagctggatgagttcctgctggagtatgacaggctggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaaggggagcctttgagaccaggtttgtgtcccaggtggagagtgacacaggagacaggaagcagatctacaacatcctgagcaccctggggctgagaccctccaccacagactgtgacattgtgaggagggcttgtgagagtgtgtctaccagggctgcccacatgtgctctgctgggctggctggggtcatcaacaggatgagggagagcaggagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggctagcaagctt

SEQ ID NO: 91 - DNA, 2019 bp, synthetic construct
  source 1..2019: mol_type = other DNA; note = Modified hGck-24
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccaccatgatctcctgctactatgaagaccatcagtgtgaggtgggcatgatagtgggcacaggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctcagagcagctgagaacaaggggagcctttgagaccaggtttgtgtcccaggtggagagtgacacaggtgacaggaagcagatctacaacatcctgagcaccctggggctgagaccctccaccacagactgtgacattgtgaggagggcctgtgagagtgtgtctaccagggctgcccacatgtgctctgctgggctggcaggagtcatcaacaggatgagggagagcaggagtgaggatgtaatgaggatcactgtgggagtggatggctctgtgtacaagctgcacccctcattcaaggagaggttccatgccagtgtgagaaggctgacccccagctgtgagatcacctttatagagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagattaataagctt

SEQ ID NO: 92 - DNA, 2019 bp, synthetic construct
  source 1..2019: mol_type = other DNA; note = Modified hGck-25
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccactatgatctcctgctactatgaagaccatcagtgtgaggtgggcatgattgtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggagagctggtgagacttgtgctgctcaggctggtggatgaaaacctgctcttccatggggaggcctcagagcagctgaggacaaggggagcctttgagaccaggtttgtgtcccaggtggagagtgacactggagacaggaagcagatctacaacatcctgagcaccctggggctgaggccctctaccacagactgtgacatagtgagaagggcctgtgagtctgtgtctaccagggctgcccacatgtgctctgctgggctggctggagtcatcaacagaatgagggagagcagaagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagctgccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagatgcataagctt

SEQ ID NO: 93 - DNA, 2019 bp, synthetic construct
  source 1..2019: mol_type = other DNA; note = Modified hGck-26
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggccgcatgtgtgtcaatactgagtggggcgcctttggggactctggagagctggatgagttcctgctggagtatgaccgcctggtggatgagagctcagcaaacccaggtcagcagctgtatgagaagctcataggtggcaagtacatgggagagctggtgcggcttgtgctgctcaggttggtggatgaaaacctgctcttccatggggaggccagtgagcagctgcgcacacgcggagcctttgagacgcgctttgtgtcacaagtggagtctgacacaggagaccgcaagcagatctacaacatcctgagcacactggggctgcggccctccaccactgactgtgacattgtgcgccgcgcctgtgagagtgtgtctacgcgcgctgcgcacatgtgctcagcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagagtactaagctt

SEQ ID NO: 94 - DNA, 2019 bp, synthetic construct
  source 1..2019: mol_type = other DNA; note = Modified hGck-27
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgcgggatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggccgcatgtgtgtcaatacagagtggggcgcctttggggacagcggagagctggatgagttcctgctggagtatgaccgcctggtggatgagagctcagcaaacccaggtcagcagctgtatgagaagctcataggtggcaagtacatgggagagctggtgcggcttgtgctgctcaggctggtggatgaaaacctgctcttccatggggaggcctctgagcagctgcgcacacgcggagcctttgagacgcgctttgtgagccaggtggagagtgacacaggagaccgcaagcagatctacaacatcctgagcaccctggggctgcggcccagcaccactgactgtgacattgtgcgccgcgcctgtgagtcagtgtctacgcgcgctgcgcacatgtgcagcgcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagggtaccaagctt

SEQ ID NO: 95 - DNA, 2019 bp, synthetic construct
  source 1..2019: mol_type = other DNA; note = Modified hGck-28
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggcaggatgtgtgtcaatactgagtggggagcctttggggactctggagagctggatgagttcctgctggagtatgacagactggtggatgagagctcagcaaacccaggtcagcagctgtatgagaagctcataggtggcaagtacatgggagagctggtgaggcttgtgctgctcaggttggtggatgaaaacctgctcttccatggggaggccagtgagcagctgagaacaaggggagcctttgagacaagatttgtgtcacaggtggagtctgacacaggagacaggaagcagatctacaacatcctgagcaccctggggctgagaccctccaccactgactgtgacattgtgaggagagcctgtgagagtgtgtctaccagggctgctcacatgtgctcagcagggctggctggagtcatcaacagaatgagggagagcagatctgaggatgtaatgaggatcactgtgggtgtggatggcagtgtgtacaagctgcaccccagcttcaaggagagattccatgcctcagtgaggaggctgacccccagctgtgagatcaccttcattgagtctgaggagggcagtggcagaggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagaatattaagctt

SEQ ID NO: 96 - DNA, 2019 bp, synthetic construct
  source 1..2019: mol_type = other DNA; note = Modified hGck-29
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgagagatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggcagaatgtgtgtcaatacagagtggggagcctttggggactctggagagctggatgagttcctgctggagtatgacagactggtggatgagagctcagcaaacccaggtcagcagctgtatgagaagctcataggtggcaagtacatgggagagctggtgagacttgtgctgctcaggctggtggatgaaaacctgctcttccatggggaggccagtgagcagctgagaacaagaggagcctttgagaccagatttgtgagccaggtggagtcagacacaggagacagaaagcagatctacaacatcctgagcaccctggggctgagacccagcaccactgactgtgacattgtgagaagagcctgtgagtctgtgtctaccagagctgcccacatgtgcagtgctgggctggctggagtcatcaacagaatgagagagagcagatcagaggatgtaatgagaatcactgtgggtgtggatggctctgtgtacaagctgcaccccagcttcaaggagagattccatgccagtgtgagaaggctgacccccagctgtgagatcacctttattgagtcagaggagggcagtggcagaggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagttaattaagctt

SEQ ID NO: 97 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagggatccaagctt

SEQ ID NO: 98 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggaattcaagctt

SEQ ID NO: 99 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagcatatgaagctt

SEQ ID NO: 100 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggatatcaagctt

SEQ ID NO: 101 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagactagtaagctt

SEQ ID NO: 102 - DNA, 293 bp, synthetic construct
  source 1..293: mol_type = other DNA; note = 3'UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagtctagaaagctt

SEQ ID NO: 103 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggctagcaagctt

SEQ ID NO: 104 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagattaataagctt

SEQ ID NO: 105 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagatgcataagctt

SEQ ID NO: 106 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagagtactaagctt

SEQ ID NO: 107 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagggtaccaagctt

SEQ ID NO: 108 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagaatattaagctt

SEQ ID NO: 109 - DNA, 293 bp, synthetic construct
  source 1..293: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagttaattaagctt

SEQ ID NO: 110 - DNA, 333 bp, synthetic construct
  source 1..333: mol_type = other DNA; note = ORF
atggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 111 - DNA, 333 bp, synthetic construct
  source 1..333: mol_type = other DNA; note = ORF
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 112 - DNA, 333 bp, synthetic construct
  source 1..333: mol_type = other DNA; note = ORF
atggccctgtggatgcgcctcctgcccctgctggcgctgctggccctctggggacctgacccagccgcagcctttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactag

SEQ ID NO: 113 - DNA, 333 bp, synthetic construct
  source 1..333: mol_type = other DNA; note = ORF
atggccctgtggatgagactcctgcccctgctggctctgctggccctctggggacctgacccagctgcagcctttgtgaaccaacacctgtgtggctcacacctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactag

SEQ ID NO: 114 - DNA, 333 bp, synthetic construct
  source 1..333: mol_type = other DNA; note = hIns (B10H) ORF
atggccctgtggatgcggctcttgccccttctggctttactggcactatggggccctgacccagcggccgctttcgtgaaccagcacctctgcggaagccacctggtcgaggccttgtacctggtttgtggggaaagaggttttttctataccccgaggacaaagcgcgaggcagaagaccttcaagtgggccaggtagagctcggagggggccccggtgccggatccctgcagcctctggctctcgaagggtctcgacaaaaacgtggcatcgtggagcagtgctgtacttcaatttgcagtttgtaccagcttgagaattattgtaactga

SEQ ID NO: 115 - DNA, 333 bp, synthetic construct
  source 1..333: mol_type = other DNA; note = hIns (B10H) ORF
atggccctgtggatgcggctcttgccccttctggctttactggcactatggggccctgacccagcggccgcttttgtgaaccagcacctctgcggatctcacctggttgaggccttgtacctggtttgtggggaaagaggttttttctataccccaaggacaaagcgcgaggcagaagaccttcaagtgggccaggtagagctgggagggggcccgggtgcaggatccctgcagcctctggctctagaagggtctagacaaaaaaggggcatagtggagcagtgctgtacttcaatttgcagtttgtaccagcttgagaattattgtaactga

SEQ ID NO: 116 - DNA, 333 bp, synthetic construct
  source 1..333: mol_type = other DNA; note = hIns (B10H) ORF
atggccctgtggatgagactcttgccccttctggctttactggcactatggggccctgacccagcagctgcttttgtgaaccagcacctctgtggatctcacctggttgaggccttgtacctggtttgtggggaaagaggttttttctataccccaaggacaaagagggaggcagaagaccttcaagtgggccaggtagagctgggagggggccctggtgcaggatccctgcagcctctggctctagaagggtctagacaaaaaaggggcatagtggagcagtgctgtacttcaatttgcagtttgtaccagcttgagaattattgtaactga

SEQ ID NO: 117 - DNA, 348 bp, synthetic construct
  source 1..348: mol_type = other DNA; note = ORF
atgaacagcttcagcacaagcgccttcggccccgtggctttctctctgggactgctgctggtgctgcctgccgctttccctgccccttttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 118 - DNA, 348 bp, synthetic construct
  source 1..348: mol_type = other DNA; note = ORF
atgaacagcttcagcacaagcgcctttggcccagtggctttctctctgggactgctgctggtgctgcctgccgctttccctgccccttttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactag

SEQ ID NO: 119 - DNA, 348 bp, synthetic construct
  source 1..348: mol_type = other DNA; note = ORF
atgaacagcttcagcacaagtgcctttggcccagtggctttctctctgggactgctgctggtgctgcctgcagctttccctgccccttttgtgaaccaacacctgtgtggctcagatctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggtccaggcagaagagaggcattgtggaacaatgctgtaccagcatctgctccctctaccagctggagaactactgcaactag

SEQ ID NO: 120 - DNA, 348 bp, synthetic construct
  source 1..348: mol_type = other DNA; note = ORF
atgaacagcttcagcacaagcgccttcggccccgtggctttctctctgggactgctgctggtgctgcctgccgctttccctgccccttttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 121 - DNA, 348 bp, synthetic construct
  source 1..348: mol_type = other DNA; note = ORF
atgaacagcttcagcacaagcgcctttggcccagtggctttctctctgggactgctgctggtgctgcctgccgctttccctgccccttttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactag

SEQ ID NO: 122 - DNA, 348 bp, synthetic construct
  source 1..348: mol_type = other DNA; note = ORF
atgaacagcttcagcacaagtgcctttggcccagtggctttctctctgggactgctgctggtgctgcctgcagctttccctgccccttttgtgaaccaacacctgtgtggctcacacctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggtccaggcagaagagaggcattgtggaacaatgctgtaccagcatctgctccctctaccagctggagaactactgcaactag

SEQ ID NO: 123 - DNA, 953 bp, synthetic construct
  source 1..953: mol_type = other DNA; note = Modified hIns 22
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 124-126 - skipped (3 entries)

SEQ ID NO: 127 - DNA, 953 bp, synthetic construct
  source 1..953: mol_type = other DNA; note = Unmodified hIns 2 (B10H)
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 128 - DNA, 953 bp, synthetic construct
  source 1..953: mol_type = other DNA; note = Modified hIns 27
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 129 - DNA, 953 bp, synthetic construct
  source 1..953: mol_type = other DNA; note = Modified hIns 28
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcacacctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 130-132 - skipped (3 entries)

SEQ ID NO: 133 - DNA, 968 bp, synthetic construct
  source 1..968: mol_type = other DNA; note = Modified hIns 32
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgccttcggccccgtggctttctctctgggactgctgctggtgctgcctgccgctttccctgccccttttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 134 - DNA, 968 bp, synthetic construct
  source 1..968: mol_type = other DNA; note = Modified hIns 33
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgcctttggcccagtggctttctctctgggactgctgctggtgctgcctgccgctttccctgccccttttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 135 - DNA, 968 bp, synthetic construct
  source 1..968: mol_type = other DNA; note = Modified hIns 34
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagtgcctttggcccagtggctttctctctgggactgctgctggtgctgcctgcagctttccctgccccttttgtgaaccaacacctgtgtggctcagatctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggtccaggcagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 136 - DNA, 968 bp, synthetic construct
  source 1..968: mol_type = other DNA; note = Modified hIns 35
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgccttcggccccgtggctttctctctgggactgctgctggtgctgcctgccgctttccctgccccttttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 137 - DNA, 968 bp, synthetic construct
  source 1..968: mol_type = other DNA; note = Modified hIns 36
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgcctttggcccagtggctttctctctgggactgctgctggtgctgcctgccgctttccctgccccttttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 138 - DNA, 968 bp, synthetic construct
  source 1..968: mol_type = other DNA; note = Modified hIns 37
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagtgcctttggcccagtggctttctctctgggactgctgctggtgctgcctgcagctttccctgccccttttgtgaaccaacacctgtgtggctcacacctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggtccaggcagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 139-144 - skipped (6 entries)

SEQ ID NO: 145 - protein, 110 aa, synthetic construct
  source 1..110: note = mutant ins B10H
MALWMRLLPLLALLALWGPDPAAAFVNQHLCGSHLVEALYLVCGERGFFYTPRTKREAEDLQVGQVELGGGPGAGSLQPLALEGSRQKRGIVEQCCTSICSLYQLENYCN

SEQ ID NO: 146 - skipped

SEQ ID NO: 147 - DNA, 333 bp, synthetic construct
  source 1..333: mol_type = other DNA; note = wildtype sequence
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 148 - DNA, 350 bp, synthetic construct
  source 1..350: mol_type = other DNA; note = 5' UTR
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgcc

SEQ ID NO: 149 - DNA, 363 bp, synthetic construct
  source 1..363: mol_type = other DNA; note = 3' UTR
acgcagctgctagctaatcgataccgtcgacctcgaggaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 150-159 - skipped (10 entries)

SEQ ID NO: 160 - DNA, 683 bp, synthetic construct
  source 1..683: mol_type = other DNA; note = Unmodified hIns_2 (B10D)
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 161 - DNA, 957 bp, synthetic construct
  source 1..957: mol_type = other DNA; note = Unmodified hIns_3 (B10D)
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 162 - DNA, 1401 bp, synthetic construct
  source 1..1401: mol_type = other DNA; note = ORF
atggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtga

SEQ ID NO: 163 - DNA, 2016 bp, synthetic construct
  source 1..2016: mol_type = other DNA; note = Unmodified hGck_2
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcat

SEQ ID NO: 164 - DNA, 2025 bp, synthetic construct
  source 1..2025: mol_type = other DNA; note = Unmodified hGck_3
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 165 - protein, 24 aa, synthetic construct
MALWMRLLPLLALLALWGPDPAAA

SEQ ID NO: 166 - protein, 29 aa, synthetic construct
MNSFSTSAFGPVAFSLGLLLVLPAAFPAP

SEQ ID NO: 167 - protein, 26 aa, synthetic construct
MLRGPGPGLLLLAVQCLGTAVPSTGA

SEQ ID NO: 168 - DNA, 2022 bp, synthetic construct
  source 1..2022: mol_type = other DNA; note = hGck
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggccgcatgtgtgtcaatactgagtggggcgcctttggggactctggagagctggatgagttcctgctggagtatgaccgcctggtggatgagagctcagcaaacccaggtcagcagctgtatgagaagctcataggtggcaagtacatgggagagctggtgcggcttgtgctgctcaggttggtggatgaaaacctgctcttccatggggaggccagtgagcagctgcgcacacgcggagcctttgagacgcgctttgtgtcacaagtggagtctgacacaggagaccgcaagcagatctacaacatcctgagcacactggggctgcggccctccaccactgactgtgacattgtgcgccgcgcctgtgagagtgtgtctacgcgcgctgcgcacatgtgctcagcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaattgcattcattttatgtttcaggttcagggggagatgtgggaggttttttaaagcaagtaaaacctctacaaatgtggtaaaatccgataagggactagagcatggctacgta

SEQ ID NO: 169 - DNA, 292 bp, synthetic construct
  source 1..292: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaattgcattcattttatgtttcaggttcagggggagatgtgggaggttttttaaagcaagtaaaacctctacaaatgtggtaaaatccgataagggactagagcatggctacgta

SEQ ID NO: 170 - DNA, 968 bp, synthetic construct
  source 1..968: mol_type = other DNA; note = Modified hIns_41
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgcctttggcccagtggctttctctctgggactgctgctggtgctgcctgccgctttccctgccccttttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgta

SEQ ID NO: 171 - DNA, 295 bp, synthetic construct
  source 1..295: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgta